(19) United States
(12) Patent Application Publication
(10) Pub. No.: US 2023/0242552 A1
(43) Pub. Date: Aug. 3, 2023

(54) Nitrogen-Containing Heterocyclic Compound, Preparation Method Therefor and Use Thereof

(71) Applicant: 280 Bio, Inc, South San Francisco, CA (US)

(72) Inventors: Zusheng XU, Shanghai (CN); Yangtong LOU, Shanghai (CN); Qingrui SUN, Shanghai (CN); Tiegang XIE, Shanghai (CN); Jian SHEN, Shanghai (CN); Xin JIN, Shanghai (CN)

(73) Assignee: 280 Bio, Inc, South San Francisco, CA (US)

(21) Appl. No.: 18/160,560

(22) Filed: Jan. 27, 2023

(30) Foreign Application Priority Data
Jan. 30, 2022 (CN) CN202210113261.9
Aug. 24, 2022 (CN) CN202211020274.8

(51) Int. Cl. C07D 519/00; A61P 35/00; A61P 1/00; C07D 471/04; A61P 11/00; A61P 17/00

(52) U.S. Cl. CPC C07D 519/00; A61P 35/00; A61P 1/00; C07D 471/04; A61P 11/00; A61P 17/00

(57) ABSTRACT

Disclosed is a nitrogen-containing heterocyclic compound, a preparation method therefor and use thereof. The present disclosure provides a nitrogen-containing heterocyclic compound of formula I, a pharmaceutically acceptable salt thereof, a stereoisomer thereof, a tautomer thereof or an isotopically labeled compound thereof, and the nitrogen-containing heterocyclic compound is expected to treat and/or prevent various RAS-related diseases.

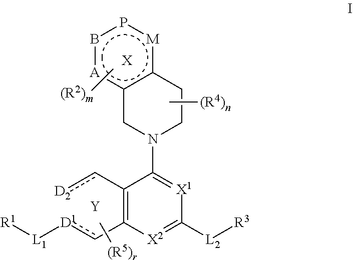

### TECHNICAL FIELD

The present disclosure relates to a nitrogen-containing heterocyclic compound, a preparation method therefor and use thereof.

### BACKGROUND

Ras (Rat sarcoma viral oncogene) was first found in rat sarcoma. There are three members in the ras gene family of mammals, namely H-ras (HRAS), K-ras (KRAS) and N-ras (NRAS), where the fourth exon of K-ras has variants A and B. Ras gene is widely found in various eukaryotes such as mammals, fruit flies, fungi, nematodes and yeasts, and is expressed at various levels in different tissues, where H-Ras is mainly expressed in skin and skeletal muscle, K-Ras is mainly expressed in colon and thymus, and N-Ras is expressed at a high level in testis. Ras protein regulates and controls signal transduction by switching the binding to GTP/GDP as a molecular switch in cell signal transduction, thereby regulating the life processes such as proliferation, differentiation, senescence and apoptosis of cells.

The mutant RAS are closely associated with the occurrence and development of human tumors, and are present in about 30% of human tumors. KRAS mutation is most common and accounts for approximately 85% of cases, and NRAS and HRAS account for 12% and 3%, respectively. KRAS mutation is mainly found in pancreatic, colorectal and lung cancers, NRAS mutation is common in melanoma and acute myelogenous leukemia, and HRAS mutation is common in bladder and head and neck cancers. Ras proto-oncogene mutation occurs mainly by point mutation. More than 150 different Ras point mutations have been found, with mutations in the glycine at positions 12 and 13 and glutamine at position 61 being the most common.

For decades, efforts have been made to develop small molecule inhibitors targeting Ras. Scientists have been hoping to develop competitive inhibitors of GTP that act directly on the Ras protein. However, this has not been successful because of the strong affinity between GTP and Ras (pmol/L level), the high concentration of GTP in cells (0.5 mM), the lack of a pocket in the RAS protein structure that facilitates the binding of small molecules, and the like. In recent years, some advances have been made in drug development using the allosteric site of K-Ras G12C mutant. In 2013, a team of researchers reported the discovery about K-Ras G12C small molecule inhibitors (Nature, 2013, 503, 548-551). They identified a novel binding pocket located below the molecular switch II region from K-Ras G12C mutant. Those inhibitors bind to the allosteric pocket and covalently bind to nearby Cys12, thereby selectively inhibiting the activation of K-Ras G12C. Other researchers have reported KRAS inhibitors with cellular activity (Science, 2016, 351, 604-608). Compound AMG510 from Amgen, studied in a clinical trial began in 2018, is the first small molecule inhibitor directly targeting KRAS to enter clinical studies, and has received accelerated marketing approval from the U.S. FDA in May 2021.

In conclusion, after decades of unremitting efforts, the understanding of Ras has been gradually improved, but only drugs for KRAS G12C mutation have been marketed so far, and there is no particularly effective treatment for other different mutations. The search for compounds with better inhibitory effects on Ras remains a hot and difficult research area in new drug development.

### Content of the Present Invention

The technical problem to be solved by the present disclosure is the lack of effective drugs serving as Ras inhibitors for clinical treatment in the prior art. Therefore, the present disclosure provides a nitrogen-containing heterocyclic compound, a preparation method therefor and use thereof, and the nitrogen-containing heterocyclic compound is expected to treat and/or prevent various RAS-related diseases.

The present disclosure solves the above problem by the following technical schemes.

The present disclosure provides a nitrogen-containing heterocyclic compound of formula I, a pharmaceutically acceptable salt thereof, a stereoisomer thereof, a tautomer thereof or an isotopically labeled compound thereof:

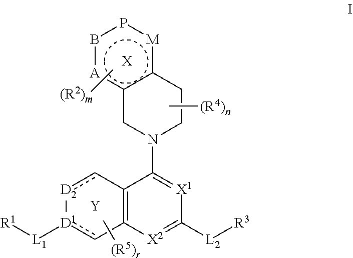

wherein [symbol] represents a single or double bond;

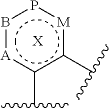

is nitrogen-containing 6-membered heteroaryl; A, B, P and M are independently CH or N, and at least one is N;

m is 0, 1, 2 or 3;

R² is —CN, C₁₋₆ alkyl, C₂₋₆ alkenyl, C₂₋₆ alkynyl, C₁₋₆ alkyl substituted with one or more R²⁻¹, halogen, —OR^{2a}, —C(═O)R^{2b}, —NR^{2c1}R^{2c2}, —C(═O)OR^{2d}, —C(═O)NR^{2e1}R^{2e2}, C₃₋₁₀ cycloalkyl, C₃₋₁₀ cycloalkyl substituted with one or more R²-2, “4- to 10-membered heterocycloalkyl containing 1-3 heteroatoms independently selected from O and N”, “4- to 10-membered heterocycloalkyl containing 1-3 heteroatoms independently selected from O and N” substituted with one or more R²⁻³, C₆₋₂₀ aryl, C₆₋₂₀ aryl substituted with one or more R²⁻⁴, “5- to 12-membered heteroaryl containing 1-4 heteroatoms independently selected from O, S and N”, or “5- to 12-membered heteroaryl containing 1-4 heteroatoms independently selected from O, S and N” substituted with one or more R²-5; provided that when multiple substituents are present, the substituents are the same or different;

R²⁻¹, R²⁻², R²⁻³, R²⁻⁴ and R²⁻⁵ are independently halogen, hydroxyl, cyano, C₁₋₆ alkyl, C₂₋₆ alkenyl, C₂₋₆ alkynyl, C₁₋₆ alkyl-O—, —C(═O)R³¹, —NR³²R³³, —C(═O)OR³⁴ or —C(═O)NR³⁵R³⁶.

R^{2a}, R^{2b}, R^{2c1}, R^{2c2}, R^{2a}, R^{2e1} and R^{2e2} are independently hydrogen or C₁₋₆ alkyl;

R³¹, R³², R³³, R³⁴, R³⁵ and R³⁶ are independently hydrogen or C₁₋₆ alkyl;

n is 0, 1, 2, 3, 4, 5 or 6;

R⁴ is independently C₁₋₆ alkyl, C₁₋₆ alkyl substituted with one or more R⁴⁻¹, C₁₋₆ alkyl-O—, O═, —C(═O)OR^{4a} or —C(═O)NR^{4b}R^{4c}; or, when n is 2, 3, 4, 5 or 6, two optional R⁴ are connected, together with the atoms on the ring to which they are attached, independently form 3- to 8-membered carbocyclic ring or 3- to 8-membered heterocyclic ring containing 1-3 heteroatoms independently selected from O, S and N;

R⁴⁻¹ is independently halogen, cyano, hydroxyl, C₁₋₆ alkyl-O—, —NR⁴ⁱR^{4j}, —C(═O)OR^{4d} or —C(═O)NR^{4e}R^{4f};

R^{4a}, R^{4b}, R^{4c}, R^{4d}, R^{4e}, R^{4f}, R⁴ⁱ and R^{4j} are independently hydrogen or C₁₋₆ alkyl;

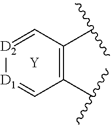

is phenyl, “5- to 7-membered heterocycloalkenyl containing 1-3 heteroatoms independently selected from O, S and N”, “5- to 7-membered heteroaryl containing 1-3 heteroatoms independently selected from O, S and N” or 5- to 7-membered cycloalkenyl; wherein D¹ is C, CH or N; D₂ is

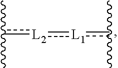

wherein Z₁ and Z₂ are independently a bond (for example, when Z₁ is a bond, D₂ is

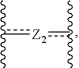

When Z₂ is a bond, D₂ is

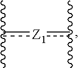

when Z₁ and Z₂ are independently a bond, D₂ is

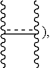

### CH, CH₂, O, S, N or NH;

r is 0, 1, 2, 3, 4, 5 or 6;

R⁵ is independently halogen or C₁₋₆ alkyl;

L₁ is a bond, —C(═O)— or C₁₋₆ alkylene;

R¹ is C₆₋₂₀ aryl, C₈₋₁₁ benzocycloalkenyl, “5 to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;

X¹ and X² are independently CR^{b} or N, and X¹ and X² are not both CR^{b};

R^{b}, R¹⁻¹ and R¹⁻² are independently halogen, —OR^{c}, cyano, —C(═O)R¹¹, —NR¹²R¹³, —C(═O)OR¹⁴, —C(═O)NR¹⁵R¹⁶, C₁₋₆ alkyl, C₂₋₆ alkenyl, C₂₋₆ alkynyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R¹⁻¹, C₁₋₆ alkyl-O substituted with one or more R¹⁻¹⁻², C₃₋₁₀ cycloalkyl substituted with one or more R¹⁻¹⁻³, “5 to 7-membered heterocycloalkyl containing 1 or 2 heteroatoms independently selected from O and N” substituted with one or more R¹⁻¹⁻⁴, C₆₋₂₀ aryl substituted with one or more R¹⁻¹⁻⁵, or “5- to 7-membered heteroaryl containing 1 or 2 heteroatoms independently selected from O and N” substituted with one or more R¹⁻¹⁻⁶; provided that when multiple substituents are present, the substituents are the same or different; or, when the number of R¹⁻¹ or R¹⁻² is more than one, two optional R¹⁻¹ or two optional R¹⁻² are connected, together with the atoms on the ring to which they are attached, independently form 3- to 8-membered cyclic olefin;

R^{c}, R¹² and R¹³ are independently hydrogen, C₁₋₆ alkyl, C(═O)R^{c1}, —C(═O)OR^{c2}, or —C(═O)NR^{3c}R^{c4}; R^{c1}, R^{c2}, R^{c3} and R^{c4} are independently hydrogen, C₁₋₆ alkyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R⁴⁻¹, C₃₋₁₀ cycloalkyl substituted with one or more R¹⁻¹⁻², “5- to 7-membered heterocycloalkyl containing 1 or 2 heteroatoms independently selected from O and N” substituted with one or more R¹⁻¹⁻³, C₆₋₂₀ aryl substituted with one or more R¹⁻¹⁻⁴, or, “5- to 7-membered heteroaryl containing 1 or 2 heteroatoms independently selected from O and N” substituted with one or more R⁴⁻¹⁻⁵; provided that when multiple substituents are present, the substituents are the same or different;

R¹⁻¹⁻¹, R¹⁻¹⁻², R¹⁻¹⁻³, R¹⁻¹⁻⁴, R¹⁻¹⁻⁵, R¹⁻¹⁻⁶, R¹⁻¹⁻¹, R¹⁻¹⁻², R¹⁻¹⁻³, R⁴⁻¹⁻⁴ and R¹⁻¹⁻⁵ are independently cyano, halogen, hydroxyl, C₁₋₆ alkyl-O—, C₁₋₆ alkyl, —C(═O)R²¹, —NR²²R²³, —C(═O)OR²⁴ or —C(═O)NR²⁵R²⁶;

R¹¹, R²¹, R²², R²³, R¹⁴, R²⁴, R¹⁵, R²⁵, R¹⁶ and R²⁶ are independently hydrogen or C₁₋₆ alkyl;

L₂ is a bond, C₁₋₆ alkylene, —C(═O)—, —O(R^{L-1})ₙ₁—, —S(R^{L-2})ₙ₂— or —NR^{L-3}(R^{L-4})ₙ₃—; R^{L-1}, R^{L-2} and R^{L-4} are independently C₁₋₆ alkylene; R^{L-3} is hydrogen or C₁₋₆ alkyl; n1, n2 and n3 are independently 0 or 1;

R³ is C₃₋₁₂ cycloalkyl, C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻², C₁₋₆ alkyl, C₁₋₆ alkyl substituted with one or more R³⁻³, —OR^{d}, —SR^{d1}, —NR^{e1}R^{e2} or —C(═O)NR^{e3}R^{e4}; provided that when multiple substituents are present, the substituents are the same or different;

R³⁻¹, R³⁻² and R³⁻³ are independently C₁₋₆ alkyl, C₁₋₆ alkyl substituted with one or more R³⁻¹⁻¹ hydroxyl, C₁₋₆ alkyl-O—, halogen, O═, —NR^{e5}R^{e6} or —C(═O)NR^{e7}R^{e8};

R^{d}, R^{d1}, R^{e1}, R^{e2}, R^{e3} and R^{e4} are independently hydrogen, C₁₋₆ alkyl, C₃₋₁₀ cycloalkyl, “4- to 10-membered heterocycloalkyl containing 1-3 heteroatoms independently selected from O and N”, or C₁₋₆ alkyl substituted with one or more R³⁻¹⁻²;

R³⁻¹, R³⁻² and R³⁻¹⁻² are independently deuterium(D), cyano, halogen, hydroxyl, C₁₋₆ alkyl-O—, —C(═O)R^{e9}, —NR^{e10}R^{e11}, —C(═O)OR^{e12} or —C(═O)NR^{e13}R^{e14};

R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl.

In a certain embodiment, in the nitrogen-containing heterocyclic compound of formula I, a pharmaceutically acceptable salt thereof, a stereoisomer thereof, a tautomer thereof or an isotopically labeled compound thereof, wherein,

“ [symbol] ” represents a single or double bond;

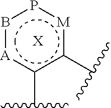

is nitrogen-containing 6-membered heteroaryl, P is N, A, B and M are independently CH;

m is 0, 1, 2 or 3;

R² is —CN, C₁₋₆ alkyl, C₂₋₆ alkenyl, C₂₋₆ alkynyl, C₁₋₆ alkyl substituted with one or more R²⁻¹, halogen, —OR^{2a}, —C(═O)R^{2b}, —NR^{2c1}R^{2c2}, —C(═O)OR^{2d}, —C(═O)NR^{2e1}R^{2e2}, C₃₋₁₀ cycloalkyl, C₃₋₁₀ cycloalkyl substituted with one or more R²-2, “4- to 10-membered heterocycloalkyl containing 1-3 heteroatoms independently selected from O and N”, “4- to 10-membered heterocycloalkyl containing 1-3 heteroatoms independently selected from O and N” substituted with one or more R²⁻³, C₆₋₂₀ aryl, C₆₋₂₀ aryl substituted with one or more R²⁻⁴, “5- to 12-membered heteroaryl containing 1-4 heteroatoms independently selected from O, S and N”, or “5- to 12-membered heteroaryl containing 1-4 heteroatoms independently selected from O, S and N” substituted with one or more R²-5; provided that when multiple substituents are present, the substituents are the same or different;

R²⁻¹, R²⁻², R²⁻³, R²⁻⁴ and R²⁻⁵ are independently halogen, hydroxyl, cyano, C₁₋₆ alkyl, C₂₋₆ alkenyl, C₂₋₆ alkynyl, C₁₋₆ alkyl-O—, —C(═O)R³¹, —NR³²R³³, —C(═O)OR³⁴ or —C(═O)NR³⁵R³⁶;

R^{2a}, R^{2b}, R^{2c1}, R^{2c2}, R^{2a}, R^{2e1} and R^{2e2} are independently hydrogen or C₁₋₆ alkyl;

R³¹, R³², R³³, R³⁴, R³⁵ and R³⁶ are independently hydrogen or C₁₋₆ alkyl;

n is 0, 1, 2, 3, 4, 5 or 6;

R⁴ is independently C₁₋₆ alkyl, C₁₋₆ alkyl substituted with one or more R⁴⁻¹, C₁₋₆ alkyl-O—, O═, —C(═O)OR^{4a} or —C(═O)NR^{4I}R^{4c}; or, when n is 2, 3, 4, 5 or 6, two optional R⁴ are connected, together with the atoms on the ring to which they are attached, independently form 3- to 8-membered carbocyclic ring or 3- to 8-membered heterocyclic ring containing 1-3 heteroatoms independently selected from O, S and N;

R⁴⁻¹ is independently halogen, cyano, hydroxyl, C₁₋₆ alkyl-O—, —NR⁴ⁱR^{4j}, —C(═O)OR^{4d} or —C(═O)NR^{4e}R^{4f};

R^{4a}, R^{4b}, R^{4c}, R^{4d}, R^{4e}, R^{4f}, R⁴ⁱ and R^{4j} are independently hydrogen or C₁₋₆ alkyl;

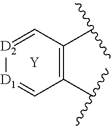

is phenyl, “5- to 7-membered heterocycloalkenyl containing 1-3 heteroatoms independently selected from O, S and N”, “5- to 7-membered heteroaryl containing 1-3 heteroatoms independently selected from O, S and N” or 5- to 7-membered cycloalkenyl; wherein D¹ is C, CH or N; D₂ is

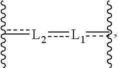

wherein Z₁ and Z₂ are independently a bond, CH, CH₂, O, S, N or NH;

r is 0, 1, 2, 3, 4, 5 or 6;

R⁵ is independently halogen or C₁₋₆ alkyl;

L₁ is a bond, —C(═O)— or C₁₋₆ alkylene;

R¹ is C₆₋₂₀ aryl, C₈₋₁₁ benzocycloalkenyl, “5 to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;

X¹ and X² are independently CR^{b} or N, and X¹ and X² are not both CR^{b};

R^{b}, R¹⁻¹ and R¹⁻² are independently halogen, —OR^{c}, cyano, —C(═O)R¹¹, —NR¹²R¹³, —C(═O)OR¹⁴, —C(═O)NR¹⁵R¹⁶, C₁₋₆ alkyl, C₂₋₆ alkenyl, C₂₋₆ alkynyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R¹⁻¹, C₁₋₆ alkyl-O substituted with one or more R¹⁻¹⁻², C₃₋₁₀ cycloalkyl substituted with one or more R¹⁻¹⁻³, “5 to 7-membered heterocycloalkyl containing 1 or 2 heteroatoms independently selected from O and N” substituted with one or more R¹⁻¹⁻⁴, C₆₋₂₀ aryl substituted with one or more R¹⁻¹⁻⁵, or “5- to 7-membered heteroaryl containing 1 or 2 heteroatoms independently selected from O and N” substituted with one or more R¹⁻¹⁻⁶; provided that when multiple substituents are present, the substituents are the same or different; or, when the number of R¹⁻¹ or R¹⁻² is more than one, two optional R¹⁻¹ or two optional R¹⁻² are connected, together with the atoms on the ring to which they are attached, independently form 3- to 8-membered cyclic olefin;

R^{c}, R¹² and R¹³ are independently hydrogen, C₁₋₆ alkyl, C(═O)R^{c1}, —C(═O)OR^{c2}, or —C(═O)NR^{3c}R^{c4}; R^{c1}, R^{c2}, R^{c3} and R^{c4} are independently hydrogen, C₁₋₆ alkyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R⁴⁻¹, C₃₋₁₀ cycloalkyl substituted with one or more R¹⁻¹⁻², “5- to 7-membered heterocycloalkyl containing 1 or 2 heteroatoms independently selected from O and N” substituted with one or more R¹⁻¹⁻³, C₆₋₂₀ aryl substituted with one or more R¹⁻¹⁻⁴, or, “5- to 7-membered heteroaryl containing 1 or 2 heteroatoms independently selected from O and N” substituted with one or more R¹⁻¹⁻⁵; provided that when multiple substituents are present, the substituents are the same or different;

R¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R¹⁻¹⁻², R¹⁻¹⁻³, R⁴⁻¹⁻⁴ and R¹⁻¹⁻⁵ are independently cyano, halogen, hydroxyl, C₁₋₆ alkyl-O—, C₁₋₆ alkyl, —C(═O)R²¹, —NR²²R²³, —C(═O)OR²⁴ or —C(═O)NR²⁵R²⁶;

R¹¹, R²¹, R²², R²³, R¹⁴, R²⁴, R¹⁵, R²⁵, R¹⁶ and R²⁶ are independently hydrogen or C₁₋₆ alkyl;

L₂ is a bond, C₁₋₆ alkylene, —C(═O)—, —O(R^{L-1})ₙ₁—, —S(R^{L-2})ₙ₂— or —NR^{L-3}(R^{L-4})ₙ₃—; R^{L-1}, R^{L-2} and R^{L-4} are independently C₁₋₆ alkylene; R^{L-3} is hydrogen or C₁₋₆ alkyl; n1, n2 and n3 are independently 0 or 1;

R³ is C₃₋₁₂ cycloalkyl, C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻², C₁₋₆ alkyl, C₁₋₆ alkyl substituted with one or more R³⁻³, —OR^{d}, —SR^{d1}, —NR^{e1}R^{e2} or —C(═O)NR^{e3}R^{e4}; provided that when multiple substituents are present, the substituents are the same or different;

R³⁻¹, R³⁻² and R³⁻³ are independently C₁₋₆ alkyl, C₁₋₆ alkyl substituted with one or more R³⁻¹⁻¹ hydroxyl, C₁₋₆ alkyl-O—, halogen, O═, —NR^{e5}R^{e6} or —C(═O)NR^{e7}R^{e8};

R^{d}, R^{d1}, R^{e1}, R^{e2}, R^{e3} and R^{e4} are independently hydrogen, C₁₋₆ alkyl, C₃₋₁₀ cycloalkyl, “4- to 10-membered heterocycloalkyl containing 1-3 heteroatoms independently selected from O and N”, or C₁₋₆ alkyl substituted with one or more R³⁻¹⁻².

R³⁻¹⁻¹ and R³⁻¹⁻² are independently deuterium, cyano, halogen, hydroxyl, C₁₋₆ alkyl-O—, —C(═O)R^{e9}, —NR^{e10}R^{e11}, —C(═O)OR^{e12} or —C(═O)NR^{e13}R^{e14};

R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl.

In a certain embodiment, in the nitrogen-containing heterocyclic compound of formula I, a pharmaceutically acceptable salt thereof, a stereoisomer thereof, a tautomer thereof or an isotopically labeled compound thereof, wherein,

“ [symbol] ” represents a single or double bond;

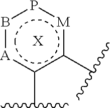

is nitrogen-containing 6-membered heteroaryl; P is N, A, B and M are independently CH, or, A is N, P, B and M are independently CH, or, B is N, A, P and M are independently CH;

m is 0 or 1;

R² is halogen or —OR^{2a}; R^{2a} is C₁₋₆ alkyl;

n is 0 or 1;

R⁴ is C₁₋₆ alkyl;

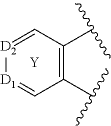

is phenyl, “6-membered heterocycloalkenyl containing 1 heteroatom selected from O or N”, or, “5- to 7-membered heteroaryl containing 1-3 heteroatoms independently selected from O, S and N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond; or D¹ is C, and in D₂, of Z₁ and Z₂, one is N, the other is a bond;

r is 0 or 1;

R⁵ is independently halogen;

L₁ is a bond;

R¹ is C₆₋₂₀ aryl substituted with one or more R¹⁻¹, “5- to 12-membered heteroaryl containing 1-4 heteroatoms independently selected from O, S and N” or “5- to 12-membered heteroaryl containing 1-4 heteroatoms independently selected from O, S and N” substituted with one or more R¹⁻²; provided that when multiple substituents are present, the substituents are the same or different;

R¹⁻¹ is independently halogen, —OR^{c}, C₂₋₆ alkynyl, C₁₋₆ alkyl-O substituted with one or more R¹⁻¹⁻²;

R^{c} is hydrogen;

R¹⁻¹⁻² is independently C₁₋₆ alkyl-O—;

R¹⁻² is independently halogen or —NR¹²R¹³;

R¹² and R¹³ are independently hydrogen;

X¹ and X² are independently N;

L₂ is —O(R^{L-1})ₙ₁—; R^{L-1} is independently C₁₋₆ alkylene; n1 is 1;

R³ is “5- to 6-membered heterocycloalkyl containing 1-2 heteroatoms independently selected from O and N” substituted with one or more R³⁻² or —NR^{e1}R^{e2};

R³⁻², R^{e1} and R^{e2} are independently C₁₋₆ alkyl;

when

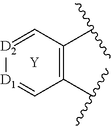

is “6-membered heterocycloalkenyl containing 1 heteroatom selected from O or N”, D¹ is CH, and in D₂, of Z₁ and Z₂, one is O, the other is a bond,

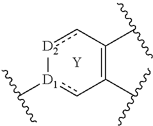

is

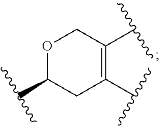 [SMILES: *C1=C(*)C[C@@H](*)OC1]

when n is 1,

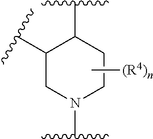

is

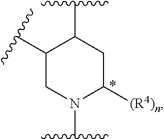

wherein,

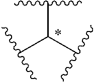 [SMILES: *C(*)*]

represents R configuration, S configuration or a mixture thereof.

In a certain embodiment, in the nitrogen-containing heterocyclic compound of formula I, a pharmaceutically acceptable salt thereof, a stereoisomer thereof, a tautomer thereof or an isotopically labeled compound thereof, wherein,

“ [symbol] ” represents a single or double bond;

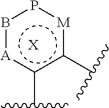

is nitrogen-containing 6-membered heteroaryl; P is N, A, B and M are independently CH, or, A is N, P, B and M are independently CH, or, B is N, A, P and M are independently CH;

m is 0 or 1;

R² is halogen or —OR^{2a}; R^{2a} is C₁₋₆ alkyl;

n is 0 or 1;

R⁴ is C₁₋₆ alkyl;

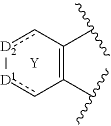

is phenyl, “6-membered heterocycloalkenyl containing 1 heteroatom selected from O or N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond;

r is 0;

L₁ is a bond;

R¹ is C₆₋₂₀ aryl substituted with one or more R¹⁻¹, “5- to 12-membered heteroaryl containing 1-4 heteroatoms independently selected from O, S and N” or “5- to 12-membered heteroaryl containing 1-4 heteroatoms independently selected from O, S and N” substituted with one or more R¹⁻²; provided that when multiple substituents are present, the substituents are the same or different;

R¹⁻¹ is independently halogen, —OR^{c}, C₂₋₆ alkynyl, C₁₋₆ alkyl-O substituted with one or more R¹⁻¹⁻²;

R^{c} is hydrogen;

R¹⁻¹⁻² is independently C₁₋₆ alkyl-O—;

R¹⁻² is independently halogen or —NR¹²R¹³;

R¹² and R¹³ are independently hydrogen;

X¹ and X² are independently N;

L₂ is —O(R^{L-1})ₙ₁—; R^{L-1} is independently C₁₋₆ alkylene; n1 is 1;

R³ is “5- to 6-membered heterocycloalkyl containing 1-2 heteroatoms independently selected from O and N” substituted with one or more R³⁻² or —NR^{e1}R^{e2};

R³⁻², R^{e1} and R^{e2} are independently C₁₋₆ alkyl;

when

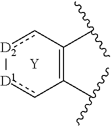

is “6-membered heterocycloalkenyl containing 1 heteroatom selected from O or N”, D¹ is CH, and in D₂, of Z₁ and Z₂, one is O, the other is a bond,

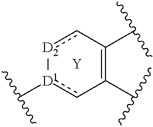

is

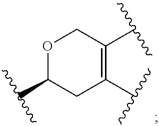 [SMILES: *C1=C(*)C[C@@H](*)OC1]

when n is 1,

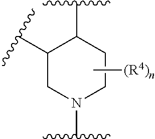

is

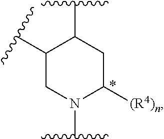

wherein,

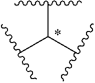 [SMILES: *C(*)*]

represents R configuration, S configuration or a mixture thereof.

In a certain embodiment, in the nitrogen-containing heterocyclic compound of formula I, a pharmaceutically acceptable salt thereof, a stereoisomer thereof, a tautomer thereof or an isotopically labeled compound thereof, wherein

“ [symbol] ” represents a single or double bond;

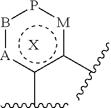

is nitrogen-containing 6-membered heteroaryl; P is N, A, B and M are independently CH, or, A is N, P, B and M are independently CH, or, B is N, A, P and M are independently CH;

m is 0 or 1;

R² is halogen or —OR^{2a}; R^{2a} is C₁₋₆ alkyl;

n is 0 or 1;

R⁴ is C₁₋₆ alkyl;

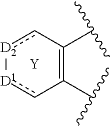

is phenyl, “6-membered heterocycloalkenyl containing 1 heteroatom selected from O or N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond;

r is 0;

L₁ is a bond;

R¹ is C₆₋₂₀ aryl substituted with one or more R¹⁻¹; provided that when multiple substituents are present, the substituents are the same or different;

R¹⁻¹ is independently halogen, —OR^{c}, C₂₋₆ alkynyl, C₁₋₆ alkyl-O substituted with one or more R¹⁻¹⁻²

R^{c} is hydrogen;

R¹⁻¹⁻² is independently C₁₋₆ alkyl-O—;

X¹ and X² are independently N;

L₂ is —O(R^{L-1})ₙ₁—; R^{L-1} is independently C₁₋₆ alkylene; n1 is 1;

R³ is “5- to 6-membered heterocycloalkyl containing 1 heteroatom being N” substituted with one or more R³⁻²;

R³⁻² is independently C₁₋₆ alkyl;

when

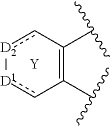

is “6-membered heterocycloalkenyl containing 1 heteroatom selected from O or N”, D is CH, and in D₂, of Z₁ and Z₂, one is O, the other is a bond,

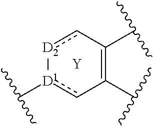

is

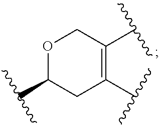 [SMILES: *C1=C(*)C[C@@H](*)OC1]

when n is 1,

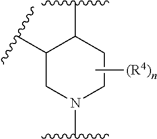

is

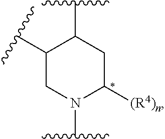

wherein,

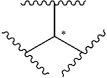 [SMILES: *C(*)*]

represents R configuration, S configuration or a mixture thereof.

In a certain embodiment, in the nitrogen-containing heterocyclic compound of formula I, a pharmaceutically acceptable salt thereof, a stereoisomer thereof, a tautomer thereof or an isotopically labeled compound thereof, wherein

“ [symbol] ” represents a single or double bond;

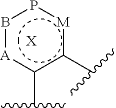

is nitrogen-containing 6-membered heteroaryl; P is N, A, B and M are independently CH, or, A is N, P, B and M are independently CH, or, B is N, A, P and M are independently CH;

m is 0 or 1;

R² is halogen or —OR^{2a}; R^{2a} is C₁₋₆ alkyl;

n is 0 or 1;

R⁴ is C₁₋₆ alkyl;

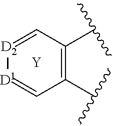

is “6-membered heterocycloalkenyl containing 1 heteroatom being N”, wherein D¹ is N, and in D₂, of Z₁ and Z₂, one is CH₂, the other is a bond;

r is 0;

L₁ is a bond;

R¹ is C₆₋₂₀ aryl substituted with one or more R¹⁻¹; provided that when multiple substituents are present, the substituents are the same or different;

R¹⁻¹ is independently halogen, —OR^{c} or C₂₋₆ alkynyl; R^{c} is hydrogen;

X¹ and X² are independently N;

L₂ is —O(R^{L-1})ₙ₁—; R^{L-1} is independently C₁₋₆ alkylene; n1 is 1;

R³ is “5- to 6-membered heterocycloalkyl containing 1 heteroatom being N” substituted with one or more R³⁻²;

R³⁻² is independently C₁₋₆ alkyl.

In a certain embodiment, in the nitrogen-containing heterocyclic compound of formula I, a pharmaceutically acceptable salt thereof, a stereoisomer thereof, a tautomer thereof or an isotopically labeled compound thereof, wherein

“ [symbol] ” represents a single or double bond;

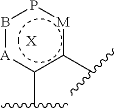

is nitrogen-containing 6-membered heteroaryl; P is N, A, B and M are independently CH;

m is 0 or 1;

R² is halogen or —OR^{2a}; R^{2a} is C₁₋₆ alkyl;

n is 0 or 1;

R⁴ is C₁₋₆ alkyl;

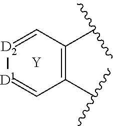

is “6-membered heterocycloalkenyl containing 1 heteroatom being N”, wherein D¹ is N, and in D₂, of Z₁ and Z₂, one is CH₂, the other is a bond;

r is 0;

L₁ is a bond;

R¹ is C₆₋₂₀ aryl substituted with one or more R¹⁻¹; provided that when multiple substituents are present, the substituents are the same or different;

R¹⁻¹ is independently halogen, —OR^{c} or C₂₋₆ alkynyl; R^{c} is hydrogen;

X¹ and X² are independently N;

L₂ is —O(R^{L-1})ₙ₁—; R^{L-1} is independently C₁₋₆ alkylene; n1 is 1;

R³ is “5-membered heterocycloalkyl containing 1 heteroatom being N” substituted with one or more R³⁻²;

R³⁻² is independently C₁₋₆ alkyl;

when n is 1,

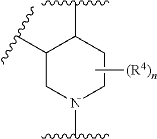

is

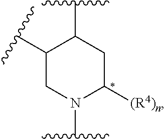

wherein,

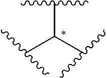 [SMILES: *C(*)*]

represents R configuration, S configuration or a mixture thereof.

In a certain embodiment, with regard to a nitrogen-containing heterocyclic compound represented by formula I, a pharmaceutically acceptable salt thereof, a stereoisomer thereof, a tautomer thereof or an isotopic compound thereof, some groups are defined as follows, and the unmentioned group definitions are as described in any one of the embodiments of the present disclosure (this content is hereinafter referred to simply as “in a certain embodiment”), wherein, R³⁻¹⁻¹ is independently deuterium(D).

In a certain embodiment, R³⁻¹⁻² is independently deuterium(D).

In a certain embodiment, in

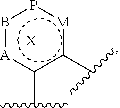

P is N, A, B and M are independently CH, or, A is N, P, B and M are independently CH, or, B is N, A, P and M are independently CH.

In a certain embodiment, m is 0 or 1.

In a certain embodiment, R² is-CN, C₁₋₆ alkyl, C₁₋₆ alkyl substituted with one or more R²⁻¹, halogen, —OR^{2a}, —NR^{2c1}R^{2c2}, —C(═O)NR^{2e1}R^{2e2}, C₃₋₁₀ cycloalkyl, C₆₋₂₀ aryl, “5- to 12-membered heteroaryl containing 1-4 heteroatoms independently selected from O, S and N”, for example, —OR^{2a} or halogen.

In a certain embodiment, R^{2a} is C₁₋₆ alkyl or hydrogen, for example, C₁₋₆ alkyl.

In a certain embodiment, R²⁻¹ is hydroxyl.

In a certain embodiment, R^{2c1}, R^{2c2}, R^{2e} and R^{2e2} are independently hydrogen.

In a certain embodiment, n is 0 or 1.

In a certain embodiment, R⁴ is independently C₁₋₆ alkyl, C₁₋₆ alkyl substituted with one or more R⁴⁻¹, or C₁₋₆ alkyl-O—, for example, C₁₋₆ alkyl.

In a certain embodiment, R⁴⁻¹ is independently cyano.

In a certain embodiment, D¹ is C, CH or N.

In a certain embodiment, in D₂, of Z₁ and Z₂, one is CH, N, CH₂ or O, the other one is a bond.

In a certain embodiment, r is 0 or 1.

In a certain embodiment, when r is 1,

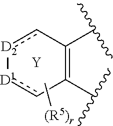

is

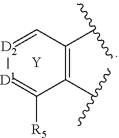

In a certain embodiment,

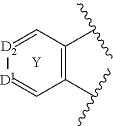

is phenyl, “6-membered heterocycloalkenyl containing 1 heteroatom selected from O or N”, or, “5- to 7-membered heteroaryl containing 1-3 heteroatoms independently selected from O, S and N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond; or D¹ is C, and in D₂, of Z₁ and Z₂, one is N, the other is a bond.

In a certain embodiment,

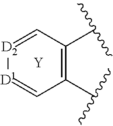

is phenyl, or “6-membered heterocycloalkenyl containing 1 heteroatom selected from O or N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond.

In a certain embodiment, R⁵ is independently halogen.

In a certain embodiment, X¹ and X² are independently N.

In a certain embodiment, L₁ is a bond or —C(═O)—, for example, a bond.

In a certain embodiment, R¹ is C₆₋₂₀ aryl substituted with one or more R¹⁻¹, “5- to 12-membered heteroaryl containing 1-4 heteroatoms independently selected from O, S and N” or “5- to 12-membered heteroaryl containing 1-4 heteroatoms independently selected from O, S and N” substituted with one or more R¹⁻².

In a certain embodiment, R¹⁻¹ is independently halogen, —OR^{c}, C₂₋₆ alkynyl, C₁₋₆ alkyl-O substituted with one or more R¹⁻¹⁻²

In a certain embodiment, R^{c} is hydrogen.

In a certain embodiment, R¹⁻¹⁻² is independently C₁₋₆ alkyl-O.

In a certain embodiment, R¹-2 is independently —NR¹²R¹³.

In a certain embodiment, R¹² and R¹³ are independently hydrogen.

In a certain embodiment, L₂ is a bond or —O(R^{L-1})ₙ₁—.

In a certain embodiment, R^{L-1} is independently C₁₋₆ alkylene.

In a certain embodiment, n1 is 0 or 1;

In a certain embodiment, R³ is C₃₋₁₂ cycloalkyl substituted with one or more R³⁻¹, “4- to 12-membered heterocycloalkyl containing 1-2 heteroatoms independently selected from 0 and N”, “4- to 12-membered heterocycloalkyl containing 1-2 heteroatoms independently selected from 0 and N” substituted with one or more R³⁻², C₁₋₆ alkyl or —NR^{e1}R^{e2}; for example, “4- to 12-membered heterocycloalkyl containing 1-3 heteroatoms independently selected from 0 and N”, “4- to 12-membered heterocycloalkyl containing 1-3 heteroatoms independently selected from 0 and N” substituted with one or more R³⁻², C₁₋₆ alkyl or —NR^{e1}R^{e2}, for another example, “5- to 6-membered heterocycloalkyl containing 1 heteroatom being N” substituted with one or more R³⁻²

In a certain embodiment, R³ is “5- to 6-membered heterocycloalkyl containing 1-2 heteroatoms independently selected from 0 and N” substituted with one or more R³⁻² or —NR^{e1}R^{e2}

In a certain embodiment, R³⁻² is independently C₁₋₆ alkyl, halogen or O=(oxo), for example, C₁₋₆ alkyl.

In a certain embodiment, R^{e1} and R^{e2} are independently C₁₋₆ alkyl.

In a certain embodiment, when m is 1,

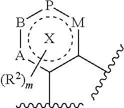

is

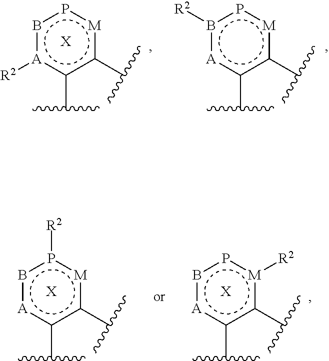

for example,

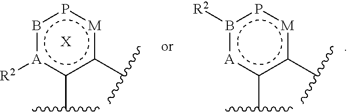

In a certain embodiment, when m is 1, P is N, and, A, B and M are CH, is

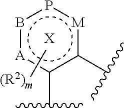

is

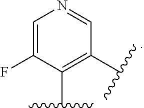 [SMILES: *c1cncc(F)c1*]

In a certain embodiment, when n is 1,

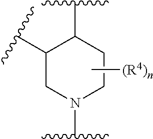

is

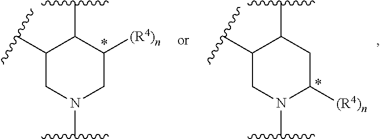

wherein,

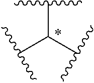 [SMILES: *C(*)*]

represents R configuration, S configuration or a mixture thereof. When

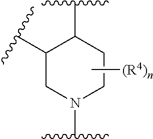

is

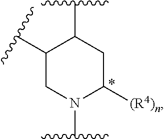

R⁴ is C₁₋₆ alkyl substituted with one or more R⁴⁻¹, or C₁₋₆ alkyl-O—; When

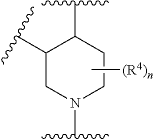

is

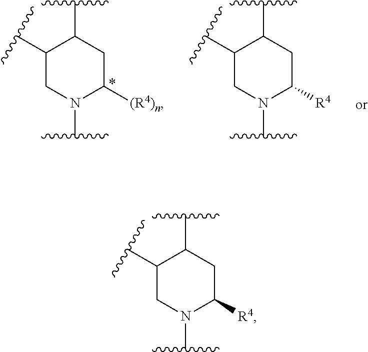

and n is 1, R⁴ is C₁₋₆ alkyl.

In a certain embodiment, when n is 1,

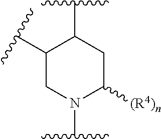

is

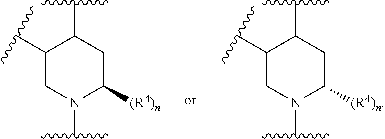

In a certain embodiment, in

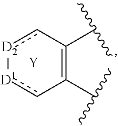

D¹ is C, and in D₂, of Z₁ and Z₂, one is CH or N, the other is a bond; or, D¹ is CH, and in D₂, of Z₁ and Z₂, one is O, the other is a bond; or, D¹ is N, and in D₂, of Z₁ and Z₂, one is CH₂, the other is a bond; or, D¹ is C, and in D₂, of Z₁ and Z₂, one is N, the other is a bond.

In a certain embodiment, in

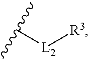

when L₂ is a bond, R³ is “4- to 12-membered heterocycloalkyl containing 1-3 heteroatoms independently selected from O, S and N”; or, when L₂ is —O(R^{L-1})ₙ₁—, R³ is C₃₋₁₂ cycloalkyl substituted with one or more R³⁻¹, “4- to 12-membered heterocycloalkyl containing 1-2 heteroatoms independently selected from O and N” substituted with one or more R³⁻², C₁₋₆ alkyl or —NR^{e1}R^{e2}; for example, “4- to 12-membered heterocycloalkyl containing 1-2 heteroatoms independently selected from O and N” substituted with one or more R³⁻², C₁₋₆ alkyl or —NR^{e1}R^{e2}; preferably, when R³ is “4 to 12-membered heterocycloalkyl containing 1-2 heteroatoms independently selected from O and N” substituted with one or more R³⁻², R³⁻² is C₁₋₆ alkyl.

In a certain embodiment, in

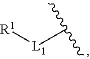

when L₁ is a bond, R¹ is C₆₋₂₀ aryl substituted with one or more R¹⁻¹, or “5- to 12-membered heteroaryl containing 1-4 heteroatoms independently selected from O, S and N” substituted with one or more R¹⁻²; or, when L₁ is —C(═O)—, R¹ is C₆₋₂₀ aryl substituted with one or more R¹⁻¹.

In a certain embodiment, in

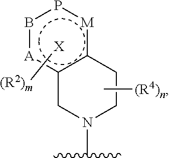

n is 1,

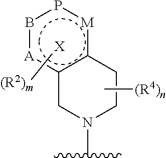

is

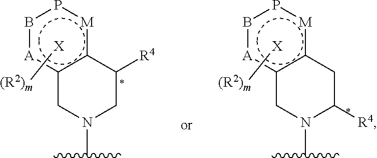

for example,

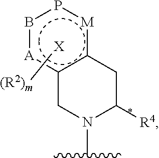

wherein,

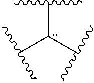 [SMILES: *C(*)*]

represents R configuration, S configuration or a mixture thereof.

In a certain embodiment, when m is 0, n is 1, and

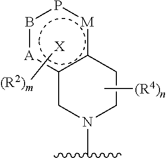

is

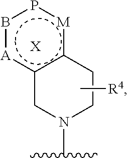

R⁴ is C₁₋₆ alkyl; preferably,

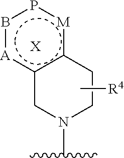

is

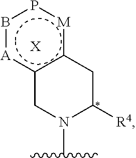

for example,

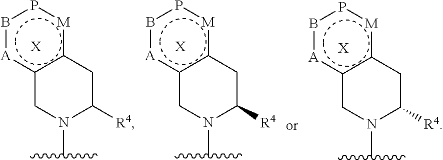

In a certain embodiment,

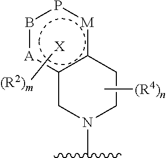

is

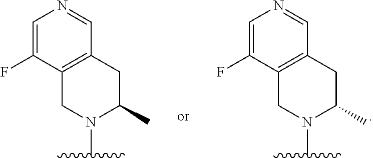 [SMILES: *N1Cc2c(F)cncc2C[C@H]1C ; *N1Cc2c(F)cncc2C[C@@H]1C]

or

In a certain embodiment, when R² is C₁₋₆ alkyl or C₁₋₆ alkyl substituted with one or more R²⁻¹, the C₁₋₆ alkyl and the C₁₋₆ alkyl in the C₁₋₆ alkyl substituted with one or more R²⁻¹ are C₁₋₄ alkyl, for example, methyl, ethyl, n-propyl, isopropyl, n-butyl, sec-butyl, isobutyl or tert-butyl, for another example, methyl.

In a certain embodiment, when R² is C₂₋₆ alkenyl, the C₂₋₆ alkenyl is C₂₋₃ alkenyl, for example, vinyl, propenyl or allyl.

In a certain embodiment, when R² is C₂₋₆ alkynyl, the C₂₋₆ alkynyl is C₂₋₃ alkynyl, for example, ethynyl, propynyl or propargyl.

In a certain embodiment, when R² is halogen, the halogen is fluorine, chlorine, bromine or iodine, for example, bromine.

In a certain embodiment, when R² is C₃₋₁₀ cycloalkyl or C₃₋₁₀ cycloalkyl substituted with one or more R²⁻², the C₃₋₁₀ cycloalkyl and the C₃₋₁₀ cycloalkyl in the C₃₋₁₀ cycloalkyl substituted with one or more R²⁻² are C₃₋₁₀ cycloalkyl, for example, cyclohexyl, cyclopentyl, cyclobutyl or cyclopropyl, for another example, cyclopropyl.

In a certain embodiment, when R² is “4- to 10-membered heterocycloalkyl containing 1-3 heteroatoms independently selected from O and N” or “4- to 10-membered heterocycloalkyl containing 1-3 heteroatoms independently selected from O and N” substituted with one or more R²⁻³, the “4- to 10-membered heterocycloalkyl containing 1-3 heteroatoms independently selected from O and N” and the “4- to 10-membered heterocycloalkyl containing 1-3 heteroatoms independently selected from O and N” in the “4- to 10-membered heterocycloalkyl containing 1-3 heteroatoms independently selected from O and N” substituted with one or more R²⁻³ can be 4- to 6-membered heterocycloalkyl containing 1-3 heteroatoms independently selected from O and N, can also be 4- to 6-membered heterocycloalkyl containing 1 heteroatom of O or N.

In a certain embodiment, when R² is C₆₋₂₀ aryl or C₆₋₂₀ aryl substituted with one or more R²⁻⁴, the C₆₋₂₀ aryl and the C₆₋₂₀ aryl in the C₆₋₂₀ aryl substituted with one or more R²⁻⁴ are C₆₋₁₀ aryl, for example, phenyl or naphthyl, for another example, phenyl.

In a certain embodiment, when R² is “5- to 12-membered heteroaryl containing 1-4 heteroatoms independently selected from O, S and N” or “5- to 12-membered heteroaryl containing 1-4 heteroatoms independently selected from O, S and N” substituted with one or more R²-5, the “5- to 12-membered heteroaryl containing 1-4 heteroatoms independently selected from O, S and N” and the “5- to 12-membered heteroaryl containing 1-4 heteroatoms independently selected from O, S and N” in the “5- to 12-membered heteroaryl containing 1-4 heteroatoms independently selected from O, S and N” substituted with one or more R²⁻⁵ are 5 to 6-membered heteroaryl containing 1 heteroatom of O, S or N, for example, pyridinyl, and for another example,

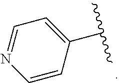 [SMILES: *c1ccncc1]

In a certain embodiment, when R²⁻¹, R²⁻², R²⁻³, R²⁻⁴ and R²⁻⁵ are independently halogen, the halogen is fluorine, chlorine, bromine or iodine.

In a certain embodiment, when R²⁻¹, R²⁻², R²⁻³, R²⁻⁴ and R²⁻⁵ are independently C₁₋₆ alkyl, the C₁₋₆ alkyl is C₁₋₄ alkyl, for example, methyl, ethyl, n-propyl, isopropyl, n-butyl, sec-butyl, isobutyl or tert-butyl, for another example, methyl.

In a certain embodiment, when R²⁻¹, R²⁻², R²⁻³, R²⁻⁴ and R²⁻⁵ are independently C₂₋₆ alkenyl, the C₂₋₆ alkenyl is C₂₋₃ alkenyl, for example, vinyl, propenyl or allyl.

In a certain embodiment, when R²⁻¹, R²⁻², R²⁻³, R²⁻⁴ and R²⁻⁵ are independently C₂₋₆ alkynyl, the C₂₋₆ alkynyl is C₂₋₃ alkynyl, for example, ethynyl, propynyl or propargyl.

In a certain embodiment, when R²⁻¹, R²⁻², R²⁻³, R²⁻⁴ and R²⁻⁵ are independently C₁₋₆ alkyl-O—, the C₁₋₆ alkyl in the C₁₋₆ alkyl-O— is C₁₋₄ alkyl, for example, methyl, ethyl, n-propyl, isopropyl, n-butyl, sec-butyl, isobutyl or tert-butyl.

In a certain embodiment, when R^{2a}, R^{2b}, R^{2c1}, R^{2c2}, R^{2d}, R^{2e1} and R^{2e2} are independently C₁₋₆ alkyl, the C₁₋₆ alkyl is C₁₋₄ alkyl, for example, methyl, ethyl, n-propyl, isopropyl, n-butyl, sec-butyl, isobutyl or tert-butyl, for another example, methyl.

In a certain embodiment, when R³¹, R³², R³³, R³⁴, R³⁵ and R³⁶ are independently C₁₋₆ alkyl, the C₁₋₆ alkyl is methyl, ethyl, n-propyl, isopropyl, n-butyl, sec-butyl, isobutyl, or tert-butyl, for another example, methyl.

In a certain embodiment, when R⁴ is C₁₋₆ alkyl or C₁₋₆ alkyl substituted with one or more R⁴⁻¹, the C₁₋₆ alkyl and the C₁₋₆ alkyl in the C₁₋₆ alkyl substituted with one or more R⁴⁻¹ are C₁₋₄ alkyl, for example, methyl, ethyl, n-propyl, isopropyl, n-butyl, sec-butyl, isobutyl or tert-butyl, for another example, methyl.

In a certain embodiment, when R⁴ is C₁₋₆ alkyl-O—, the C₁₋₆ alkyl in the C₁₋₆ alkyl-O— is C₁₋₆ alkyl, for example, methyl, ethyl, n-propyl, isopropyl, n-butyl, sec-butyl, isobutyl or tert-butyl, for another example, methyl.

In a certain embodiment, when n is 2, 3, 4, 5 or 6, and two optional R⁴ are connected, together with the atoms on the ring to which they are attached, independently form 3- to 8-membered carbocyclic ring, the 3- to 8-membered carbocyclic ring is 3- to 6-membered carbocyclic ring, and the carbocyclic ring is a monocyclic or bridged cycloalkyl.

In a certain embodiment, when n is 2, 3, 4, 5 or 6, and two optional R⁴ are connected, together with the atoms on the ring to which they are attached, independently form “3- to 8-membered heterocyclic ring containing 1-3 heteroatoms independently selected from O, S and N”, the “3- to 8-membered heterocyclic ring containing 1-3 heteroatoms independently selected from O, S and N” can be “3- to 6-membered heterocyclic ring containing 1-3 heteroatoms independently selected from O, S and N”, it can also be 3- to 6-membered heterocyclic ring containing 1 heteroatom of O, S or N.

In a certain embodiment, when R⁴⁻¹ is independently halogen, the halogen is fluorine, chlorine, bromine or iodine.

In a certain embodiment, when R⁴⁻¹ is independently C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl.

In a certain embodiment, when R^{4a}, R^{4b}, R^{4c}, R^{4d}, R^{4e}, R^{4f}, R⁴ⁱ and R^{4j} are independently C₁₋₆ alkyl, the C₁₋₆ alkyl is C₁₋₄ alkyl, for example, methyl, ethyl, n-propyl, isopropyl, n-butyl, sec-butyl, isobutyl or tert-butyl.

In a certain embodiment, when

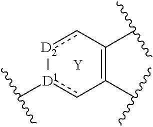

is “5- to 7-membered heterocycloalkenyl containing 1-3 heteroatoms independently selected from O, S and N”, the “5- to 7-membered heterocycloalkenyl containing 1-3 heteroatoms independently selected from O, S and N” is “5- to 7-membered heterocycloalkenyl containing 1-3 heteroatoms independently selected from O and N”, for example, “5- to 6-membered heterocycloalkenyl containing 1-2 heteroatoms independently selected from O and N”, for another example, 6-membered heterocycloalkenyl containing 1 heteroatom being O or N, or, 5- to 6-membered heterocycloalkenyl containing 1 heteroatom being O or N; for yet another example,

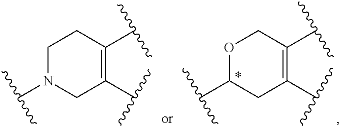 [SMILES: *C1=C(*)CN(*)CC1 ; *C1=C(*)CC(*)OC1]

still for another example

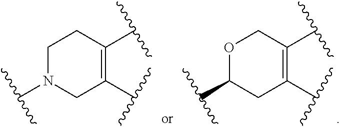 [SMILES: *C1=C(*)CN(*)CC1 ; *C1=C(*)C[C@@H](*)OC1]

In a certain embodiment, when

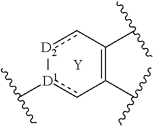

is “5- to 7-membered heteroaryl containing 1-3 heteroatoms independently selected from O, S and N”, the “5- to 7-membered heteroaryl containing 1-3 heteroatoms independently selected from O, S” and N is “6-membered heteroaryl containing 1-2 heteroatoms independently selected from O, S and N”, for example, “6-membered heteroaryl containing 1 heteroatom being N”, for another example, pyridinyl, for another example,

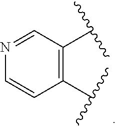 [SMILES: *c1ccncc1*]

In a certain embodiment, when

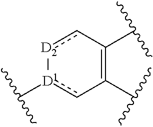

is 5- to 7-membered cycloalkenyl, the 5- to 7-membered cycloalkenyl is 5- to 6-membered cycloalkenyl, for example, cyclopentenyl or cyclohexenyl.

In a certain embodiment, when R⁵ is independently halogen, the halogen is fluorine, chlorine, bromine or iodine, for example, fluorine.

In a certain embodiment, when R⁵ is independently C₁₋₆ alkyl, the C₁₋₆ alkyl is C₁₋₄ alkyl, for example, methyl, ethyl, n-propyl, isopropyl, n-butyl, sec-butyl, isobutyl or tert-butyl.

In a certain embodiment, when L₁ is C₁₋₆ alkylene, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—.

In a certain embodiment, when R¹ is C₆₋₂₀ aryl or C₆₋₂₀ aryl substituted with one or more R¹⁻¹, the C₆₋₂₀ aryl and the C₆₋₂₀ aryl in the C₆₋₂₀ aryl substituted with one or more R¹⁻¹ are C₆₋₁₀ aryl, for example, phenyl or naphthyl.

In a certain embodiment, when R¹ is 5- to 12-membered heteroaryl containing “1-4 heteroatoms independently selected from O, S and N” or “5- to 12-membered heteroaryl containing 1-4 heteroatoms independently selected from O, S and N” substituted with one or more R¹⁻², the “5- to 12-membered heteroaryl containing 1-4 heteroatoms independently selected from O, S and N” and the “5- to 12-membered heteroaryl containing 1-4 heteroatoms independently selected from O, S and N” in the “5- to 12-membered heteroaryl containing 1-4 heteroatoms independently selected from O, S and N” substituted with one or more R¹⁻² are “5- to 10-membered heteroaryl containing 1-2 heteroatoms independently selected from O, S and N”, for example, “9-membered heteroaryl containing 2 heteroatoms independently selected from O, S and N”, further, benzothiazolyl, still further,

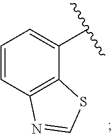 [SMILES: *c1cccc2ncsc12]

for another example, “10-membered heteroaryl containing 1 heteroatom of N”, further, quinolinyl or isoquinolinyl.

In a certain embodiment, in R¹, when “5- to 12-membered heteroaryl containing 1-4 heteroatoms independently selected from O, S and N” in the “5- to 12-membered heteroaryl containing 1-4 heteroatoms independently selected from O, S and N” substituted with one or more R¹⁻² is benzothiazolyl, the R¹ is

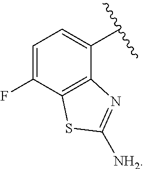 [SMILES: *c1ccc(F)c2sc(N)nc12]

In a certain embodiment, when R¹ is C₈₋₁₁ benzocycloalkenyl, the C₈₋₁₁ benzocycloalkenyl is benzocyclobutenyl, benzocyclopentenyl or benzocyclohexenyl.

In a certain embodiment, when R^{b}, R¹⁻¹ and R¹⁻² are independently halogen, the halogen is fluorine, chlorine, bromine or iodine, for example, fluorine or chlorine.

In a certain embodiment, when R^{b}, R¹⁻¹ and R¹⁻² are independently C₁₋₆ alkyl or C₁₋₆ alkyl substituted with one or more R¹⁻¹⁻¹, the C₁₋₆ alkyl and the C₁₋₆ alkyl in the C₁₋₆ alkyl substituted with one or more R¹⁻¹-1 are C₁₋₆ alkyl, for example, methyl, ethyl, n-propyl, isopropyl, n-butyl, sec-butyl, isobutyl or tert-butyl.

In a certain embodiment, when R^{b}, R¹⁻¹ and R¹⁻² are independently C₁₋₆ alkyl substituted with one or more R¹⁻¹⁻¹, the more R¹⁻¹⁻¹ is two or three R¹⁻¹⁻¹.

In a certain embodiment, when R^{b}, R¹⁻¹ and R¹⁻² are independently C₂₋₆ alkenyl, the C₂₋₆ alkenyl is C₂₋₃ alkenyl, for example, vinyl, propenyl or allyl.

In a certain embodiment, when R^{b}, R¹⁻¹ and R¹⁻² are independently C₂₋₆ alkynyl, the C₂₋₆ alkynyl is C₂₋₃ alkynyl, for example, ethynyl, propynyl or propargyl.

In a certain embodiment, when R^{b}, R¹⁻¹ and R¹⁻² are independently C₁₋₆ alkyl-O— or C₁₋₆ alkyl-O-substituted with one or more R¹⁻¹⁻², the C₁₋₆ alkyl and the C₁₋₆ alkyl in the C₁₋₆ alkyl-O— substituted with one or more R¹⁻¹⁻² is C₁₋₄ alkyl, for example, methyl, ethyl, n-propyl, isopropyl, n-butyl, sec-butyl, isobutyl or tert-butyl, for another example, methyl.

In a certain embodiment, when R^{b}, R¹⁻¹ and R¹⁻² are independently C₃₋₁₀ cycloalkyl or C₃₋₁₀ cycloalkyl substituted with one or more R¹⁻¹⁻³, the C₃₋₁₀ cycloalkyl and the C₃₋₁₀ cycloalkyl in the C₃₋₁₀ cycloalkyl substituted with one or more R¹⁻¹-3 are C₃₋₆ cycloalkyl, for example, cyclohexyl, cyclopentyl, cyclobutyl or cyclopropyl, for another example, cyclopropyl.

In a certain embodiment, when R^{b}, R¹⁻¹ and R¹⁻² are independently “5- to 7-membered heterocycloalkyl containing 1 or 2 heteroatoms independently selected from O and N” or “5- to 7-membered heterocycloalkyl containing 1 or 2 heteroatoms independently selected from O and N” substituted with one or more R¹⁻¹⁻⁴, the “5- to 7-membered heterocycloalkyl containing 1 or 2 heteroatoms independently selected from O and N” and the “5- to 7-membered heterocycloalkyl containing 1 or 2 heteroatoms independently selected from O and N” in the “5- to 7-membered heterocycloalkyl containing 1 or 2 heteroatoms independently selected from O and N” substituted with one or more R¹⁻¹⁻⁴ are “5- to 6-membered heterocycloalkyl containing 1 or 2 heteroatoms independently selected from O and”, for example, 5- to 6-membered heterocycloalkyl containing 1 heteroatom of O or N.

In a certain embodiment, when R^{b}, R¹⁻¹ and R¹⁻² are independently C₆₋₂₀ aryl or C₆₋₂₀ aryl substituted with one or more R¹⁻¹⁻⁵, the C₆₋₂₀ aryl and the C₆₋₂₀ aryl in the C₆₋₂₀ aryl substituted with one or more R¹⁻¹-5 are C₆₋₁₀ aryl, for example, phenyl or naphthyl.

In a certain embodiment, when R^{b}, R¹⁻¹ and R¹⁻² are independently “5- to 7-membered heteroaryl containing 1 or 2 heteroatoms independently selected from O and N” or “5- to 7-membered heteroaryl containing 1 or 2 heteroatoms independently selected from O and N” substituted with one or more R¹⁻¹⁻⁶, the “5- to 7-membered heteroaryl containing 1 or 2 heteroatoms independently selected from O and N” and “the 5- to 7-membered heteroaryl containing 1 or 2 heteroatoms independently selected from O and N” in the “5 to 7-membered heteroaryl containing 1 or 2 heteroatoms independently selected from O and N” substituted with one or more R¹⁻¹⁻⁶ are “5- to 6-membered heteroaryl containing 1-2 heteroatom independently selected from O or N”, for example, “5- to 6-membered heteroaryl containing 1 heteroatom of O or N”.

In a certain embodiment, when two optional R¹⁻¹ or two optional R¹⁻² are connected, together with the atoms on the ring to which they are attached, independently form 3- to 8-membered cyclic olefin, the 3- to 8-membered cyclic olefin is cyclobutene, cyclopentene or cyclohexene.

In a certain embodiment, when R^{c}, R¹² and R¹³ are independently C₁₋₆ alkyl, the C₁₋₆ alkyl is methyl, ethyl, n-propyl, isopropyl, n-butyl, sec-butyl, isobutyl or tert-butyl.

In a certain embodiment, when R^{c}, R^{c2}, R^{c3} and R^{c4} are independently C₁₋₆ alkyl or C₁₋₆ alkyl substituted with one or more R⁴⁻¹⁻¹, the C₁₋₆ alkyl and the C₁₋₆ alkyl in the C₁₋₆ alkyl substituted with one or more R⁴⁻¹⁻¹ are methyl, ethyl, n-propyl, isopropyl, n-butyl, sec-butyl, isobutyl or tert-butyl.

In a certain embodiment, when R^{c}, R^{c2}, R^{c3} and R^{c4} are independently C₃₋₁₀ cycloalkyl or C₃₋₁₀ cycloalkyl substituted with one or more R¹⁻¹⁻², the C₃₋₁₀ cycloalkyl and the C₃₋₁₀ cycloalkyl in the C₃₋₁₀ cycloalkyl substituted with one or more R¹⁻¹⁻² are cyclohexyl, cyclopentyl, cyclobutyl or cyclopropyl.

In a certain embodiment, when R^{c}, R^{c2}, R^{c3} and R^{c4} are independently C₆₋₂₀ aryl or C₆₋₂₀ aryl substituted with one or more R¹⁻¹⁻⁴, the C₆₋₂₀ aryl and the C₆₋₂₀ aryl in the C₆₋₂₀ aryl substituted with one or more R⁴⁻¹⁻⁴ are C₆₋₁₀ aryl, for example, phenyl or naphthyl.

In a certain embodiment, when R¹⁻¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R¹⁻¹⁻², R¹⁻¹⁻³, R⁴⁻¹⁴ and R⁴⁻¹⁻⁵ are independently halogen, the halogen is fluorine, chlorine, bromine or iodine, for example, fluorine.

In a certain embodiment, when R¹⁻¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R¹⁻¹⁻², R¹⁻¹⁻³, R⁴⁻¹⁴ and R⁴⁻¹⁻⁵ are independently C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl.

In a certain embodiment, when R¹⁻¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R¹⁻¹⁻², R¹⁻¹⁻³, R⁴⁻¹⁴ and R⁴⁻¹⁻⁵ are independently C₁₋₆ alkyl, the C₁₋₆ alkyl is methyl, ethyl, n-propyl, isopropyl, n-butyl, sec-butyl, isobutyl or tert-butyl.

In a certain embodiment, when R¹¹, R²¹, R²², R²³, R¹⁴, R²⁴, R¹⁵, R²⁵, R¹⁶ and R²⁶ are independently C₁₋₆ alkyl, the C₁₋₆ alkyl is methyl, ethyl, n-propyl, isopropyl, n-butyl, sec-butyl, isobutyl or tert-butyl.

In a certain embodiment, when L₂ is C₁₋₆ alkylene, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—.

In a certain embodiment, when R^{L-1}, R^{L-2} or R^{L-4} is independently C₁₋₆ alkylene, the C₁₋₆ alkylene is C₁₋₄ alkylene, for example, —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—, for another example, —CH₂—, —CH₂CH₂—, —CH₂CH₂CH₂—, —CH(CH₃)CH₂—.

In a certain embodiment, when R^{L-3} is C₁₋₆ alkyl, the C₁₋₆ alkyl is C₁₋₄ alkyl, for example, methyl, ethyl, n-propyl, isopropyl, n-butyl, sec-butyl, isobutyl or tert-butyl.

In a certain embodiment, when R³ is C₃₋₁₂ cycloalkyl or C₃₋₁₂ cycloalkyl substituted with one or more R³⁻¹, the C₃₋₁₂ cycloalkyl and the C₃₋₁₂ cycloalkyl in the C₃₋₁₂ cycloalkyl substituted with one or more R³⁻¹ is C₃₋₁₀ cycloalkyl, the C₃₋₁₂ cycloalkyl is a monocyclic alkyl, a bridged cycloalkyl or a spiral cycloalkyl, for example, cyclopropyl.

In a certain embodiment, when R³ is “4- to 12-membered heterocycloalkyl containing 1-3 heteroatoms independently selected from O, S and N”, the “4- to 12-membered heterocycloalkyl containing 1-3 heteroatoms independently selected from O, S and N” is “4- to 8-membered heterocycloalkyl containing 1-3 heteroatoms independently selected from O, S and N”, it can also be “5- to 8-membered heterocycloalkyl containing 1-2 heteroatoms independently selected from O and N”.

In a certain embodiment, when R³ is “4- to 12-membered heterocycloalkyl containing 1-3 heteroatoms independently selected from O, S and N”, the “4- to 12-membered heterocycloalkyl containing 1-3 heteroatoms independently selected from O, S and N” is a monocyclic heterocycloalkyl, a spiral heterocycloalkyl or a fused heterocycloalkyl; for example, azetidinyl, pyrrolidinyl, tetrahydrofuryl, hexahydro-1H-pyrrolizinyl, 7-azaspiro[3,5]nonanyl, 3-azaspiro[5,5]undecanyl, morphinyl or piperidinyl, for another example,

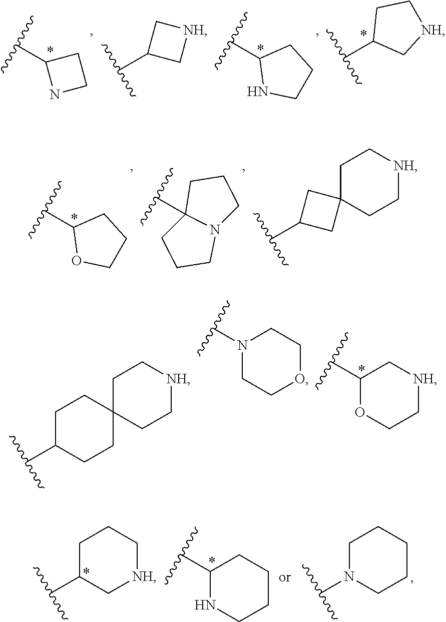

and yet for another example,

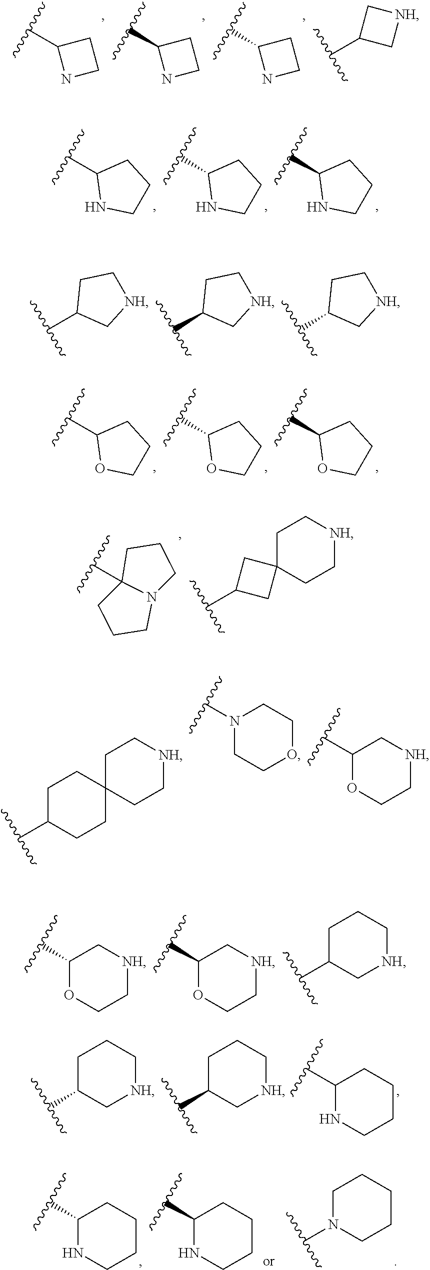

In a certain embodiment, when R³ is “4- to 12-membered heterocycloalkyl containing 1-3 heteroatoms independently selected from O, S and N” substituted with one or more R³⁻², the “4- to 12-membered heterocycloalkyl containing 1-3 heteroatoms independently selected from O, S and N” in the “4 to 12-membered heterocycloalkyl containing 1-3 heteroatoms independently selected from O, S and N” substituted with one or more R³⁻² is “5- to 11-membered heterocycloalkyl containing 1-3 heteroatoms independently selected from O, S and N”, for example, “5- to 11-membered heterocycloalkyl containing 1-2 heteroatoms independently selected from O and N”.

In a certain embodiment, when R³ is “4- to 12-membered heterocycloalkyl containing 1-3 heteroatoms independently selected from O, S and N” substituted with one or more R³⁻², the “4- to 12-membered heterocycloalkyl containing 1-3 heteroatoms independently selected from O, S and N” is a monocyclic heteroalkyl, a spiral heterocycloalkyl or fused heterocycloalkyl; the “4- to 12-membered heterocycloalkyl containing 1-3 heteroatoms independently selected from O, S and N” substituted with one or more R³⁻² is for example,

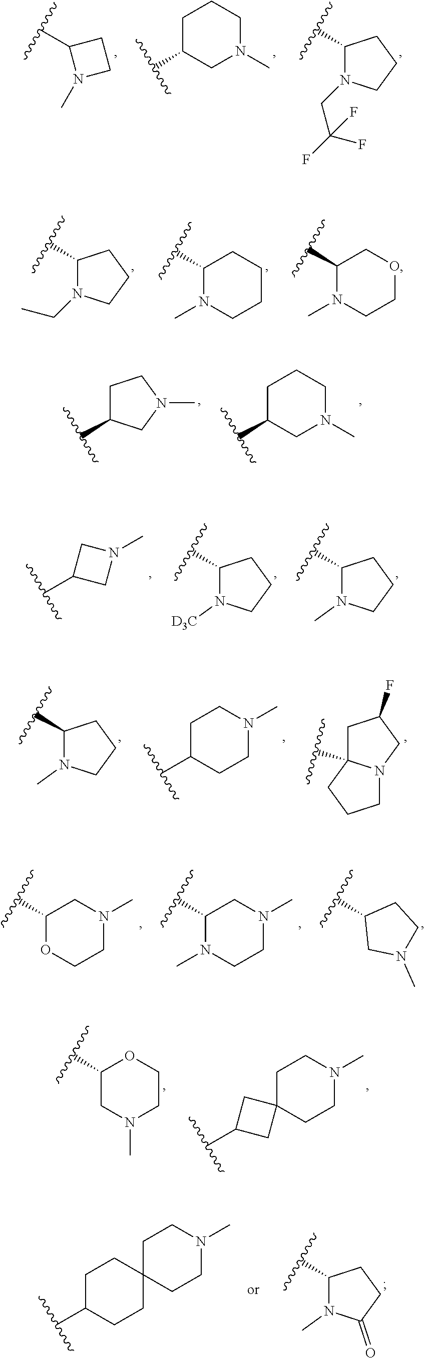

another example

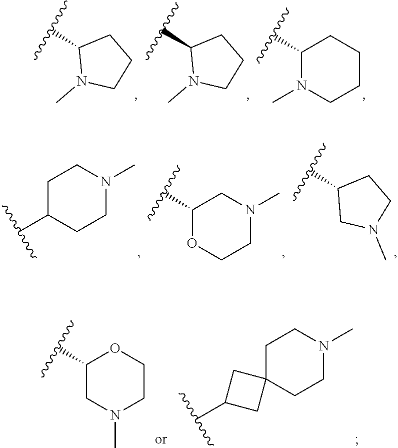

and yet for another example,

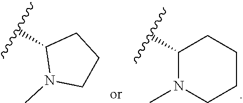

In a certain embodiment, when R³ is “5- to 6-membered heterocycloalkyl containing 1-2 heteroatoms independently selected from O and N” substituted with one or more R³⁻², the R³ is

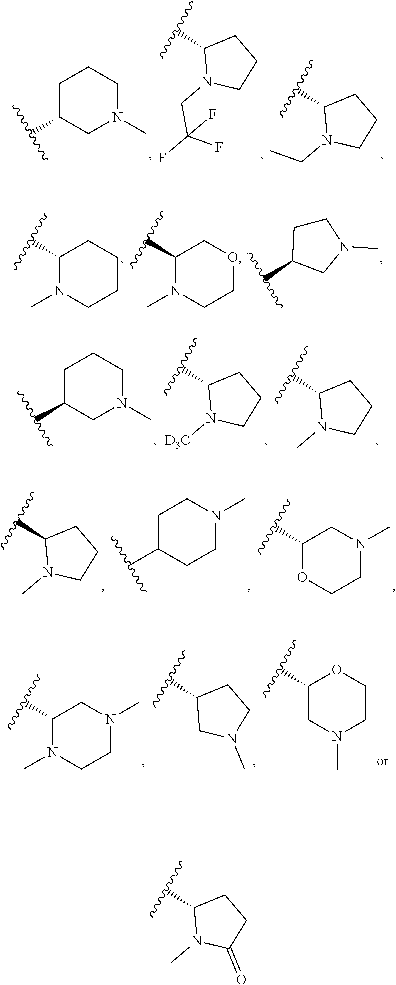

In a certain embodiment, when R³ is “5- to 6-membered heterocycloalkyl containing 1 heteroatom of N” substituted with one or more R³², the R³ is

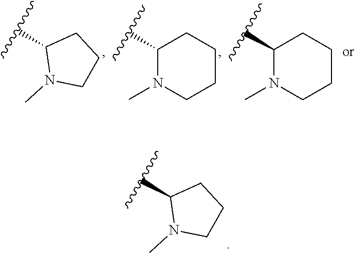

In a certain embodiment, when R³ is “5-membered heterocycloalkyl containing 1 heteroatom being N” substituted with one or more R³⁻², the R³ is

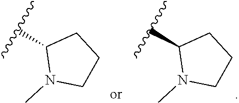

In a certain embodiment, when R³ is C₁₋₆ alkyl or C₁₋₆ alkyl substituted with one or more R³⁻³, the C₁₋₆ alkyl and the C₁₋₆ alkyl in the C₁₋₆ alkyl substituted with one or more R³⁻³ are C₁₋₄ alkyl, for example, methyl, ethyl, n-propyl, isopropyl, n-butyl, sec-butyl, isobutyl or tert-butyl, for another example, methyl.

In a certain embodiment, when R³⁻¹, R³⁻² and R³⁻³ are independently C₁₋₆ alkyl or C₁₋₆ alkyl substituted with one or more R³⁻¹, the C₁₋₆ alkyl and the C₁₋₆ alkyl in the C₁₋₆ alkyl substituted with one or more R³⁻¹⁻¹ are C₁₋₄ alkyl, for example, methyl, ethyl, n-propyl, isopropyl, n-butyl, sec-butyl, isobutyl or tert-butyl, for another example, methyl.

In a certain embodiment, when R³⁻¹, R³⁻² and R³⁻³ are independently C₁₋₆ alkyl-O—, the C₁₋₆ alkyl in the C₁₋₆ alkyl-O— is C₁₋₄ alkyl, for example, methyl, ethyl, n-propyl, isopropyl, n-butyl, sec-butyl, isobutyl or tert-butyl, for another example, methyl.

In a certain embodiment, when R³⁻¹, R³⁻² and R³⁻³ are independently halogen, the halogen is fluorine, chlorine, bromine or iodine, for example, fluorine.

In a certain embodiment, when R^{d}, R^{d1}, R^{e1}, R^{e2}, R^{e3} and R^{e4} are independently C₁₋₆ alkyl or C₁₋₆ alkyl substituted with one or more R³⁻¹⁻², the C₁₋₆ alkyl and the C₁₋₆ alkyl in the C₁₋₆ alkyl substituted with one or more R³⁻¹⁻² are C₁₋₄ alkyl, for example, methyl, ethyl, n-propyl, isopropyl, n-butyl, sec-butyl, isobutyl or tert-butyl, for another example, methyl.

In a certain embodiment, when R^{d}, R^{d1}, R^{e1}, R^{e2}, R^{e3} and R^{e4} are independently C₃₋₁₀ cycloalkyl, the C₃₋₁₀ cycloalkyl is C₃₋₆ cycloalkyl, for example, cyclohexyl, cyclopentyl, cyclobutyl or cyclopropyl, for another example, cyclopropyl, for another example, cyclopropyl.

In a certain embodiment, when R^{d}, R^{d1}, R^{e1}, R^{e2}, R^{e3} and R^{e4} are independently “4- to 10-membered heterocycloalkyl containing 1-3 heteroatoms independently selected from O and N”, the “4- to 10-membered heterocycloalkyl containing 1-3 heteroatoms independently selected from O and N” is “4- to 6-membered heterocycloalkyl containing 1-3 heteroatoms independently selected from O and N”, for example, “4- to 6-membered heterocycloalkyl containing 1-2 heteroatoms independently selected from O and N”.

In a certain embodiment, when R³⁻¹⁻¹ and R³⁻¹⁻² are independently halogen, the halogen is fluorine, chlorine, bromine or iodine.

In a certain embodiment, when R³⁻¹⁻¹ and R³⁻¹⁻² are independently C₁₋₆ alkyl-O—, the C₁₋₆ alkyl in the C₁₋₆ alkyl-O— is C₁₋₄ alkyl, for example, methyl, ethyl, n-propyl, isopropyl, n-butyl, sec-butyl, isobutyl or tert-butyl.

In a certain embodiment, when R^{e5}, R^{e6}, R^{e7}, R^{e8}, R^{e9}, R^{e10}, R^{e11}, R^{e12}, R^{e13} and R^{e14} are independently C₁₋₆ alkyl, the C₁₋₆ alkyl is C₁₋₄ alkyl, for example, methyl, ethyl, n-propyl, isopropyl, n-butyl, sec-butyl, isobutyl or tert-butyl.

In a certain embodiment,

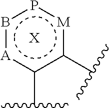

is

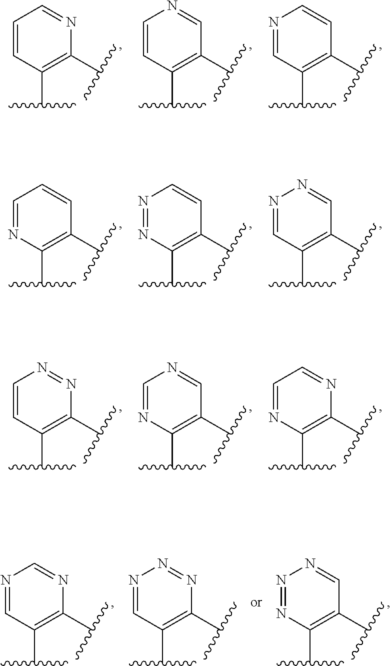

for example,

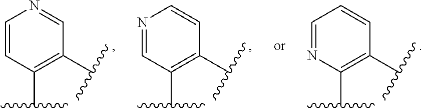 [SMILES: *c1ccncc1* ; *c1ccncc1* ; *c1cccnc1*]

In a certain embodiment, R⁴ is —CH₃,

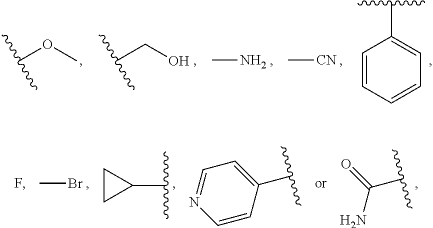

for example,

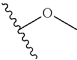 [SMILES: *OC]

### or F.

In a certain embodiment, R⁴ is —CH₃,

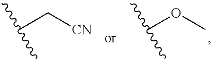

for example, —CH₃.

In a certain embodiment,

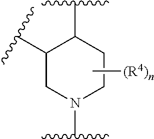

is

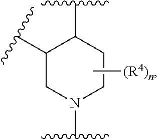

for example,

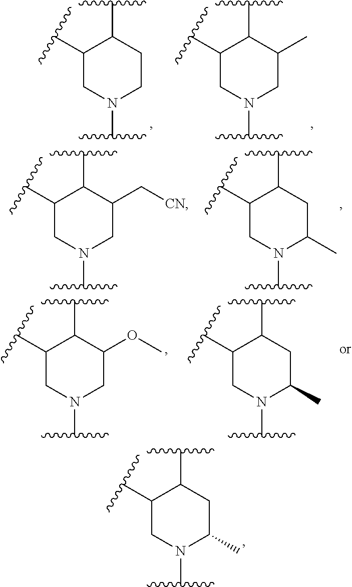

for another example

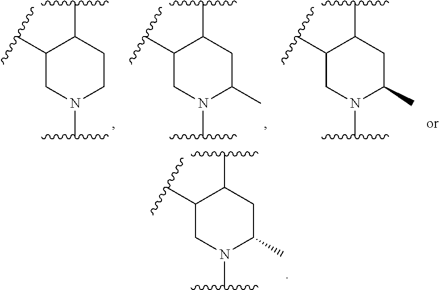

In a certain embodiment,

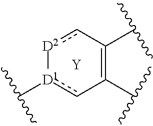

is

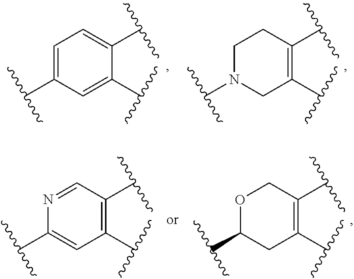 [SMILES: *c1ccc(*)c(*)c1 ; *C1=C(*)CN(*)CC1 ; *c1cc(*)c(*)cn1 ; *C1=C(*)C[C@@H](*)OC1]

for example

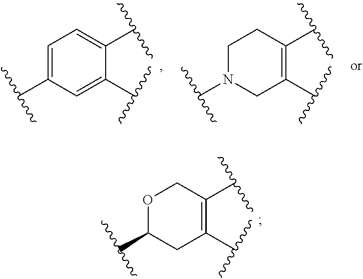 [SMILES: *c1ccc(*)c(*)c1 ; *C1=C(*)CN(*)CC1 ; *C1=C(*)C[C@@H](*)OC1]

for another example

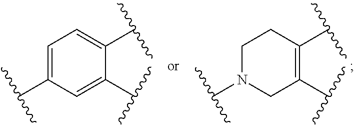 [SMILES: *c1ccc(*)c(*)c1 ; *C1=C(*)CN(*)CC1]

and yet for another example

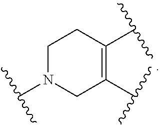 [SMILES: *C1=C(*)CN(*)CC1]

In a certain embodiment,

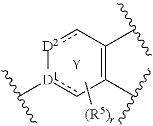

is

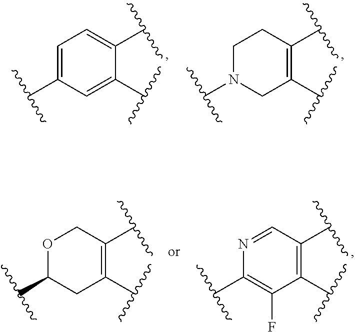 [SMILES: *c1ccc(*)c(*)c1 ; *C1=C(*)CN(*)CC1 ; *C1=C(*)C[C@@H](*)OC1 ; *c1cnc(*)c(F)c1*]

for example

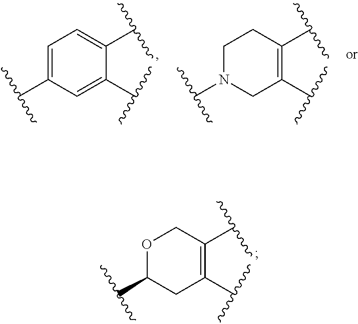 [SMILES: *c1ccc(*)c(*)c1 ; *C1=C(*)CN(*)CC1 ; *C1=C(*)C[C@@H](*)OC1]

for another example

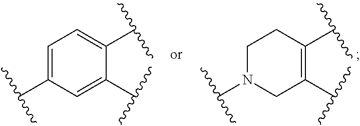 [SMILES: *c1ccc(*)c(*)c1 ; *C1=C(*)CN(*)CC1]

and yet for another example,

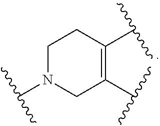 [SMILES: *C1=C(*)CN(*)CC1]

In a certain embodiment,

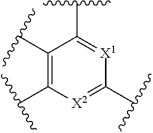

is

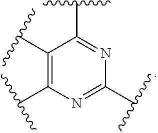 [SMILES: *c1nc(*)c(*)c(*)n1]

In a certain embodiment,

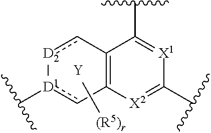

is

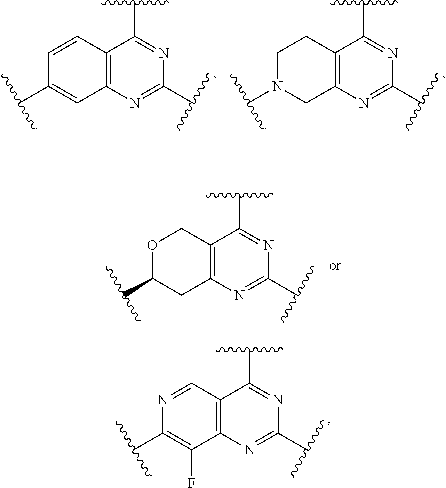 [SMILES: *c1ccc2c(*)nc(*)nc2c1 ; *c1nc(*)c2c(n1)CN(*)CC2 ; *c1nc(*)c2c(n1)C[C@@H](*)OC2 ; *c1nc(*)c2cnc(*)c(F)c2n1]

for example, phenyl,

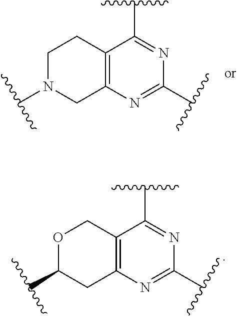 [SMILES: *c1nc(*)c2c(n1)CN(*)CC2 ; *c1nc(*)c2c(n1)C[C@@H](*)OC2]

In a certain embodiment, R^{b}, R¹⁻¹ and R¹⁻² are independently hydroxyl, amino, ethynyl, F, Cl, CF₃, methyl, methyl-O—, cyclopropyl or

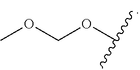 [SMILES: *OCOC]

In a certain embodiment, R¹⁻¹ is hydroxyl, amino, ethynyl, F, Cl or

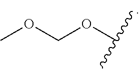 [SMILES: *OCOC]

In a certain embodiment, R¹ is

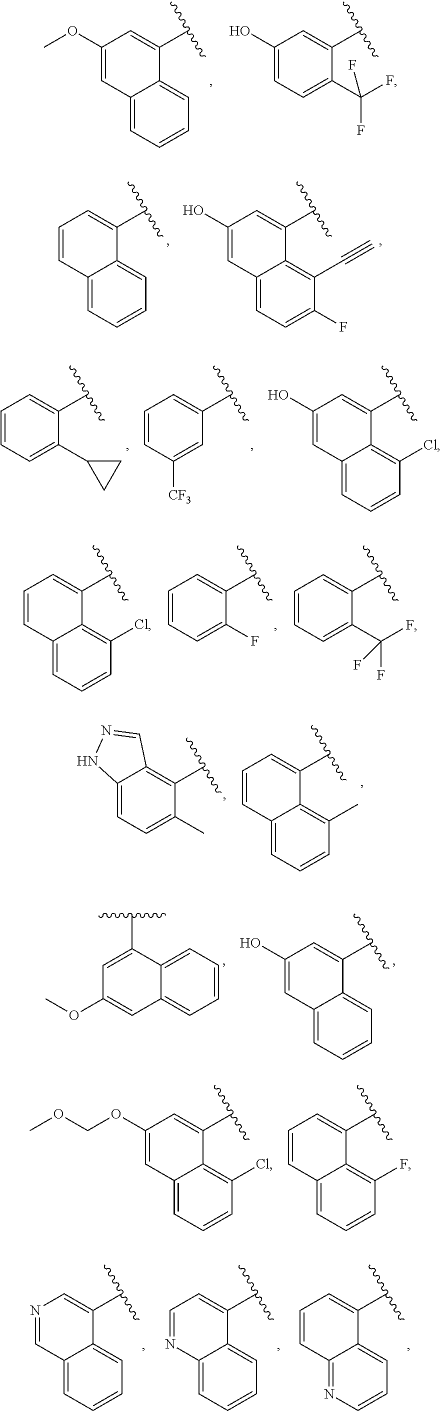

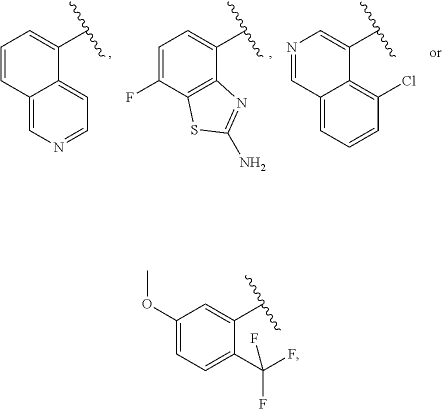

for example

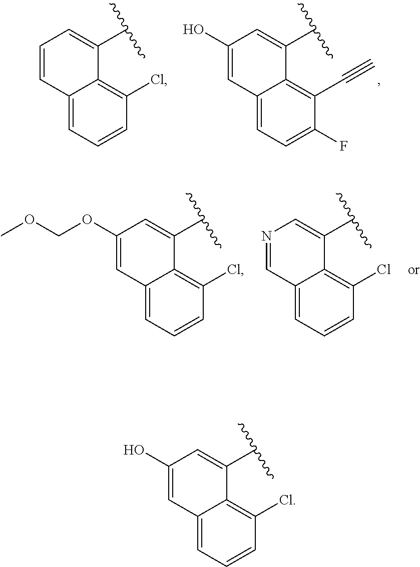

In a certain embodiment,

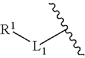

is

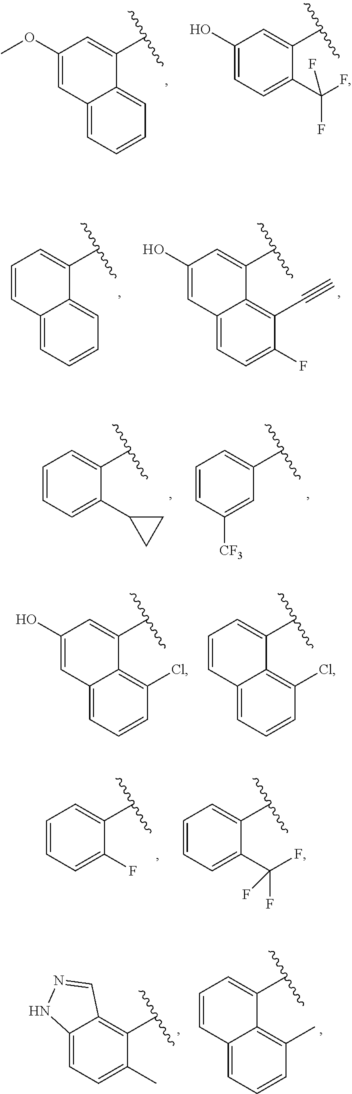

for example

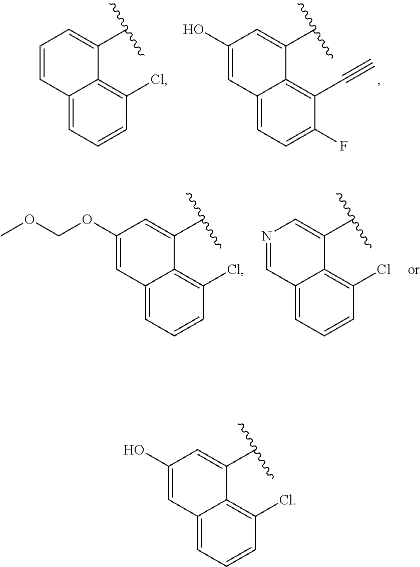

In a certain embodiment, L₂ is

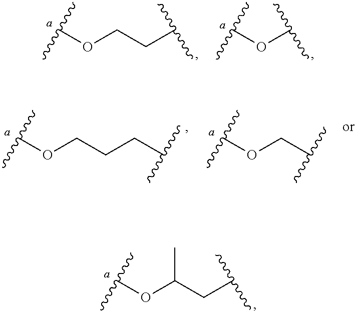

wherein position a is connected with position b of

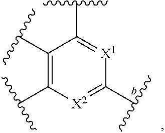

for example,

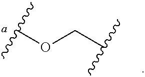 [SMILES: *CO*]

In a certain embodiment, R³⁻¹⁻¹ and R³⁻¹⁻² are independently deuterium (D), F or —N(CH₃)₂.

In a certain embodiment, R³⁻¹, R³⁻² and R³⁻³ are independently F, O═,

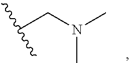 [SMILES: *CN(C)C]

methyl, ethyl,

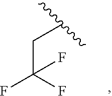 [SMILES: *CC(F)(F)F]

—CD₃, —N(CH₃)₂, for example, methyl.

In a certain embodiment, R³ is methyl,

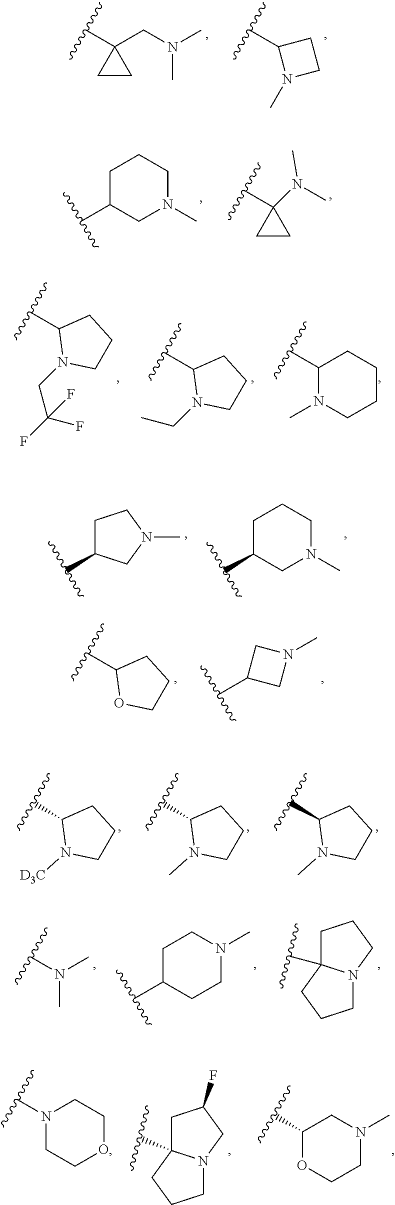

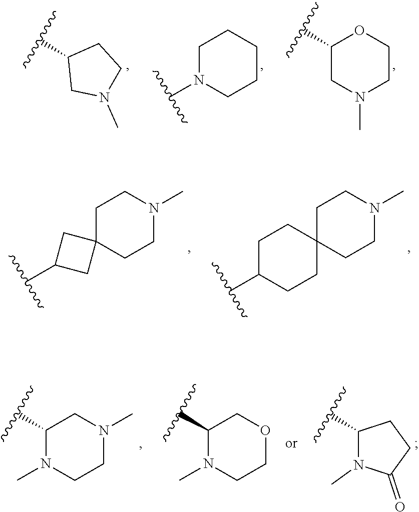

for example

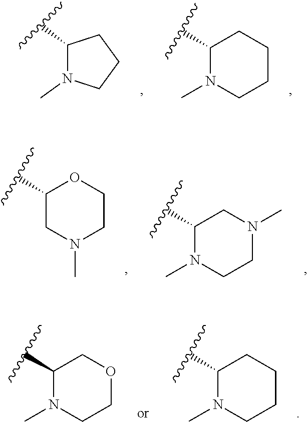

In a certain embodiment,

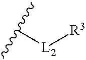

is

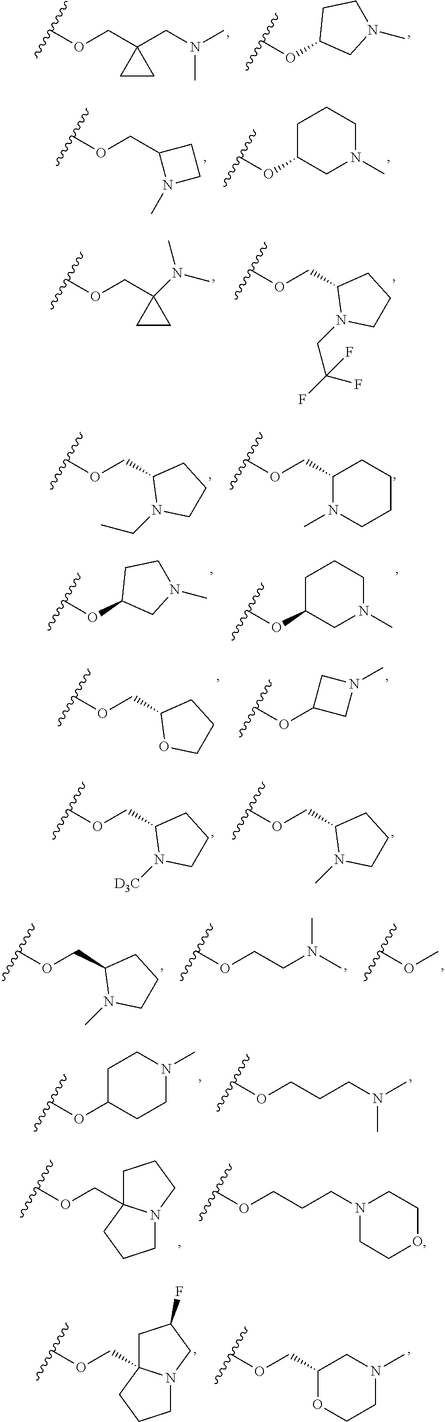

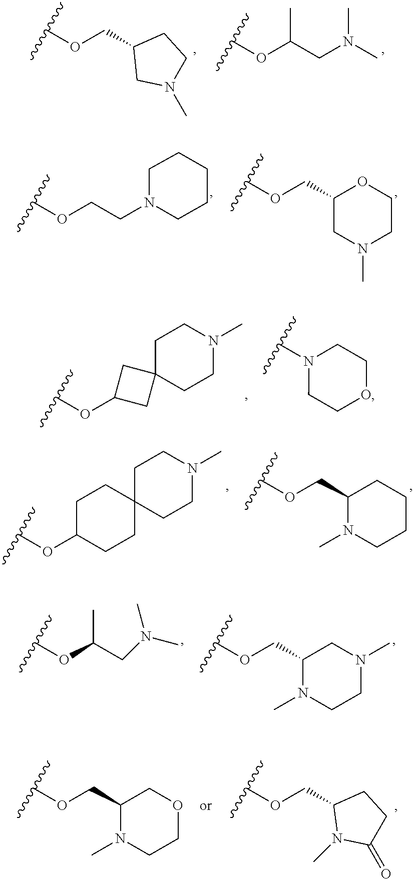

for example,

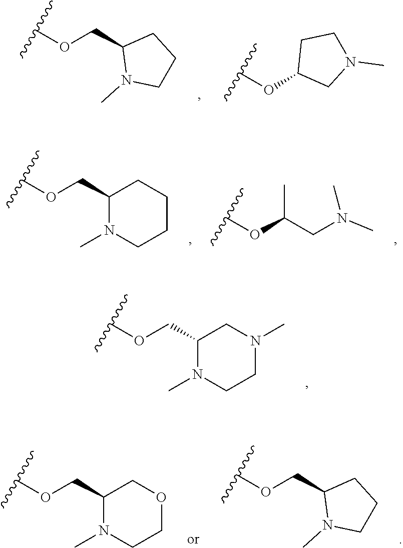

In a certain embodiment, the nitrogen-containing heterocyclic compound of formula I has any one of the following structures:

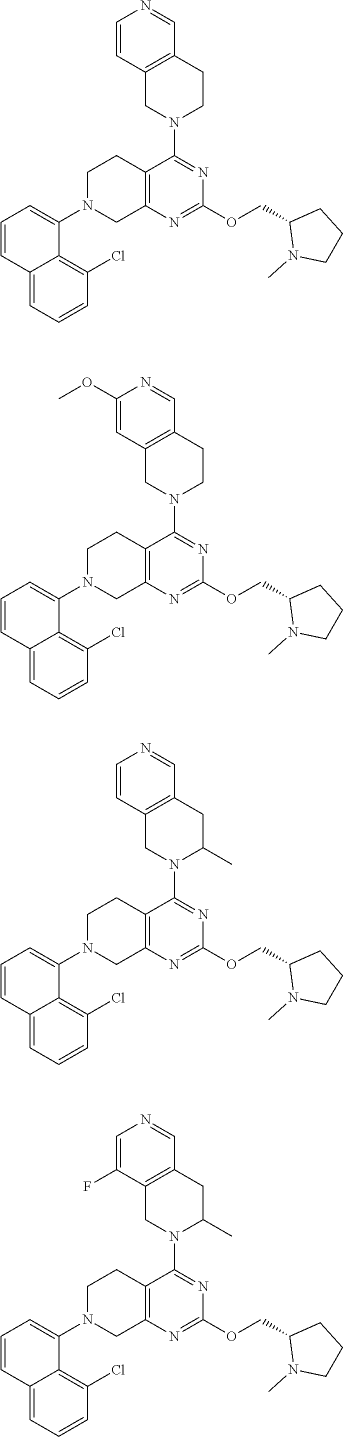 [SMILES: CN1CCC[C@H]1COc1nc2c(c(N3CCc4cnccc4C3)n1)CCN(c1cccc3cccc(Cl)c13)C2 ; COc1cc2c(cn1)CCN(c1nc(OC[C@@H]3CCCN3C)nc3c1CCN(c1cccc4cccc(Cl)c14)C3)C2 ; CC1Cc2cnccc2CN1c1nc(OC[C@@H]2CCCN2C)nc2c1CCN(c1cccc3cccc(Cl)c13)C2 ; CC1Cc2cncc(F)c2CN1c1nc(OC[C@@H]2CCCN2C)nc2c1CCN(c1cccc3cccc(Cl)c13)C2]

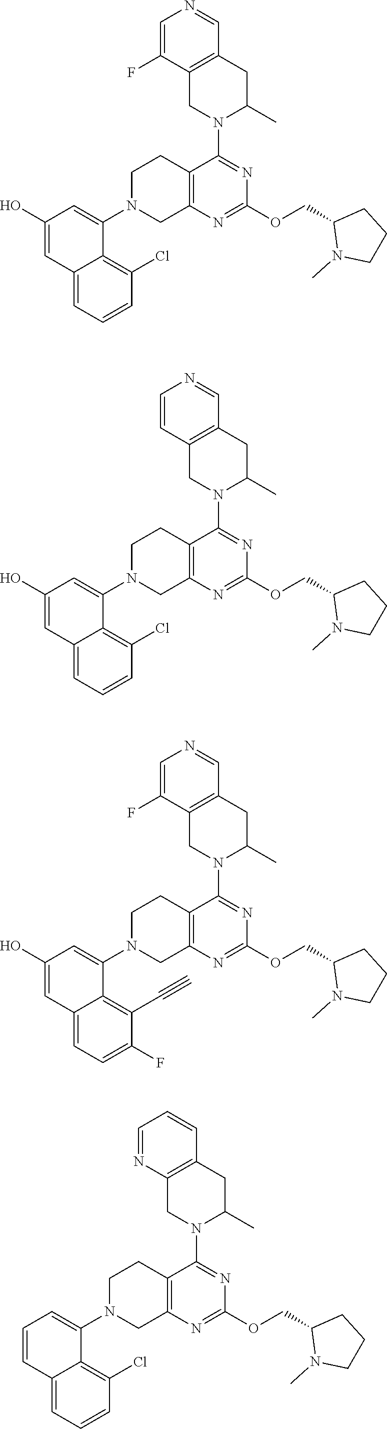 [SMILES: CC1Cc2cncc(F)c2CN1c1nc(OC[C@@H]2CCCN2C)nc2c1CCN(c1cc(O)cc3cccc(Cl)c13)C2 ; CC1Cc2cnccc2CN1c1nc(OC[C@@H]2CCCN2C)nc2c1CCN(c1cc(O)cc3cccc(Cl)c13)C2 ; C#Cc1c(F)ccc2cc(O)cc(N3CCc4c(nc(OC[C@@H]5CCCN5C)nc4N4Cc5c(F)cncc5CC4C)C3)c12 ; CC1Cc2cccnc2CN1c1nc(OC[C@@H]2CCCN2C)nc2c1CCN(c1cccc3cccc(Cl)c13)C2]

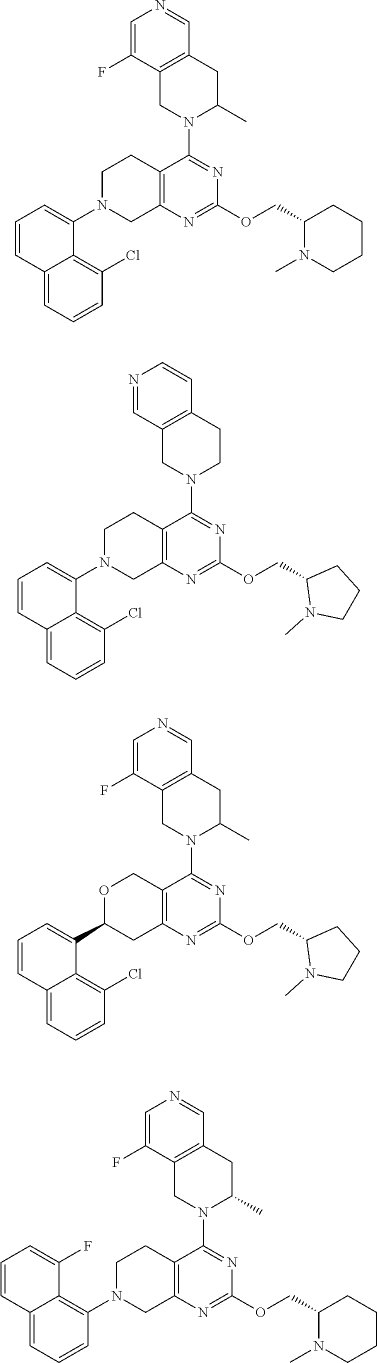 [SMILES: CC1Cc2cncc(F)c2CN1c1nc(OC[C@@H]2CCCCN2C)nc2c1CCN(c1cccc3cccc(Cl)c13)C2 ; CN1CCC[C@H]1COc1nc2c(c(N3CCc4ccncc4C3)n1)CCN(c1cccc3cccc(Cl)c13)C2 ; CC1Cc2cncc(F)c2CN1c1nc(OC[C@@H]2CCCN2C)nc2c1CO[C@H](c1cccc3cccc(Cl)c13)C2 ; C[C@H]1Cc2cncc(F)c2CN1c1nc(OC[C@@H]2CCCCN2C)nc2c1CCN(c1cccc3cccc(F)c13)C2]

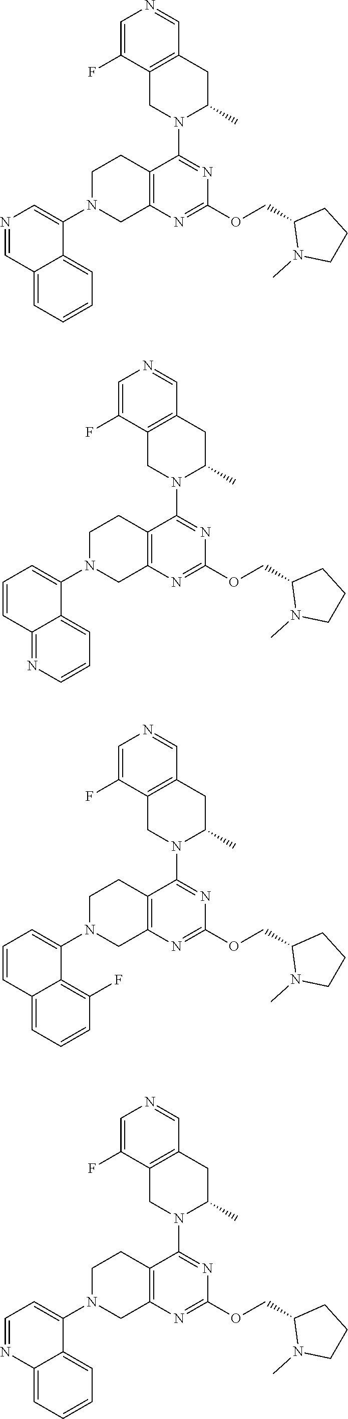 [SMILES: C[C@H]1Cc2cncc(F)c2CN1c1nc(OC[C@@H]2CCCN2C)nc2c1CCN(c1cncc3ccccc13)C2 ; C[C@H]1Cc2cncc(F)c2CN1c1nc(OC[C@@H]2CCCN2C)nc2c1CCN(c1cccc3ncccc13)C2 ; C[C@H]1Cc2cncc(F)c2CN1c1nc(OC[C@@H]2CCCN2C)nc2c1CCN(c1cccc3cccc(F)c13)C2 ; C[C@H]1Cc2cncc(F)c2CN1c1nc(OC[C@@H]2CCCN2C)nc2c1CCN(c1ccnc3ccccc13)C2]

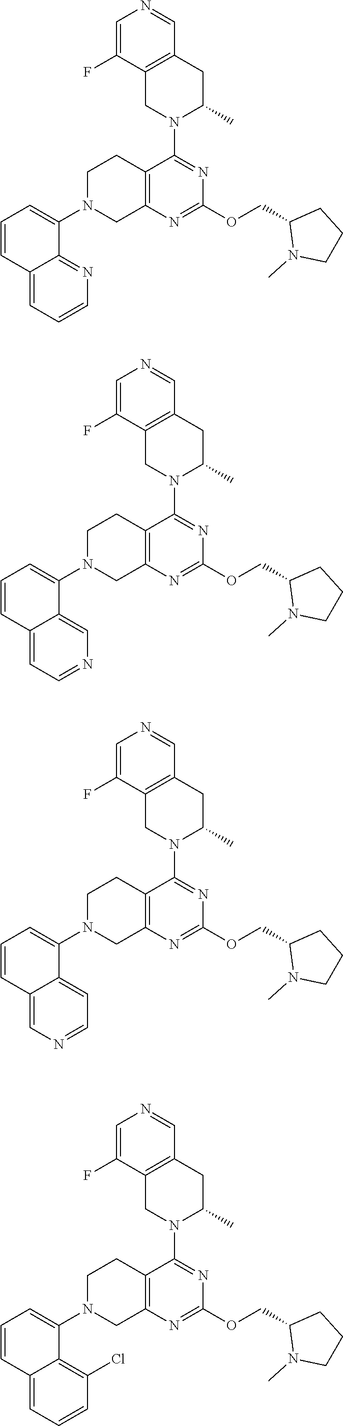 [SMILES: C[C@H]1Cc2cncc(F)c2CN1c1nc(OC[C@@H]2CCCN2C)nc2c1CCN(c1cccc3cccnc13)C2 ; C[C@H]1Cc2cncc(F)c2CN1c1nc(OC[C@@H]2CCCN2C)nc2c1CCN(c1cccc3ccncc13)C2 ; C[C@H]1Cc2cncc(F)c2CN1c1nc(OC[C@@H]2CCCN2C)nc2c1CCN(c1cccc3cnccc13)C2 ; C[C@H]1Cc2cncc(F)c2CN1c1nc(OC[C@@H]2CCCN2C)nc2c1CCN(c1cccc3cccc(Cl)c13)C2]

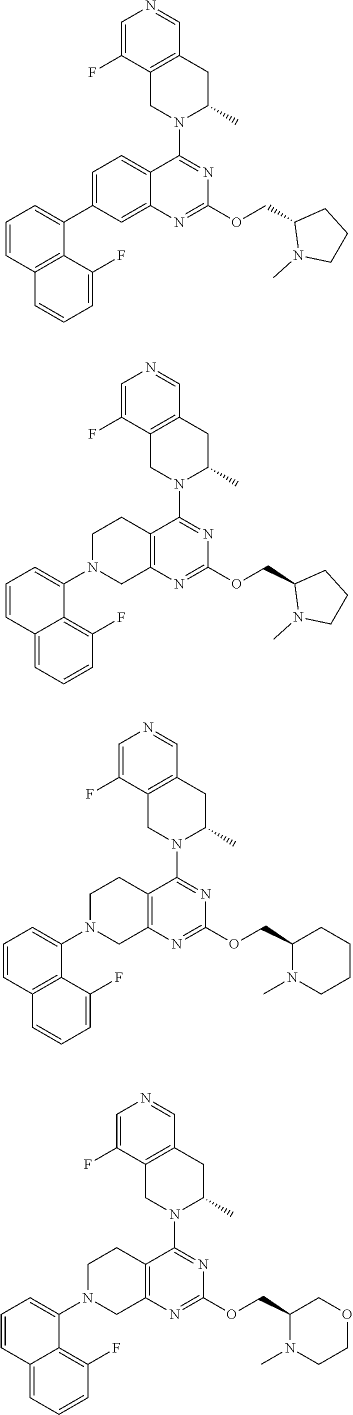 [SMILES: C[C@H]1Cc2cncc(F)c2CN1c1nc(OC[C@@H]2CCCN2C)nc2cc(-c3cccc4cccc(F)c34)ccc12 ; C[C@H]1Cc2cncc(F)c2CN1c1nc(OC[C@H]2CCCN2C)nc2c1CCN(c1cccc3cccc(F)c13)C2 ; C[C@H]1Cc2cncc(F)c2CN1c1nc(OC[C@H]2CCCCN2C)nc2c1CCN(c1cccc3cccc(F)c13)C2 ; C[C@H]1Cc2cncc(F)c2CN1c1nc(OC[C@H]2COCCN2C)nc2c1CCN(c1cccc3cccc(F)c13)C2]

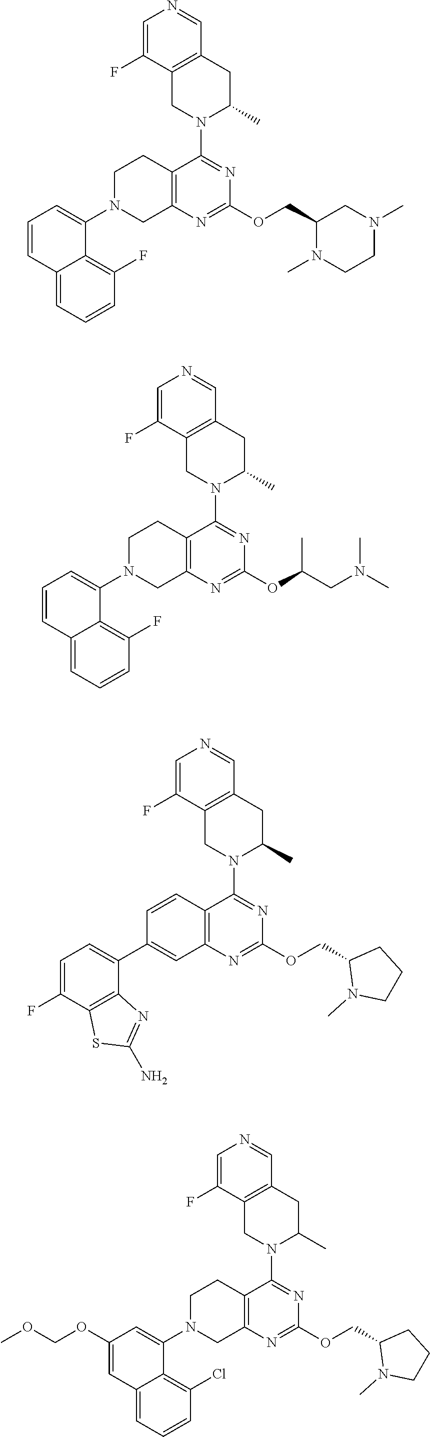

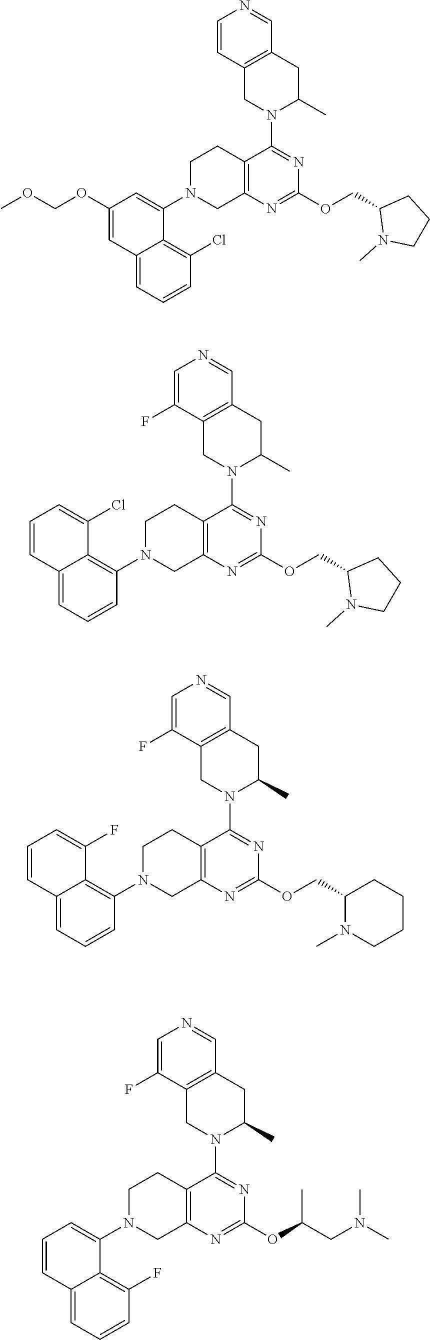 [SMILES: COCOc1cc(N2CCc3c(nc(OC[C@@H]4CCCN4C)nc3N3Cc4ccncc4CC3C)C2)c2c(Cl)cccc2c1 ; CC1Cc2cncc(F)c2CN1c1nc(OC[C@@H]2CCCN2C)nc2c1CCN(c1cccc3cccc(Cl)c13)C2 ; C[C@@H]1Cc2cncc(F)c2CN1c1nc(OC[C@@H]2CCCCN2C)nc2c1CCN(c1cccc3cccc(F)c13)C2 ; C[C@@H]1Cc2cncc(F)c2CN1c1nc(O[C@@H](C)CN(C)C)nc2c1CCN(c1cccc3cccc(F)c13)C2]

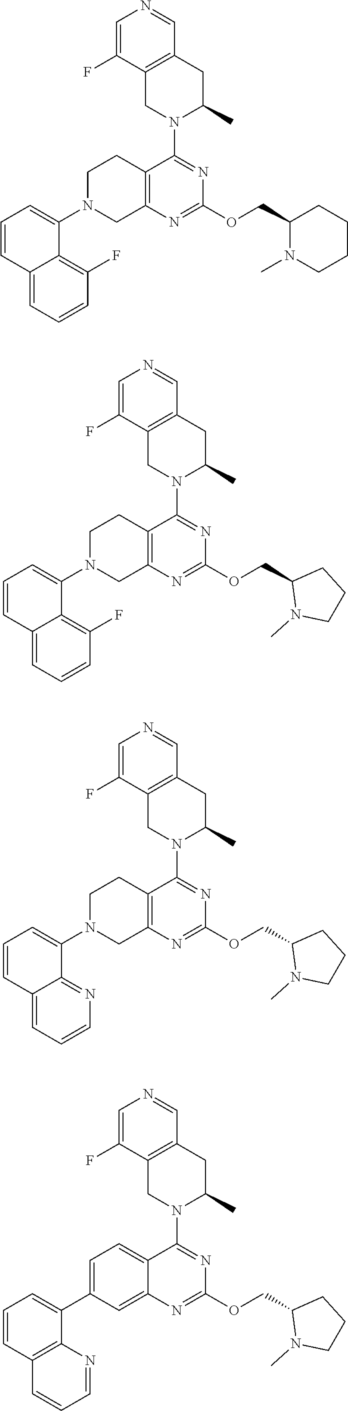 [SMILES: C[C@@H]1Cc2cncc(F)c2CN1c1nc(OC[C@H]2CCCCN2C)nc2c1CCN(c1cccc3cccc(F)c13)C2 ; C[C@@H]1Cc2cncc(F)c2CN1c1nc(OC[C@H]2CCCN2C)nc2c1CCN(c1cccc3cccc(F)c13)C2 ; C[C@@H]1Cc2cncc(F)c2CN1c1nc(OC[C@@H]2CCCN2C)nc2c1CCN(c1cccc3cccnc13)C2 ; C[C@@H]1Cc2cncc(F)c2CN1c1nc(OC[C@@H]2CCCN2C)nc2cc(-c3cccc4cccnc34)ccc12]

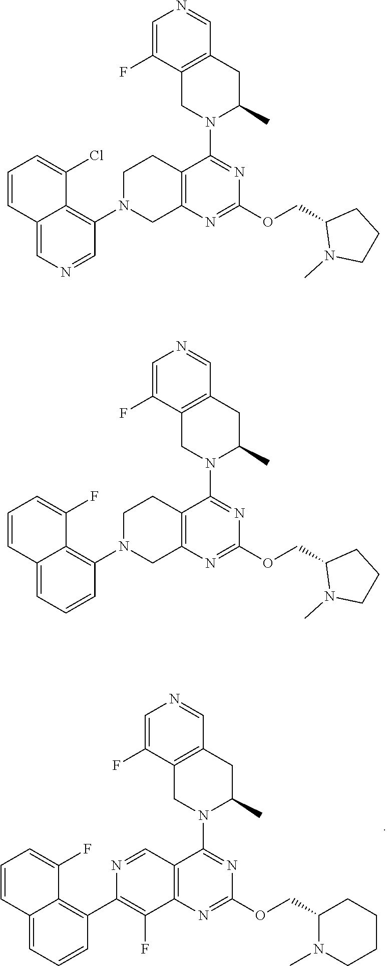 [SMILES: C[C@@H]1Cc2cncc(F)c2CN1c1nc(OC[C@@H]2CCCN2C)nc2c1CCN(c1cncc3cccc(Cl)c13)C2 ; C[C@@H]1Cc2cncc(F)c2CN1c1nc(OC[C@@H]2CCCN2C)nc2c1CCN(c1cccc3cccc(F)c13)C2 ; C[C@@H]1Cc2cncc(F)c2CN1c1nc(OC[C@@H]2CCCCN2C)nc2c(F)c(-c3cccc4cccc(F)c34)ncc12]

In a certain embodiment, the pharmaceutically acceptable salt of the nitrogen-containing heterocyclic compound of formula I is trifluoroacetate having any one of the following structures:

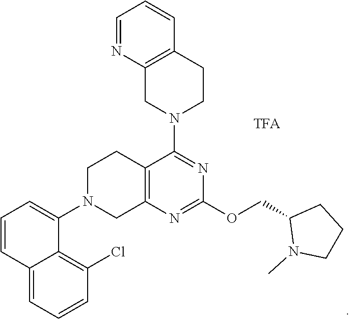 [SMILES: CN1CCC[C@H]1COc1nc2c(c(N3CCc4cccnc4C3)n1)CCN(c1cccc3cccc(Cl)c13)C2]

In a certain embodiment, the nitrogen-containing heterocyclic compound of formula I has any one of the following structures:

compound

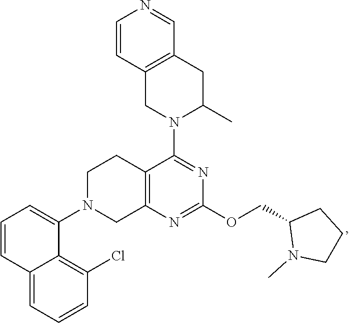 [SMILES: CC1Cc2cnccc2CN1c1nc(OC[C@@H]2CCCN2C)nc2c1CCN(c1cccc3cccc(Cl)c13)C2]

which has a retention time of 2.10 min under the following conditions: instrument: UPCC; chromatographic column: IH 4.6*100 mm, 5 um (Daicel); column temperature: 40° C.; mobile phase: CO₂/MeOH[0.2% NH₃(7M in MeOH)]=75/25; flow rate: 3.0 mL/min; wavelength: 214 n; back pressure: 2000 psi;

compound

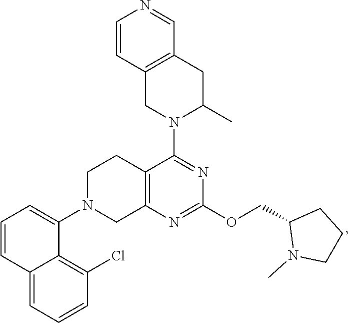 [SMILES: CC1Cc2cnccc2CN1c1nc(OC[C@@H]2CCCN2C)nc2c1CCN(c1cccc3cccc(Cl)c13)C2]

which has a retention time of 2.66 min under the following conditions: instrument: UPCC; chromatographic column: IH 4.6*100 mm, 5 um (Daicel); column temperature: 40° C.; mobile phase: CO₂/MeOH[0.2% NH₃(7M in MeOH)]=75/25; flow rate: 3.0 mL/min; wavelength: 214 n; back pressure: 2000 psi;

compound

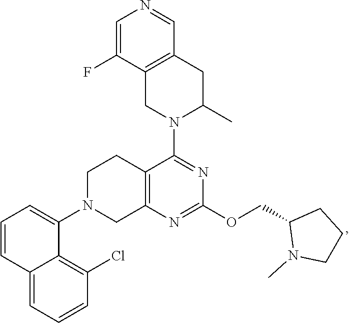 [SMILES: CC1Cc2cncc(F)c2CN1c1nc(OC[C@@H]2CCCN2C)nc2c1CCN(c1cccc3cccc(Cl)c13)C2]

which has a retention time of 15.7 min under the following conditions: instrument: Agilent1200; chromatographic column: IG 4.6*250 mm, 5 um (Daicel); column temperature: 40° C.; mobile phase: n-Hexane (0.1% DEA EtOH (0.1% DEA)=70:30; flow rate: 1.0 m/min; wavelength: 254 nm&214 nm;

compound

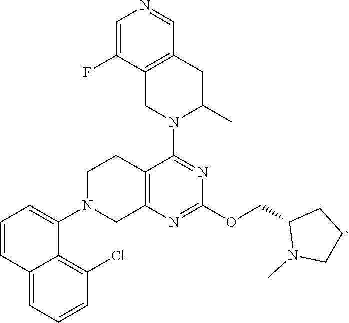 [SMILES: CC1Cc2cncc(F)c2CN1c1nc(OC[C@@H]2CCCN2C)nc2c1CCN(c1cccc3cccc(Cl)c13)C2]

which has a retention time of 20.39 min under the following conditions: instrument: Agilent 1200; chromatographic column: IG 4.6*250 mm, 5 um (Daicel); column temperature: 40° C.; mobile phase: n-Hexane (0.1% DEA):EtOH (0.1% DEA)=70:30; flow rate: 1.0 mL/min; wavelength: 254 nm&214 nm;

compound

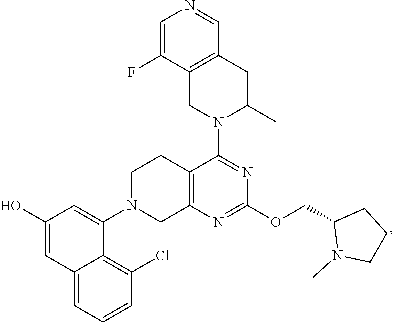 [SMILES: CC1Cc2cncc(F)c2CN1c1nc(OC[C@@H]2CCCN2C)nc2c1CCN(c1cc(O)cc3cccc(Cl)c13)C2]

which has a retention time of 1.25 min under the following conditions: instrument: UPCC; chromatographic column: AD-3 4.6*100 mm, 3 μm (Daicel); column temperature: 40° C.; mobile phase: CO₂/IPA[1% NH₃(7M in MeOH)]=55/45; flow rate: 3.0 m/min; wavelength: 214 nm; back pressure: 2000 psi;

compound

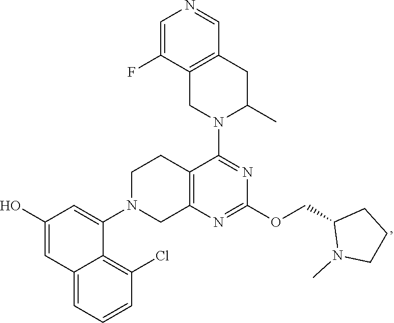 [SMILES: CC1Cc2cncc(F)c2CN1c1nc(OC[C@@H]2CCCN2C)nc2c1CCN(c1cc(O)cc3cccc(Cl)c13)C2]

which has a retention time of 1.99 min under the following conditions: instrument: UPCC; chromatographic column: AD-3 4.6*100 mm, 3 μm (Daicel); column temperature: 40° C.; mobile phase: CO₂/IPA[1% NH₃(7M in MeOH)]=55/45; flow rate: 3.0 m/min; wavelength: 214 nm; back pressure: 2000 psi;

compound

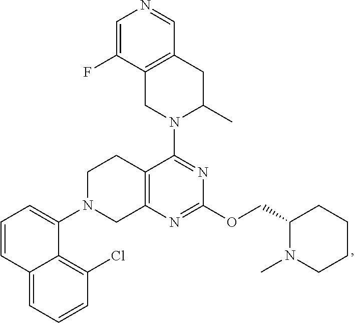 [SMILES: CC1Cc2cncc(F)c2CN1c1nc(OC[C@@H]2CCCCN2C)nc2c1CCN(c1cccc3cccc(Cl)c13)C2]

which has a retention time of 2.20 min under the following conditions: instrument: UPCC; chromatographic column: AS-H 4.6*100 mm, 5 μm (Daicel); column temperature: 40° C.; mobile phase: CO₂/EtOH [1% NH₃(7M in MeOH)]=80/20; flow rate: 3.0 m/min; wavelength: 214 nm; back pressure: 2000 psi;

compound

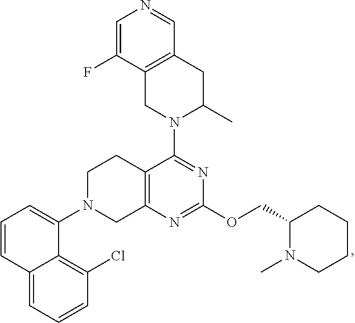 [SMILES: CC1Cc2cncc(F)c2CN1c1nc(OC[C@@H]2CCCCN2C)nc2c1CCN(c1cccc3cccc(Cl)c13)C2]

which has a retention time of 2.89 min under the following conditions: instrument: UPCC; chromatographic column: AS-H 4.6*100 mm, 5 μm (Daicel); column temperature: 40° C.; mobile phase: CO₂/EtOH [1% NH₃(7M in MeOH)]=80/20; flow rate: 3.0 m/min; wavelength: 214 nm; back pressure: 2000 psi;

compound

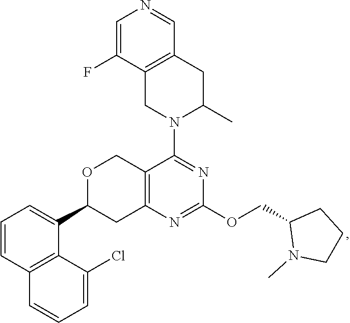 [SMILES: CC1Cc2cncc(F)c2CN1c1nc(OC[C@@H]2CCCN2C)nc2c1CO[C@H](c1cccc3cccc(Cl)c13)C2]

which has a retention time of 0.875 min under the following conditions: instrument: UPCC; chromatographic column: OJ-3 4.6*100 mm, 3 um (Daicel); column temperature: 40° C.; mobile phase: CO₂/Methanol[0.2% NH₃(7M in Methanol)]=55/45; flow rate: 3.0 m/min; wavelength: 214 nm; back pressure: 2000 psi;

compound

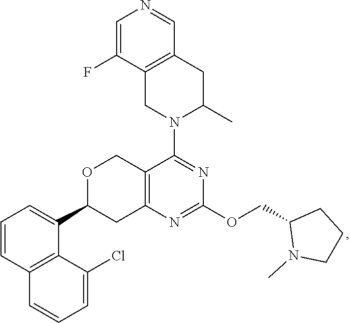 [SMILES: CC1Cc2cncc(F)c2CN1c1nc(OC[C@@H]2CCCN2C)nc2c1CO[C@H](c1cccc3cccc(Cl)c13)C2]

which has a retention time of 1.881 min under the following conditions: instrument: UPCC; chromatographic column: OJ-3 4.6*100 mm, 3 um (Daicel); column temperature: 40° C.; mobile phase: CO₂/Methanol[0.2% NH₃(7M in Methanol)]=55/45; flow rate: 3.0 mL/min; wavelength: 214 nm; back pressure: 2000 psi.

In a certain embodiment, the nitrogen-containing heterocyclic compound of formula I has any one of the following structures:

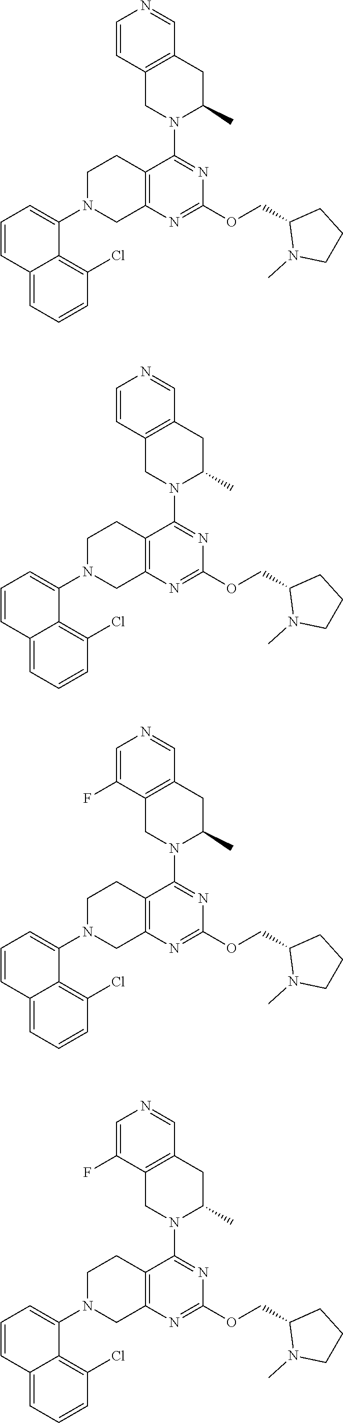 [SMILES: C[C@@H]1Cc2cnccc2CN1c1nc(OC[C@@H]2CCCN2C)nc2c1CCN(c1cccc3cccc(Cl)c13)C2 ; C[C@H]1Cc2cnccc2CN1c1nc(OC[C@@H]2CCCN2C)nc2c1CCN(c1cccc3cccc(Cl)c13)C2 ; C[C@@H]1Cc2cncc(F)c2CN1c1nc(OC[C@@H]2CCCN2C)nc2c1CCN(c1cccc3cccc(Cl)c13)C2 ; C[C@H]1Cc2cncc(F)c2CN1c1nc(OC[C@@H]2CCCN2C)nc2c1CCN(c1cccc3cccc(Cl)c13)C2]

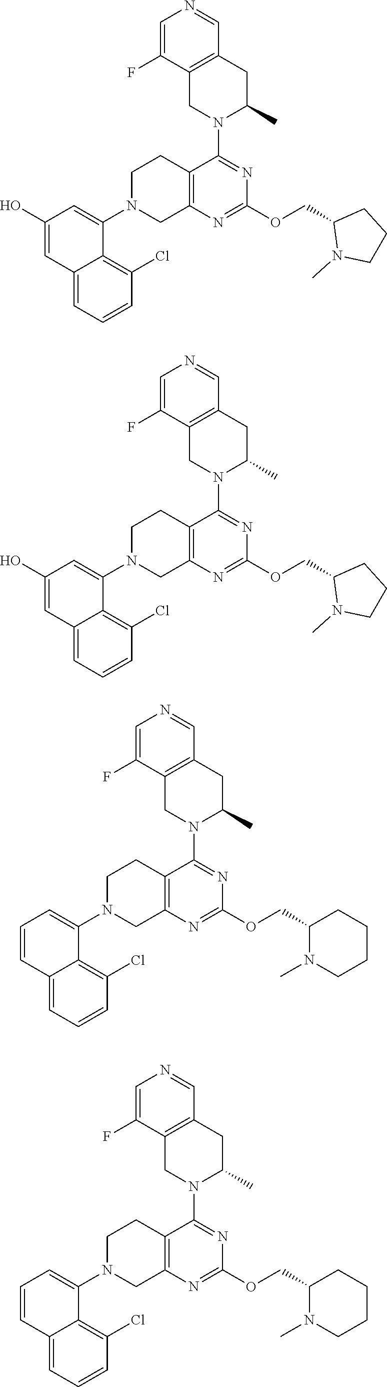 [SMILES: C[C@@H]1Cc2cncc(F)c2CN1c1nc(OC[C@@H]2CCCN2C)nc2c1CCN(c1cc(O)cc3cccc(Cl)c13)C2 ; C[C@H]1Cc2cncc(F)c2CN1c1nc(OC[C@@H]2CCCN2C)nc2c1CCN(c1cc(O)cc3cccc(Cl)c13)C2 ; C[C@@H]1Cc2cncc(F)c2CN1c1nc(OC[C@@H]2CCCCN2C)nc2c1CCN(c1cccc3cccc(Cl)c13)C2 ; C[C@H]1Cc2cncc(F)c2CN1c1nc(OC[C@@H]2CCCCN2C)nc2c1CCN(c1cccc3cccc(Cl)c13)C2]

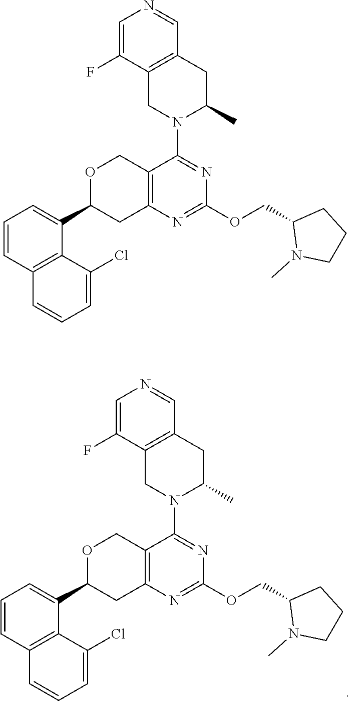 [SMILES: C[C@@H]1Cc2cncc(F)c2CN1c1nc(OC[C@@H]2CCCN2C)nc2c1CO[C@H](c1cccc3cccc(Cl)c13)C2 ; C[C@H]1Cc2cncc(F)c2CN1c1nc(OC[C@@H]2CCCN2C)nc2c1CO[C@H](c1cccc3cccc(Cl)c13)C2]

The disclosure also provides a method for preparing the nitrogen-containing heterocyclic compound of formula I described above, which is route I, route II, route III, route IV or route V.

Route I, the synthetic method is as follows:

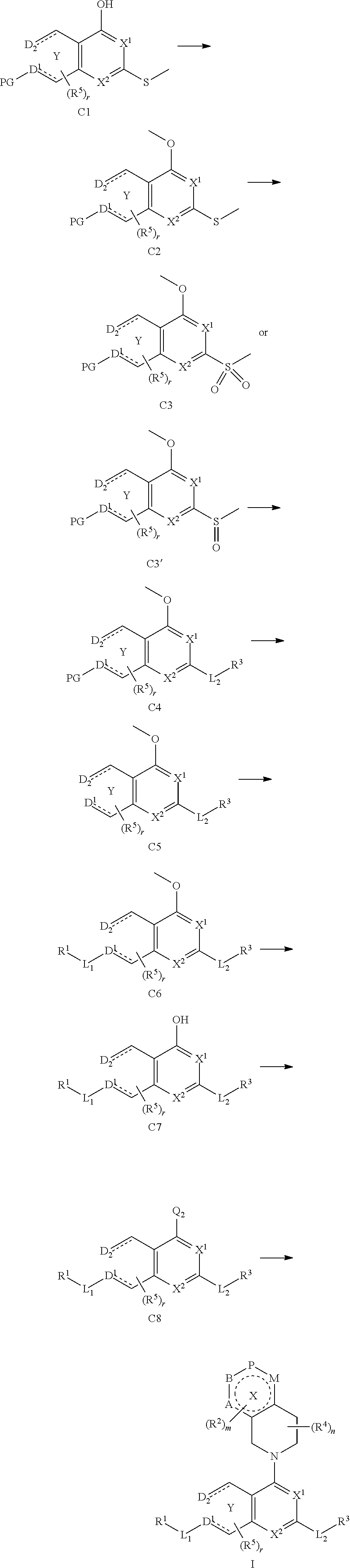

Wherein R¹, R², R³, R⁴, R⁵, A, B, P, M, D¹, D₂, L₁, L₂, X¹, X², m, n and r are as defined above, and Q₂ is independently a leaving group, such as OTf or Cl; PG is H or amino protecting group, such as THP, Boc or Cbz; the route I comprises the following steps: compound C1 is protected with Me group, C2 is oxidized to obtain C3 or C3′, C3 or C3′ is converted to C4 by nucleophilic substitution, C4 is converted to C5 by removing the protecting group, C5 is converted to C6 by nucleophilic substitution or coupling reaction, C6 is converted to C7 by removing Me group, hydroxyl in C7 was converted to a leaving group to obtain C8, and C8 is converted to compound I by nucleophilic substitution.

In the above route, if protecting groups on the R¹ group have a clear advantage, it can enable the chemical reaction to proceed in a simple manner, also remove the protecting groups after introducing the R¹ group. These protecting groups include: the protecting group TIPS on the R¹ group.

Route II, the synthetic method is as follows:

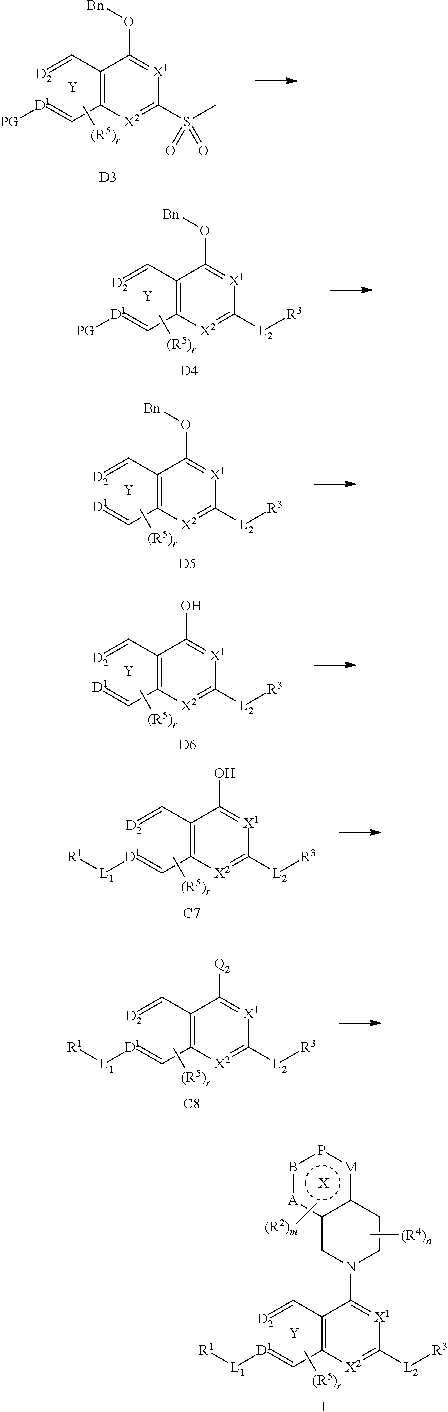

Wherein R¹, R², R³, R⁴, R⁵, A, B, P, M, D¹, D₂, L₁, L₂, X¹, X², m, n and r are as defined above, and Q₂ is independently a leaving group, such as OTf or Cl; PG is H or amino protecting group, such as THP, Boc or Cbz; the route II comprises the following steps: compound D3 is converted to D4 by nucleophilic substitution, D4 is removed protecting group to obtain D5, D5 is removed benzyl group to obtain D6, D6 is converted to C7 by nucleophilic substitution or coupling reaction, then hydroxyl in C7 is converted to a leaving group to obtain C8, and C8 is converted to compound I by nucleophilic substitution.

Route III, the synthetic method is as follows:

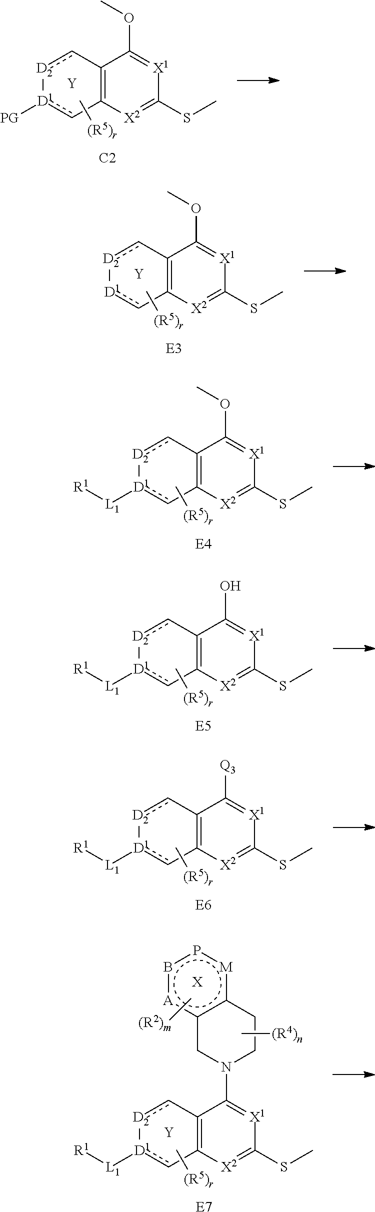

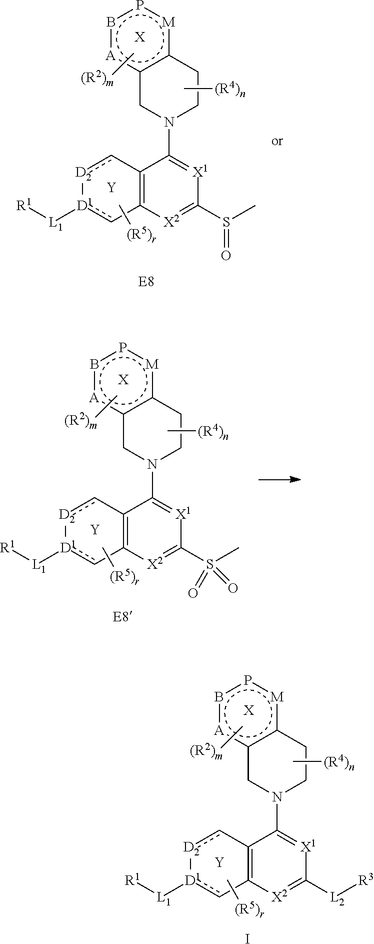

Wherein R¹, R², R³, R⁴, R⁵, A, B, P, M, D¹, D₂, L₁, L₂, X¹, X², m, n and r are as defined above, and Q3 is independently a leaving group, such as OTf or Cl; PG is H or amino protecting group, such as THP, Boc or Cbz; the route III comprises the following steps: C2 is removed protecting group to obtain E3, E3 is converted to E4 by nucleophilic substitution or coupling reaction, E4 is converted to E5 by removing methyl group, then hydroxyl in E5 was converted to a leaving group to obtain E6, E6 is converted to E7 by nucleophilic substitution, E7 is oxidized to obtain E8 or E8′, and E8 or E8′ is converted to compound I by nucleophilic substitution.

Route IV, the synthetic method is as follows:

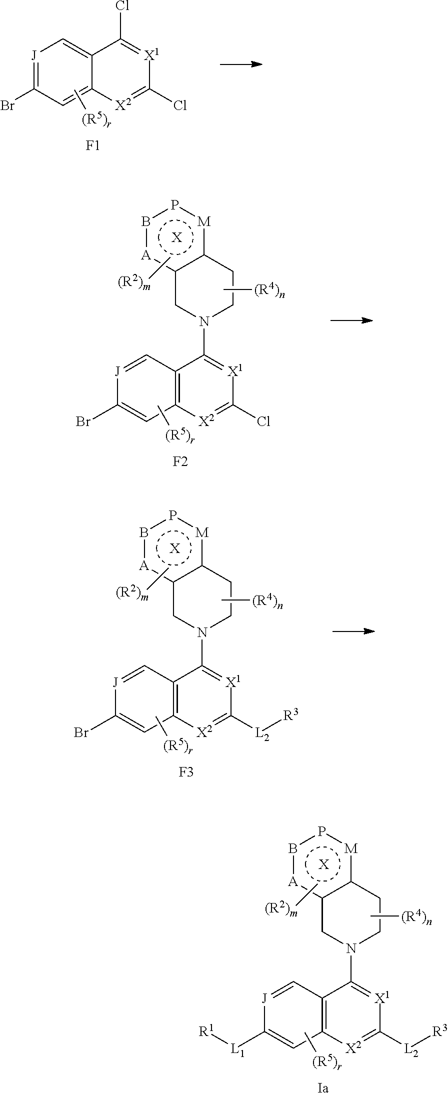

Wherein R¹, R², R³, R⁴, R⁵, A, B, P, M, D¹, D₂, L₁, L₂, X¹, X², m, n and r are as defined above, and J is CH or N; the route IV comprises the following steps: F1 is converted to F2 by nucleophilic substitution, F2 is converted to F3 by nucleophilic substitution, and F3 is converted to Ia by coupling reaction.

Route V, the synthetic method is as follows:

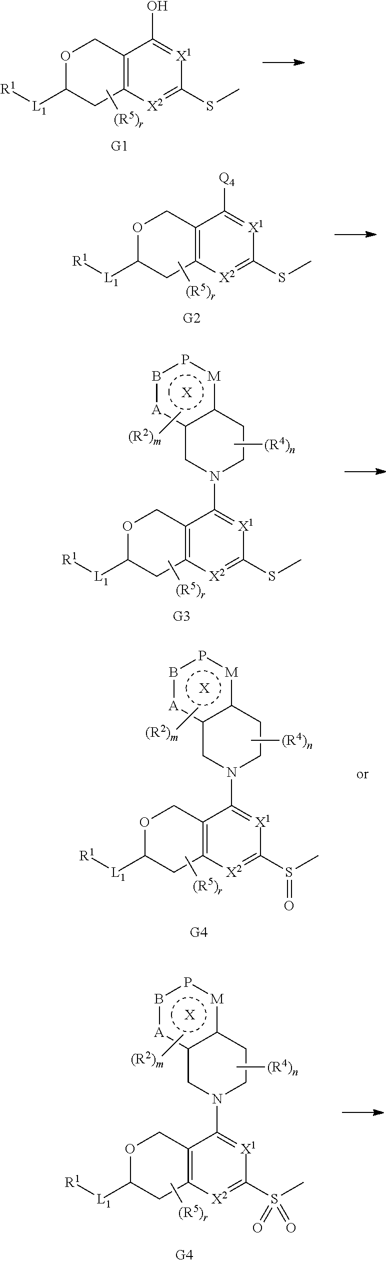

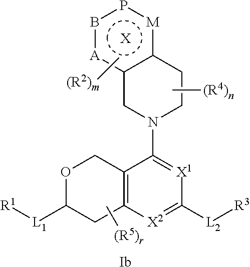

Wherein R¹, R², R³, R⁴, R⁵, A, B, P, M, D¹, D₂, L₁, L₂, X¹, X², m, n and r are as defined above, and Q₄ is independently a leaving group, such as OTf or Cl; the route V comprises the following steps: hydroxyl in G1 was converted to a leaving group to obtain G2, G2 is converted to G3 by nucleophilic substitution, G3 is oxidized to obtain G4 or G4′, and G4 or G4′ is converted to Ib by nucleophilic substitution.

The disclosure also provides a compound,

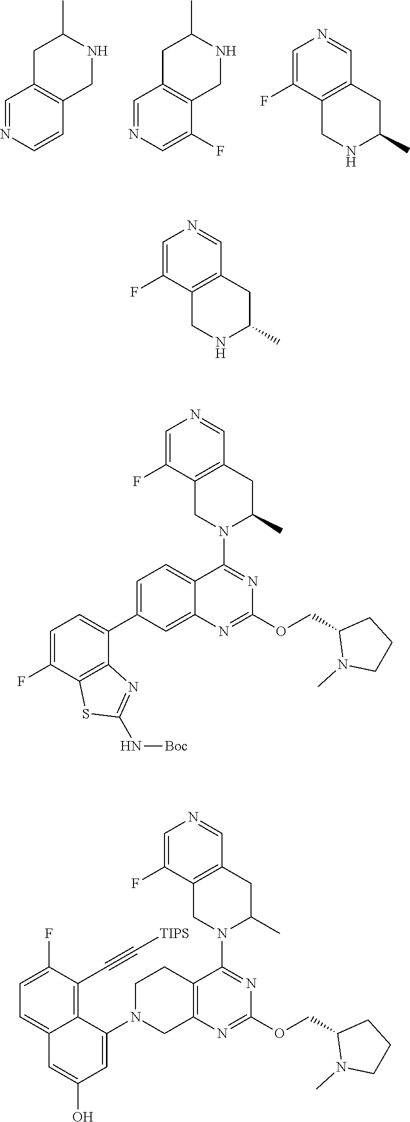

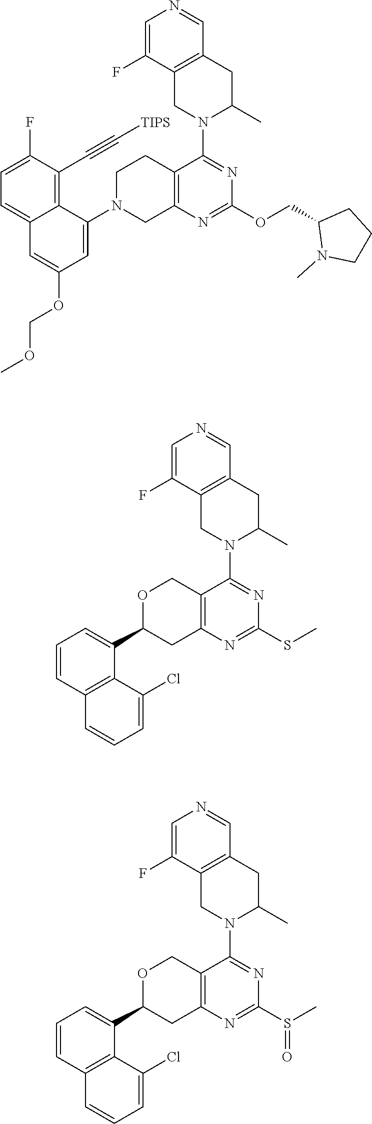

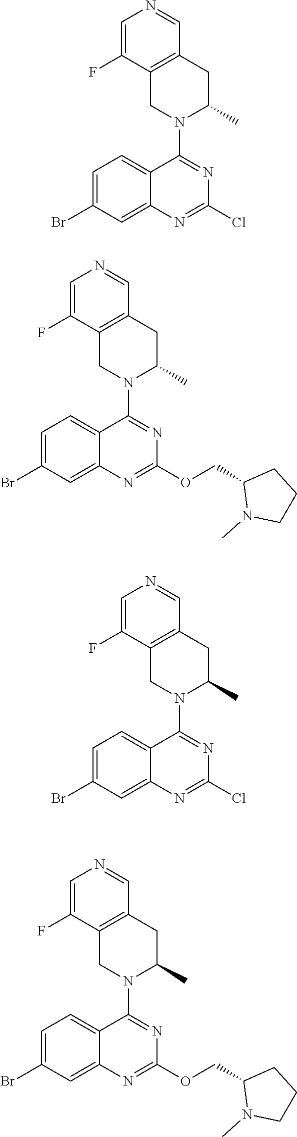 [SMILES: C[C@H]1Cc2cncc(F)c2CN1c1nc(Cl)nc2cc(Br)ccc12 ; C[C@H]1Cc2cncc(F)c2CN1c1nc(OC[C@@H]2CCCN2C)nc2cc(Br)ccc12 ; C[C@@H]1Cc2cncc(F)c2CN1c1nc(Cl)nc2cc(Br)ccc12 ; C[C@@H]1Cc2cncc(F)c2CN1c1nc(OC[C@@H]2CCCN2C)nc2cc(Br)ccc12]

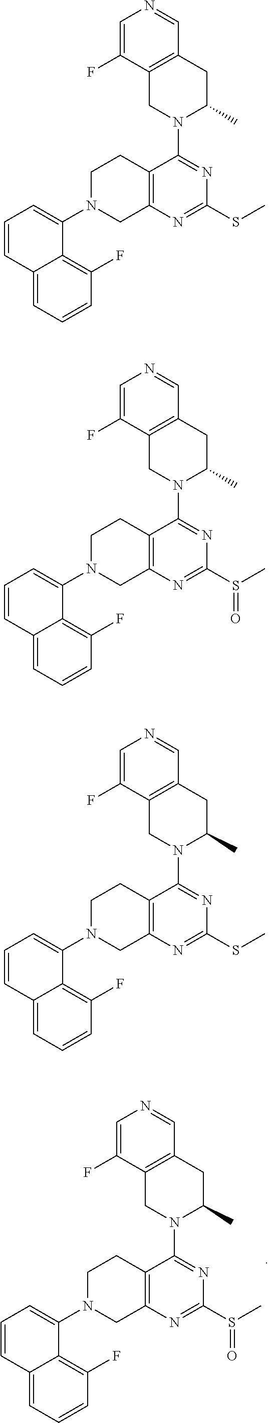 [SMILES: CSc1nc2c(c(N3Cc4c(F)cncc4C[C@@H]3C)n1)CCN(c1cccc3cccc(F)c13)C2 ; C[C@H]1Cc2cncc(F)c2CN1c1nc(S(C)=O)nc2c1CCN(c1cccc3cccc(F)c13)C2 ; CSc1nc2c(c(N3Cc4c(F)cncc4C[C@H]3C)n1)CCN(c1cccc3cccc(F)c13)C2 ; C[C@@H]1Cc2cncc(F)c2CN1c1nc(S(C)=O)nc2c1CCN(c1cccc3cccc(F)c13)C2]

The present disclosure also provides a pharmaceutical composition comprising a substance A and a pharmaceutically acceptable excipient, wherein the substance A is a therapeutically effective amount of the nitrogen-containing heterocyclic compound of formula I, the pharmaceutically acceptable salt thereof, the stereoisomer thereof, the tautomer thereof or the isotopically labeled compound thereof as described above.

The present disclosure also provides a method for inhibiting RAS in a subject thereof, comprising: administering a therapeutically effective amount of a substance A, wherein the substance A is the nitrogen-containing heterocyclic compound of formula I, the pharmaceutically acceptable salt thereof, the stereoisomer thereof, the tautomer thereof or the isotopically labeled compound thereof as described above.

In the method for inhibiting RAS in a subject, the RAS is a wild type or a mutant RAS; The mutant RAS is, for example, a KRAS, HRAS or NRAS mutation, wherein the KRAS mutation can be a G12, G13 or Q61 mutation, for example, KRAS G12C, KRAS G12D, KRAS G12S, KRAS G12A, KRAS G12V or KRAS G13D, and for another example, KRAS G12C, KRAS G12D or KRAS G12V; the HRAS mutation can be a G12, G13 or Q61 mutation, for example, HRAS G12C, HRAS G12D, HRAS G12S, HRAS G12A, HRAS G12V or HRAS G13D; the NRAS mutation can be a G12, G13 or Q61 mutation, for example, NRAS G12C, NRAS G12D, NRAS G12S, NRAS G12A, NRAS G12V or NRAS G13D; the mutant RAS is, for another example, KRAS G12C.

The present disclosure also provides a method for treating or preventing an RAS-related disease in a subject in need thereof, comprising: administering an effective amount of a substance A, wherein the substance A is the nitrogen-containing heterocyclic compound of formula I, the pharmaceutically acceptable salt thereof, the stereoisomer thereof, the tautomer thereof or the isotopically labeled compound thereof as described above.

In the method for treating or preventing an RAS-related disease in a subject, the mutant RAS is a KRAS, HRAS or NRAS mutation, wherein the KRAS mutation is G12, G13, Q61 mutation, for example, KRAS G12C, KRAS G12D, KRAS G12S, KRAS G12A, KRAS G12V KRAS G13D, KRAS G12C, KRAS G12D or KRAS G12V; the HRAS mutation is a G12, G13 or Q61 mutation; the NRAS mutation is a G12, G13 or Q61 mutation. The RAS-related disease is cancer; the cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

The present disclosure also provides a method for treating or preventing a cancer in a subject in need thereof, comprising: administering an effective amount of a substance A, wherein the substance A is the nitrogen-containing heterocyclic compound of formula I, the pharmaceutically acceptable salt thereof, the stereoisomer thereof, the tautomer thereof or the isotopically labeled compound thereof as described above.

The cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

The term “pharmaceutically acceptable salt” refers to a salt prepared from compounds of the present disclosure with relatively non-toxic, pharmaceutically acceptable acids or bases. When compounds of the present disclosure contain relatively acidic functional groups, base addition salts can be obtained by contacting the neutral form of such compounds with a sufficient amount of pharmaceutically acceptable bases, either in pure solution or a suitable inert solvent. The pharmaceutically acceptable base addition salts include but are not limited to: lithium salt, sodium salt, potassium salt, calcium salt, aluminum salt, magnesium salt, zinc salt, bismuth salt, ammonium salt and diethanolamine salt. When compounds of the present disclosure contain relatively basic functional groups, acid addition salts can be obtained by contacting the neutral form of such compounds with a sufficient amount of pharmaceutically acceptable acids, either in pure solution or a suitable inert solvent. The pharmaceutically acceptable acids include inorganic acids, and the inorganic acids include but are not limited to: hydrochloric acid, hydrobromic acid, hydroiodic acid, nitric acid, carbonic acid, phosphoric acid, phosphorous acid and sulfuric acid. The pharmaceutically acceptable acids include organic acids, and the organic acids include but are not limited to: acetic acid, propionic acid, oxalic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, salicylic acid, tartaric acid, methanesulfonic acid, isonicotinic acid, acidic citric acid, oleic acid, tannic acid, pantothenic acid, hydrogen tartrate, ascorbic acid, gentisic acid, fumaric acid, gluconic acid, saccharic acid, formic acid, ethanesulfonic acid, pamoic acid (i.e., 4,4′-methylene-bis(3-hydroxy-2-naphthoic acid)) and amino acid (such as glutamic acid and arginine). When compounds of the present disclosure contain relatively acidic functional groups and relatively basic functional groups, such compounds can be converted into base addition salts or acid addition salts. For details, reference can be made to Berge et al., “Pharmaceutical Salts”, Journal of Pharmaceutical Science 66: 1-19 (1977), or Handbook of Pharmaceutical Salts: Properties, Selection, and Use (P. Heinrich Stahl and Camille G. Wermuth, ed., Wiley-VCH, 2002).

The term “stereoisomer” refers to an isomer in which the atoms or atomic groups in a molecule have the same interconnection order but different spatial arrangements, such as cis-trans isomers, optical isomers or atropisomers. These stereoisomers can be separated, purified and enriched by means of asymmetric synthesis methods or chiral separation methods (including but not limited to thin layer chromatography, rotation chromatography, column chromatography, gas chromatography and high-pressure liquid chromatography) or can also be obtained by means of chiral resolution via forming bonds (chemical bonding, etc.) or forming salts (physical bonding) with other chiral compounds, etc.

The term “tautomer” refers to a functional group isomer resulting from the rapid movement of an atom in two positions in a molecule. For example, acetone and 1-propene-2-ol can be converted into each other by the rapid movement of hydrogen atoms on oxygen and α-carbon.

The term “isotopic compound” refers to a compound in which one or more atoms are substituted with one or more atoms having a specific atomic mass or mass number. Examples of isotopes that can be incorporated into compounds of the present disclosure include, but are not limited to, isotopes of hydrogen, carbon, nitrogen, oxygen, fluorine, sulfur and chlorine (e.g., 2H, 3H, 13C, 14C, 15N, 18O, 17O, 18F, 35S and 36Cl). The isotopic compounds of the present disclosure can generally be prepared by substituting non-isotopically-labeled reagents with isotopically-labeled reagents according to the methods described herein.

The term “halogen” refers to fluorine, chlorine, bromine or iodine.

The term “alkyl” refers to a linear or branched alkyl group having a specified number of carbon atoms. Examples of alkyl include methyl, ethyl, n-propyl, isopropyl, n-butyl, tert-butyl, isobutyl, sec-butyl, n-pentyl, n-hexyl, n-heptyl, n-octyl, and the like.

The term “alkylene” refers to a linking group between two other species, which may be linear or branched. Examples include, but are not limited to, —CH₂—, —CH₂CH₂—, —CH₂CH₂CH₂CH(CH₃)— and —CH₂CH(CH₂CH₃)CH₂—.

The term “alkoxy” refers to group —O-Rx, wherein RX is alkyl as defined above.

The terms “cycloalkyl” and “carbocyclic ring” refer to a saturated cyclic group consisting only of carbon atoms having a specified number of carbon atoms (e.g., C₃-C₆), which is a monocyclic, bridged or spiro ring. The cycloalkyl includes, but is not limited to, cyclopropyl, cyclobutyl, cyclopentyl, and cyclohexyl.

The term“aryl” refers to an aromatic group consisting of carbon atoms, each ring having aromaticity. For example, phenyl or naphthyl.

The term “heteroaryl” refers to a cyclic group having a specified number of ring atoms (e.g., 5-12 members), a specified number of heteroatoms (e.g., 1, 2, or 3) and specified heteroatom species (one or more of N, O and S), which is monocyclic or polycyclic, and has at least one aromatic ring (according to the Hckel's rule). Heteroaryls are linked to other fragments of the molecule through aromatic or non-aromatic rings. Heteroaryls include, but are not limited to, furyl, pyrrolyl, thienyl, pyrazolyl, imidazolyl, oxazolyl, thiazolyl, pyridinyl, pyrimidinyl, and indolyl.

The terms “heterocyclyl”, “heterocycle” or “heterocycloalkyl” refer to a cyclic group having a specified number of ring atoms (e.g., 3-8 members), a specified number of heteroatoms (e.g., 1, 2, or 3) and specified heteroatom species (one or more of N, O and S), which is monocyclic, bridged, or spiro, and where each ring is saturated. Heterocycloalkyls include, but are not limited to, azetidinyl, tetrahydropyrrolyl, tetrahydrofuryl, morpholinyl, piperidinyl, and the like.

The term “hydroxyl” refers to a —OH group.

The term “cyano” refers to a —CN group.

The term “oxo” refers to a ═O group.

Substituted “Cₓ₁-C_{y1}” groups with specified numbers of carbon atoms (x1 and y1 are integers), for example, “Cₓ₁-C_{y1}” alkyl, “Cₓ₁-C_{y1}” cycloalkyl, “Cₓ₁-C_{y1}” cycloalkenyl, “Cₓ₁-C_{y1}” alkoxyl, “Cₓ₁-C_{y1}” alkenyl, “Cₓ₁-C_{y1}” alkynyl, “Cₓ₁-C_{y1}” aryl, “Cₓ₁-C_{y1}” heteroaryl, or “Cₓ₁-C_{y1}” heterocyclyl, all represent numbers of carbon atoms excluding substituents, e.g., a C₁-C₆ alkyl represents a C₁-C₆ alkyl excluding substituents.

The above preferred conditions may be combined arbitrarily to obtain preferred embodiments of the present disclosure without departing from the general knowledge in the art.

The reagents and starting materials used in the present disclosure are commercially available.

The positive/progressive effects of the present disclosure are as follows: the present disclosure provides a nitrogen-containing heterocyclic compound, a method for preparing the same and use thereof, wherein the nitrogen-containing heterocyclic compound has a favorable inhibiting effect on cells with various effect on various cells with mutations such as KRAS G12C and/or KRAS G12D, and is prospective for treating and/or preventing various diseases mediated by Ras.

### DETAILED DESCRIPTION OF THE EMBODIMENTS

The present disclosure is further illustrated by the following examples, which are not intended to limit the present disclosure. Experimental procedures without specified conditions in the following examples were performed in accordance with conventional procedures and conditions, or in accordance with instructions.

In the present disclosure, room temperature refers to ambient temperature, or 10-35° C. Overnight refers to 8-15 hours. Reflux refers to the reflux temperature of a solvent at atmospheric pressure.

The following is a list of abbreviations used in the examples:

DMF N, N-dimethylformamide

DIPEA diisopropylethylamine

Pd(PPh₃)₄ tetrakis(triphenylphosphine)palladium

Pd(dppf)Cl₂ [1,1′-bis(diphenylphosphino)ferrocene]palladium(II) dichloride dichloromethane complex

Pd₂(dba)₃ tris(dibenzylideneacetone)dipalladium

m-CPBA m-chloroperoxybenzoic acid

RuPhos 2-dicyclohexylphosphino-2′,6′-diisopropoxybiphenyl

XPhos 2-dicyclohexylphosphino-2′,4′,6′-triisopropylbiphenyl

TIPS Triisopropylsilyl

Tf Trifluoromethanesulfonyl

THE Tetrahydrofuran

THP 2-Tetrahydropyran

TFA Trifluoroacetic acid

MOM Methoxymethyl

BINAP racemic-2,2′-Bis(diphenylphosphino)-1,1 binaphthyl

### Synthetic Route of Intermediate I-1

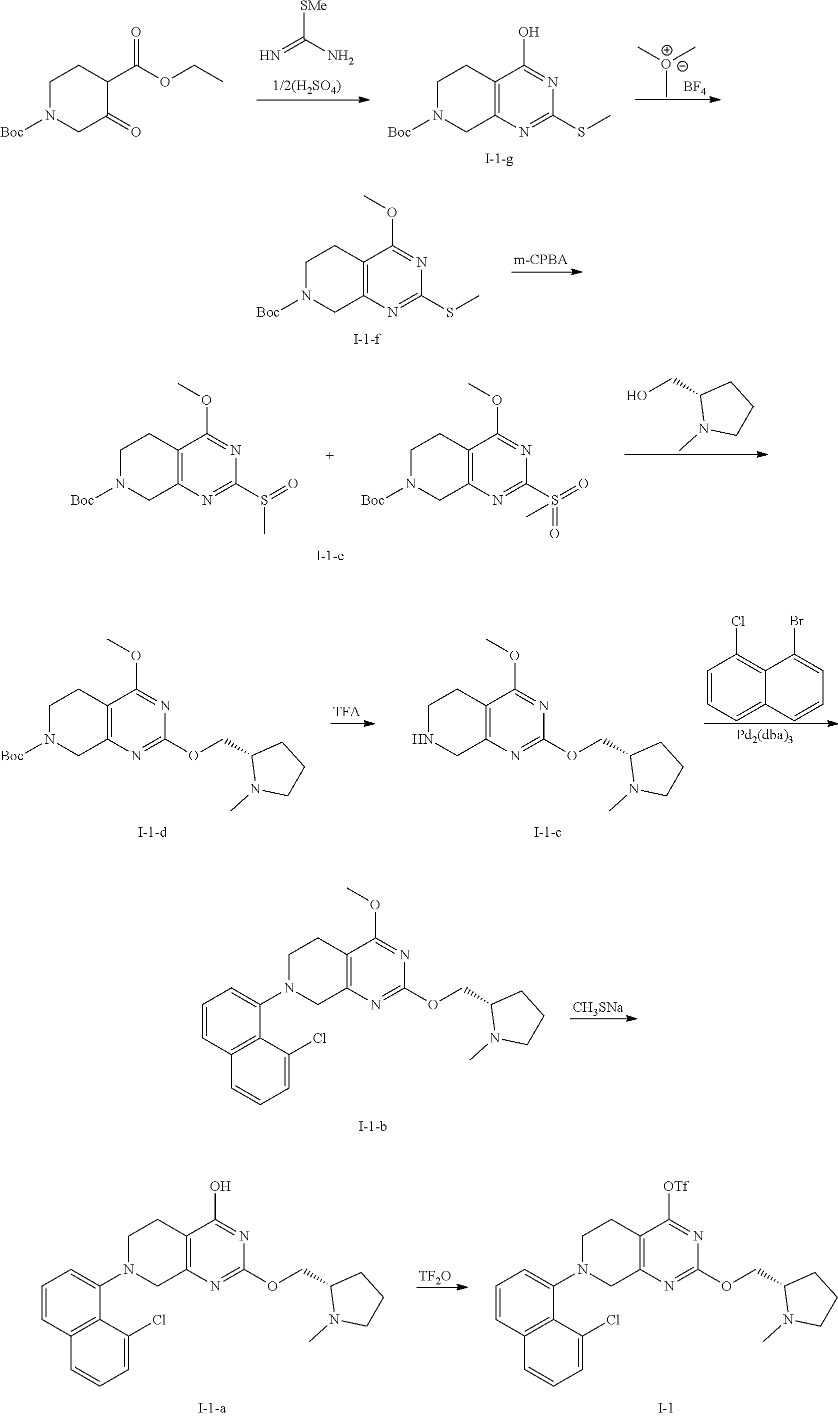

### Synthesis of Compound I-1-g

Ethyl 1-N-tert-butoxycarbonyl-3-oxopiperidine-4-carboxylate (36 g, 132.69 mmol), methanol (500 mL) and 2-methyl-2-thiopseudourea sulfate (44.9 g, 239.11 mmol) were added to a reaction flask. In an ice-cold water bath, sodium methoxide (35.9 g, 664.2 mmol) was added and the mixture was stirred at room temperature overnight under nitrogen atmosphere. The next day, the mixture was adjusted to pH 6 with 1 M hydrochloric acid in an ice-cold water bath, and filtered. The filter cake was dried in air to obtain compound I-1-g (53 g) as a white solid, which was used in the next step without purification. LC-MS (ESI): m/z=298.2 (M+H)⁺.

### Synthesis of Compound I-1-f

The crude product of I-1-g (4.40 g, 14.8 mmol) was dissolved in dichloromethane (600 mL). Trimethyloxonium tetrafluoroborate (2.40 g, 16.30 mmol) was added at room temperature and the mixture was stirred for 2 hours under nitrogen atmosphere. The reaction was quenched by addition of saturated aqueous sodium bicarbonate (150 mL). Dichloromethane (150 mL×2) was added for extraction, and the organic phase was washed with brine (100 mL×2), and purified by column chromatography (mobile phase: ethyl acetate/petroleum ether=1/5) to obtain compound I-1-f (2.10 g, 46%). LC-MS (ESI): m/z 312.1 (M+H)⁺.

### Synthesis of Compound I-1-e

Compound I-1-f (2.10 g, 6.75 mmol) was dissolved in ethyl acetate (50 mL) and 85% pure m-CPBA (3.42 g, 16.88 mmol) was added at room temperature. After the mixture was stirred at 0° C. for 3 hours, saturated sodium bicarbonate solution (180 mL) was added to quench the reaction. Ethyl acetate (500 mL×2) was added for extraction, and the organic phase was concentrated and purified by column chromatography (mobile phase: ethyl acetate/petroleum ether=1/3) to obtain compound I-1-e (2.10 g, 91%). LC-MS (ESI): m/z 328.1 (M+H)⁺, m/z 343.1 (M+H)⁺.

### Synthesis of Compound I-1-d

In an ice-cold water bath, to a solution compound I-1-e (2.10 g, 6.12 mmol) in toluene (60 mL) were serially added N-methyl-L-prolinol (0.85 g, 7.35 mmol) and sodium tert-butoxide (0.71 g, 7.35 mmol). After the mixture was stirred in an ice-cold water bath and nitrogen atmosphere for 0.5 hours, water (10 mL) was added to quench the reaction. Ethyl acetate (30 mL×2) was added for extraction, and the organic phase was concentrated and purified by column chromatography (mobile phase: methanol/dichloromethane=1/20) to obtain compound I-1-d (2.20 g, 95%). LC-MS (ESI): m/z 379.2 (M+H)⁺.

### Synthesis of Compound I-1-c

Compound I-1-d (2.20 g, 5.81 mmol) was added to a mixed solution of dichloromethane (50 mL) and trifluoroacetic acid (5 mL) and the mixture was stirred at room temperature for 3 hours. The reaction mixture was concentrated, and saturated aqueous sodium bicarbonate (150 mL) was added to quench the reaction. Solid sodium sulfate was added to the aqueous solution, and a mixed solution (methanol/dichloromethane=1/20; 330 mL×3) was added for extraction. The organic phase was dried over sodium sulfate, and concentrated to obtain compound I-1-c (1.83 g, 100%). LC-MS (ESI): m/z 279.1 (M+H)⁺.

### Synthesis of Compound I-1-b

Compound I-1-c (1.60 g, 5.76 mmol), 1-bromo-8-chloronaphthalene (1.70 g, 7.08 mmol), RuPhos (0.50 g, 1.07 mmol), Pd₂(dba)₃ (0.50 g, 0.55 mmol), Cs₂CO₃ (6.80 g, 20.90 mmol) were added to toluene (100 mL), and the mixture was heated to 100° C. and stirred for 3 hours under nitrogen atmosphere. The reaction mixture was cooled to room temperature. Water (100 mL) was added to quench the reaction and ethyl acetate (100 mL×3) was added for extraction. The organic phase was concentrated and purified by column chromatography (mobile phase: methanol/dichloromethane=1/20) to obtain compound I-1-b (1.23 g, 49%). LC-MS (ESI): m/z 439.2 (M+H)⁺.

### Synthesis of Compound I-1-a

Compound I-1-b (1.23 g, 2.81 mmol) and sodium thiomethoxide (0.78 g, 11.23 mmol) were added to DMF (30 mL). The mixture was heated to 60° C. and stirred for 3 hours under nitrogen atmosphere. The reaction was quenched by adding water (120 mL) and diluted hydrochloric acid (6 mL, 12 mmol), and ethyl acetate (100 mL×3) was added for extraction. The organic phase was concentrated and purified by column chromatography (mobile phase: methanol/dichloromethane=1/10) to obtain compound I-1-a (0.89 g, 75%). LC-MS (ESI): m/z 425.1 (M+H)⁺.

### Synthesis of Compound I-1

I-1-a (0.89 g, 2.10 mmol) and triethylamine (0.64 g, 6.3 mmol) were dissolved in dichloromethane (60 mL) and the temperature was reduced to −40° C. under nitrogen atmosphere. Trifluoromethanesulfonic anhydride (1.06 g, 3.80 mmol) was slowly dropwise added. After the addition, the mixture was stirred at −40° C. for 0.5 hours. The reaction was quenched by addition of saturated sodium bicarbonate solution (100 mL). Dichloromethane (100 mL×2) was added for extraction, and the organic phase was concentrated and purified by column chromatography (mobile phase: methanol/dichloromethane=1/20) to obtain compound I-1 (0.76 g, 65%). LC-MS (ESI): m/z 557.1 (M+H)⁺.

### Example 1: Synthetic Route of Compound 1

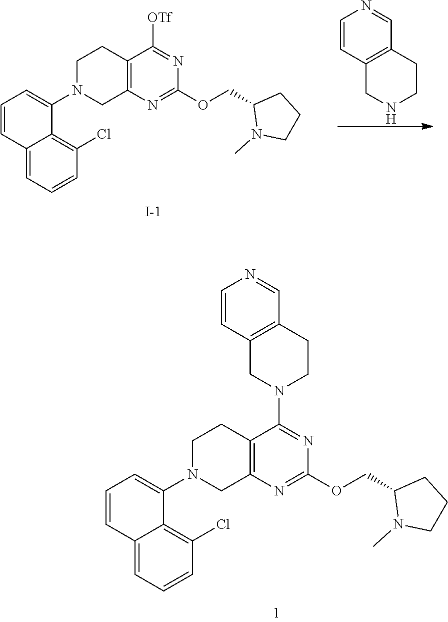 [SMILES: CN1CCC[C@H]1COc1nc2c(c(OS(=O)(=O)C(F)(F)F)n1)CCN(c1cccc3cccc(Cl)c13)C2 ; c1cc2c(cn1)CCNC2 ; [I-] ; CN1CCC[C@H]1COc1nc2c(c(N3CCc4cnccc4C3)n1)CCN(c1cccc3cccc(Cl)c13)C2]

### Synthesis of Compound 1

To a reaction vial were added I-1 (50 mg, 0.09 mmol), 1,2,3,4-tetrahydro-2,6-naphthyridine (18 mg, 0.135 mmol), N,N-diisopropylethylamine (23 mg, 0.18 mmol) and DMF (3 mL) respectively. The mixture was reacted at room temperature for 1 h. After the reaction was completed, the reaction mixture was directly purified by Prep-HPLC to obtain compound 1 (25 mg, 52%) as a white solid. LC-MS (ESI): m/z 541.0 (M+H)⁺; ¹H NMR (400 MHz, CDCl₃): δ 8.45 (1H, s), 8.42 (1H, d, J=5.2 Hz), 7.77 (1H, dd, J=8.0 Hz, 1.2 Hz), 7.63 (1H, d, J=8 Hz), 7.54 (1H, dd, J=7.2 Hz, 0.8 Hz), 7.46 (1H, t J=8.0 Hz), 7.35 (1H, t, J=8.0 Hz), 7.25 (1H, dd, J=7.6 Hz, 0.8 Hz), 7.11 (1H, d, J=4.8 Hz), 4.70 (2H, s), 4.46-4.42 (2H, m), 4.21-4.15 (1H, m), 4.06-3.99 (1H, m), 3.87 (1H, d, J=18 Hz), 3.62-3.48 (2H, m), 3.29-3.09 (4H, m), 3.00-2.91 (1H, m), 2.76-2.61 (1H, m), 2.66-2.59 (1H, m), 2.51 (3H, s), 2.37-2.22 (1H, m), 2.15-2.02 (1H, m), 1.91-1.71 (3H, m).

### Example 2: Synthetic Route of Compound 2

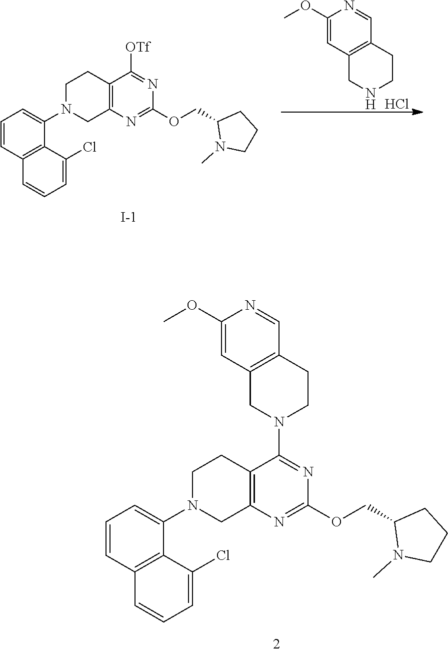 [SMILES: CN1CCC[C@H]1COc1nc2c(c(OS(=O)(=O)C(F)(F)F)n1)CCN(c1cccc3cccc(Cl)c13)C2 ; COc1cc2c(cn1)CCNC2 ; Cl ; [I-] ; COc1cc2c(cn1)CCN(c1nc(OC[C@@H]3CCCN3C)nc3c1CCN(c1cccc4cccc(Cl)c14)C3)C2]

### Synthesis of Compound 2

I-1 (20 mg, 0.036 mmol), 7-methoxy-1,2,3,4-tetrahydro-2,6-naphthyridine hydrochloride (15 mg, 0.072 mmol), DIPEA (14 mg, 0.108 mmol) and DMF (2 mL) were combined in a reaction vial. The mixture was reacted at room temperature for 1 h. After the reaction was completed, the reaction mixture was directly purified by Prep-HPLC preparation to obtain compound 2 (18.7 mg, 91%) as a white solid. LC-MS (ESI): m/z 571.2 (M+H)⁺; ¹H NMR (400 MHz, CDCl₃): δ 8.00 (1H, s), 7.76 (1H, dd, J=8.4 Hz, 0.8 Hz), 7.63 (1H, dd, J=8.4 Hz, 0.8 Hz), 7.53 (1H, dd, J=7.2 Hz, 1.2 Hz), 7.45 (1H, t, J=8 Hz), 7.34 (1H, t, J=8 Hz), 7.23 (1H, dd, J=7.6 Hz, 0.8 Hz), 6.58 (1H, s), 4.66 (2H, s), 4.48-4.39 (2H, m), 4.22-4.15 (1H, m), 4.01-3.95 (1H, m), 3.94 (3H, s), 3.86 (1H, d, J=17.6 Hz), 3.60-3.45 (2H, m), 3.29-3.09 (3H, m), 3.09-3.00 (1H, m), 2.92-2.83 (1H, m), 2.76-2.67 (1H, m), 2.64-2.57 (1H, m), 2.51 (3H, d, J=2.8 Hz), 2.34-2.25 (1H, m), 2.14-2.07 (1H, m), 1.86-1.75 (3H, m).

### Example 3: Synthetic Route of Compound 3

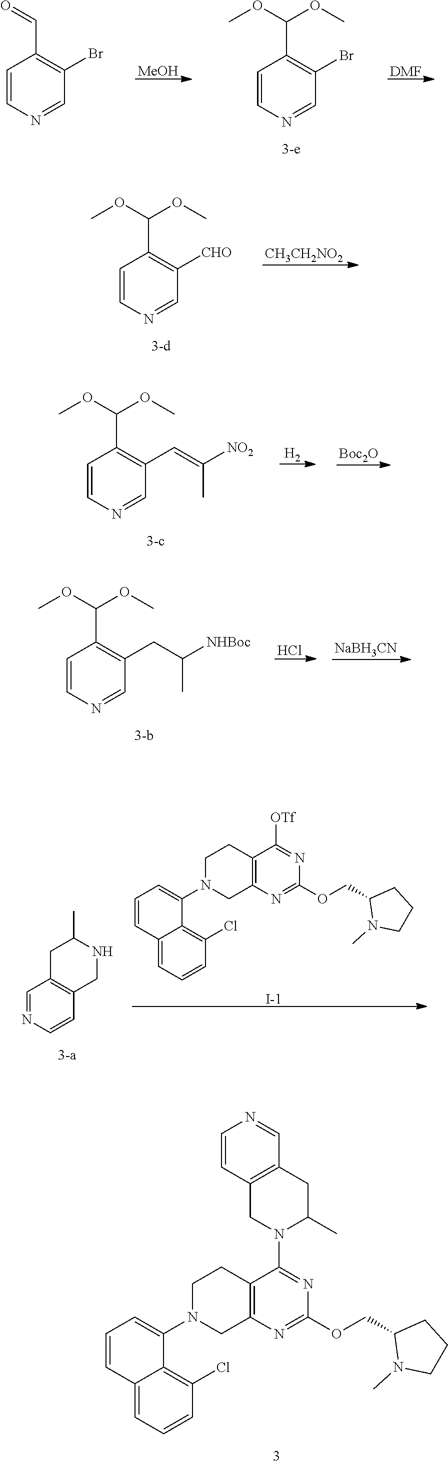

### Synthesis of Compound 3-e

A solution of 3-bromo-4-pyridinecarboxaldehyde (5.0 g, 26.88 mmol) in methanol (120 mL) in a reaction flask was stirred at room temperature for 10 min, then was slowly added concentrated sulfuric acid (10.6 g, 106 mmol). The mixture was stirred at 50° C. for 22 hours. The reaction mixture was concentrated and added to saturated aqueous sodium bicarbonate solution (400 mL). Extracted with ethyl acetate (200 mL) and concentrated to give compound 3-e (4.8 g, 77%). LC-MS (ESI): m/z 231.7 (M+H)⁺.

### Synthesis of Compound 3-d

3-e (4.8 g, 20.7 mmol) was added to a reaction flask containing dry tetrahydrofuran (150 mL) and the resulting mixture was stirred at −78° C. for 10 min. At this temperature, n-butyllithium (15 mL, 2.5 M, 37.5 mmol) was slowly added to the above mixture. After addition, the reaction mixture was stirred at −78° C. for 2 hours. Dried DMF (8 mL) was added to the above mixture, and the mixture continued stirring for 2 hours. The reaction was quenched by adding saturated ammonium chloride (180 mL) and extracted with ethyl acetate (100 mL*3). The organic phase was concentrated and purified by column chromatography (mobile phase: petroleum ether/ethyl acetate 3/1 to 1/1) to give compound 3-d (3.6 g, 96%).

### Synthesis of Compound 3-c

To 3-d (3.6 g, 19.9 mmol) in a reaction vial was added nitroethane (20 mL) and ammonium acetate (0.57 g, 7.30 mmol), respectively, and the resulting mixture was stirred at 100° C. for 16 h. The reaction mixture was concentrated and purified by column chromatography (mobile phase: petroleum ether/ethyl acetate 3/1 to 1/1) to give compound 3-c (2.6 g, 55%). LC-MS (ESI): m/z 239.1 (M+H)⁺.

### Synthesis of Compound 3-b

3-c (0.48 g, 2.0 mmol) and Raney nickel (1 g) were added to a reaction flask containing methanol (15 mL). After degassed and purged with hydrogen, the mixture stirred at 30° C. for 60 min under hydrogen balloon (˜1 L). Ammonia methanol (4 mL, 7 M, 28.0 mmol) was added to the reaction mixture and stirred at 30° C. for 13 hours. Then Boc₂O (1.26 g, 6.0 mmol) was added to the reaction mixture and stirred for 2 hours. The reaction was filtered, the filtrate was concentrated and purified by column chromatography (mobile phase: petroleum ether/ethyl acetate 1/1 to 2/3) to give compound 3-b (0.28 g, 45%). LC-MS (ESI): m/z 311.7 (M+H)

### Synthesis of Compound 3-a

To a reaction flask containing 3-b (0.28 g, 0.90 mmol) was added HCl/1,4-dioxane (4.0 mL, 4 M, 16.0 mmol) and hydrochloric acid (2.0 mL, 6 M, 12.0 mmol), respectively, and the mixture was stirred for 2 h at 10° C. The reaction mixture was blown dry with nitrogen and methanol (20 mL) was added to the residue. The mixture was stirred at 0° C. for 10 minutes, then was added Sodium cyanoborohydride (1.0 g, 15.9 mmol), and continued stirring for 2 hours. The reaction mixture was concentrated at room temperature and purified by column chromatography (mobile phase: dichloromethane/Ammonia methanol(7M) 20/1 to 10/1) to afford compound 3-a (56 mg, 42%).

### Synthesis of Compound 3

3-a (24 mg, 0.16 mmol), DMSO (4 mL), I-1 (100 mg, 0.18 mmol) and DIPEA (260 mg, 2.00 mmol) were added into a reaction flask respectively and the mixture was stirred at 50° C. for 4 h under argon. The reaction mixture was quenched by adding brine (30 mL) and extracted with ethyl acetate (30 mL*2). The organic phase was concentrated to dryness and the residue was purified by column chromatography (mobile phase: methanol/dichloromethane 0/100 to 1/20), and the sample was lyophilized to give white solid compound 3 (27.5 mg, 31%). LC-MS (ESI): m/z 555.3 (M+H)⁺; ¹H NMR (400 MHz, CD₃OD): δ 8.35 (1H, d, J=17.6 Hz), 8.32 (1H, s), 7.81 (1H, d, J=8.4 Hz), 7.66 (1H, t, J=8.0 Hz), 7.20-7.55 (5H, m), 4.20-4.83 (5H, m), 3.36-3.89 (3H, m), 2.89-3.30 (4H, m), 2.56-2.83 (6H, m), 2.00-2.33 (1H, m), 1.53-1.93 (3H, m), 1.15-1.40 (4H, m).

### Synthesis of Compound 3-1 and 3-2

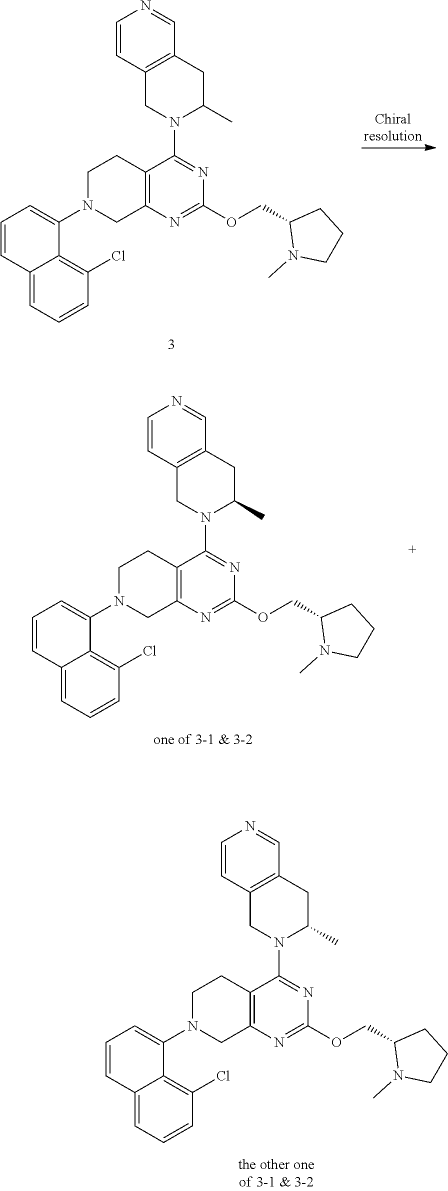 [SMILES: CC1Cc2cnccc2CN1c1nc(OC[C@@H]2CCCN2C)nc2c1CCN(c1cccc3cccc(Cl)c13)C2 ; C[C@@H]1Cc2cnccc2CN1c1nc(OC[C@@H]2CCCN2C)nc2c1CCN(c1cccc3cccc(Cl)c13)C2 ; C[C@H]1Cc2cnccc2CN1c1nc(OC[C@@H]2CCCN2C)nc2c1CCN(c1cccc3cccc(Cl)c13)C2]

Compound 3 (22.5 mg) was purified by chiral resolution to give compound 3-1 (8.8 mg, 39%) and compound 3-2(11 mg, 49%).

| Conditions for chiral analysis | Conditions for chiral preparation |
|---|---|
| Instrument: UPCC | Instrument: SFC-150 (Thar, Waters) |
| Column: IH 4.6*100 mm, 5 um | Column: IH 20*250 mm, 10 um |
| (Daicel) | (Daicel) |
| Column temperature: 40° C. | Column temperature: 35° C. |
| Mobile phase: CO₂/MeOH[0.2% | Mobile phase: CO₂/MeOH[0.2% |
| NH₃(7M in MeOH)] = 75/25 | NH₃(7M in MeOH)] = 65/35 |
| Flow rate: 3.0 mL/min | Flow rate: 100 g/min |
| Wavelength: 214 nm | Back pressure: 100 bar |
| Back pressure: 2000 psi | Wavelength: 214 nm |
| | Circulation: 4.5 min |
| | Sample solution: 22.5 mg in 10 mL of |
| | methanol |
| 3-1: the retention time was 2.10 | |
| min; d.e % = 100.0%; | |
| 3-2: the retention time was 2.66 | |
| min; d.e % = 99.0%. | |

Compound 3-1: LC-MS (ESI): m/z 555.3 (M+H)+; ¹H NMR (400 MHz, MeOD): δ 8.35 (1H, d, J=23.6 Hz), 8.32 (1H, s), 7.82 (1H, d, J=6.4 Hz), 7.67 (1H, t, J=6.8 Hz), 7.20-7.55 (5H, in), 4.20-4.83 (5H, in), 3.36-3.89 (3H, m), 2.89-3.30 (3H, in), 2.62-2.83 (3H, m), 2.40-2.62 (3H, m), 2.00-2.33 (2H, m), 1.53-1.93 (3H, m), 1.15-1.40 (4H, m).

Compound 3-2: LC-MS (ESI): m/z 555.3 (M+H)⁺; ¹H NMR (400 MHz, CD₃OD): δ 8.36 (1H, d, J=22.8 Hz), 8.33 (1H, s), 7.82 (1H, d, J=8.0 Hz), 7.68 (1H, t, J=7.6 Hz), 7.20-7.55 (5H, m), 4.20-4.83 (5H, m), 3.36-3.89 (3H, m), 2.89-3.30 (3H, m), 2.56-2.83 (6H, m), 1.50-2.33 (5H, m), 1.15-1.40 (4H, m).

### Example 4: Synthetic Route of Compound 4

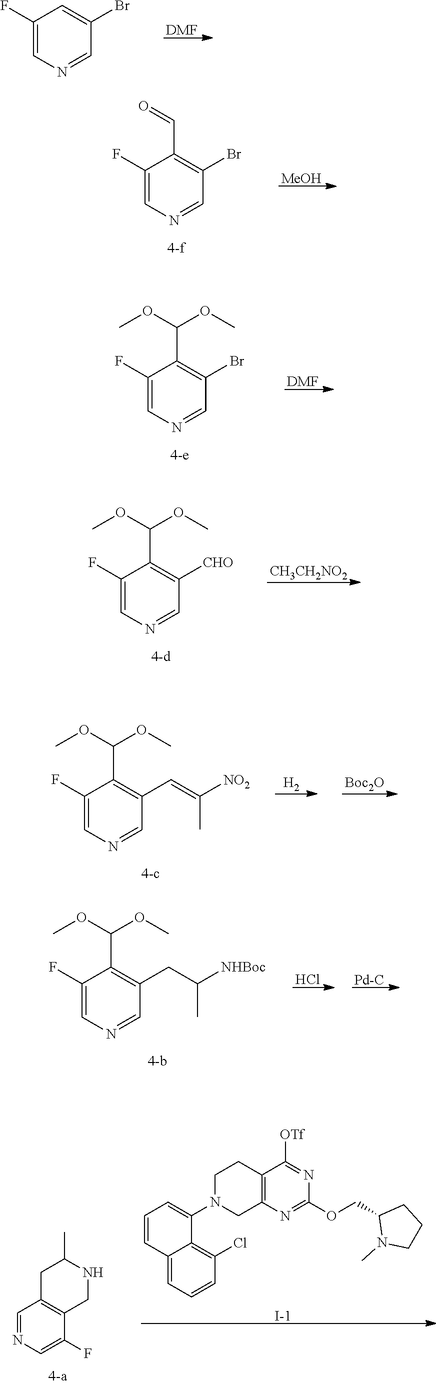

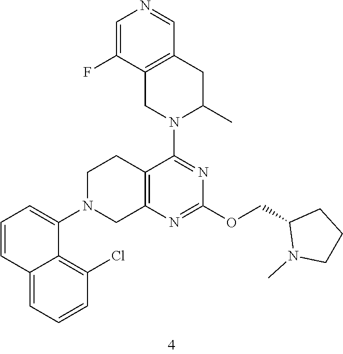 [SMILES: CC1Cc2cncc(F)c2CN1c1nc(OC[C@@H]2CCCN2C)nc2c1CCN(c1cccc3cccc(Cl)c13)C2]

### Synthesis of Compound 4-f

3-Bromo-5-fluoropyridine (23 g, 130.7 mmol) was added to a reaction flask containing dry tetrahydrofuran (400 mL) and stirred at −78° C. for 10 min. LDA (100 mL, 2M, 200.0 mmol) was added slowly to the above mixture. After addition, the reaction was stirred at −78° C. for 2 h. Then dried DMF (32 mL) was added to the reaction, afterwards, the reaction was stirred for 1 hour. The reaction was quenched by adding diluted hydrochloric acid (100 mL, 2M, 200 mmol) and extracted with ethyl acetate (200 mL*3). The organic phase was concentrated and purified by column chromatography (mobile phase: petroleum ether/ethyl acetate 3/1 to 1/1) to give compound 4-f (21 g, 79%). LC-MS (ESI): m/z 224.0 (M+NH₄)

### Synthesis of Compound 4-e

To a reaction flask containing 4-f (21.0 g, 102.9 mmol) was added methanol (200 mL), and the resulting mixture was stirred at room temperature for 10 min. Concentrated sulfuric acid (18.3 g, 190.5 mmol) was slowly added to the above mixture, and the mixture was stirred at 50° C. for 18 hours. The mixture was concentrated, and the residue was added to saturated aqueous sodium bicarbonate solution (400 mL) and extracted with ethyl acetate (200 mL). The organic phase was concentrated to give compound 4-e (21 g, 81%). LC-MS (ESI): m/z 250.1 (M+H)⁺.

### Synthesis of Compound 4-d

4-e (10.5 g, 42.0 mmol) was added into a reaction flask containing dry tetrahydrofuran (150 mL) and the resulting mixture was stirred at −78° C. for 10 min. n-Butyllithium (21.8 mL, 54.5 mmol) was added slowly to the mixture. After addition, the reaction mixture was stirred at −78° C. for 2 hours, then was added dried DMF (20 mL) and stirred for 1 hour. The reaction was quenched by adding saturated ammonium chloride (100 mL) and extracted with ethyl acetate (100 mL*3). The organic phase was concentrated and purified by column chromatography (mobile phase: petroleum ether/ethyl acetate 3/1 to 1/1) to give compound 4-d (5.6 g, 67%). LC-MS (ESI): m/z 200.1 (M+H)⁺.

### Synthesis of Compound 4-c

To a reaction vial containing 4-d (5.6 g, 28.1 mmol) was added nitroethane (35 mL) and ammonium acetate (1.73 g, 22.5 mmol), respectively, and the resulting mixture was stirred at 100° C. for 16 h. The reaction mixture was concentrated and purified by column chromatography (mobile phase: petroleum ether/ethyl acetate 5/1 to 3/1) to give compound 4-c (3.4 g, 47%). LC-MS (ESI): m/z 257.1 (M+H)⁺.

### Synthesis of Compound 4-b

4-c (3.4 g, 13.3 mmol) and Raney nickel (2 g) were added to a reaction flask containing methanol (40 mL). Degassed and purged with hydrogen gas, the mixture was stirred at 30° C. for 60 min under hydrogen balloon (˜1.5 L). Then ammonia in methanol (5 mL, 7 M, 35.0 mmol) was added to the reaction mixture and stirred at 30° C. for 12 hours. The reaction mixture was filtered, and the filtrate was concentrated, and the residue was dissolved in tetrahydrofuran (20 mL) and added Boc₂O (8.3 g, 39.8 mmol) and DIPEA (5.14 g, 39.8 mmol). After addition, the reaction mixture was stirred for 2 hours. The reaction mixture was concentrated and purified by column chromatography (mobile phase: dichloromethane/methanol (containing NH₃) 100/1 to 40/1) to give compound 4-b (2.3 g, 53%). LC-MS (ESI): m/z 329.6 (M+H)⁺.

### Synthesis of Compound 4-a

To a reaction flask containing 4-b (2.3 g, 7.0 mmol) was added HCl/1,4-dioxane (8.0 mL, 4 M, 32.0 mmol) and hydrochloric acid (16 mL, 6 M, 96.0 mmol) and the resulting mixture was stirred at 10° C. for 12 h. The reaction mixture was blown dry with nitrogen and was added methanol (20 mL), dry palladium carbon (387 mg, 10%), and ammonia in methanol (10 mL, 7 M, 70 mmol) were added to the residue. Degassed and purged with hydrogen (1.5 L) for three times and the mixture was stirred at room temperature for 1 h. Filtered, and the filtrate was concentrated, and the residue was purified by column chromatography (mobile phase: dichloromethane/methanol; amine 40/1) to obtain Compound 4-a (750 mg, 65%). IC-MS (ESI): m/z 167.2 (M+H)⁺.

### Synthesis of Compound 4

To a reaction flask were added 4-a (60 mg, 0.36 mmol), DMSO (5 mL), I-1 (220 mg, 0.40 mmol) and DIPEA (373 mg, 2.90 mmol), respectively, and the mixture was stirred at 70° C. for 4 h under nitrogen protection. The reaction mixture was quenched by adding brine (20 mL) and extracted with ethyl acetate (20 mL*3). The organic phase was concentrated to dryness, purified by column chromatography (mobile phase: methanol/dichloromethane 0/100 to 1/10), concentrated and lyophilized to give solid compound 4 (70 mg, 34%). LC-MS (ESI): m/z 573.3 (M+H)⁺; ¹H NMR (400 MHz, CDCl₃): δ 8.28 (1H, d, J=7.2 Hz), 8.25 (1H, d, J=24.0 Hz), 7.76 (1H, d, J=7.2 Hz), 7.33-7.66 (3H, m), 7.66 (1H, t, J=8.0 Hz), 7.28 (1H, s), 4.54-4.86 (3H, m), 4.10-4.50 (2H, m), 3.27-3.92 (4H, m), 2.49-3.33 (8H, m), 1.78-2.46 (5H, m), 1.15-1.40 (4H, m).

### Synthesis of Compound 4-1 and 4-2

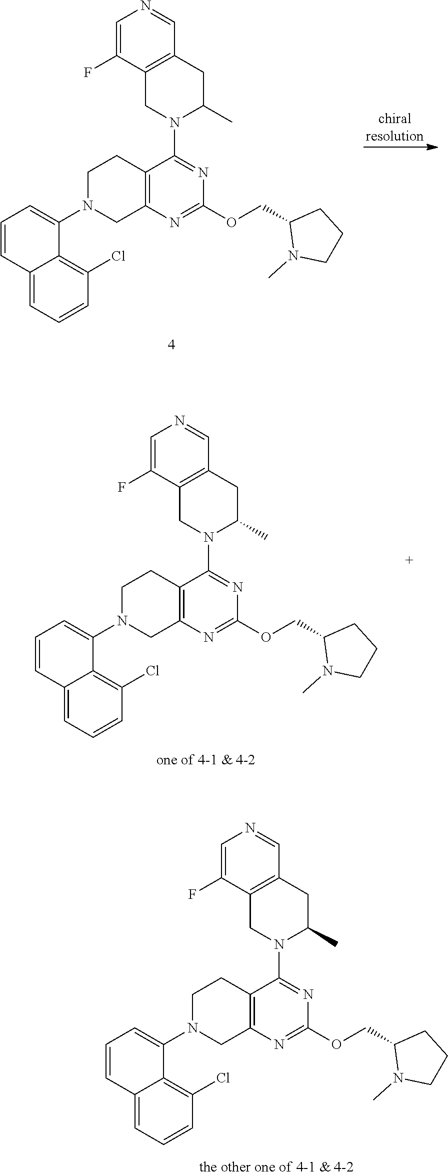 [SMILES: CC1Cc2cncc(F)c2CN1c1nc(OC[C@@H]2CCCN2C)nc2c1CCN(c1cccc3cccc(Cl)c13)C2 ; C[C@H]1Cc2cncc(F)c2CN1c1nc(OC[C@@H]2CCCN2C)nc2c1CCN(c1cccc3cccc(Cl)c13)C2 ; C[C@@H]1Cc2cncc(F)c2CN1c1nc(OC[C@@H]2CCCN2C)nc2c1CCN(c1cccc3cccc(Cl)c13)C2]

Compound 4 (66 mg) was purified by chiral resolution to give compound 4-1 (29 mg, 44%) and compound 4-2 (35 mg, 53%).

| Conditions for chiral analysis | Conditions for chiral preparation |
|---|---|
| Instrument: Agilent1200 | Instrument: Gilson-281 |
| Column: IG 4.6*100 mm, 5 um | Column: IG 20*250 mm, 10 um |
| (Daicel) | (Daicel) |
| Column temperature: 40° C. | Column temperature: 35° C. |
| Mobile phase: n-Hexane (0.1% | Mobile phase: nHexane(0.1% |
| DEA):EtOH (0.1% DEA) = 70:30 | DEA):EtOH(0.1% DEA) = 70:30 |
| Flow rate: 1.0 mL/min | Flow rate: 50 mL/min |
| Wavelength: 254 nm & 214 nm | Wavelength: 214 nm |
| | Circulation: 33 min |
| | Sample solution: 66 mg in 10 mL of |
| | methanol and dichloromethane |
| 4-1: the retention time was 15.7 | |
| min; d.e % = 100.0%; | |
| 4-2: the retention time was 20.39 | |
| min; d.e % = 98.3%. | |

Compound 4-1: LC-MS (ESI): m/z 573.3 (M+H)⁺; ¹H NMR (400 MHz, CD₃OD): δ 8.28 (1H, s), 8.24 (1H, d, J=26.8 Hz), 7.82 (1H, d, J=7.2 Hz), 7.68 (1H, t, J=7.2 Hz), 7.45-7.56 (2H, m), 7.20-7.41 (2H, m), 4.18-4.83 (5H, m), 3.35-3.81 (3H, m), 2.93-3.33 (3H, m), 2.63-2.90 (3H, m), 2.46-2.60 (3H, m), 2.33-2.45 (1H, m), 1.93-2.18 (1H, m), 1.60-1.90 (3H, m), 1.17-1.36 (4H, m).

Compound 4-2: LC-MS (ESI): m/z 573.3 (M+H)⁺; ¹H NMR (400 MHz, CD₃OD): δ 8.29 (1H, s), 8.24 (1H, d, J=26.8 Hz), 7.82 (1H, d, J=8.0 Hz), 7.68 (1H, t, J=7.2 Hz), 7.45-7.56 (2H, m), 7.20-7.41 (2H, m), 4.21-4.83 (5H, m), 3.35-3.81 (3H, m), 2.93-3.33 (3H, m), 2.61-2.90 (3H, m), 2.45-2.60 (3H, m), 2.28-2.45 (1H, m), 1.93-2.18 (1H, m), 1.60-1.90 (3H, m), 1.15-1.36 (4H, m).

### Example 5: Synthetic Route of Compound 5

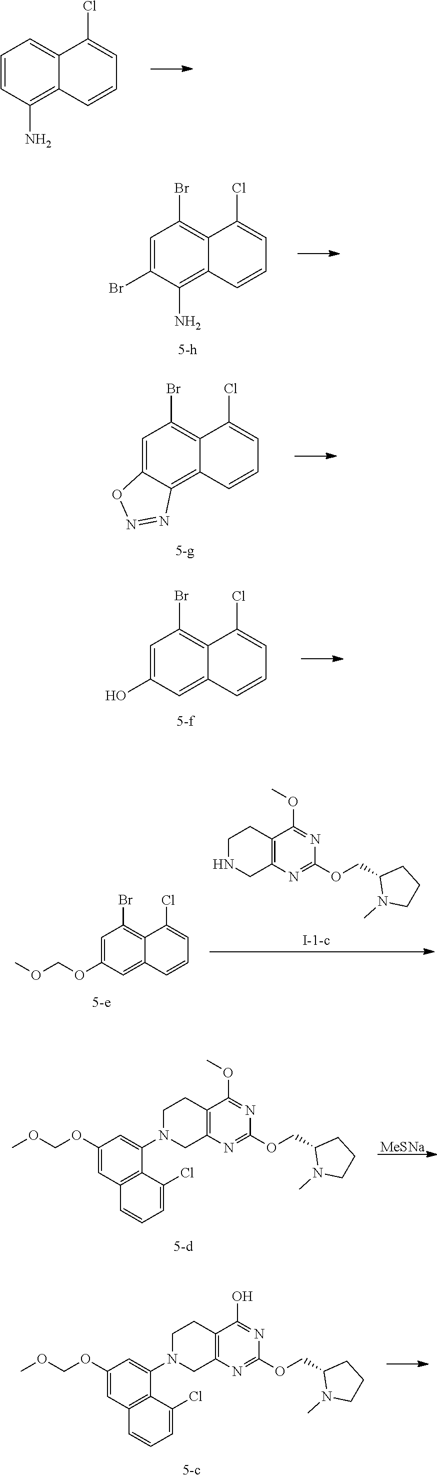

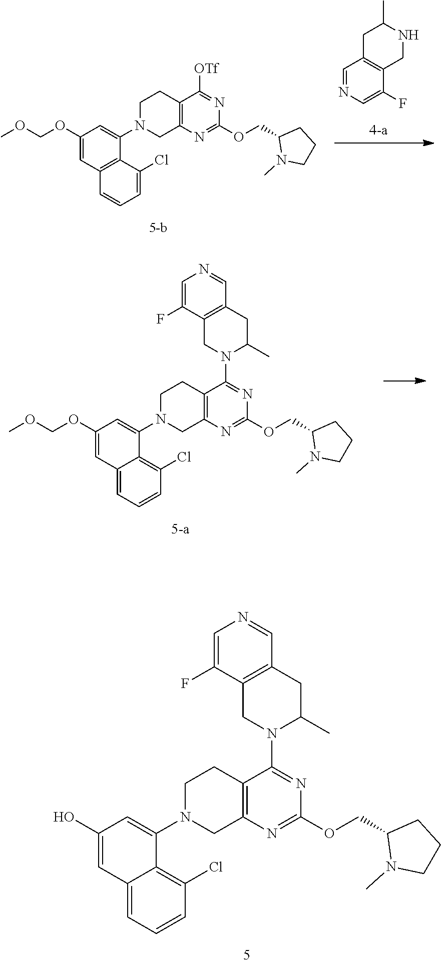 [SMILES: COCOc1cc(N2CCc3c(nc(OC[C@@H]4CCCN4C)nc3OS(=O)(=O)C(F)(F)F)C2)c2c(Cl)cccc2c1 ; CC1Cc2cncc(F)c2CN1 ; COCOc1cc(N2CCc3c(nc(OC[C@@H]4CCCN4C)nc3N3Cc4c(F)cncc4CC3C)C2)c2c(Cl)cccc2c1 ; CC1Cc2cncc(F)c2CN1c1nc(OC[C@@H]2CCCN2C)nc2c1CCN(c1cc(O)cc3cccc(Cl)c13)C2]

### Synthesis of Compound 5-h

A solution of 1-amino-5-chloronaphthalene (10 g, 56.30 mmol) in glacial acetic acid (480 mL) in a reaction flask was slowly added bromine (6.4 mL, 123.2 mmol) under an ice water bath, and stirred at 70° C. for 1 hour. The reaction was cooled in an ice-water bath, filtered and the filter cake was washed with acetic acid. 15% aqueous sodium hydroxide (about 100 mL) was added to the filter cake to adjust the pH to 7, extracted by dichloromethane (300 mL*2), dried over anhydrous sodium sulfate, filtered and removed the solvent to give compound 5-h (12.4 g, 66%) as a dark solid, which was used directly in the next step without purification. ¹H NMR (400 MHz, CDCl₃): δ 7.94 (1H, s), 7.77 (1H, dd, J=8.4 Hz, 1.2 Hz), 7.65 (1H, dd, J=7.6 Hz, J=1.2 Hz), 7.36 (1H, dd, J=8.4 Hz, 7.6 Hz), 4.65 (2H, brs).

### Synthesis of Compound 5-g

5-h (12.4 g, 36.97 mol), glacial acetic acid (236 mL), and propionic acid (30 mL) were combined in a reaction flask, and was added sodium nitrite (3.85 g, 55.86 mmol) under cooling in an ice-water bath. After addition, the resulting mixture was stirred at the same temperature for 30 min and at room temperature for 1 hr. Quenched with water (300 mL), filtered, the filter cake was washed with water, the filter cake was dissolved in ethyl acetate, dried over anhydrous sodium sulfate, filtered and the filtrate was concentrated to dryness to give compound 5-g (9.2 g, 88%) as a dark solid. LC-MS (ESI): m/z=282.7 (M+H)⁺.

### Synthesis of Compound 5-f

5-g (9.2 g, 32.45 mmol), ethanol (200 mL) and tetrahydrofuran (100 mL) were combined in reaction flask, and was slowly added sodium borohydride (2.48 g, 65.25 mmol) in an ice-water bath. After addition, the mixture was stirred at room temperature for 2 h, quenched with 10% aqueous potassium bisulfate, was removed ethanol and tetrahydrofuran by rotary, and the residue was extracted with ethyl acetate (500 mL*2), dried, filtered, and the filtrate was concentrated to dryness, and purified by column chromatography (mobile phase: ethyl acetate/petroleum ether 0/100 to 30/70) to give compound 5-f (2.5 g, 30%) as a dark solid. IC-MS (ESI): m/z=254.7 (M−H)⁻.

### Synthesis of Compound 5-e

To a reaction flask was added 5-f (1.5 g, 5.83 mmol), dichloromethane (50 mL), DIPEA (2.4 mL, 14.65 mmol), then the resulting mixture was added methoxymethyl bromide (0.86 mL, 10.55 mmol) dropwise under an ice water bath. After addition, the mixture was stirred at this temperature for 30 min, quenched with water, extracted with dichloromethane (50 mL*3), concentrated to dryness and purified on a silica column (mobile phase: ethyl acetate/petroleum ether 0/100 to 5/95) to give compound 5-e (1.5 g, 85%) as a brown solid. ¹H NMR (400 MHz, CDCl₃): δ 7.68 (1H, d, J=2.4 Hz), 7.65 (1H, dd, J=8 Hz, J=0.8 Hz), 7.50 (1H, dd, J=7.6 Hz, J=1.2 Hz), 7.37 (1H, d, J=2.4 Hz), 7.30 (1H, t, J=8 Hz), 5.26 (2H, s), 3.51 (3H, s).

### Synthesis of Compound 5-d

To a reaction flask was added 5-e (2.59 g, 8.59 mmol), I-1-c (1.6 g, 5.75 mmol), 1,4-dioxane (80 mL), cesium carbonate (9.37 g, 28.8 mmol), RuPhos (0.537 g, 1.15 mmol) and Pd₂(dba)₃ (0.528 g, 0.58 mmol). Degassed and purged with nitrogen for three times, the mixture was stirred at 100° C. overnight under nitrogen protection, and the next day, the reaction mixture was concentrated to dryness and purified over the column (mobile phase: ammonia-methanol/dichloromethane 0/100 to 5/95) to give compound 5-d (2.1 g, 73%) as a brown solid. LC-MS (ESI): m/z=499.2 (M+H)⁺.

### Synthesis of Compound 5-c

5-d (2.1 g, 4.21 mmol), DMF (20 mL) and sodiumthiomethoxide (1.5 g, 21.08 mmol) were combined in a reaction flask, and the mixture was stirred at 60° C. for 1 h under nitrogen. The reaction was cooled to room temperature, quenched with water, pH was adjusted to 6 with 1 M hydrochloric acid, extracted with ethyl acetate (100 mL*5). The organic phase was dried, removed ethyl acetate and DMF, and purified by column chromatography (mobile phase: ammonia methanol/dichloromethane 0/100 to 5/95) to give compound 5-c (1.5 g, 74%) as a light brown solid. LC-MS (ESI): m/z=485.1 (M+H)⁺.

### Synthesis of Compound 5-b

5-c (500 mg, 1.03 mmol), dichloromethane (50 mL) and triethylamine (430 uL, 3.09 mmol) were combined in a reaction vial, and was added trifluoromethanesulfonic anhydride (260 uL, 1.55 mmol) dropwise under dry-ice cooling. After addition, the mixture was stirred at this temperature for 30 min, quenched with saturated sodium bicarbonate solution, extracted with dichloromethane (50 mL*2), dried over anhydrous sodium sulfate, filtered, concentrated to dryness and purified by column chromatography (mobile phase: methanol/dichloromethane 0/100 to 3/97) to give compound 5-b (550 mg, 86%) as a light brown solid. LC-MS (ESI): m/z=617.1 (M+H)⁺.

### Synthesis of Compound 5-a

To a reaction flask were added 4-a (120 mg, 0.72 mmol), DMAC (8 mL), 5-b (535 mg, 0.87 mmol) and DIPEA (1724 mg, 5.78 mmol) and the mixture was stirred at 60° C. for 8 h under nitrogen protection. The reaction was quenched by adding brine (30 mL) and extracted with ethyl acetate (50 mL*3). The organic phase was concentrated to dryness, purified by column chromatography (mobile phase: methanol (containing NH₃)/dichloromethane 1/30 to 1/20) and lyophilized by concentration to give solid compound 5-a (220 mg, 48%). LC-MS (ESI): m/z 633.3 (M+H)⁺.

### Synthesis of Compound 5

To a vial was added 5-a (220 mg, 0.35 mmol) and acetonitrile (9 mL) and the mixture was stirred for 10 min under nitrogen protection in an ice-salt bath (−5° C.). HCl/1,4-Dioxane (3 mL, 4 M, 12 mmol) was slowly added dropwise to the mixture and continued stirring for 45 min. The reaction was quenched by adding ammonia methanol (30 mL, 7 M, 210 mmol) and concentrated. Washed by (Methanol (containing NH₃)/dichloromethane 1/10 (30 mL*3)) and concentrated. Purified by column chromatography (mobile phase: methanol (containing NH₃)/dichloromethane 1/30 to 1/10), concentrated and lyophilized to give solid compound 5 (120 mg, 59%). LC-MS (ESI): m/z 589.2 (M+H)⁺; ¹H NMR (400 MHz, CD₃OD): δ 8.28 (1H, s), 8.24 (1H, d, J=27.2 Hz), 7.57 (1H, d, J=9.2 Hz), 7.20-7.33 (2H, m), 6.80-7.00 (2H, m), 4.30-4.83 (5H, m), 3.30-3.81 (3H, m), 3.01-3.33 (3H, m), 2.67-2.90 (3H, m), 2.26-2.60 (4H, m), 1.93-2.20 (1H, m), 1.60-1.90 (3H, m), 1.25-1.36 (3H, m), 1.19 (1H, d, J=6.8 Hz).

### Synthesis of Compound 5-1 and 5-2

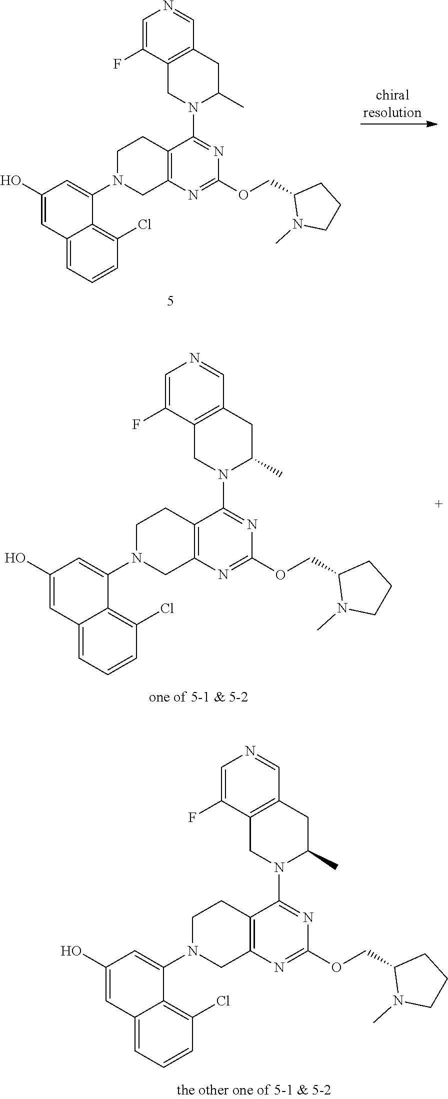 [SMILES: CC1Cc2cncc(F)c2CN1c1nc(OC[C@@H]2CCCN2C)nc2c1CCN(c1cc(O)cc3cccc(Cl)c13)C2 ; C[C@H]1Cc2cncc(F)c2CN1c1nc(OC[C@@H]2CCCN2C)nc2c1CCN(c1cc(O)cc3cccc(Cl)c13)C2 ; C[C@@H]1Cc2cncc(F)c2CN1c1nc(OC[C@@H]2CCCN2C)nc2c1CCN(c1cc(O)cc3cccc(Cl)c13)C2]

Compound 5 (116 mg) was purified by chiral resolution to give compound 5-1 (34 mg, 29%) and compound 5-2 (38 mg, 33%).

| Conditions for chiral analysis | Conditions for chiral preparation |
|---|---|
| Instrument: UPCC | Instrument: SFC-80 (Thar, Waters) |
| Column: AD-3 4.6*100 mm, 3 μm | Columm: AD 20*250 mm, 10 um |
| (Daicel) | (Daicel) |
| Column temperature: 40° C. | Column temperature: 35° C. |
| Mobile phase: CO₂/IPA[1% | Mobile phase: CO₂/IPA(0.2% |
| NH₃(7M in MeOH)] = 55/45 | NH₃(7M in MeOH)] = 50/50 |
| Flow rate: 3.0 mL/min | Flow rate: 100 g/min |
| Wavelength: 214 nm | Pressure: 100 bar |
| Pressure: 2000 psi | Wavelength: 214 nm |
| | Circulation: 6.0 min |
| | Sample solution: 116 mg in 30 mL of |
| | methanol |
| 5-1: the retention time was 1.25 | |
| min; d.e % = 100.0%; | |
| 5-2: the retention time was 1.99 | |
| min; d.e % = 98.1%. | |

Compound 5-1: LC-MS (ESI): m/z 589.2 (M+H)⁺; ¹H NMR (400 MHz, CD₃OD): δ 8.27 (1H, s), 8.23 (1H, d, J=24.4 Hz), 7.56 (1H, d, J=8.0 Hz), 7.18-7.30 (2H, m), 6.80-6.96 (1H, m), 4.18-4.83 (6H, m), 3.30-3.81 (3H, m), 2.93-3.33 (3H, m), 2.63-2.90 (3H, m), 2.41-2.57 (3H, m), 2.26-2.41 (1H, m), 1.93-2.18 (1H, m), 1.60-1.90 (3H, m), 1.12-1.36 (4H, m).

Compound 5-2: LC-MS (ESI): m/z 589.3 (M+H)⁺; ¹H NMR (400 MHz, CD₃OD): δ 8.28 (1H, s), 8.24 (1H, d, J=26.0 Hz), 7.57 (1H, d, J=8.0 Hz), 7.20-7.33 (2H, m), 6.80-7.00 (1H, m), 4.30-4.83 (6H, m), 3.31-3.81 (3H, m), 3.01-3.33 (3H, m), 2.63-2.90 (3H, m), 2.44-2.58 (3H, m), 2.55-2.44 (1H, m), 1.93-2.20 (1H, m), 1.59-1.90 (3H, m), 1.14-1.36 (4H, m).

### Example 6: Synthetic Route of Compound 6

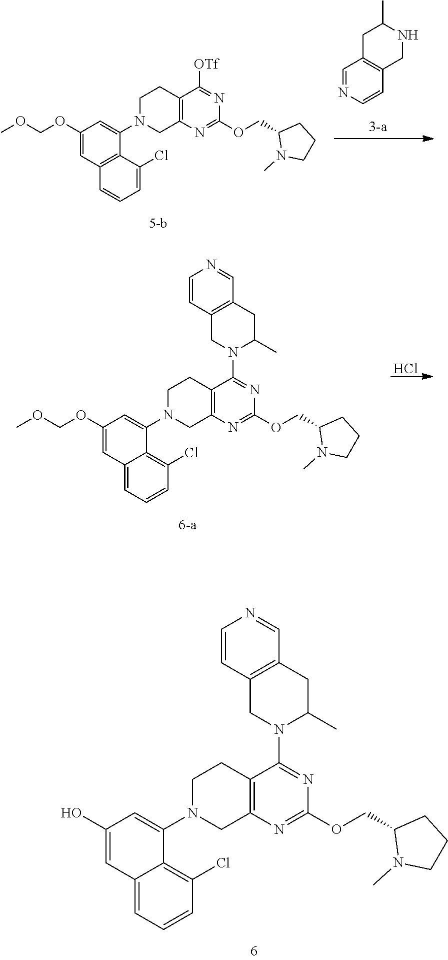 [SMILES: COCOc1cc(N2CCc3c(nc(OC[C@@H]4CCCN4C)nc3OS(=O)(=O)C(F)(F)F)C2)c2c(Cl)cccc2c1 ; CC1Cc2cnccc2CN1 ; COCOc1cc(N2CCc3c(nc(OC[C@@H]4CCCN4C)nc3N3Cc4ccncc4CC3C)C2)c2c(Cl)cccc2c1 ; Cl ; CC1Cc2cnccc2CN1c1nc(OC[C@@H]2CCCN2C)nc2c1CCN(c1cc(O)cc3cccc(Cl)c13)C2]

### Synthesis of Compound 6-a

To a reaction flask were added 3-a (100 mg, 0.68 mmol), DMAC (8 mL), 5-b (479 mg, 0.78 mmol) and DIPEA (697 mg, 5.4 mmol), and the mixture was stirred at 60° C. for 3 h under nitrogen protection. The reaction mixture was quenched with brine (30 mL) and extracted with ethyl acetate (50 mL*3). The organic phase was concentrated to dryness, purified by column chromatography (mobile phase: methanol (containing NH₃/dichloromethane 1/30 to 1/20) and concentrated lyophilized to give solid compound 6-a (170 mg, 41%). LC-MS (ESI): m/z 615.3 (M+H)⁺.

### Synthesis of Compound 6

6-a (170 mg, 0.28 mmol) and acetonitrile (9 mL) were added separately into a reaction flask, and the resulting mixture was stirred for 10 min under an ice-salt bath (about −5° C.), and HCl/1,4-dioxane (3 mL, 12 mmol) was added slowly dropwise to the above mixture under nitrogen protection. After addition, the reaction was stirred for 45 minutes. The reaction was quenched by adding ammonia-methanol (30 mL, 7 M, 210 mmol) to the reaction and concentrated. Slurry with methanol (containing NH₃)/dichloromethane 1/10 (30 mL*3), filter, and concentrate the organic phase with filtrate. Column chromatography (mobile phase: methanol; NH₃/dichloromethane 1/30 to 1/10), concentrated and lyophilized to give solid compound 6 (90 mg, 57%). LC-MS (ESI): m/z 571.2 (M+H)⁺; ¹H NMR (400 MHz, CD₃OD): δ 8.38 (1H, s), 8.33 (1H, s), 8.32 (1H, s), 7.57 (1H, d, J=8.4 Hz), 7.19-7.33 (3H, m), 6.88 (1H, d, J=26.8 Hz), 4.71-4.90 (2H, m), 4.20-4.45 (3H, m), 3.29-3.70 (3H, m), 3.00-3.33 (3H, m), 2.27-2.90 (7H, m), 1.60-2.18 (4H, m), 1.13-1.38 (4H, m).

### Example 7: Synthetic Route of Compound 7

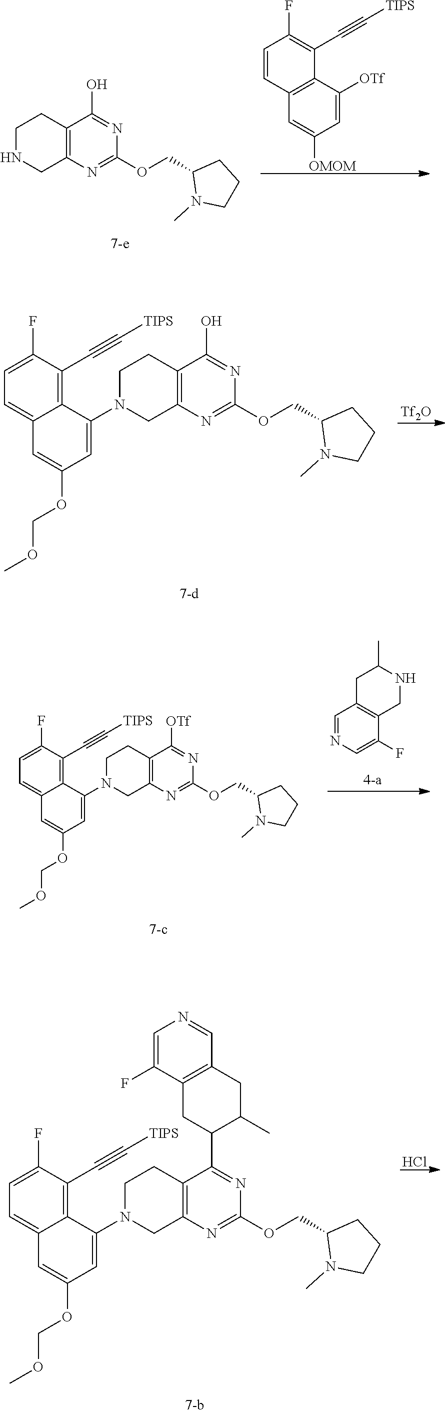

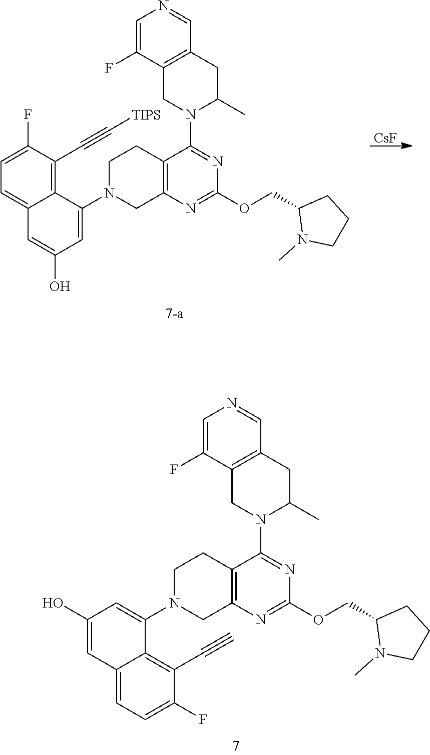

### Synthesis of Compound 7-d

7-e (400 mg, 1.51 mmol), 7-fluoro-3-(methoxymethoxy)-8-((triisopropylmethylsilyl)ethynyl)naphthalen-1-yl trifluoromethanesulfonate (889 mg, 1.66 mmol), toluene (30 mL), sodium tert-butoxide (432 mg, 4.50 mmol), Pd₂(dba)₃ (137 mg, 0.15 mmol), and BINAP (186 mg, 0.30 mmol) were combined in a reaction flask. Degassed and purged with N₂, the reaction was stirred at 110° C. for 3 h under N₂ protection. The reaction mixture was concentrated and purified by column chromatography(mobile phase: DCM/MeOH 10/0 to 10/1) to give compound 7-d (400 mg, 41%). LC-MS (ESI): m/z 649.5 (M+H)⁺.

### Synthesis of Compound 7-c

To a reaction vial were added 7-d (240 mg, 0.37 mmol), DIPEA (382 mg, 3.0 mmol) and dichloromethane (60 mL), and Tf₂O (188 mg, 0.67 mmol) was slowly added dropwise to the above mixture under nitrogen protection in a dry ice bath (about −40° C.) for 10 min. After addition, the reaction mixture was stirred for 45 min. The reaction mixture was quenched by adding saturated sodium bicarbonate (60 mL) and extracted with dichloromethane (60 mL*3). The organic phase was concentrated, purified by column chromatography (mobile phase: methanol/dichloromethane 1/20 to 1/10), concentrated and lyophilized to give white solid compound 7-c (190 mg, 66%). LC-MS (ESI): m/z 781.7 (M+H)⁺.

### Synthesis of Compound 7-b

7-c (120 mg, 0.15 mmol), DMAC (5 mL), 4-a (33 mg, 0.2 mmol) and DIPEA (99 mg, 0.77 mmol) were combined in a reaction vial, and the resulting mixture was stirred at 70° C. for 8 h under nitrogen protection. The reaction mixture was quenched by adding brine (20 mL) to the reaction mixture and extracted with ethyl acetate (20 mL*3). The organic phase was concentrated to dryness, purified by column chromatography (mobile phase: methanol (containing NH₃)/dichloromethane 1/30 to 1/10), concentrated and lyophilized to give a white solid compound 7-b (90 mg, 73%). LC-MS (ESI): m/z 798.0 (M+H)⁺.

### Synthesis of Compound 7-a

7-b (90 mg, 0.11 mmol) and acetonitrile (8 mL) were added to the reaction flask, and stirred in an ice-salt bath (−5° C.) for 10 min. Then HCl/1,4-dioxane (2 mL, 4 M, 8 mmol) was slowly added dropwise to the above mixture under nitrogen protection. After addition, the reaction mixture was stirred for 50 min. The reaction mixture was quenched with ammonia-methanol (30 mL, 7 M, 210 mmol) and concentrated. The reaction mixture was pulsed with methanol (containing NH₃)/dichloromethane 1/10 (30 mL*3), filtered, the filtrate was concentrated and passed through column chromatography (mobile phase: methanol (containing NH₃)/dichloromethane 1/30 to 1/10), concentrated and lyophilized to give white solid compound 7-a (70 mg, 82%). LC-MS (ESI): m/z 753.9 (M+H)⁺.

### Synthesis of Compound 7

Cesium fluoride (230 mg, 1.51 mmol) was added to a solution of 7-a (70 mg, 0.09 mmol) in DMF (2 mL) while stirring at room temperature under nitrogen protection, and the reaction mixture was stirred for 14 hours. The reaction mixture was quenched with water (10 mL), extracted with ethyl acetate (20 mL*3), concentrated, purified by column chromatography (mobile phase: methanol (containing NH₃)/dichloromethane 1/30 to 1/10), concentrated and lyophilized to give white solid compound 7 (4.3 mg, 7.8%). LC-MS (ESI): m/z 597.4 (M+H)⁺; ¹H NMR (400 MHz, CDCl₃): δ 8.20-8.33 (2H, m), 7.40-7.62 (1H, m), 7.01-7.20 (1H, m), 6.79-7.00 (2H, m), 4.01-4.92 (5H, m), 3.00-3.60 (5H, m), 2.34-2.93 (8H, m), 1.67-2.31 (5H, m), 1.11-1.38 (4H, m).

### Example 8: Synthetic Route of Compound 8

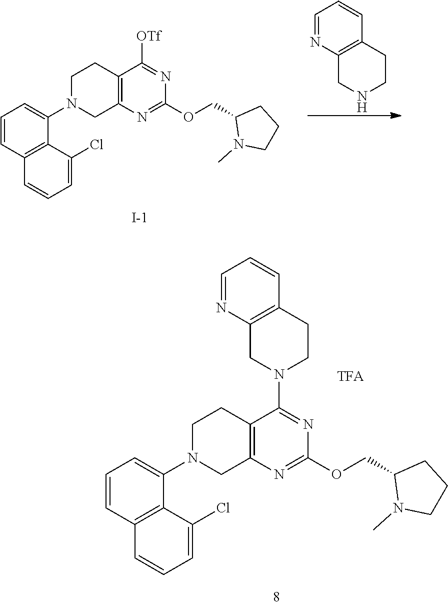 [SMILES: CN1CCC[C@H]1COc1nc2c(c(OS(=O)(=O)C(F)(F)F)n1)CCN(c1cccc3cccc(Cl)c13)C2 ; c1cnc2c(c1)CCNC2 ; [I-] ; CN1CCC[C@H]1COc1nc2c(c(N3CCc4cccnc4C3)n1)CCN(c1cccc3cccc(Cl)c13)C2]

### Synthesis of Compound 8

I-1 (20 mg, 0.036 mmol), DMF (2 mL), 5,6,7,8-tetrahydro-[1,7]naphthyridine (10 mg, 0.072 mmol) and DIPEA (30 uL, 0.18 mmol) were combined in a reaction vial, and the mixture was stirred at room temperature for 1 h under nitrogen protection. The reaction mixture was purified directly by preparative HPLC (mobile phase containing trifluoroacetic acid) and lyophilized to give compound 8 (20 mg, 87%) as a light brown solid. LC-MS (ESI): m/z=541.3 (M+H)⁺; ¹H NMR (400 MHz, CD₃OD): S 8.57 (1H, d, J=5.6 Hz), 8.10 (1H, d, J=8 Hz), 7.85 (1H, d, J=8 Hz), 7.72 (1H, d, J=7.6 Hz), 7.67-7.61 (1H, m), 7.58-7.48 (2H, m), 7.42-7.33 (2H, m), 5.20 (1H, d, J=17.6 Hz), 5.09 (1H, d, J=17.6 Hz), 4.88-4.79 (2H, m), 4.71-4.60 (1H, m), 4.42-4.31 (2H, m), 4.00-3.69 (4H, m), 3.68-3.59 (1H, m), 3.49-3.35 (1H, m), 3.29-3.19 (2H, m), 3.16-3.01 (4H, m), 2.87-2.77 (1H, m), 2.49-2.35 (1H, m), 2.29-1.98 (3H, m).

### Example 9: Synthetic Route of Compound 9

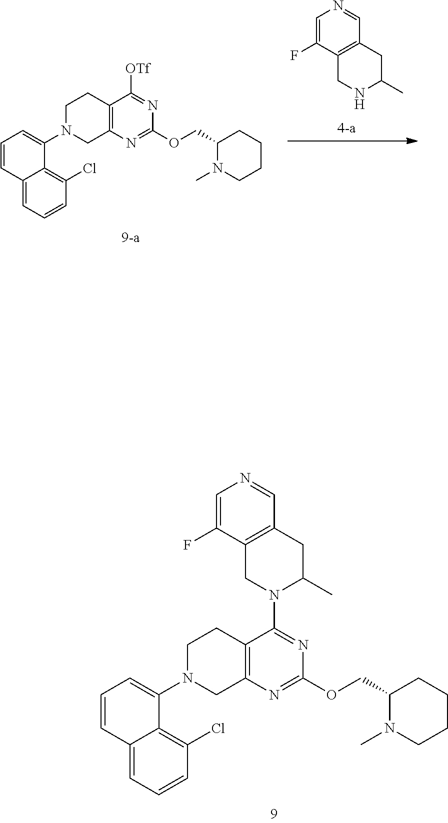 [SMILES: CN1CCCC[C@H]1COc1nc2c(c(OS(=O)(=O)C(F)(F)F)n1)CCN(c1cccc3cccc(Cl)c13)C2 ; CC1Cc2cncc(F)c2CN1 ; CC1Cc2cncc(F)c2CN1c1nc(OC[C@@H]2CCCCN2C)nc2c1CCN(c1cccc3cccc(Cl)c13)C2]

### Synthesis of Compound 9-a

Compound 9-a was synthesized by replacing N-methyl-L-prolinol with (S)-(1-methylpiperidin-2-yl)methanol according to the synthetic route of I-1.

### Synthesis of Compound 9

9-a (140 mg, 0.25 mmol), DMAC (3 mL), 4-a (49 mg, 0.29 mmol) and DIPEA (158 mg, 1.23 mmol) were combined in a reaction flask, and the mixture was stirred at 60° C. for 3 h under nitrogen protection. The reaction mixture was quenched by adding brine (20 mL), the solid was filtered out and purified by column chromatography (mobile phase: methanol (containing NH₃)/dichloromethane 1/20 to 1/10) and concentrated to give compound 9 (55 mg, 38%).

### Synthesis of Compounds 9-1 and 9-2

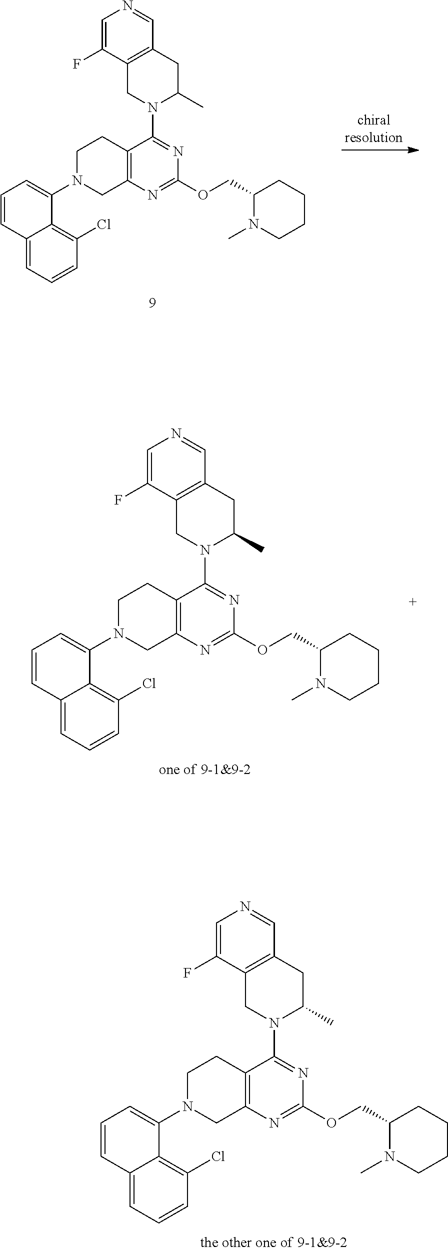 [SMILES: CC1Cc2cncc(F)c2CN1c1nc(OC[C@@H]2CCCCN2C)nc2c1CCN(c1cccc3cccc(Cl)c13)C2 ; C[C@@H]1Cc2cncc(F)c2CN1c1nc(OC[C@@H]2CCCCN2C)nc2c1CCN(c1cccc3cccc(Cl)c13)C2 ; C[C@H]1Cc2cncc(F)c2CN1c1nc(OC[C@@H]2CCCCN2C)nc2c1CCN(c1cccc3cccc(Cl)c13)C2]

Compound 9(51 mg) was purified by chiral resolution to give compound 9-1 (7.5 mg, 15%) and compound 9-2 (7.7 mg, 15%).

| Conditions for chiral analysis | Conditions for chiral preparation |
|---|---|
| Instrument: UPCC | Instrument: SFC-150 (Thar, Waters) |
| Column: AS-H 4.6*100 mm, 5 μm | Column: AS 20*250 mm, 10 um |
| (Daicel) | (Daicel) |
| Column temperature: 40° C. | Column temperature: 35° C. |
| Mobile phase: CO₂/MeOH[1% | Mobile phase: CO₂/MeOH[0.5% |
| NH₃(7M in MeOH)] = 80/20 | NH₃(7M in MeOH)] = 70/30 |
| Flow rate: 3.0 mL/min | Flow rate: 100 g/min |
| Wavelength: 214 nm | Pressure: 100 bar |
| Pressure: 2000 psi | Wavelength: 214 nm |
| | Circulation: 5.2 min |
| | Sample solution: 51 mg in 10 mL of |
| | methanol |
| 9-1: the retention time was 2.20 | |
| min; d.e % = 100.0%; | |
| 9-2: the retention time was 2.89 | |
| min; d.e % = 99.7%. | |

Compound 9-1: LC-MS (ESI): m/z 587.3 (M+H)⁺; ¹H NMR (400 MHz, DMSO-d₆): δ 8.38 (1H, d, J=2.8 Hz), 8.29 (1H, d, J=24.8 Hz), 7.88-7.95 (1H, m), 7.74 (1H, t, J=7.6 Hz), 7.25-7.65 (4H, m), 4.50-4.77 (2H, m), 4.00-4.44 (4H, m), 3.39-3.59 (1H, m), 2.95-3.27 (3H, m), 2.52-2.90 (3H, m), 2.06-2.33 (4H, m), 1.92-2.05 (1H, m), 1.59-1.75 (2H, m), 1.03-1.59 (8H, m).

Compound 9-2: LC-MS (ESI): m/z 587.3 (M+H)⁺; ¹H NMR (400 MHz, CD₃OD): δ 8.28 (1H, s), 8.24 (1H, d, J=26.8 Hz), 7.82 (1H, dd, J₁=1.2 Hz, J₂=8.0 Hz), 7.68 (1H, t, J=7.2 Hz), 7.40-7.56 (2H, m), 7.20-7.40 (2H, m), 4.24-4.83 (5H, m), 3.35-3.84 (3H, m), 2.62-3.33 (5H, m), 2.06-2.45 (5H, m), 1.50-1.95 (4H, m), 1.10-1.50 (6H, m).

### Example 10: Synthetic Route of Compound 10

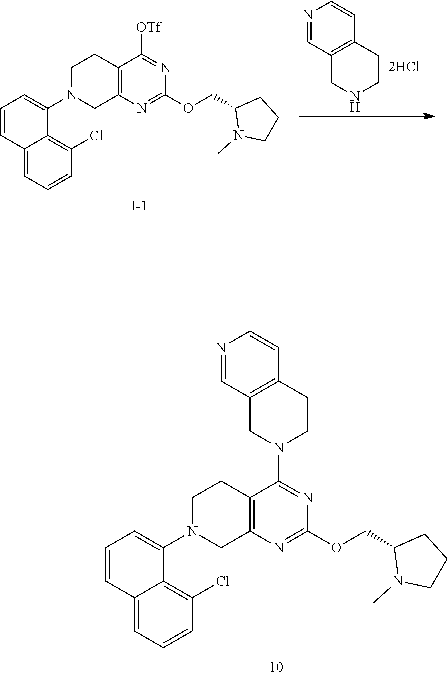 [SMILES: CN1CCC[C@H]1COc1nc2c(c(OS(=O)(=O)C(F)(F)F)n1)CCN(c1cccc3cccc(Cl)c13)C2 ; c1cc2c(cn1)CNCC2 ; [I-] ; CN1CCC[C@H]1COc1nc2c(c(N3CCc4ccncc4C3)n1)CCN(c1cccc3cccc(Cl)c13)C2]

### Synthesis of Compound 10

I-1 (20 mg, 0.036 mmol), DMF (2 mL), 1,2,3,4-tetrahydro-2,7-naphthyridine hydrochloride (15 mg, 0.072 mmol) and DIPEA (0.5 mL) were combined in a reaction vial, and the resulting mixture was stirred at room temperature overnight under nitrogen. The next day, the reaction mixture was purified directly by prep-HPLC (ammonium bicarbonate) and lyophilized to give compound 10 (18 mg, 93%) as a white solid. LC-MS (ESI): m/z=541.0 (M+H)⁺; ¹H NMR (400 MHz, CDCl₃): δ 8.44 (1H, s), 8.39 (1H, d, J=5.2 Hz), 7.75 (1H, d, J=8.4 Hz), 7.61 (1H, d, J=7.6 Hz), 7.52 (1H, d, J=7.2 Hz), 7.44 (1H, t, J=8.0 Hz), 7.33 (1H, t, J=8.0 Hz), 7.22 (1H, d, J=7.6 Hz), 7.10 (1H, d, J=4.8 Hz), 4.78-4.65 (2H, m), 4.58-4.47 (1H, m), 4.42 (1H, d, J=18.0 Hz), 4.29-4.17 (1H, m), 4.03-3.92 (1H, m), 3.85 (1H, d, J=18.0 Hz), 3.61-3.48 (2H, m), 3.29-3.06 (4H, m), 2.98-2.75 (2H, m), 2.65-2.51 (4H, m), 2.47-2.31 (1H, m), 2.18-2.05 (1H, m), 1.98-1.75 (3H, m).

Synthetic route of intermediate I-2

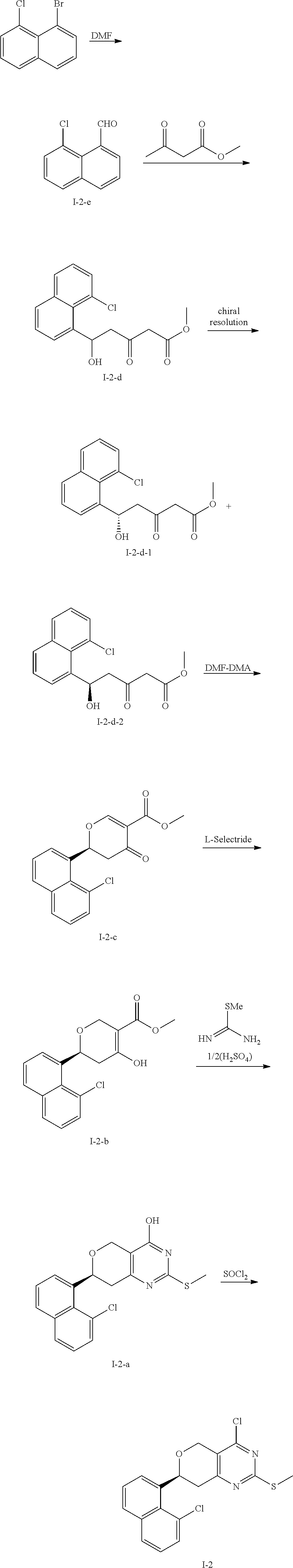

### Synthesis of compound I-2-e

n-BuLi (2.5 M, 1.66 mL, 4.14 mmol) was added dropwise to a solution of compound 1-bromo-8-chloronaphthalene (500 mg, 2.07 mmol) in THF (20 mL) at −78° C. under nitrogen protection. After the addition, the mixture was stirred at −78° C. for 10 min, and then was added DMF (800 μL, 10.35 mmol) dropwise at −78° C. After addition, the reaction mixture was stirred at −78° C. for 30 min, then stirred at room temperature for 2 h. The reaction was quenched with 50 mL of saturated ammonium chloride solution and extracted with ethyl acetate (50 mL*2). The organic phases were washed with brine (50 mL*2), dried over anhydrous sodium sulfate, filtered and concentrated to give the crude product. The crude product was purified by flash separation column (EA/PE=1/10) to give compound I-2-e (330 mg, 84% yield) as a white solid. LC-MS (ESI): m/z=191.0 [M+H]*; ¹H NMR (400 MHz, CDCl₃): δ 11.31 (s, 1H), 8.03 (dd, 1H, J₁=1.2 Hz, J₂=8.4 Hz), 7.92 (dd, 1H, J=1.2 Hz, J₂=7.2 Hz), 7.86 (1H, J=8.4 Hz), 7.70 (dd, 1H, J=1.2 Hz, J₂=7.6 Hz), 7.59 (t, 1H, J=7.6 Hz), 7.47 (t, 1H, J=8 Hz).

### Synthesis of compound I-2-d

Methyl acetoacetate (543 μL, 5.04 mmol) was added into a solution of NaH (60%, 242 mg, 6.05 mmol) in THF (6 mL) under nitrogen at room temperature, and the mixture was stirred at room temperature under nitrogen for 30 min, then was added n-BuLi (2.5 M, 2.4 mL, 6.05 mmol) dropwise at −15° C. to −10° C. After addition, the mixture was kept at this temperature for 30 min, and then was added a solution of compound I-2-e (320 mg, 1.68 mmol) in THF (10 mL) dropwise. After addition, the mixture was stirred at low temperature (−10° C.˜0° C.) for 2 h. The reaction was quenched with saturated ammonium chloride solution (50 mL), and then extracted with ethyl acetate (50 mL*2). The organic phase was washed with brine (50 mL*2), dried over anhydrous sodium sulfate, filtered and concentrated to obtain the crude product, which was purified by flash column (EA/DCM=1/10) to give compound I-2-d (510 mg, 99% yield) as a white solid. LC-MS (ESI): m/z=329.1 [M+Na]*; ¹H NMR (400 MHz, CDCl₃): δ 8.06 (d, 1H, J=6.4 Hz), 7.79 (d, 2H, J=8 Hz), 7.58 (dd, 1H, J=7.6 Hz, J₂=1.6 Hz), 7.53 (t, 1H, J=7.6 Hz), 7.34 (t, 1H, J=7.6 Hz), 6.91 (dd, 1H, J=9.2 Hz, J₂=2.4 Hz), 3.74 (s, 3H), 3.54 (s, 2H), 3.36 (dd, 1H, J₁=18 Hz, J₂=1.6 Hz), 3.24 (d, 1H, J=3.6 Hz), 2.85-2.75 (in, 11H).

### Synthesis of compound I-2-d-1 and I-2-d-2

Amplification the preparation of compound I-2-d (8.5 g, 27.8 mmol), which was purified by chiral resolution to give compound I-2-d-1 (2.5 g, 29%) as white solid and compound I1-2-d-2 (2.6 g, 31%) as white solid.

| Conditions for chiral analysis | Conditions for chiral preparation |
|---|---|
| Instrument: SFC Method Station | Instrument: SFC-150 (Thar, |
| (Thar, Waters) | Waters) |
| Column: AD-H 4.6*100 mm, 5 um | Column: AD 20*250 mm, 10 um |
| (Daicel) | (Daicel) |
| Column temperature: 40° C. | Column temperature: 35° C. |
| Mobile phase: CO₂/Ethanol (1% | Mobile phase: CO₂/Ethanol (0.2% |
| Ammonia in Methanol) = 75/25 | Ammonia in Methanol) = 65/35 |
| Flow rate: 4.0 mL/min | Flow rate: 100 g/min |
| Wavelength: 254 nm | Pressure: 100 bar |
| Pressure: 120 bar | Wavelength: 214 nm |
| | Circulation: 5.0 min |
| | Sample solution: 8.5 g in 150 mL of |
| | methanol and dichloromethane |
| I-2-d-1: the retention time was 1.57 | |
| min; e.e. % = 100.0%; | |
| I-2-d-2: the retention time was 2.33 | |
| min; e.e. % = 99.12%. | |

I-2-d-1: LC-MS (ESI): m/z = 329.1 [M + Na]⁺.
I-2-d-2: LC-MS (ESI): m/z = 329.1 [M + Na]⁺.

### Synthesis of Compound 1-2-c

DMF-DMA (1.2 mL, 9.0 mmol) was added to a solution of compound I-2-d-1 (2.3 g, 7.5 mmol) in DCM (80 mL) at room temperature under nitrogen. Stirred at room temperature for 45 min, The above mixture was added BF₃·Et₂O (1.2 mL, 9.0 mmol). After addition, the mixture was stirred at room temperature for 1 h, quenched with saturated sodium bicarbonate solution, extracted with dichloromethane (100 mL*2), the organic phase was washed with saturated saline (100 mL*2), dried over anhydrous sodium sulfate, filtered and concentrated to obtain the crude compound I-2-c (2.0 g, 84%), which was directly used in the next step. LC-MS (ESI): m/z=317.1 [M+1]⁺.

### Synthesis of Compound 1-2-b

Lithium tri-sec-butylborohydride (1 M in THF, 6.95 mL, 6.95 mmol) was added dropwise o to a solution of compound I-2-c (2.0 g, 6.31 mmol) in THF (60 mL) at room temperature at −78° C. under nitrogen. After addition, the mixture was stirred at −78° C. for 1 h, then was quenched with 1 M hydrochloric acid solution (20 mL), extracted with ethyl acetate (100 mL*2). The organic phase was washed with saturated saline (50 mL*2), dried over anhydrous sodium sulfate, filtered and concentrated to obtain the crude product, which was purified by fast separation column (PE/EA=0˜15%) to give compound 1-2-b (1.8 g, 89%) as a yellow oil. LC-MS (ESI): m/z=319.0 [M+1]⁺.

### Synthesis of Compound 1-2-a

Sodium methanol (1.27 g, 23.5 mmol) and compound 2-methyl-2-mercapto sulfate urea (1.18 g, 4.24 mmol) were added sequentially to a solution of compound I-2-b (1.5 g, 4.71 mmol) in methanol (30 mL) at 0° C. under nitrogen. After addition, the mixture was stirred at room temperature for 20 hours. The reaction mixture was adjusted to pH 5 with 1 M dilute hydrochloric acid, solid was precipitated, filtered and the filter cake was washed with a mixture of ethyl acetate (20 mL) and petroleum ether (20 mL), the solid was collected and dried at vacuum to give crude I-2-a (0.65 g, 39%) as a white solid. LC-MS (ESI): m/z=359.1 [M+1]*.

### Synthesis of Compound 1-2

Sulfoxide chloride (9.3 g, 78.0 mmol) was added dropwise to a solution of I-2-a (4.0 g, 11.1 mmol) in DMF (40 mL) and DCM (20 mL) in an ice-water bath. After addition, the reaction mixture was stirred in an ice-water bath for 4 hours. The reaction mixture was slowly added dropwise into 60 mL of water while keeping the internal temperature from 0 to 10° C. The mixture was extracted with DCM. The organic phase was washed with saturated sodium bicarbonate, washed with water, concentrated, slurred with n-heptane, cooled to 0˜10° C., filtered and dried to give compound I-2 (3.2 g, 76%). LC-MS (ESI): m/z=377.0 [M+H]*. The structure of this compound was determined by single crystal resolution.

### Example 11: Synthetic Route of Compound 11

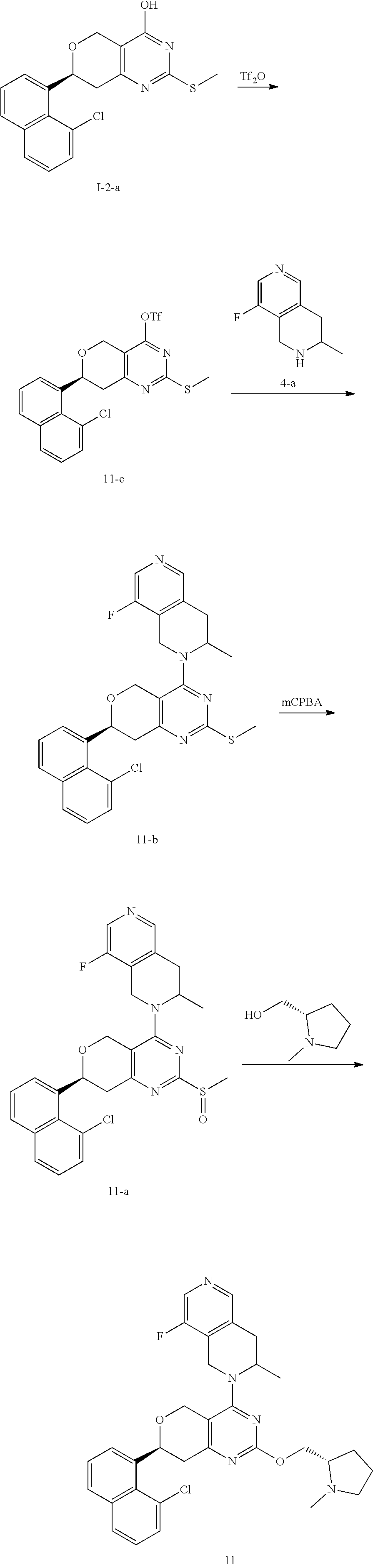

### Synthesis of Compound 11-c

N,N-diisopropylethylamine (72 mg, 0.56 mmol) was added to a solution of I-2-a (100 mg, 0.28 mmol) in DCM (15 mL) in ice-water bath, and then trifluoromethanesulfonic anhydride (142 mg, 0.50 mmol) was added slowly to the above mixture. After addition, the reaction was stirred for 1 hour in ice-water bath. When completed, the reaction was diluted with dichloromethane, washed with water, brine, dried over anhydrous Na₂SO₄, filtered and concentrated. The crude product was purified by flash chromatography (mobile phase: petroleum ether/ethyl acetate to 10/1) to give 11-c (124 mg, 91%) as a white solid. LC-MS (ESI): m/z 491.1 (M+H)⁺.

### Synthesis of Compound 11-b

N,N-diisopropylethylamine (65 mg, 0.50 mmol) and 4-a (42 mg, 0.25 mmol) were added to a solution of 11-c (124 mg, 0.25 mmol) in DMSO (6 mL) at room temperature. After addition, the reaction mixture was stirred at 100° C. for 1 hour. When completed, the reaction mixture was diluted with ethyl acetate, washed with water and brine sequentially, dried over anhydrous Na₂SO₄, filtered and concentrated to dryness. The crude product was purified by flash chromatography (mobile phase: dichloromethane/methanol 1/0 to 10/1) to give 11-b (100 mg, 78%) as a yellow solid. LC-MS (ESI): m/z 507.2 (M+H)⁺.

### Synthesis of Compound 11-a

m-chloroperoxybenzoic acid (85%, 60 mg, 0.3 mmol) was added to a solution of compound 11-b (100 mg, 0.2 mmol) in ethyl acetate (10 mL) under ice bath, then and the mixture was stirred for 2 h under ice bath conditions. When completed, the reaction was added saturated aqueous sodium bicarbonate to neutralize, separated and the aqueous phase was extracted with ethyl acetate. The organic phases were combined, dried over anhydrous Na₂SO₄, filtered and evaporated to obtain crude 11-a (102 mg) as a yellow solid, which was used directly in the next step without purification. LC-MS (ESI): m/z 523.2 (M+H)⁺.

### Synthesis of Compound 11

Sodium tert-butoxide (38 mg, 0.39 mmol) was added to a solution of 11-a (102 mg, 0.2 mmol) and N-methyl-L-prolinol (33 mg, 0.29 mmol) in toluene (6 mL) in an ice-water bath. After addition, the reaction was stirred in an ice-water bath for 2 hours. When completed, the reaction was concentrated under reduced pressure, diluted with ethyl acetate, washed with water, brine, dried over anhydrous Na₂SO₄, filtered and concentrated to dryness, and the crude product was purified by fast chromatography (mobile phase: dichloromethane/methanol I/O to 10/1) to give compound 11 (34 mg, 30%) as a white solid. LC-MS (ESI): m/z 574.2 (M+H)⁺.

### Synthetic Routes of Compounds 11-1 and 11-2

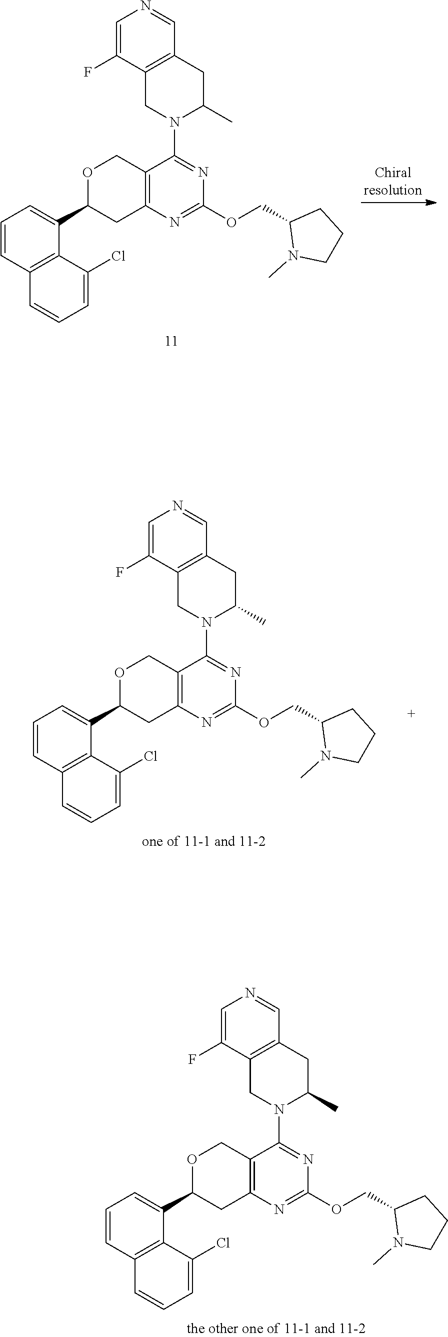 [SMILES: CC1Cc2cncc(F)c2CN1c1nc(OC[C@@H]2CCCN2C)nc2c1CO[C@H](c1cccc3cccc(Cl)c13)C2 ; C[C@H]1Cc2cncc(F)c2CN1c1nc(OC[C@@H]2CCCN2C)nc2c1CO[C@H](c1cccc3cccc(Cl)c13)C2 ; C[C@@H]1Cc2cncc(F)c2CN1c1nc(OC[C@@H]2CCCN2C)nc2c1CO[C@H](c1cccc3cccc(Cl)c13)C2]

Compound 11 (31 mg) was purified by chiral resolution and Pre-HPLC to give compound 11-1 (2.1 mg) and compound 11-2 (6.1 mg), both are white solid.

| Conditions for chiral analysis | Conditions for chiral preparation |
|---|---|
| Instrument: UPCC (Waters) | Instrument: SFC-150 (Waters) |
| Column: OJ-3 4.6*100 mm, 3 um | Column: OJ 20*250 mm, 10 um |
| (Daicel) | (Daicel) |
| Column temperature: 40° C. | Column temperature: 35° C. |
| Mobile phase: CO₂/Methanol[0.2% | Mobile phase: CO₂/Methanol[0.2% |
| NH₃(7M in Methanol)] = 55/45 | NH₃(7M in Methanol)] = 40/60 |
| Flow rate: 3.0 mL/min | Flow rate: 120 g/min |
| Wavelength: 214 nm | Pressure: 100 bar |
| Pressure: 2000 psi | Wavelength: 214 nm |
| | Circulation: 4.5 min |
| | Sample solution: 31 mg dissolved in |
| | 20 mL Methanol |
| 11-1: the retention time was 0.875 | |
| min; e.e. % = 100.0%; | |
| 11-2: the retention time was 1.881 | |
| min; e.e. % = 99.12%. | |

11-1: LC-MS (ESI): m/z 574.2 (M + H)⁺.
11-2: LC-MS (ESI): m/z 574.1 (M + H)⁺.

### Synthesis of Compound 4-a-1 4-a-2

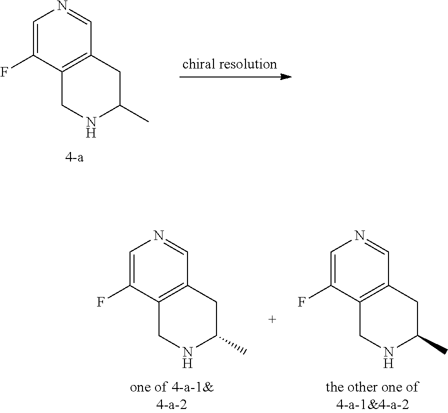

4-a was prepared and purified by chiral resolution to obtain 4-a-1 (2.3 g, 49%), 4-a-2 (1.6 g, 34%).

| Conditions for chiral analysis | Conditions for chiral preparation |
|---|---|
| Instrument: UPCC (Waters) | Instrument: SFC-150 (Waters) |
| Column: AD-3 4.6*100 mm, 3 um | Column: AD 20*250 mm, 10 um |
| (Daicel) | (Daicel) |
| Column temperature: 40° C. | Column temperature: 35° C. |
| Mobile phase: CO₂/Methanol[0.2% | Mobile phase: CO₂/Methanol[0.2% |
| NH₃(7M in Methanol)] = 90/10 | NH₃(7M in Methanol)] = 80/20 |
| Flow rate: 3.0 mL/min | Flow rate: 100 g/min |
| Wavelength: 214 nm | Pressure: 100 bar |
| Pressure: 2000 psi | Wavelength: 214 nm |
| | Circulation: 4.5 min |
| | Sample solution: 5600 mg dissolved |
| | in 120 mL Methanol |
| 4-a-1: the retention time was 1.504 | |
| min; e.e. % = 97.14%; LC-MS | |
| (ESI): m/z 167.2 (M + H)⁺. | |
| 4-a-2: the retention time was 1.780 | |
| min; e.e. % = 97.12%. LC-MS | |
| (ESI): m/z 167.2 (M + H)⁺. | |

Synthetic route of compound 4′

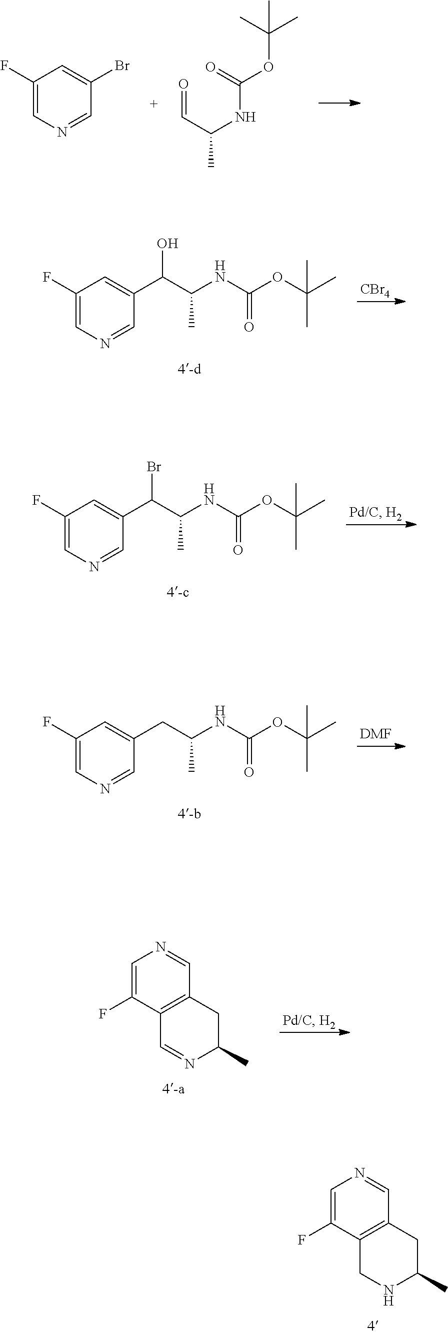

### Synthesis of Compound 4-d′

A solution of isopropylmagnesium chloride-lithium chloride (1.3 M, 18.2 mL, 23.66 mmol) in tetrahydrofuran was slowly added dropwise to a solution of 5-bromo-3-fluoropyridine (4064 mg, 23.09 mmol) in tetrahydrofuran (50 mL) in a reaction flask at 0° C. under nitrogen protection. After addition, the mixture was stirred at 0° C. for 2 h. The reaction mixture was added Boc-D-alaninal (1000 mg, 5.77 mmol) and stirred for 1 h. The reaction was quenched with saturated aqueous ammonium chloride (100 mL), extracted with ethyl acetate (100 mL*3), the organic phase was concentrated, and the crude product was purified by column chromatography (petroleum ether/ethyl acetate=3/1 to 1/1) to give compound 4′-d (1100 mg, 70%).

### Synthesis of Compound 4′-c

4′-d (830 mg, 3.07 mmol), triphenylphosphine (2416 mg, 9.19 mmol) and dichloromethane (80 mL) were combined in a reaction vial, and the mixture was stirred at room temperature under argon protection for 10 min, then was added carbon tetrabromide (3055 mg, 9.21 mmol) slowly. After addition, the reaction was stirred at room temperature for 2 h. The reaction mixture was concentrated and the crude product was purified by column chromatography (petroleum ether/ethyl acetate=5/1 to 3/1) to give compound 4′-c (270 mg, 26%). LC-MS (ESI): m/z 333.4 (M+H)⁺.

### Synthesis of Compound 4′-b

A solution of 4′-c (270 mg, 0.81 mmol), and palladium carbon 10% (30 mg) in methanol (20 mL) was stirred for 2 h at room temperature under hydrogen. The reaction mixture was concentrated by filtration to give compound 4′-b (220 mg, 100%). LC-MS (ESI): m/z 255.5 (M+H)⁺.

### Synthesis of Compound 4′-a

A solution of 4′-b (208 mg, 0.82 mmol) in tetrahydrofuran (30 mL) was stirred under nitrogen protection at −78° C. for 10 min. Kept at this temperature, the solution was added slowly a solution of lithium diisopropylamine in tetrahydrofuran (2M, 1.64 mL, 3.28 mmol) dropwise and stirred at this temperature for 2 h. Then the above mixture was added dried DMF (2 mL, 24.8 mmol) and stirred at −78° C. for 2 h. The reaction was quenched with diluted hydrochloric acid (1 M, 6 mL, 6 mmol), extracted with ethyl acetate (8 mL*3) and concentrated to give compound 4′-a (21 mg, 16%), which was used directly in the next reaction. LC-MS (ESI): m/z 165.1 (M+H)⁺.

### Synthesis of Compound 4′

A solution of 4′-a (20 mg, 0.12 mmol) and palladium carbon 10% (13 mg) in methanol (20 mL) in a reaction flask was stirred at room temperature under hydrogen (1500 mL) for 1.5 h. The reaction mixture was filtered, concentrated and the crude product was purified by column chromatography (dichloromethane/ammonium-methanol (3.5N)=30/1 to 10/1) to give compound 4′ (5.3 mg, 26%). LC-MS (ESI): m/z 167.1 (M+H)⁺.

Determination of chiral structures of compounds 4-a-1 and 4-a-2

The analysis of compounds 4′, 4-a-1 and 4-a-2 according to the chiral analysis conditions for 4-a-1 proved that compound 4′ is identical to compound 4-a-1, while compound 4-a-2 is an isomer.

Experiment 12 Synthetic Route of Compound 12

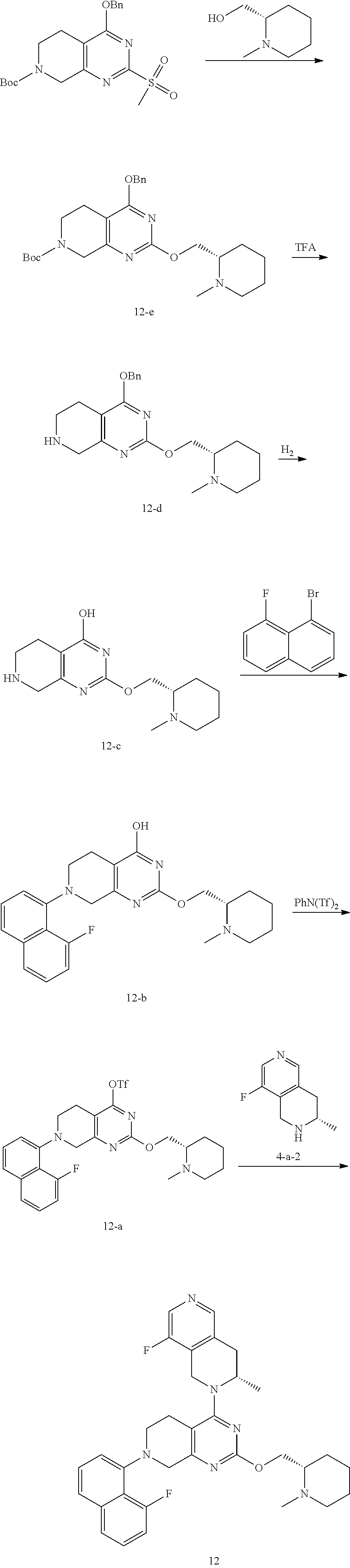

### Synthesis of Compound 12-e

(S)-1-methylpiperidine-2-methanol (1.02 g, 7.89 mmol) was added dropwise to a solution of solid sodium tert-butoxide (0.82 g, 8.53 mmol) in toluene (9 mL) in a reaction flask at 0° C. in an ice-water bath and the resulting mixture was stirred in an ice bath for half an hour after the addition. Tert-butyl-4-benzyloxy-2-methylsulfonyl-5,8-piperidino[3,4-d]-pyrimidine-7(6H)-carboxylate (3.00 g, 7.15 mmol) in toluene (24 mL) was added dropwise to the reaction in an ice-water bath, and the reaction was stirred in the ice bath until it was complete monitored by LCMS. The reaction was quenched by adding brine (40 mL) to the reaction system, extracted with ethyl acetate (40 mL), partitioned, and the organic phase was dried over anhydrous magnesium sulfate and concentrated to give compound 12-e (3.35 g) for direct use in the next step. LC-MS (ESI): m/z=469.7 (M+H)⁺.

### Synthesis of Compound 12-d

TFA (8.15 g, 71.48 mmol) was added to a solution of 12-e (3.35 g, 7.15 mmol) in toluene (18 mL). The reaction was stirred at room temperature for 1 h when by LCMS showed it was complete. The reaction was quenched by adding 5% aqueous sodium hydroxide, adjusting the pH of the system to 10, and extracted with ethyl acetate (50 mL). The organic phase was dried over Na₂SO₄ and concentrated to give compound 12-d (2.1 g), which was used directly in the next step. LC-MS (ESI): m/z=369.3 (M+H)⁺.

### Synthesis of Compound 12-c

12-d (2.1 g, 5.70 mmol) was dissolved in isopropanol (20 mL) and pure water (6 mL), to which 10% Pd/C (100 mg) was added after degassed and purged with hydrogen. The reaction was stirred at room temperature at ambient pressure of hydrogen. When completed monitored by LCMS, the reaction mixture was filtered and the filtrate was concentrated to dryness. The concentrated dry solid was slurried with a mixture of isopropanol (1 mL) and methyltetrahydrofuran (10 mL). Filtered and dried at vacuum to give compound 12-c (1.1 g, 55% total yield in three steps). LC-MS (ESI): m/z=279.2 (M+H)⁺; ¹H NMR (400 MHz, D₂O) δ 4.32 (dd, J=3.5 Hz, 12.1 Hz, 1H), 4.12(dd, J=4.0 Hz, 12.3 Hz, 1H), 3.58 (s, 2H), 3.13-3.09 (m, 1H), 2.98 (t, J=6.0 Hz, 2H), 2.96-2.92 (m, 1H), 2.72-2.65 (m, 1H), 2.55 (s, 3H), 2.33 (t, J=5.9 Hz, 2H), 1.75-1.65 (m, 3H), 1.65-1.50 (m, 2H), 1.40-1.30 (m, 1H).

### Synthesis of Compound 12-b

12-c (417 mg, 1.5 mmol), 1-bromo-8-fluoronaphthalene (338 mg, 1.5 mmol), anhydrous toluene (3 mL), tridibenzylideneacetone dipalladium (41 mg, 0.04 mmol), 4,5-bis(diphenylphosphino)-9,9-dimethylxanthene (43 mg, 0.08 mmol) and tert-butyl sodium (360 mg, 3.75 mmol) were combined in a three-necked flask. Degassed and purged with nitrogen three times, the reaction was stirred at 80° C. for two hour. Cooled to room temperature, the reaction was quenched by adding ammonium chloride solution (10 mL) while stirring, and added ethyl acetate (10 mL) for partition, the organic phase was washed with brine, dried over anhydrous sodium sulfate, filtered and the filtrate was concentrated to give compound 12-b (750 mg, 100%). LC-MS (ESI): m/z=423.3 (M+H)⁺.

### Synthesis of Compound 12-a

To a three-port vial was added 12-b (630 mg, 1.50 mmol, crude), PhN(Tf)₂ (803 mg, 2.25 mmol), anhydrous dichloromethane (5 mL), DBU (228 mg, 1.5 mmol) and DMAP (9 mg, 0.075 mmol), respectively, while controlling internal temperature below 10° C. during the addition. Degassed and purged with nitrogen three times, the reaction was stirred at 25° C. for 2 hours, then was quenched by adding ice-cold water (10 mL) was added DCM (10 mL) to extract, the organic phase was washed with brine, dried over anhydrous sodium sulfate, filtered and washed twice with dichloromethane, the filtrate was concentrated to dryness and purified by column chromatography (mobile phase: methanol/dichloromethane=0/100 to 5/95) to give compound 12-a (445 mg, 53%).). LC-MS (ESI): m/z=555.2 (M+H)⁺.

### Synthesis of Compound 12

12-a (126 mg, 0.23 mmol), DMAC (3 mL), 4-a-2 (43 mg, 0.26 mmol) and DIPEA (88 mg, 0.68 mmol) were combined in a reaction vial, the resulting mixture stirred at 70° C. for 6 h under nitrogen, quenched with brine (20 mL), filtered out the solid, and the solid was purified by column chromatography (mobile phase: methanol (containing NH₃)/dichloromethane 1/20 to 1/10) to give compound 12 (36 mg, 28%). LC-MS (ESI): m/z 571.3 (M+H)⁺; ¹H NMR (400 MHz, CD₃OD): δ 8.28 (1H, s), 8.13-8.27 (1H, m), 7.66 (1H, d, J=7.6 Hz), 7.58 (1H, d, J=8.0 Hz), 7.38-7.46 (2H, m), 7.12-7.22 (2H, m), 4.19-4.85 (6H, m), 3.89-4.16 (1H, m), 3.55-3.77 (1H, m), 3.32-3.48 (1H, m), 2.75-3.13 (4H, m), 2.55-2.70 (1H, m), 2.44 (3H, s), 2.29-2.41 (1H, m), 2.06-2.28 (1H, m), 1.74-1.93 (2H, m), 1.54-1.71 (2H, m), 1.24-1.51 (3H, m), 1.12-1.23 (2H, m).

### Experiment 13 Synthetic Route of Compound 13

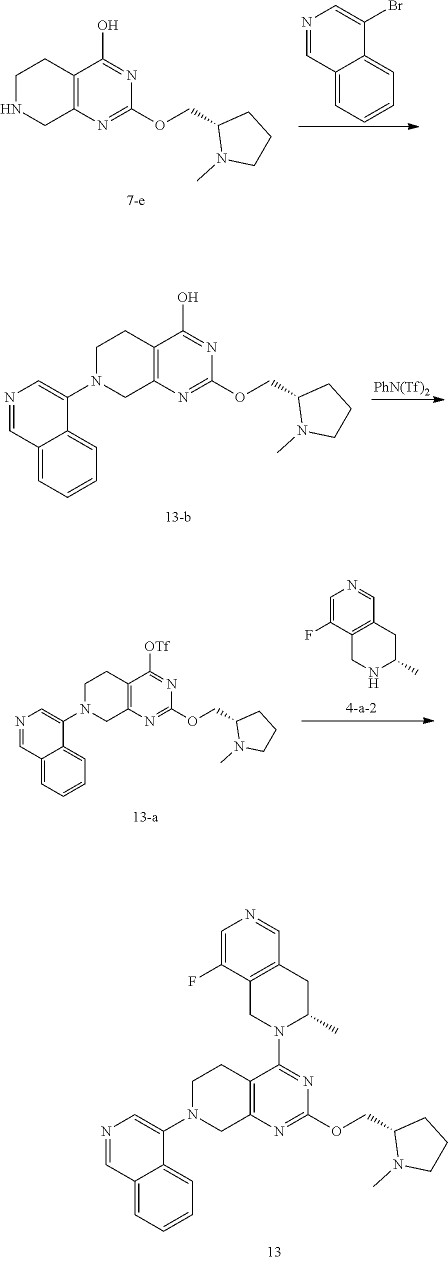

### Synthesis of Compounds 7-e

Referring to the synthesis of compound 12-c, it was synthesized using (S)-(1-methylpyrrolidine-2-methanol instead of (S)-1-methylpiperidine-2-methanol.

### Synthesis of Compound 13-b

7-e (450 mg, 1.71 mmol), toluene (20 mL), BINAP (212 mg, 0.34 mmol), Pd₂(dba)₃ (156 mg, 0.17 mmol), sodium tert-butoxide (491 mg, 5.11 mmol) and 4-bromoisoquinoline (390 mg, 1.88 mmol) were combined in a reaction vial, and the resulting mixture was stirred at 100° C. for 3 h under nitrogen. The compound 13-b (450 mg, 67%) was obtained. LC-MS (ESI): m/z 392.1 (M+H)⁺.

### Synthesis of Compound 13-a

13-b (200 mg, 0.51 mmol), DBU (77 mg, 0.51 mmol), DMAP (1.3 mg, 0.01 mmol) and dichloromethane (15 mL) were combined in a reaction vial and protected by nitrogen. After that, the resulting mixture was added N-phenylbis(trifluoromethanesulfonyl)imide (274 mg, 0.77 mmol), then was stirred for 3 hours. The reaction was quenched by adding Water (20 mL), extracted with dichloromethane (100 mL*3), and the organic phase was concentrated. The crude product was purified by column chromatography (mobile phase: methanol (dichloromethane 1/30 to 1/10) to give compound 13-a (70 mg, 26%). LC-MS (ESI): m/z 524.1 (M+H)⁺.

### Synthesis of Compound 13

13-a (70 mg, 0.13 mmol), DMAC (3 mL), 4-a-2 (29 mg, 0.17 mmol) and DIPEA (86 mg, 0.67 mmol) were combined in a reaction vial, and the resulting mixture was stirred at 70° C. for 3 h under nitrogen, then quenched with saturated saline (20 mL), filtered out the solid, and the solid was purified by column chromatography (mobile phase: methanol (containing NH₃)/DCM=1/20 to 1/10), concentrated to obtain compound 13(20 mg, 28%). LC-MS (ESI): m/z 540.3 (M+H)⁺; ¹H NMR (400 MHz, CD₃OD): δ 8.98 (1H, s), 8.23-8.30 (3H, m), 8.21 (1H, s), 8.13 (1H, d, J=8.0 Hz), 7.86 (1H, t, J=8.0 Hz), 7.26 (1H, t, J=8.0 Hz), 4.78-4.84 (1H, m), 4.55-4.68 (5H, m), 4.38-4.47 (1H, m), 4.20-4.38 (3H, m), 3.61-3.68 (1H, m), 3.34-3.38 (1H, m), 3.21-3.27 (2H, m), 3.04-3.14 (1H, m), 2.83-2.95 (2H, m), 2.73-2.82 (1H, m), 2.31-2.41 (1H, m), 2.03-2.14 (1H, m), 1.66-1.88 (3H, m), 1.25 (3H, d, J=6.8 Hz).

### Experiment 14 Synthetic Route of Compound 14

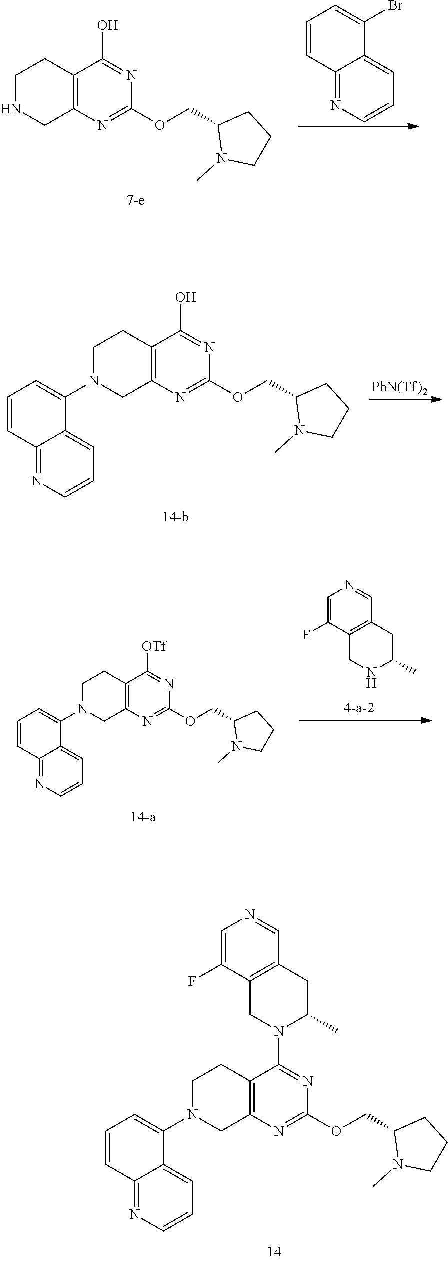

### Synthesis of Compound 14-b

7-e (360 mg, 1.36 mmol), toluene (20 mL), BINAP (169 mg, 0.27 mmol), Pd₂(dba)₃ (125 mg, 0.14 mmol), sodium tert-butoxide (392 mg, 4.09 mmol) and 5-bromoquinoline (311 mg, 1.50 mmol) were combined in a reaction vial, and the resulting mixture was stirred at 110° C. for 3 h under an atmosphere of nitrogen, filtered, concentrated organic phase, purified by column chromatography (mobile phase: methanol (with NH₃)/dichloromethane 1/20 to 1/10), concentrated to give compound 14-b (210 mg, 39%) LC-MS (ESI): m/z 392.1 (M+H)⁺.

### Synthesis of Compound 14-a

A mixture of 14-b (210 mg, 0.54 mmol), DBU (82 mg, 0.54 mmol), DMAP (1.3 mg, 0.01 mmol) in dichloromethane (15 mL) in a reaction vial was stirred at room temperature for 10 min under nitrogen protection. Then was added N-phenylbis(trifluoromethanesulfonyl)imide (287 mg, 0.81 mmol) and stirred for 3 hours. Quenched by Water (20 mL), extracted with dichloromethane (60 mL*3), the organic phase was dried over sodium sulfate, and concentrated. The crude product was purified by column chromatography (mobile phase: methanol (dichloromethane 1/30 to 1/10) to give compound 14-a (162 mg, 57%). LC-MS (ESI): m/z 524.2 (M+H)⁺.

### Synthesis of Compound 14

A mixture of 14-a (120 mg, 0.23 mmol), DMAC (3 mL), 4-a-2 (50 mg, 0.30 mmol) and DIPEA (148 mg, 1.15 mmol) in a reaction vial was stirred at 70° C. for 3 h under nitrogen, then quenched with brine (20 mL), filtered out the solid, and the crude product was column chromatographed (mobile phase: methanol (containing NH₃)/dichloromethane 1/20 to 1/10), concentrated compound 14 (50 mg, 40%), LC-MS (ESI): m/z 540.2 (M+H)⁺; ¹H NMR (400 MHz, CD₃OD): δ 8.83-8.86 (1H, m), 8.73 (1H, d, J=8.4 Hz), 8.27 (2H, d, J=10.8 Hz), 7.69-7.80 (2H, m), 7.54-7.59 (1H, m), 7.33 (1H, d, J=7.2 Hz), 4.99 (2H, d, J=18.4 Hz), 4.78-4.85 (1H, m), 4.62 (1H, d, J=18.8 Hz), 4.38-4.44 (1H, m), 4.28-4.33 (1H, m), 4.13-4.26 (2H, m), 3.45-3.57 (1H, m), 3.16-3.28 (2H, m), 3.06-3.13 (1H, m), 2.69-2.90 (3H, m), 2.51 (3H, s), 2.33-2.41 (1H, m), 2.06-2.14 (1H, m), 1.66-1.88 (3H, m), 1.24 (3H, d, J=6.8 Hz).

### Experiment 15 Synthetic Route of Compound 15

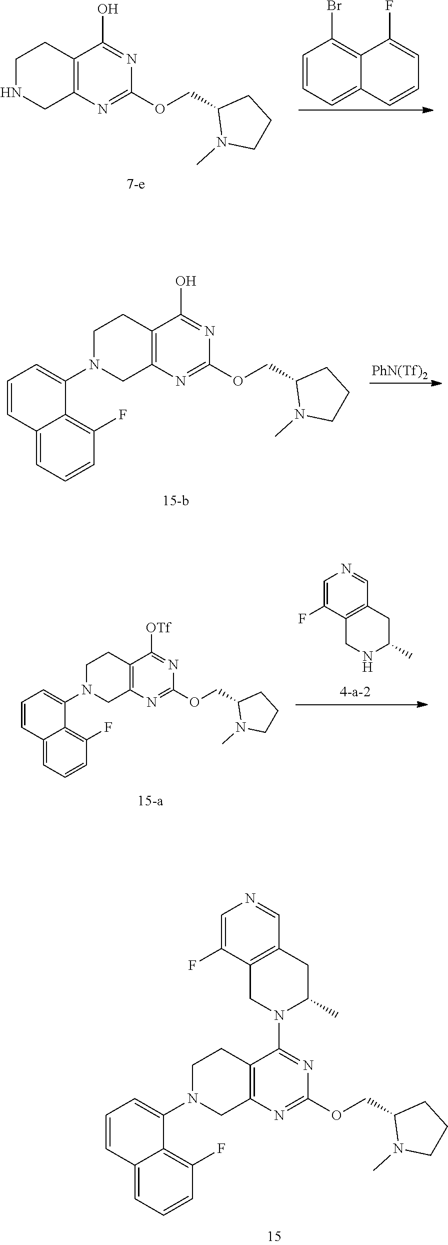

### Synthesis of Compound 15-b

7-e (200 mg, 0.76 mmol), toluene (40 mL), BINAP (94 mg, 0.15 mmol), Pd₂(dba)₃ (69 mg, 0.08 mmol), sodium tert-butoxide (363 mg, 3.78 mmol) and 1-bromo-8-fluoronaphthalene (200 mg, 0.89 mmol) were combined in a reaction vial, and the mixture was stirred at 110° C. for 3 hours under the protection of N₂. The reaction mixture was filtered, and the filtrate was concentrated to dryness and purified by column chromatography (mobile phase: methanol (with NH₃)/dichloromethane 1/20 to 1/10) and concentrated to give compound 15-b (190 mg, 61%). LC-MS (ESI): m/z 409.2 (M+H)⁺.

### Synthesis of Compound 15-a

15-b (150 mg, 0.33 mmol), DBU (75 mg, 0.49 mmol), DMAP (1.2 mg, 0.01 mmol) and dichloromethane (15 mL) were combined in a reaction vial and stirred at room temperature for 10 min under nitrogen protection. N-phenylbis(trifluoromethanesulfonyl)imide (262 mg, 0.73 mmol) was added to the above mixture and stirred for 3 hours. The reaction was quenched with water (20 mL), extracted with dichloromethane (100 mL*3), the organic phase was concentrated and purified by column chromatography (mobile phase: methanol/dichloromethane 1/30 to 1/10) to give compound 15-a (120 mg, 45%). LC-MS (ESI): m/z 541.6 (M+H)⁺.

### Synthesis of Compound 15

A mixture of 15-a (120 mg, 0.22 mmol), DMAC (3 mL), 4-a-2 (48 mg, 0.29 mmol) and DIPEA (143 mg, 1.11 mmol) in a reaction vial was stirred at 70° C. for 3 h under nitrogen, quenched with saturated salt water (20 mL), filtered out the solid, and the crude product was purified by column chromatography (mobile phase: methanol (containing NH₃)/dichloromethane 1/20 to 1/10), concentrated compound 15 (31 mg, 25%), LC-MS (ESI): m/z 557.2 (M+H)⁺; ¹H NMR (400 MHz, CD₃OD): δ 8.13-8.29 (2H, s), 7.68 (1H, d, J=8.0 Hz), 7.60 (1H, d, J=8.0 Hz), 7.36-7.48 (2H, m), 7.13-7.25 (2H, m), 4.19-4.75 (6H, m), 3.91-4.12 (1H, m), 3.55-3.80 (1H, m), 3.33-3.48 (1H, m), 3.00-3.21 (2H, m), 2.78-2.98 (3H, m), 2.39-2.70 (5H, m), 2.00-2.21 (1H, m), 1.75-1.90 (3H, m), 1.14-1.33 (3H, m).

### Experiment 16 Synthesis of Compound 16

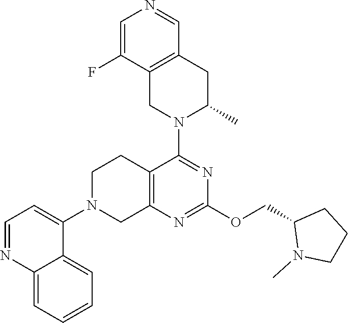 [SMILES: C[C@H]1Cc2cncc(F)c2CN1c1nc(OC[C@@H]2CCCN2C)nc2c1CCN(c1ccnc3ccccc13)C2]

Referring to the synthesis of compound 14, 4-bromoquinoline was used instead of 5-bromoquinoline for the synthesis of compound 16. LC-MS (ESI): m/z 540.3 (M+H)⁺; ¹H NMR (400 MHz, CD₃OD): δ 8.65 (1H, d, J=5.2 Hz), 8.27 (2H, d, J=10.4 Hz), 8.19 (1H, d, J=8.4 Hz), 7.98 (1H, d, J=8.4 Hz), 7.76 (1H, t, J=7.6 Hz), 7.61 (1H, t, J=8.4 Hz), 7.10 (1H, d, J=4.8 Hz), 4.99 (2H, d, J=18.4 Hz), 4.79-4.85 (2H, m), 4.62 (2H, d, J=18.8 Hz), 4.26-4.45 (4H, m), 3.81-3.93 (1H, m), 3.03-3.14 (1H, m), 2.84-2.94 (2H, m), 2.70-2.80 (1H, m), 2.50 (3H, s), 2.30-2.38 (1H, m), 2.03-2.14 (1H, m), 1.78-1.86 (2H, m), 1.66-1.78 (1H, m), 1.24 (3H, d, J=6.8 Hz).

### Experiment 17 Synthesis of Compound 17

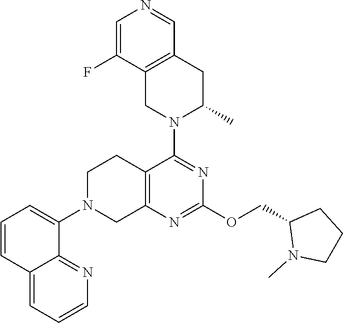 [SMILES: C[C@H]1Cc2cncc(F)c2CN1c1nc(OC[C@@H]2CCCN2C)nc2c1CCN(c1cccc3cccnc13)C2]

Referring to the synthesis of compound 14, 8-bromoquinoline was used instead of 5-bromoquinoline for the synthesis of compound 17. LC-MS (ESI): m/z 540.2 (M+H)⁺; H NMR (400 MHz, CD₃OD): δ 8.90 (1H, d, J=4.0 Hz), 8.34-8.27 (3H, m), 7.62-7.53 (3H, m), 7.37 (1H, d, J=8.0 Hz), 5.05-4.79 (3H, m), 4.68-4.21 (5H, m), 3.91-3.88 (1H, m), 3.26-3.06 (2H, m), 2.901-2.72 (3H, m), 2.51 (3H, s), 2.38-2.32 (1H, m), 2.13-2.03 (1H, m), 1.87-1.68 (3H, m), 1.35-1.24 (4H, m).

### Experiment 18 Synthesis of Compound 18

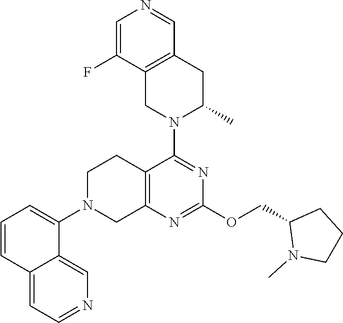 [SMILES: C[C@H]1Cc2cncc(F)c2CN1c1nc(OC[C@@H]2CCCN2C)nc2c1CCN(c1cccc3ccncc13)C2]

Referring to the synthesis of compound 14, 8-bromoisoquinoline was used instead of 5-bromoquinoline for the synthesis of compound 18. LC-MS (ESI): m/z 540.3 (M+H)⁺.

### Experiment 19 Synthesis of Compound 19

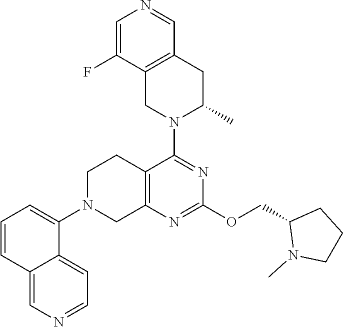 [SMILES: C[C@H]1Cc2cncc(F)c2CN1c1nc(OC[C@@H]2CCCN2C)nc2c1CCN(c1cccc3cnccc13)C2]

Referring to the synthesis of compound 14, 5-bromoisoquinoline was used instead of 5-bromoquinoline for the synthesis of compound 19. LC-MS (ESI): m/z 540.3 (M+H)⁺.

### Experiment 20 Synthetic Route of Compound 20

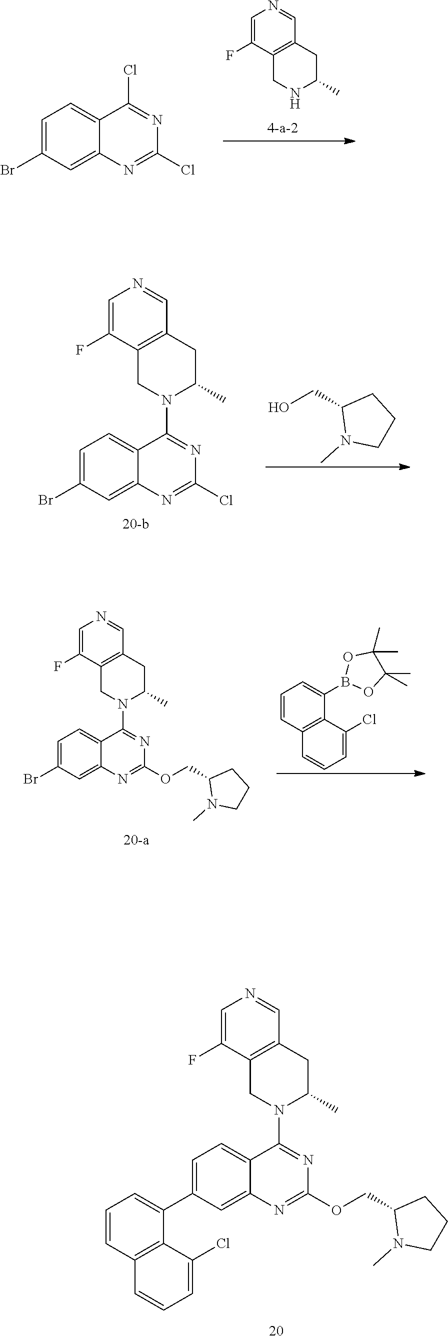

### Synthesis of Compound 20-b

To a solution of 2,4-dichloro-7-bromoquinazoline (200 mg, 0.72 mmol) and 4-a-2 (144 mg, 0.86 mmol) in tetrahydrofuran (10 mL) at room temperature was added N,N-diisopropylethylamine (465 mg, 3.60 mmol) and the mixture was stirred for 3 h. The mixture was added water and extracted with ethyl acetate (50 mL) and the organic phase was dried with The organic phase was dried over anhydrous sodium sulfate, filtered and the filtrate was concentrated, and the crude product was purified by column flash (mobile phase: EA/PE, 0-30%) to give 20-b (176 mg, 61%). LC-MS (ESI): m/z 406.9 (M+H)⁺.

### Synthesis of Compound 20-a

Sodium tert-butoxide (83 mg, 0.86 mmol) was added to a solution of 20-b (176 mg, 0.43 mmol) and N-methyl-L-prolinol (149 mg, 1.30 mmol) in toluene (6 mL) in an ice-water bath, and the mixture was stirred at 0 degree C. for 2 h. When completed, the reaction was added water and extracted with ethyl acetate (30 mL*2), and the combined organic phases were dried over anhydrous sodium sulfate, filtered and concentrated, and the crude product was purified by column flash (mobile phase: 0-100%, dichloromethane:methanol=10:1/dichloromethane) to give 20-a (106 mg, 50%). LC-MS (ESI): m/z 486.0 (M+H)⁺.

### Synthesis of Compound 20

20-a (30 mg, 0.062 mmol), 8-chloro-1-naphthaleneboronic acid pinacol ester (18 mg, 0.062 mmol), Pd(PPh₃)₄(7 mg, 0.006 mmol), cesium carbonate (40 mg, 0.12 mmol), 1,4-dioxane (8 mL) and water (2 mL) were combined in a 100 mL aubergine vial. Degassed and purged with nitrogen several times, the mixture was stirred at 90 degrees overnight. Concentrated to remove the solvent, added water, extracted with ethyl acetate (30 mL*2), and the combined organic phases were dried over anhydrous sodium sulfate, filtered, and concentrated to dryness. The crude product was purified by column flash (mobile phase: 0-100%, dichloromethane:methanol=10:1/dichloromethane) to give 20 (13.7 mg, 39%). LC-MS (ESI): m/z 568.2 (M+H)

Experiment 21 Synthetic Route of Compound 21

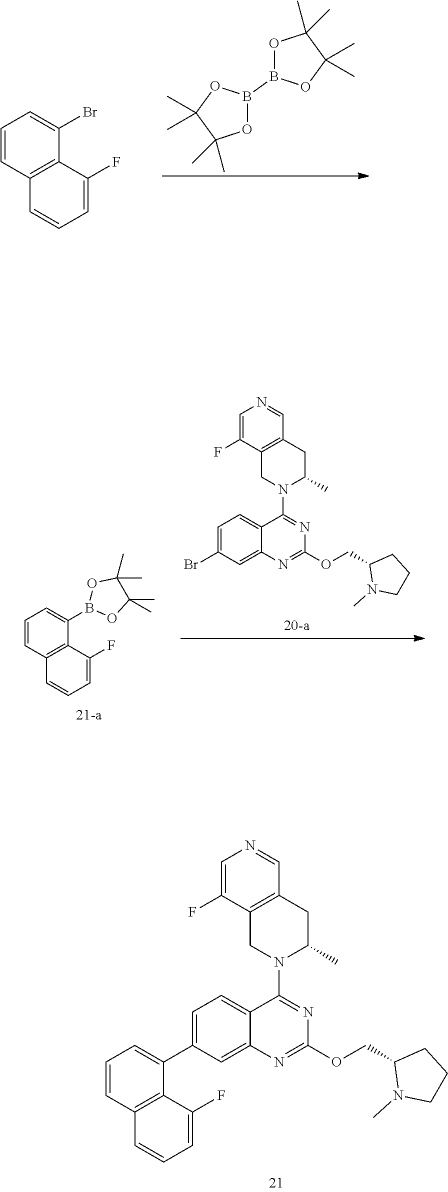

### Synthesis of Compound 21-a

To a 100 mL aubergine flask was added 1-bromo-8-fluoronaphthalene (100 mg, 0.44 mmol), diboronic acid pinacol ester (135 mg, 0.53 mmol), Pd(dppf)Cl₂ (33 mg, 0.044 mmol), potassium acetate (87 mg, 0.89 mmol) and 1,4-dioxane (10 mL). Degassed and purged with N₂ several times, the mixture was stirred at 90 degrees overnight. Concentrated to remove the solvent, added water, extracted with ethyl acetate (30 mL*2), and the combined organic phases were dried over anhydrous sodium sulfate, filtered, and the filtrate was concentrated to dryness. The crude product was purified on a silica column (mobile phase: EA/PE, 0-20%) to give compound 21-a (94 mg, 78%).

### Synthesis of Compound 21

To a 100 mL eggplant vial was added 20-a (30 mg, 0.062 mmol), 21-a (20 mg, 0.074 mmol), Pd(PPh₃)₄(7 mg, 0.006 mmol), cesium carbonate (40 mg, 0.12 mmol), 1,4-dioxane (8 mL) and water (2 mL). Degassed and purged with N₂ several times, the mixture was stirred at 90 degrees overnight. Concentrated to remove the solvent, added water, extracted with ethyl acetate (30 mL*2), and the combined organic phases were dried over anhydrous sodium sulfate, filtered, and the filtrate was concentrated to dryness. The crude product was purified by column flash (mobile phase: 0-100%, dichloromethane:methanol=10:1/dichloromethane) to give 21 (13.8 mg, 40%). LC-MS (ESI): m/z 552.3 (M+H)⁺.

### Experiment 22 Synthesis of Compound 22

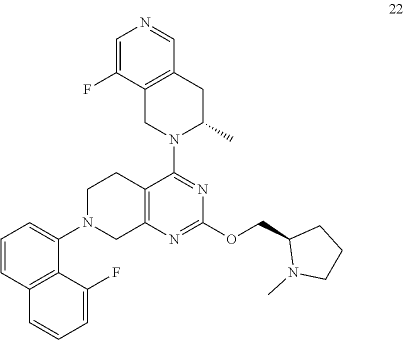 [SMILES: C[C@H]1Cc2cncc(F)c2CN1c1nc(OC[C@H]2CCCN2C)nc2c1CCN(c1cccc3cccc(F)c13)C2]

Referring to the synthetic route of compound 12, compound 22 was synthesized using (R)-N-methyl-2-pyrrolidinemethanol instead of (S)-1-methylpiperidine-2-methanol and tert-butyl 4-benzyloxy-2-methylsulfonyl-5,8-piperidino[3,4-d]-pyrimidine-7(6H)-carboxylate was used as starting material. LC-MS (ESI): m/z 557.3 (M+H)+; ¹H NMR (400 MHz, CD₃OD): δ 8.29 (2H, s), 7.68-7.58 (2H, m), 7.46-7.40 (2H, m), 7.20-7.14 (2H, m), 4.83-4.66 (2H, m), 4.43-4.25 (3H, m), 4.01-3.40 (3H, m), 3.22-2.85 (5H, m), 2.66-2.54 (5H, m), 2.20-1.80 (4H, m), 1.38-1.18 (4H, m).

### Experiment 23 Synthesis of Compound 23

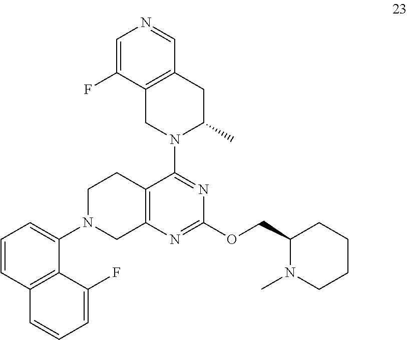 [SMILES: C[C@H]1Cc2cncc(F)c2CN1c1nc(OC[C@H]2CCCCN2C)nc2c1CCN(c1cccc3cccc(F)c13)C2]

Referring to the synthetic route of compound 12, compound 23 was synthesized using (R)-1-methylpiperidine-2-methanol instead of (S)-1-methylpiperidine-2-methanol, and tert-butyl 4-benzyloxy-2-methylsulfonyl-5,8-piperidino[3,4-d]-pyrimidine-7(6H)-carboxylate was used as starting material. LC-MS (ESI): m/z 571.3 (M+H)⁺.

### Experiment 24 Synthetic Route of Compound 24

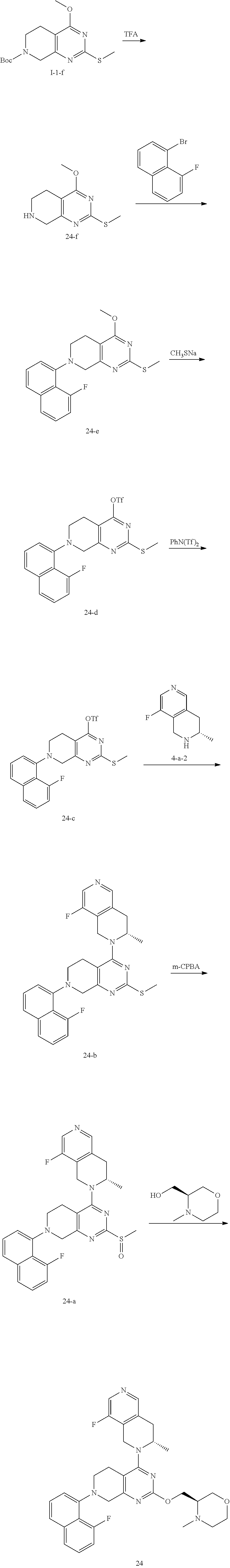

### Synthesis of Compound 24-f

Compound I-1-f (18.30 g, 58.84 mmol) was added to a mixed solution of dichloromethane (80 mL) and trifluoroacetic acid (40 mL) and the resulting mixture was stirred at room temperature for 3 hours. The reaction mixture was concentrated, quenched with saturated sodium bicarbonate (200 mL), the aqueous solution was added sodium sulfate solid, extracted with dichloromethane (300*3 mL), and dried over sodium sulfate. Concentration to give compound 24-f (14.00 g, 100%). LC-MS (ESI): m/z 212.1 (M+H)⁺.

### Synthesis of Compound 24-e

24-f (1500 mg, 7.10 mmol), 1-bromo-8-fluoronaphthalene (1758 mg, 7.81 mmol), toluene (30 mL), sodium tert-butoxide (2047 mg, 21.30 mmol), Pd₂(dba)₃ (650 mg, 0.71 mmol), BINAP (884 mg, 1.42 mmol) were combined in a reaction vial. Degassed and purged with N₂, the reaction was stirred at 100° C. for 3 h. The reaction mixture was concentrated and purified by column chromatography (mobile phase: PE/EA 10/0 to 10/1) to give compound 24-e (1600 mg, 63%). LC-MS (ESI): m/z 356.0 (M+H)⁺.

### Synthesis of Compound 24-d

To a reaction vial was added 24-e (1600 mg, 4.50 mmol), DMF (10 mL), and sodium thiomethoxide (3155 mg, 45.02 mmol). Degassed and purged with N₂, the reaction was stirred at 60° C. for 2 hours. The reaction was cooled, was added water (100 mL) and adjusted to acidic with hydrochloric acid, and a solid was precipitated and filtered. The filter cake was washed with water and dried to give compound 24-d (920 mg, 60%). LC-MS (ESI): m/z 342.1 (M+H)⁺.

### Synthesis of Compound 24-c

To a reaction vial was added 24-d (200 mg, 0.59 mmol), N-phenylbis(trifluoromethanesulfonyl)imide (314 mg, 0.88 mmol), DBU (134 mg, 0.88 mmol), DMAP (7 mg, 0.06 mmol), and DCM (20 mL). Degassed and purged with N₂, the reaction was stirred at room temperature for 2 hours. The reaction was quenched with water, extracted with DCM, the organic phases were combined, dried and concentrated, and purified by column chromatography (mobile phase: PE/EA 10/0 to 10/1) to give compound 24-c (130 mg, 47%). LC-MS (ESI): m/z 474.0 (M+H)⁺.

### Synthesis of Compound 24-b

To a reaction vial was added 24-c (130 mg, 0.27 mmol), 4-a-2 (46 mg, 0.28 mmol), DIPEA (71 mg, 0.55 mmol), and DMSO (1 mL). Degassed and purged with N₂, the reaction was stirred at 70° C. for 8 hours. The reaction was cooled, added water and extracted with a mixed DCM:MeOH=10:1 solution, the organic phases were combined, dried and concentrated, and purified over the column (mobile phase: DCM/MeOH 10/0 to 10/1) to give compound 24-b (130 mg, 97%). LC-MS (ESI): m/z 490.1 (M+H)⁺.

### Synthesis of Compound 24-a

To a reaction vial was added 24-b (30 mg, 0.06 mmol), m-chloroperoxybenzoic acid (16 mg, 0.09 mmol) and DCM (10 mL). Degassed and purged with N₂, the reaction was stirred at room temperature for 1 hour. The reaction mixture was quenched with aqueous sodium bicarbonate, extracted with DCM, and the organic phases were combined and concentrated to dryness to give compound 24-a (30 mg, 98%). LC-MS (ESI): m/z 506.1 (M+H)⁺.

### Synthesis of Compound 24

To a reaction vial was added 24-a (30 mg, 0.06 mmol), (S)-4-methyl-3-morpholinemethanol (16 mg, 0.12 mmol), sodium tert-butoxide (17 mg, 0.18 mmol) and DCM (10 mL). Degassed and purged with N₂, the reaction was stirred at room temperature for half an hour. The reaction mixture was concentrated, dissolved in methanol, filtered and purified by prep-HPLC to give compound 24 (3 mg, 8.8%). LC-MS (ESI): m/z 573.2 (M+H)⁺.

### Synthesis of Compound 25

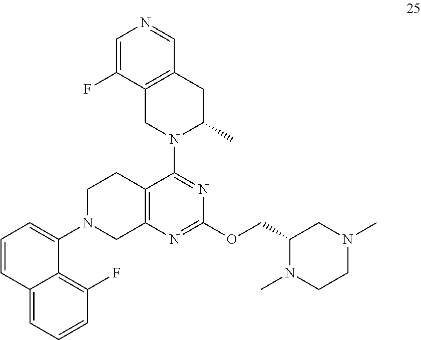 [SMILES: C[C@H]1Cc2cncc(F)c2CN1c1nc(OC[C@@H]2CN(C)CCN2C)nc2c1CCN(c1cccc3cccc(F)c13)C2]

Referring to the synthetic route of compound 24, compound 25 was synthesized using (S)-(1,4-dimethylpiperazin-2-yl)methanol instead of (S)-4-methyl-3-morpholinemethanol, and 24-a was used as starting material. LC-MS (ESI): m/z 586.2 (M+H)⁺.

### Synthesis of Compound 26

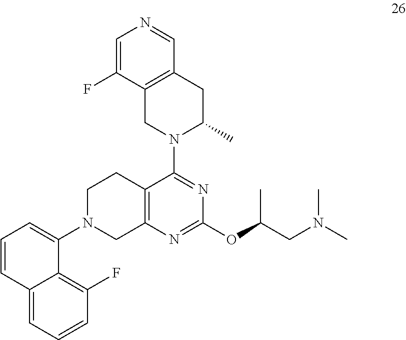 [SMILES: C[C@@H](CN(C)C)Oc1nc2c(c(N3Cc4c(F)cncc4C[C@@H]3C)n1)CCN(c1cccc3cccc(F)c13)C2]

Referring to the synthetic route of compound 24, compound 26 was synthesized using (S)-1-(dimethylamino)propan-2-ol instead of (S)-4-methyl-3-morpholinemethanol, and 24-a was used as starting material. LC-MS (ESI): m/z 545.6 (M+H)⁺.

### Experiment 27 Synthetic Route of Compound 27

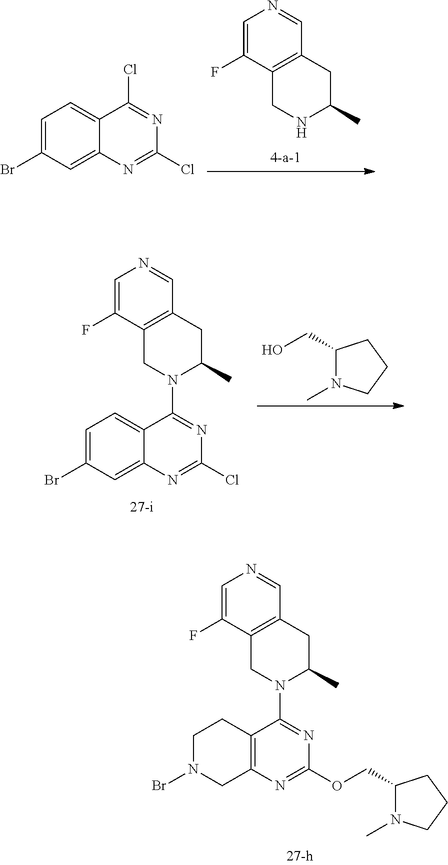

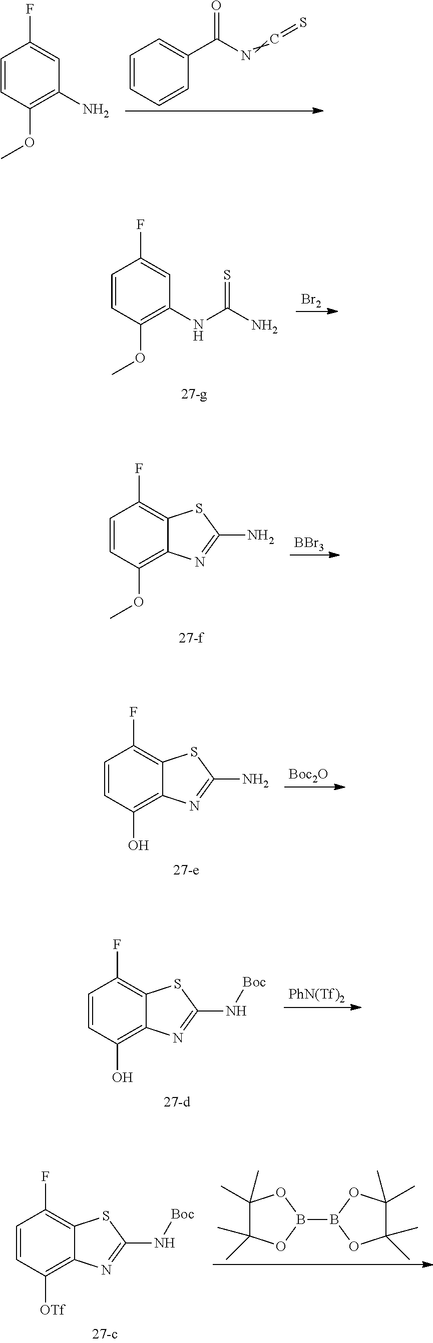

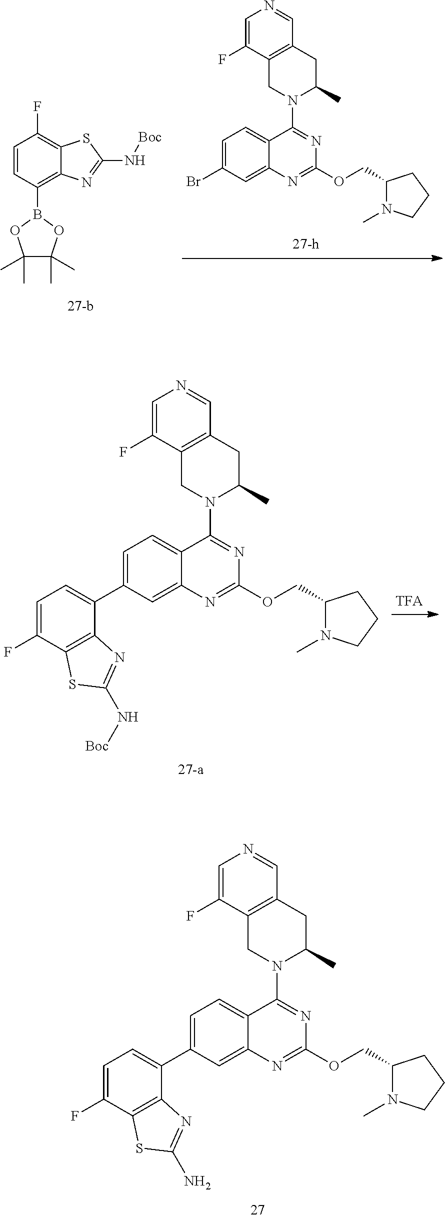 [SMILES: CC(C)(C)OC(=O)Nc1nc2c(B3OC(C)(C)C(C)(C)O3)ccc(F)c2s1 ; C[C@@H]1Cc2cncc(F)c2CN1c1nc(OC[C@@H]2CCCN2C)nc2cc(Br)ccc12 ; C[C@@H]1Cc2cncc(F)c2CN1c1nc(OC[C@@H]2CCCN2C)nc2cc(-c3ccc(F)c4sc(NC(=O)OC(C)(C)C)nc34)ccc12 ; C[C@@H]1Cc2cncc(F)c2CN1c1nc(OC[C@@H]2CCCN2C)nc2cc(-c3ccc(F)c4sc(N)nc34)ccc12]

### Synthesis of Compound 27-i

To a solution of 2,4-dichloro-7-bromoquinazoline (300 mg, 1.08 mmol) and 4-a-1 (215 mg, 1.29 mmol) in tetrahydrofuran (10 mL) was added N,N-diisopropylethylamine (698 mg, 5.4 mmol) at room temperature and the mixture was stirred at room temperature for 3 hr. Water was added and extracted with ethyl acetate (50 ml), the organic phase was dried over anhydrous sodium sulfate, filtered and the filtrate was concentrated to dryness, and the crude product was purified on a silica gel column (mobile phase: EA/PE, 0-50%) to give 27-i (251 mg, 57%). LC-MS (ESI): m/z 406.8 (M+H)⁺.

### Synthesis of Compound 27-h

Sodium tert-butoxide (118 mg, 1.23 mmol) was added to a solution of 27-i (250 mg, 0.61 mmol) and N-methyl-L-prolinol (353 mg, 3.06 mmol) in toluene (8 mL) in an ice-water bath and the mixture was stirred for 2 h at 0° C. When completed, the reaction was added water and extracted with ethyl acetate (30 ml*2), the combined organic phases were dried over anhydrous sodium sulfate, filtered and concentrated to dryness, and the crude product was purified by column chromatography (mobile phase: 0-100%, dichloromethane:methanol=10:1/dichloromethane) to give 27-h (205 mg, 69%). LC-MS (ESI): m/z 486.0 (M+H)⁺.

### Synthesis of Compound 27-g

5-Fluoro-2-methoxyaniline (9.99 g, 70.78 mmol) was slowly added to a solution of benzoyl isothiocyanate (11 g, 67.41 mmol) in tetrahydrofuran (80 mL) under nitrogen protection and in an ice-water bath while the internal reaction temperature was maintained below 10° C. After addition, the reaction temperature was slowly warmed to room temperature, stirred for 30 min, and was added sodium hydroxide solution (5 M, 16.2 mL, 80.89 mmol) and water (20 mL, 1106.55 mmol) and stirred at reflux for 3.5 h. Cooled to room temperature, the reaction mixture was added water (50 ml) and isopropyl acetate (80 mL), the pH value was adjusted to 9-10 with aqueous concentrated hydrochloric acid. The organic phase was dried over anhydrous sodium sulfate, filtered, concentrated to dryness, dissolved by adding ethyl acetate, continued to concentrate at reduced pressure, solids were precipitated during concentration, the mixture was placed at −20° C. overnight without concentrated to dryness. The mixture was warmed to room temperature, filtered, and the filter cake was washed with petroleum ether, and the filter cake was the product. The filtrate was concentrated to dryness and purified by column chromatography (silica gel, EA/PE, 0-50%) to obtain the product, which was combined with the filter cake to give 27-g (10.6 g, 79%). LC-MS (ESI): m/z 201.0 (M+H)

### Synthesis of Compound 27-f

Liquid bromine (2.43 mL, 47.38 mmol) was added slowly to 27-g (9.3 g, 46.45 mmol) of chloroform (200 mL) solution under ice bath and nitrogen protection, maintaining the internal temperature below 7° C. The reaction mixture was added in an ice bath. After addition, the reaction mixture was stirred for 30 min under ice bath conditions, then heated to reflux and stirred for 2 hours. After the reaction was completed, the solution was cooled to room temperature, rotary at reduced pressure to remove the chloroform, then added dichloromethane to dissolve, concentrated, and no longer concentrated when solids were precipitated during the concentration process, and the solution was placed in a freezer at −20° C. overnight. The solution was removed from the freezer and a large amount of solid was precipitated, filtered, and the filter cake was washed with petroleum ether to give 27-f (12.86 g) as hydrobromide, which was alkalinized to give 8.5 g of free solid. LC-MS (ESI): m/z 199.0 (M+H)⁺.

### Synthesis of Compound 27-e

Boron tribromide (1 M, dichloromethane solution, 107.2 mL, 107.2 mmol) was slowly added to a solution of 27-f (8.5 g, 42.88 mmol) in dichloromethane (200 mL) under nitrogen protection and in an acetone-dry ice bath, maintaining this temperature during the addition. After the dropwise addition, the temperature was slowly warmed to room temperature and continued stirring overnight. The reaction mixture was slowly poured into ice-water while stirring at the same time during the pouring process, and a solid was precipitated, filtered, and the filter cake and filtrate were purified respectively. The filter cake is the hydrobromide of the product, dissolved in water, was added saturated aqueous sodium bicarbonate solution, adjusted the pH to 7-8, a large amount of solids precipitated, filter, collect the filter cake, and poured the filtrate off. The first filtrate was added aqueous sodium bicarbonate solution, and adjusted the pH to 7-8. The organic phase was dried over anhydrous sodium sulfate, and the solvent was removed by concentration, and solids were precipitated in the concentration process, without removing all the solvent, filtered, and the filter cake was collected. The two filter cakes were combined and dried to give 27-e (7.4 g, 94%). LC-MS (ESI): m/z 185.0 (M+H)⁺.

### Synthesis of Compound 27-d

Boc₂O (26.31 g, 120.53 mmol) and triethylamine (11.73 mL, 84.37 mmol) were added to a solution of 27-e (7.4 g, 40.18 mmol) in tetrahydrofuran (200 mL) in an ice-water bath, followed by DMAP (0.49 g, 4.02 mmol) slowly. After addition, the reaction temperature was slowly warmed to room temperature and stirred overnight. When completed, the reaction was added water and extracted with ethyl acetate (100 ml*3), the combined organic phase was dried over anhydrous sodium sulfate, filtered and the filtrate was concentrated to dryness, the crude product was dissolved in methanol, was added potassium carbonate (16.66 g, 120.53 mmol) and stirred at room temperature for 4 h. The product was monitored as desired product, concentrated to dryness, water was added and stirred for 0.5 h. A large amount of solid was precipitated and filtered. The filter cake was dried to give 27-d (11.26 g, 99%). LC-MS (ESI): m/z 285.0 (M+H)⁺.

### Synthesis of Compound 27-c

To a 100 ml aubergine vial was added 27-d (2 g, 7.04 mmol), DBU (1.07 g, 7.04 mmol) and dichloromethane (30 mL), followed by N-phenylbis(trifluoromethanesulfonyl)imide (3.77 g, 10.55 mmol) and DMAP (0.09 g, 0.7 mmol), and the mixture was stirred overnight. The next day, the reaction mixture was added water and extracted with dichloromethane (50 ml*2), the combined organic phases were dried over anhydrous sodium sulfate, filtered and concentrated to dryness, and the crude product was purified by column chromatography (mobile phase: EA/PE, 0-20%) to give 27-c (2.28 g, 78%). LC-MS (ESI): m/z 360.9(M-56+H)⁺, 417.0 (M+H)⁺.

### Synthesis of Compound 27-b

To a 100 ml aubergine vial was added 27-c (800 mg, 1.92 mmol), bis(pinacolato) borate (4.88 g, 19.21 mmol), tetrakis(triphenylphosphine)palladium (222 mg, 0.19 mmol), potassium acetate (566 mg, 5.76 mmol) and 1,4-dioxane (20 mL). The mixture was degassed and purged with nitrogen several times and stirred at 100° C. over the weekend. The reaction was monitored to show that it was complete, concentrated to dryness, added water and extracted with ethyl acetate (50 ml*2), the combined organic phases were dried over anhydrous sodium sulfate, filtered, concentrated to dryness and the crude product was purified by column chromatography (silica gel, mobile phase: EA/PE, 0-50%) to give 27-b (0.65 g, 85%). LC-MS (ESI): m/z 313.0 (M+H)⁺ (shown as molecular weight of boronic acid).

### Synthesis of Compound 27-a

To a 100 ml aubergine vial was added 27-h (50 mg, 0.10 mmol), 27-b (49 mg, 0.12 mmol), tetrakis(triphenylphosphine)palladium (12 mg, 0.01 mmol), cesium carbonate (67 mg, 0.21 mmol), 1,4-dioxane (8 mL) and water (2 mL). The mixture was degassed and purged with nitrogen several times and stirred at 90° C. overnight. The reaction mixture was concentrated to dryness, added water and extracted with ethyl acetate (50 ml*2). The combined organic phases were dried over anhydrous sodium sulfate, filtered, concentrated to dryness and the crude product was purified by column chromatography (silica gel, mobile phase: DCM:MeOH=10:1/DCM, 0-100%) to give 27-a (54 mg, 78%). LC-MS (ESI): m/z 674.8 (M+H)⁺.

### Synthesis of Compound 27

To a solution of 27-a (54 mg, 0.08 mmol) in dichloromethane (4 mL) was added trifluoroacetic acid (1 mL) and the mixture was stirred at room temperature overnight. The reaction mixture was concentrated to dryness, added with saturated aqueous sodium bicarbonate, extracted with ethyl acetate (50 ml), the organic phase was dried over anhydrous sodium sulfate, filtered and concentrated to dryness. The crude product was prepared and purified by Pre-HPLC to give 27 (18.8 mg, 41%). LC-MS (ESI): m/z 574.1 (M+H)⁺; ¹H NMR (400 MHz, DMSO-d₆): δ 8.41-8.34 (2H, m), 8.06-8.01 (2H, m), 7.96 (2H, s), 7.79 (1H, d, J=7.6 Hz), 7.53 (1H, t, J=6.4 Hz), 7.06 (1H, t, J=8.8 Hz), 5.07-4.95 (2H, m), 4.79 (1H, d, J=18 Hz), 4.53-4.36 (1H, m), 4.23-4.15 (1H, m), 3.69-3.58 (1H, m), 3.00-2.82 (2H, m), 2.38 (3H, s), 2.23-2.13 (1H, m), 2.03-1.93 (2H, m), 1.76-1.59 (3H, m), 1.18 (3H, d, J=5.6 Hz).

### Experiment 28 Synthetic Route of Compound 28

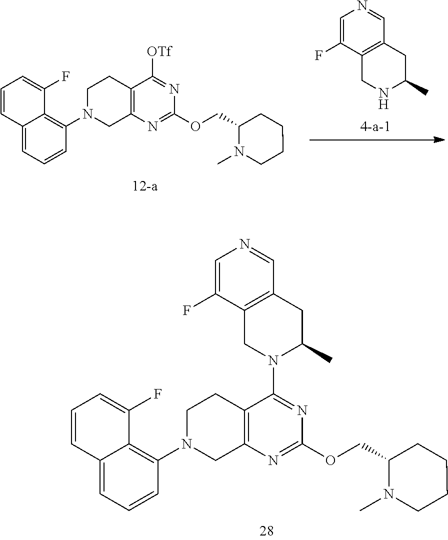

### Synthesis of Compound 28

12-a (70 mg, 0.13 mmol), DMAC (3 mL), 4-a-1 (27 mg, 0.16 mmol) and DIPEA (49 mg, 0.38 mmol) were combined in a reaction flask and the resulting mixture was stirred at 70° C. under nitrogen for 6 h. The reaction was quenched by adding water (30 mL) and extracted with ethyl acetate (30 mL*2). The organic phase was concentrated and the crude product was purified by column chromatography (mobile phase: methanol (containing NH₃)/dichloromethane 1/20 to 1/10) to give compound 28 (16.6 mg, 23%). LC-MS (ESI): m/z 571.8 (M+H)⁺; ¹H NMR (400 MHz, CD₃OD): δ 8.28 (1H, s), 8.13-8.27 (1H, m), 7.67 (1H, d, J=8.4 Hz), 7.59 (1H, d, J=8.4 Hz), 7.38-7.48 (2H, m), 7.13-7.24 (2H, m), 4.53-4.83 (2H, m), 4.19-4.51 (3H, m), 3.92-4.13 (1H, m), 3.60-3.78 (1H, m), 3.33-3.50 (1H, m), 2.95-3.28 (1H, m), 2.75-2.94 (3H, m), 2.55-2.72 (1H, m), 2.07-2.47 (5H, m), 1.51-1.93 (4H, m), 1.12-1.50 (6H, m).

### Experiment 29 Synthetic Route of Compound 29

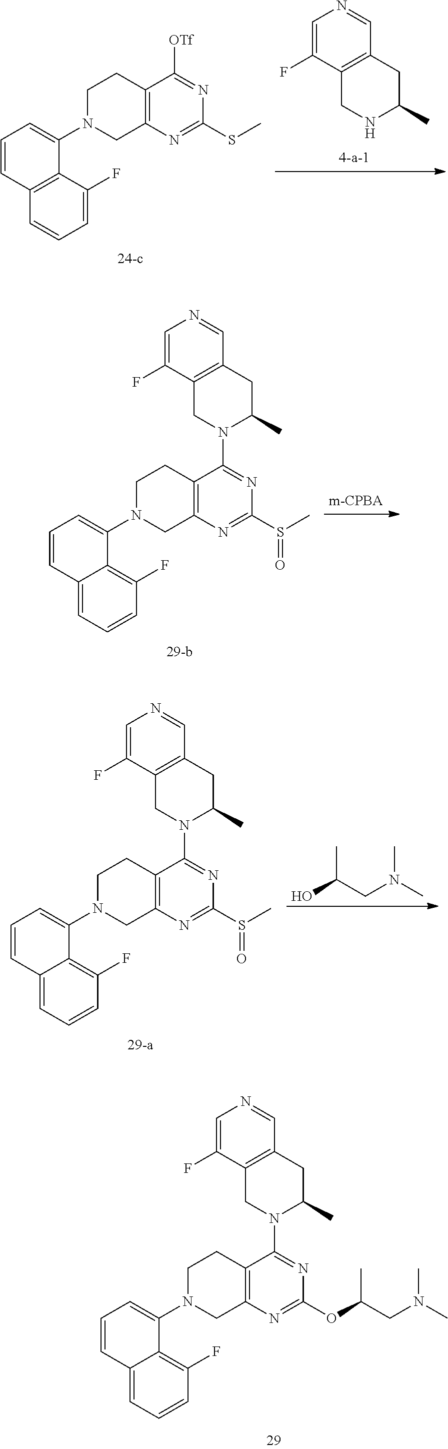

### Synthesis of Compound 29-b

24-c (190 mg, 0.40 mmol), 4-a-1 (73 mg, 0.44 mmol), DIPEA (104 mg, 0.80 mmol), and DMSO (2 mL) were combined in a reaction flask. Degassed and purged with nitrogen, the reaction was stirred at 70° C. for 8 hours. The reaction mixture was cooled, added water and extracted with DCM:MeOH=10:1 solution, the organic phases were combined, dried and concentrated, and purified on a silica column (mobile phase: DCM/MeOH 10/0 to 10/1) to give compound 29-b (150 mg, 76%). LC-MS (ESI): m/z 490.1 (M+H)⁺.

### Synthesis of Compound 29-a

To a reaction vial was added 29-b (150 mg, 0.31 mmol), m-chloroperoxybenzoic acid (79 mg, 0.46 mmol) and DCM (10 mL). Degassed and purged with nitrogen, the reaction was stirred at room temperature for 1 hour. The reaction was quenched with aqueous sodium bicarbonate, extracted with DCM, and the organic phases were combined and concentrated by drying to give compound 29-a (150 mg, 97%). LC-MS (ESI): m/z 506.1 (M+H)⁺.

### Synthesis of Compound 29

To a reaction vial was added 29-a (50 mg, 0.10 mmol), (S)-1-(dimethylamino)propan-2-ol (21 mg, 0.20 mmol), sodium tert-butoxide (29 mg, 0.30 mmol), DCM (10 mL). Degassed and purged with nitrogen, the reaction was stirred at room temperature for half an hour. The reaction mixture was mixed into silica gel and purified by column chromatography (mobile phase: DCM/MeOH 10/0 to 10/1) to give compound 29 (9 mg, 17%). LC-MS (ESI): m/z 545.7 (M+H)⁺.

### Experiment 30 Synthetic Route of Compound 30

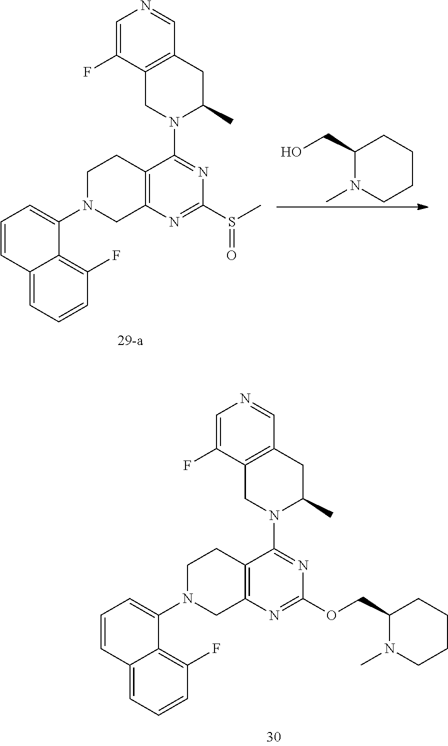

### Synthesis of Compound 30

To a reaction vial was added 29-a (50 mg, 0.10 mmol), (2R)-N-methyl-2-piperidinemethanol (26 mg, 0.20 mmol), tert-butanol sodium (29 mg, 0.30 mmol) and DCM (10 mL). Degassed and purged with nitrogen, the reaction was stirred at room temperature for half an hour. The reaction mixture was mixed into silica gel and purified by column chromatography (mobile phase: DCM/MeOH 10/0 to 10/1) to give compound 30 (10 mg, 18%). LC-MS (ESI): m/z 571.7 (M+H)⁺.

### Experiment 31 Synthesis of Compound 31

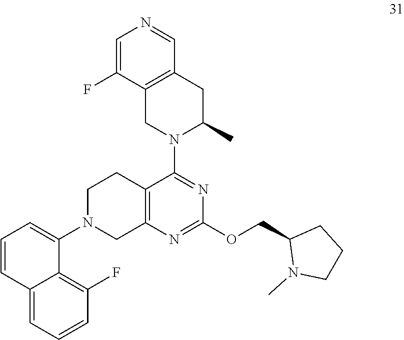 [SMILES: C[C@@H]1Cc2cncc(F)c2CN1c1nc(OC[C@H]2CCCN2C)nc2c1CCN(c1cccc3cccc(F)c13)C2]

Referring to the synthetic route of compound 30, compound 31 was synthesized using N-methyl-D-prolinol instead of (2R)-N-methyl-2-piperidinemethanol, and 29-a was used as starting material. LC-MS (ESI): m/z 557.8 (M+H)⁺.

### Experiment 32 Synthesis of Compound 32

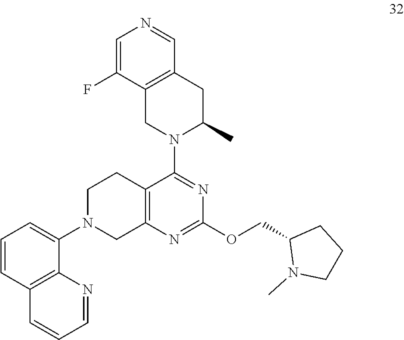 [SMILES: C[C@@H]1Cc2cncc(F)c2CN1c1nc(OC[C@@H]2CCCN2C)nc2c1CCN(c1cccc3cccnc13)C2]

Referring to the synthesis of compound 14, it was synthesized using 8-bromoisoquinoline instead of 5-bromoquinoline and 4-a-1 instead of 4-a-2. Compound 32: LC-MS (ESI): m/z 540.7 (M+H)⁺.

### Experiment 33 Synthetic Route of Compound 33

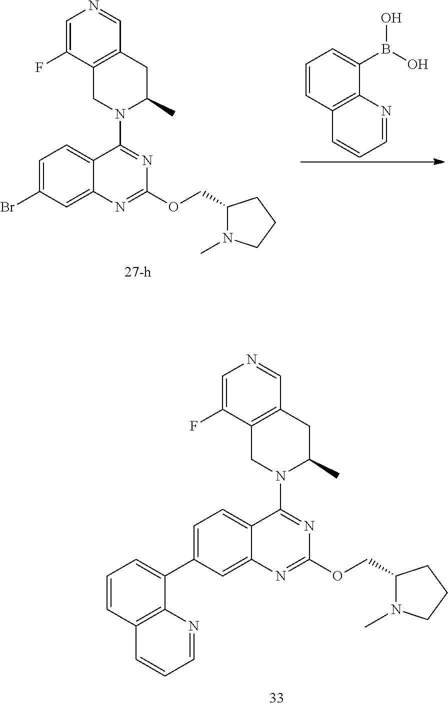 [SMILES: C[C@@H]1Cc2cncc(F)c2CN1c1nc(OC[C@@H]2CCCN2C)nc2cc(Br)ccc12 ; OB(O)c1cccc2cccnc12 ; C[C@@H]1Cc2cncc(F)c2CN1c1nc(OC[C@@H]2CCCN2C)nc2cc(-c3cccc4cccnc34)ccc12]

### Synthesis of Compound 33

To a 100 mL aubergine flask was added 27-h (40 mg, 0.082 mmol), quinoline-8-boronic acid (28 mg, 0.16 mmol), Pd(PPh₃)₄(10 mg, 0.008 mmol), sodium carbonate (26 mg, 0.25 mmol), toluene (6 mL), anhydrous ethanol (3 mL) and water (2 mL). Degassed and purged with nitrogen, the reaction was stirred at 110° C. overnight. The reaction mixture was concentrated to dryness, added water and extracted with ethyl acetate (30 mL*2), and the combined organic phases were dried over anhydrous sodium sulfate, filtered and concentrated to dryness. The crude product was purified by column chromatography (mobile phase: 0-100%, dichloromethane:methanol=10:1/dichloromethane) and Prep-HPLC to obtain 33 (24 mg, 55%). LC-MS (ESI): m/z 535.1 (M+H)⁺; ¹H NMR (400 MHz, DMSO-d₆): δ 8.96 (1H, dd, J=4.4, 2 Hz), 8.50 (1H, dd, J=8, 1.6 Hz), 8.44 (1H, s), 8.38 (1H, s), 8.13-8.09 (2H, m), 7.94 (1H, dd, J=7.2, 1.6 Hz), 7.89 (1H, d, J=1.6 Hz), 7.79-7.72 (2H, m), 7.64 (1H, q, J=4.0 Hz), 5.13-5.03 (2H, m), 4.80 (1H, d, J=18.4 Hz), 4.43 (1H, dd, J=10.8, 4.8 Hz), 4.23 (1H, dd, J=10.8, 6.4 Hz), 3.52 (1H, dd, J=16.8, 5.6 Hz), 3.03-2.96 (1H, m), 2.93 (1H, d, J=16.8 Hz), 2.67-2.59 (1H, m), 2.41 (3H, s), 2.26-2.17 (1H, m), 2.03-1.94 (1H, m), 1.76-1.62 (3H, m), 1.22 (3H, d, J=6.4 Hz).

### Synthesis of Compound 34

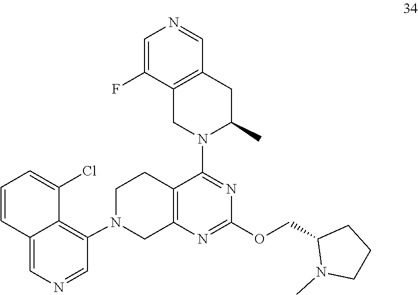 [SMILES: C[C@@H]1Cc2cncc(F)c2CN1c1nc(OC[C@@H]2CCCN2C)nc2c1CCN(c1cncc3cccc(Cl)c13)C2]

Referring to the synthesis of compound 14, compound 34 was synthesized using 4-bromo-5-chloroisoquinoline instead of 5-bromoquinoline and 4-a-1 instead of 4-a-2. Compound 34. LC-MS (ESI): m/z 574.2 (M+H)⁺.

### Synthesis of Compound 35

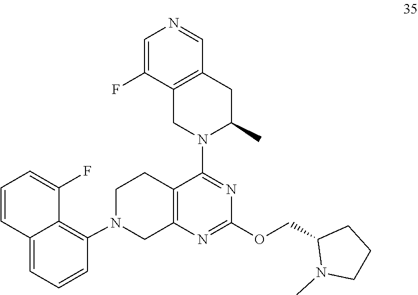 [SMILES: C[C@@H]1Cc2cncc(F)c2CN1c1nc(OC[C@@H]2CCCN2C)nc2c1CCN(c1cccc3cccc(F)c13)C2]

Referring to the synthesis of compound 15, compound 35 was synthesized using 4-a-1 instead of 4-a-2. LC-MS (ESI): m/z 557.3 (M+H)⁺.

### Experiment 36 Synthetic Route of Compound 36

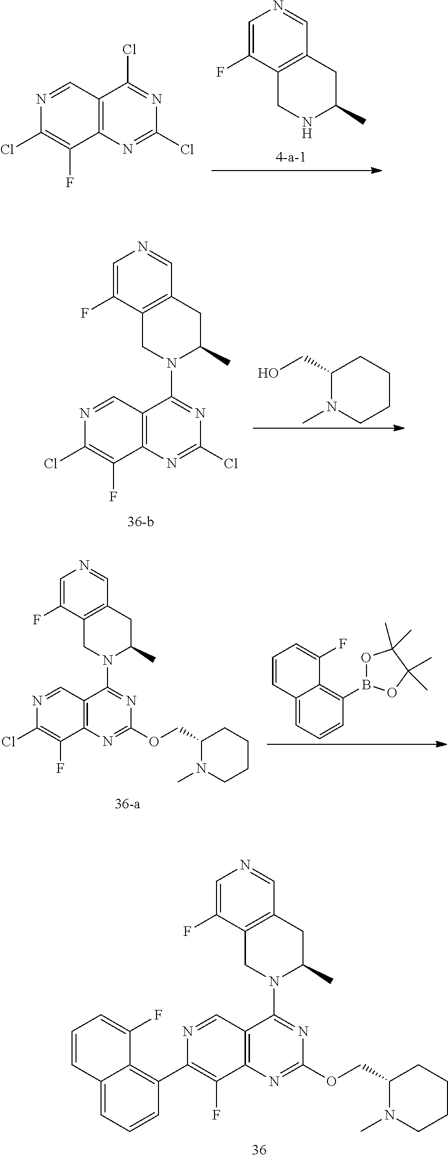

### Synthesis of Compound 36-b

DIPEA (0.21 mL, 1.19 mmol) and compound 4-a-1 (66 mg, 0.40 mmol) were added to a solution of compound 2,4,7-trichloro-8-fluoropyrido[4,3-d]pyrimidine (100 mg, 0.40 mmol) in dichloromethane (20 mL) in a reaction vial cooled in a dry ice-acetone bath. After addition, the mixture was stirred at room temperature overnight. The next day, the reaction was quenched with water, extracted with dichloromethane (50 mL*3), dried, concentrated to dryness and purified by column chromatography (mobile phase: ethyl acetate/petroleum ether 0/100 to 100/0) to give compound 36-b (135 mg, 89%). LC-MS (ESI): m/z=381.7 (M+H)⁺.

### Synthesis of Compound 36-a

Sodium tert-butoxide (85 mg, 0.88 mmol) was added to a solution of compound 36-b (135 mg, 0.35 mmol) and compound (S)-1-methylpiperidine-2-methanol (55 mg, 0.424 mmol) in dichloromethane (20 mL) in a reaction vial in an ice-water bath. After addition, the reaction mixture was stirred for 1 h. The compound 36-a (41 mg, 24%) was obtained directly by column chromatography (mobile phase: ammonia-methanol/dichloromethane 0/100 to 5/95). LC-MS (ESI): m/z=475.6 (M+H)⁺.

### Synthesis of Compound 36

Compound 36-a (40 mg, 0.084 mmol), compound 8-fluoro-1-naphthaleneboronic acid pinacol ester (46 mg, 0.17 mmol), 1,4-dioxane (15 mL), water (1.5 mL), cesium carbonate (82 mg, 0.253 mmol), Pd(PPh₃)₄ (10 mg, 0.008 mmol) were combined in a reaction vial at room temperature. Degassed and purged with nitrogen three times, the reaction was stirred at 100° C. overnight. The reaction mixture was concentrated to dryness, purified by column chromatography (mobile phase: methanol/dichloromethane 0/100 to 10/90), Prep-HPLC (ammonium bicarbonate) and lyophilized to give compound 36 (10 mg, 20%). LC-MS (ESI): m/z=585.8 (M+H)⁺; ¹H NMR (400M, CDCl₃): S 9.23-9.17 (1H, m), 8.38-8.31 (2H, m), 8.14-8.08 (1H, m), 8.06-7.74 (1H, m), 7.69-7.44 (3H, m), 7.24-6.99 (1H, m), 5.50-5.25 (3H, m), 5.23-5.03 (1H, m), 4.97-4.83 (1H, m), 4.77-4.61 (1H, m), 3.58-3.42 (2H, m), 3.35-3.17 (1H, m), 3.05-3.86 (4H, m), 2.80-2.61 (1H, m), 2.37-2.09 (2H, m), 2.07-1.94 (1H, m), 1.91-1.77 (1H, m), 1.41-1.32 (4H, m).

### Effect Example 1 Proliferation Inhibition of Compounds on RAS Cell Lines Measured by CTG

NCI-H358 is a human non-small cell lung cancer cell line with KRAS G12C mutation; AGS is a gastric cancer cell line with KRAS G12D mutation; SW480 is a colon cancer cell lines with KRAS G12V mutation; A375 is a wild type malignant melanoma cell line. The proliferation inhibitory effect of the compounds on various mutations was evaluated by determining the proliferation inhibitory activity of the compounds on these cell lines.

The assay was conducted on either 384-well plates or 96-well plates. The procedures are as follows:

The cell suspensions were added to 384- or 96-well plates (384-well plate: 40 μL; 96-well plate: 100 μL) except for peripheral wells. The plates were incubated in a carbon dioxide incubator overnight. Prepared compounds (10 concentration gradients by serial 3-fold dilution) were added to the wells. The cell plates were incubated in a carbon dioxide incubator for 120 hours. CellTiter Glo reagent (384 well plate: 25 μL; 96 well plate: 100 μL) was added to the 384- or 96-well plates. The plates were shaken for 10 minutes away from light, and incubated for 10 minutes. The plates were read by EnVision system. Inhibition curves were plotted using XLFit and IC₅₀ values were calculated. The activity results for representative compounds are shown in Tables 1 below. “IC₅₀>10 μM” is denoted by “*”, “10 μM≥IC₅₀>1 μM” is denoted by “*”, “IC₅₀≤1 μM” is denoted by “***”, “---” means no data.

TABLE 1
Proliferation inhibitory activity of representative
compounds of the present disclosure on RAS cells

| | IC₅₀ | IC₅₀ | IC₅₀ | IC₅₀ |
|---|---|---|---|---|
| Compound No. | (H358) | (AGS) | (SW480) | (A375) |
| 1 | *** | — | — | *** |
| 2 | ** | ** | — | ** |
| 3 | *** | *** | *** | *** |
| 4 | — | *** | *** | — |
| 5 | — | *** | *** | — |
| 6 | — | *** | *** | — |
| 3-1 | ** | ** | ** | ** |
| 3-2 | *** | *** | *** | *** |
| 4-1 | ** | *** | ** | ** |
| 4-2 | *** | *** | *** | *** |
| 5-1 | *** | *** | *** | *** |
| 5-2 | *** | *** | *** | *** |
| 7 | ** | *** | ** | ** |
| 9-1 | *** | *** | *** | *** |
| 9-2 | *** | *** | *** | *** |
| 11 | ** | *** | — | ** |
| 11-1 | ** | ** | — | ** |
| 11-2 | ** | *** | — | ** |
| 12 | *** | *** | *** | *** |
| 13 | ** | ** | ** | * |
| 14 | ** | ** | ** | ** |
| 15 | *** | *** | *** | ** |
| 16 | ** | ** | ** | ** |
| 17 | *** | *** | *** | ** |
| 18 | ** | ** | ** | ** |
| 19 | ** | ** | ** | * |
| 20 | ** | *** | *** | ** |
| 21 | ** | *** | ** | ** |
| 22 | *** | *** | *** | ** |
| 23 | ** | *** | ** | ** |
| 24 | ** | ** | ** | * |
| 25 | ** | ** | ** | ** |
| 26 | ** | ** | ** | ** |
| 27 | *** | *** | *** | *** |
| 28 | *** | *** | *** | *** |
| 29 | *** | *** | *** | ** |
| 30 | *** | *** | *** | *** |
| 31 | *** | *** | *** | *** |
| 32 | *** | *** | *** | *** |
| 33 | *** | *** | *** | *** |
| 34 | — | *** | — | *** |
| 35 | — | *** | — | *** |
| 36 | — | *** | — | — |

Although specific embodiments of the present disclosure have been described above, it will be appreciated by those skilled in the art that these embodiments are merely illustrative and that many changes or modifications can be made to these embodiments without departing from the principles and spirit of the present disclosure. The scope of protection of the present disclosure is therefore defined by the appended claims.

1. A nitrogen-containing heterocyclic compound of formula I, a pharmaceutically acceptable salt thereof, a stereoisomer thereof, a tautomer thereof or an isotopically labeled compound thereof: 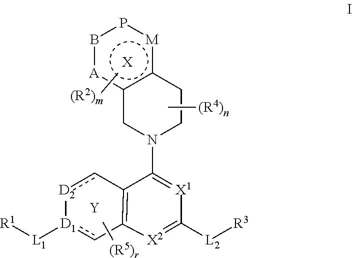
wherein “ [symbol] ” represents a single or double bond; 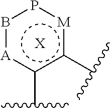
is nitrogen-containing 6-membered heteroaryl; A, B, P and M are independently CH or N, and at least one of them is N;
m is 0, 1, 2 or 3;
R² is —CN, C₁₋₆ alkyl, C₂₋₆ alkenyl, C₂₋₆ alkynyl, C₁₋₆ alkyl substituted with one or more R²⁻¹, halogen, —OR^{2a}, —C(═O)R^{2b}, —NR²ciR^{2c2}, —C(═O)OR²d, —C(═O)NR^{2e1}R^{2e2}, C₃₋₁₀ cycloalkyl, C₃₋₁₀ cycloalkyl substituted with one or more R²-2, “4- to 10-membered heterocycloalkyl containing 1-3 heteroatoms independently selected from O and N”, “4- to 10-membered heterocycloalkyl containing 1-3 heteroatoms independently selected from O and N” substituted with one or more R²⁻³, C₆₋₂₀ aryl, C₆₋₂₀ aryl substituted with one or more R²⁻⁴, “5- to 12-membered heteroaryl containing 1-4 heteroatoms independently selected from O, S and N”, or “5- to 12-membered heteroaryl containing 1-4 heteroatoms independently selected from O, S and N” substituted with one or more R²-5; provided that when multiple substituents are present, the substituents are the same or different;
R²⁻¹, R²⁻², R²⁻³, R²⁻⁴ and R²⁻⁵ are independently halogen, hydroxyl, cyano, C₁₋₆ alkyl, C₂₋₆ alkenyl, C₂₋₆ alkynyl, C₁₋₆ alkyl-O—, —C(═O)R³¹, —NR³²R³³, —C(═O)OR³⁴ or —C(═O)NR³⁵R³⁶;
R^{2a}, R^{2b}, R^{2c1}, R^{2c2}, R^{2a}, R^{2e1} and R^{2e2} are independently hydrogen or C₁₋₆ alkyl;
R³¹, R³², R³³, R³⁴, R³⁵ and R³⁶ are independently hydrogen or C₁₋₆ alkyl;
n is 0, 1, 2, 3, 4, 5 or 6;
R⁴ is independently C₁₋₆ alkyl, C₁₋₆ alkyl substituted with one or more R⁴⁻¹, C₁₋₆ alkyl-O—, O═, —C(═O)OR^{4a} or —C(═O)NR^{4b}R^{4c}; or, when n is 2, 3, 4, 5 or 6, two optional R⁴ are connected, together with the atoms on the ring to which they are attached, independently form 3- to 8-membered carbocyclic ring or 3- to 8-membered heterocyclic ring containing 1-3 heteroatoms independently selected from O, S and N;
R⁴⁻¹ is independently halogen, cyano, hydroxyl, C₁₋₆ alkyl-O—, —NR⁴ⁱR^{4j}, —C(═O)OR^{4d} or —C(═O)NR^{4e}R^{4f};
R^{4a}, R^{4b}, R^{4c}, R^{4d}, R^{4e}, R^{4f}, R⁴ⁱ and R^{4j} are independently hydrogen or C₁₋₆ alkyl; 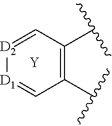
is phenyl, “5- to 7-membered heterocycloalkenyl containing 1-3 heteroatoms independently selected from O, S and N”, “5- to 7-membered heteroaryl containing 1-3 heteroatoms independently selected from O, S and N” or 5- to 7-membered cycloalkenyl; wherein D¹ is C, CH or N; D₂ is 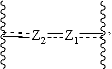
wherein Z₁ and Z₂ are independently a bond, CH, CH₂, O, S, N or NH;
r is 0, 1, 2, 3, 4, 5 or 6;
R⁵ is independently halogen or C₁₋₆ alkyl;
L₁ is a bond, —C(═O)— or C₁₋₆ alkylene;
R¹ is C₆₋₂₀ aryl, C₈₋₁₁ benzocycloalkenyl, “5- to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;
X¹ and X² are independently CR^{b} or N, and X¹ and X² are not both CR^{b};
R^{b}, R¹⁻¹ and R¹⁻² are independently halogen, —OR^{c}, cyano, —C(═O)R¹¹, —NR¹²R¹³, —C(═O)OR¹⁴, —C(═O)NR¹⁵R¹⁶, C₁₋₆ alkyl, C₂₋₆ alkenyl, C₂₋₆ alkynyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R¹⁻¹, C₁₋₆ alkyl-O substituted with one or more R¹⁻¹⁻², C₃₋₁₀ cycloalkyl substituted with one or more R¹⁻¹⁻³, “5- to 7-membered heterocycloalkyl containing 1 or 2 heteroatoms independently selected from O and N” substituted with one or more R¹⁻¹⁻⁴, C₆₋₂₀ aryl substituted with one or more R¹⁻¹-5 or “5- to 7-membered heteroaryl containing 1 or 2 heteroatoms independently selected from O and N” substituted with one or more R¹⁻¹⁻⁶; provided that when multiple substituents are present, the substituents are the same or different; or, when the number of R¹⁻¹ or R¹⁻² is more than one, two optional R¹⁻¹ or two optional R¹⁻² are connected, together with the atoms on the ring to which they are attached, independently form 3- to 8-membered cyclic olefin;
R^{c}, R¹² and R¹³ are independently hydrogen, C₁₋₆ alkyl, C(═O)R^{c1}, —C(═O)OR^{c2}, or —C(═O)NR^{c3}R^{c4};
R^{c}l, R^{c2}, R^{c3} and R^{c4} are independently hydrogen, C₁₋₆ alkyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R⁴⁻¹, C₃₋₁₀ cycloalkyl substituted with one or more R⁴⁻¹⁻², “5- to 7-membered heterocycloalkyl containing 1 or 2 heteroatoms independently selected from O and N” substituted with one or more R¹⁻¹⁻³, C₆₋₂₀ aryl substituted with one or more R¹⁻¹⁻⁴, or, “5- to 7-membered heteroaryl containing 1 or 2 heteroatoms independently selected from O and N” substituted with one or more R¹⁻¹⁻⁵; provided that when multiple substituents are present, the substituents are the same or different;
R¹⁻¹, R¹⁻¹⁻², R¹⁻¹⁻³, R¹⁻¹⁻⁴, R¹⁻¹⁻⁵, R¹⁻¹⁻⁶, R¹⁻¹⁻¹, R¹⁻¹⁻², R¹⁻¹⁻³, R⁴⁻¹⁴ and R¹⁻¹⁻⁵ are independently cyano, halogen, hydroxyl, C₁₋₆ alkyl-O—, C₁₋₆ alkyl, —C(═O)R²¹, —NR²²R²³, —C(═O)OR²⁴ or —C(═O)NR²⁵R²⁶;
R¹¹, R²¹, R²², R²³, R¹⁴, R²⁴, R¹⁶, R²⁵, R¹⁶ and R²⁶ are independently hydrogen or C₁₋₆ alkyl;
L₂ is a bond, C₁₋₆ alkylene, —C(═O)—, —O(R^{L-1})ₙ₁—, —S(R^{L-2})ₙ2- or —NR^{L-3}(R^{L-4})ₙ3-; R^{L-1}, R^{L-2} and R^{L-4} are independently C₁₋₆ alkylene; R^{L-3} is hydrogen or C₁₋₆ alkyl; n1, n2 and n3 are independently 0 or 1;
R³ is C₃₋₁₂ cycloalkyl, C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻², C₁₋₆ alkyl, C₁₋₆ alkyl substituted with one or more R³⁻³, —OR^{d}, —SR^{d1}, —NR^{e1}R^{e2} or —C(═O)NR^{e3}R^{e4}; provided that when multiple substituents are present, the substituents are the same or different;
R³⁻¹, R³⁻² and R³⁻³ are independently C₁₋₆ alkyl, C₁₋₆ alkyl substituted with one or more R³⁻¹⁻¹ hydroxyl, C₁₋₆ alkyl-O—, halogen, O═, —NR^{e5}R^{e6} or —C(═O)NR^{e7}R^{e8};
R^{d}, R^{d1}, R^{e1}, R^{e2}, R^{e3} and R^{e4} are independently hydrogen, C₁₋₆ alkyl, C₃₋₁₀ cycloalkyl, “4- to 10-membered heterocycloalkyl containing 1-3 heteroatoms independently selected from O and N”, or C₁₋₆ alkyl substituted with one or more R³⁻¹⁻²;
R³⁻¹⁻¹ and R³⁻¹⁻² are independently deuterium, cyano, halogen, hydroxyl, C₁₋₆ alkyl-O—, —C(═O)R^{e9}, —NR^{e10}R^{e11}, —C(═O)OR^{e12} or —C(═O)NR^{e13}R^{e14};
R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl.

2. The nitrogen-containing heterocyclic compound of formula I, the pharmaceutically acceptable salt thereof, the stereoisomer thereof, the tautomer thereof or the isotopically labeled compound thereof according to claim 1, wherein,
in 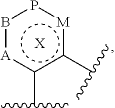
P is N, A, B and M are CH;
m is 0, 1, 2 or 3;
R² is —CN, C₁₋₆ alkyl, C₂₋₆ alkenyl, C₂₋₆ alkynyl, C₁₋₆ alkyl substituted with one or more R²⁻¹, halogen, —OR^{2a}, —C(═O)R^{2b}, —NR^{2c1}R^{2c2}, —C(═O)OR²d, —C(═O)NR^{2e1}R^{2e2}, C₃₋₁₀ cycloalkyl, C₃₋₁₀ cycloalkyl substituted with one or more R²-2, “4- to 10-membered heterocycloalkyl containing 1-3 heteroatoms independently selected from O and N”, “4- to 10-membered heterocycloalkyl containing 1-3 heteroatoms independently selected from O and N” substituted with one or more R²⁻³, C₆₋₂₀ aryl, C₆₋₂₀ aryl substituted with one or more R²⁻⁴, “5- to 12-membered heteroaryl containing 1-4 heteroatoms independently selected from O, S and N”, or “5- to 12-membered heteroaryl containing 1-4 heteroatoms independently selected from O, S and N” substituted with one or more R²-5; provided that when multiple substituents are present, the substituents are the same or different;
R²⁻¹, R²⁻², R²⁻³, R²⁻⁴ and R²⁻⁵ are independently halogen, hydroxyl, cyano, C₁₋₆ alkyl, C₂₋₆ alkenyl, C₂₋₆ alkynyl, C₁₋₆ alkyl-O—, —C(═O)R³¹, —NR³²R³³, —C(═O)OR³⁴ or —C(═O)NR³⁵R³⁶;
R^{2a}, R^{2b}, R^{2c1}, R^{2c2}, R^{2a}, R^{2e1} and R^{2e2} are independently hydrogen or C₁₋₆ alkyl;
R³¹, R³², R³³, R³⁴, R³⁵ and R³⁶ are independently hydrogen or C₁₋₆ alkyl;
n is 0, 1, 2, 3, 4, 5 or 6;
R⁴ is independently C₁₋₆ alkyl, C₁₋₆ alkyl substituted with one or more R⁴⁻¹, C₁₋₆ alkyl-O—, O═, —C(═O)OR^{4a} or —C(═O)NR^{4b}R^{4c}; or, when n is 2, 3, 4, 5 or 6, two optional R⁴ are connected, together with the atoms on the ring to which they are attached, independently form 3- to 8-membered carbocyclic ring or 3- to 8-membered heterocyclic ring containing 1-3 heteroatoms independently selected from O, S and N;
R⁴⁻¹ is independently halogen, cyano, hydroxyl, C₁₋₆ alkyl-O—, —NR⁴ⁱR^{4j}, —C(═O)OR⁴d or —C(═O)NR^{4e}R^{4f};
R^{4a}, R^{4b}, R^{4c}, R^{4d}, R^{4e}, R^{4f}, R⁴ⁱ and R^{4j} are independently hydrogen or C₁₋₆ alkyl; 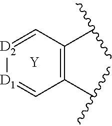
is phenyl, “5- to 7-membered heterocycloalkenyl containing 1-3 heteroatoms independently selected from O, S and N”, “5- to 7-membered heteroaryl containing 1-3 heteroatoms independently selected from O, S and N” or 5- to 7-membered cycloalkenyl; wherein D¹ is C, CH or N; D₂ is 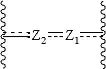
wherein Z₁ and Z₂ are independently a bond, CH, CH₂, O, S, N or NH;
r is 0, 1, 2, 3, 4, 5 or 6;
R⁵ is independently halogen or C₁₋₆ alkyl;
L₁ is a bond, —C(═O)— or C₁₋₆ alkylene;
R¹ is C₆₋₂₀ aryl, C₈₋₁₁ benzocycloalkenyl, “5- to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;
X¹ and X² are independently CR^{b} or N, and X¹ and X² are not both CR^{b};
R^{b}, R¹⁻¹ and R¹⁻² are independently halogen, —OR^{c}, cyano, —C(═O)R¹¹, —NR¹²R¹³, —C(═O)OR¹⁴, —C(═O)NR¹⁵R¹⁶, C₁₋₆ alkyl, C₂₋₆ alkenyl, C₂₋₆ alkynyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R¹⁻¹⁻¹, C₁₋₆ alkyl-O substituted with one or more R¹⁻¹⁻², C₃₋₁₀ cycloalkyl substituted with one or more R¹⁻¹⁻³, “5- to 7-membered heterocycloalkyl containing 1 or 2 heteroatoms independently selected from O and N” substituted with one or more R¹⁻¹⁻⁴, C₆₋₂₀ aryl substituted with one or more R¹⁻¹-5 or “5- to 7-membered heteroaryl containing 1 or 2 heteroatoms independently selected from O and N” substituted with one or more R¹⁻¹⁻⁶; provided that when multiple substituents are present, the substituents are the same or different; or, when the number of R¹⁻¹ or R¹⁻² is more than one, two optional R¹⁻¹ or two optional R¹⁻² are connected, together with the atoms on the ring to which they are attached, independently form 3- to 8-membered cyclic olefin;
R^{c}, R¹² and R¹³ are independently hydrogen, C₁₋₆ alkyl, C(═O)R^{c1}, —C(═O)OR^{c2}, or —C(═O)NR^{3c}R^{c4};
R^{c}l, R^{c2}, R^{c3} and R^{c4} are independently hydrogen, C₁₋₆ alkyl, C₃₋₁₀ cycloalkyl, “5- to 7-membered heterocycloalkyl containing 1 or 2 heteroatoms independently selected from O and N”, C₆₋₂₀ aryl, “5- to 7-membered heteroaryl containing 1 or 2 heteroatoms independently selected from O and N”, C₁₋₆ alkyl substituted with one or more R¹⁻¹⁻¹, C₃₋₁₀ cycloalkyl substituted with one or more R⁴⁻¹⁻², “5- to 7-membered heterocycloalkyl containing 1 or 2 heteroatoms independently selected from O and N” substituted with one or more R¹⁻¹⁻³, C₆₋₂₀ aryl substituted with one or more R¹⁻¹⁻⁴, or, “5- to 7-membered heteroaryl containing 1 or 2 heteroatoms independently selected from O and N” substituted with one or more R¹⁻¹⁻⁵; provided that when multiple substituents are present, the substituents are the same or different;
R¹⁻¹⁻¹, R¹⁻¹⁻², R¹⁻¹⁻³, R¹⁻¹⁻⁴, R¹⁻¹⁻⁵, R¹⁻¹⁻⁶, R¹⁻¹⁻¹, R¹⁻¹⁻², R¹⁻¹⁻³, R⁴⁻¹⁴ and R¹⁻¹⁻⁵ are independently cyano, halogen, hydroxyl, C₁₋₆ alkyl-O—, C₁₋₆ alkyl, —C(═O)R²¹, —NR²²R²³, —C(═O)OR²⁴ or —C(═O)NR²⁵R²⁶;
R¹¹, R²¹, R²², R²³, R¹⁴, R²⁴, R¹⁵, R²⁵, R¹⁶ and R²⁶ are independently hydrogen or C₁₋₆ alkyl;
L₂ is a bond, C₁₋₆ alkylene, —C(═O)—, —O(R^{L-1})ₙ₁—, —S(R^{L-2})ₙ2- or —NR^{L-3}(R^{L-4})ₙ₃—; R^{L-1}, R^{L-2} and R^{L-4} are independently C₁₋₆ alkylene; R^{L-3} is hydrogen or C₁₋₆ alkyl; n1, n2 and n3 are independently 0 or 1;
R³ is C₃₋₁₂ cycloalkyl, C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻², C₁₋₆ alkyl, C₁₋₆ alkyl substituted with one or more R³⁻³, —OR^{d}, —SR^{d1}, —NR^{e1}R^{e2} or —C(═O)NR^{e3}R^{e4}; provided that when multiple substituents are present, the substituents are the same or different;
R³⁻¹, R³⁻² and R³⁻³ are independently C₁₋₆ alkyl, C₁₋₆ alkyl substituted with one or more R³⁻¹⁻¹ hydroxyl, C₁₋₆ alkyl-O—, halogen, O═, —NR^{e5}R^{e6} or —C(═O)NR^{e7}R^{e8};
R^{d}, R^{d1}, R^{e1}, R^{e2}, R^{e3} and R^{e4} are independently hydrogen, C₁₋₆ alkyl, C₃₋₁₀ cycloalkyl, “4- to 10-membered heterocycloalkyl containing 1-3 heteroatoms independently selected from O and N”, or C₁₋₆ alkyl substituted with one or more R³⁻¹⁻²;
R³⁻¹⁻¹ and R³⁻¹⁻² are independently deuterium, cyano, halogen, hydroxyl, C₁₋₆ alkyl-O—, —C(═O)R^{e9}, —NR^{e10}R^{e11}, —C(═O)OR^{e12} or —C(═O)NR^{e13}R^{e14};
R^{e5}, R^{e6}, R^{e7}, R^{e8}, R^{e9}, R^{e10}, R^{e11}, R^{e12}, R^{e13} and R^{e14} are independently hydrogen or C₁₋₆ alkyl.

3. The nitrogen-containing heterocyclic compound of formula I, the pharmaceutically acceptable salt thereof, the stereoisomer thereof, the tautomer thereof or the isotopically labeled compound thereof according to claim 1, wherein, 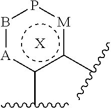
is nitrogen-containing 6-membered heteroaryl; P is N, A, B and M are independently CH, or, A is N, P, B and M are independently CH, or, B is N, A, P and M are independently CH;
and/or, m is 0 or 1;
and/or, R² is halogen or —OR²a;
and/or, R^{2a} is C₁₋₆ alkyl;
and/or, n is 0 or 1;
and/or, R⁴ is independently C₁₋₆ alkyl;
and/or, D¹ is C, CH or N;
and/or, in D₂, of Z₁ and Z₂, one is CH, CH₂ or O, the other one is a bond;
and/or, r is 0 or 1;
and/or, R⁵ is independently halogen;
and/or, X¹ and X² are independently N;
and/or, L₁ is a bond;
and/or, R¹ is C₆₋₂₀ aryl substituted with one or more R¹⁻¹, “5- to 12-membered heteroaryl containing 1-4 heteroatoms independently selected from O, S and N” or “5- to 12-membered heteroaryl containing 1-4 heteroatoms independently selected from O, S and N” substituted with one or more R¹⁻²;
and/or, R¹⁻¹ is independently halogen, —OR^{c}, C₂₋₆ alkynyl, C₁₋₆ alkyl-O substituted with one or more R¹⁻¹⁻²;
and/or, R^{c} is hydrogen;
and/or, R¹⁻¹⁻² is independently C₁₋₆ alkyl-O;
and/or, R¹⁻² is independently halogen or —NR¹²R¹¹;
and/or, R¹² and R¹³ are independently hydrogen;
and/or, L₂ is —O(R^{L-1})ₙ₁—;
and/or, R^{L-1} is independently C₁₋₆ alkylene;
and/or, n1 is 1;
and/or, R³ is “5- to 6-membered heterocycloalkyl containing 1-2 heteroatoms independently selected from O and N” substituted with one or more R³⁻² or —NR^{e1}R^{e2};
and/or, R³⁻² is independently C₁₋₆ alkyl;
and/or, R^{e1} and R^{e2} are independently C₁₋₆ alkyl;
and/or, when n is 1, 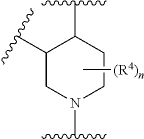
is 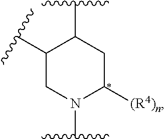
wherein, 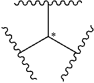 [SMILES: *C(*)*]
represents R configuration, S configuration or a mixture thereof.

4. The nitrogen-containing heterocyclic compound of formula I, the pharmaceutically acceptable salt thereof, the stereoisomer thereof, the tautomer thereof or the isotopically labeled compound thereof according to claim 1, wherein, nitrogen-containing heterocyclic compound of formula I is defined as solution 1, solution 2, solution 3, solution 4 or solution 5:
solution 1:
“ [symbol] ” represents a single or double bond; 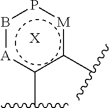
is nitrogen-containing 6-membered heteroaryl; P is N, A, B and M are independently CH, or, A is N, P, B and M are independently CH, or, B is N, A, P and M are independently CH;
m is 0 or 1;
R² is halogen or —OR^{2a}; R^{2a} is C₁₋₆ alkyl;
n is 0 or 1;
R⁴ is C₁₋₆ alkyl; 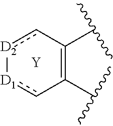
is phenyl, “6-membered heterocycloalkenyl containing 1 heteroatom selected from O or N”, or, “5- to 7-membered heteroaryl containing 1-3 heteroatoms independently selected from O, S and N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond; or D¹ is C, and in D₂, of Z₁ and Z₂, one is N, the other is a bond;
r is 0 or 1;
R⁵ is independently halogen;
L₁ is a bond;
R¹ is C₆₋₂₀ aryl substituted with one or more R¹⁻¹, “5- to 12-membered heteroaryl containing 1-4 heteroatoms independently selected from O, S and N” or “5- to 12-membered heteroaryl containing 1-4 heteroatoms independently selected from O, S and N” substituted with one or more R¹⁻²;
R¹⁻¹ is independently halogen, —OR^{c}, C₂₋₆ alkynyl, C₁₋₆ alkyl-O substituted with one or more R¹⁻¹⁻²;
R^{c} is hydrogen;
R¹⁻¹⁻² is independently C₁₋₆ alkyl-O—;
R¹⁻² is independently halogen or —NR¹²R¹³;
R¹² and R¹³ are independently hydrogen;
X¹ and X² are independently N;
L₂ is —O(R^{L-1})ₙ₁—; R^{L-1} is independently C₁₋₆ alkylene; n1 is 1;
R³ is “5- to 6-membered heterocycloalkyl containing 1-2 heteroatoms independently selected from O and N” substituted with one or more R³⁻² or —NR^{e1}R^{e2}R³⁻², R^{e1} and R^{e2} are independently C₁₋₆ alkyl;
When 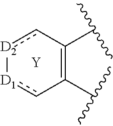
is “6-membered heterocycloalkenyl containing 1 heteroatom selected from O or N”, D¹ is CH, and in D₂, of Z₁ and Z₂, one is O, the other is a bond, 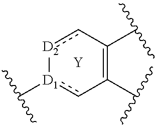
is 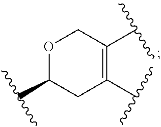 [SMILES: *C1=C(*)C[C@@H](*)OC1]
when n is 1, 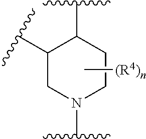
is 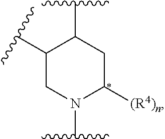
wherein, 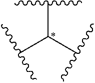 [SMILES: *C(*)*]
represents R configuration, S configuration or a mixture thereof;
solution 2:
“ [symbol] ” represents a single or double bond; 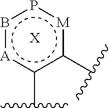
is nitrogen-containing 6-membered heteroaryl; P is N, A, B and M are independently CH, or, A is N, P, B and M are independently CH, or, B is N, A, P and M are independently CH;
m is 0 or 1;
R² is halogen or —OR^{2a}; R^{2a} is C₁₋₆ alkyl;
n is 0 or 1;
R⁴ is C₁₋₆ alkyl; 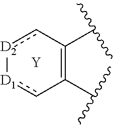
is phenyl, “6-membered heterocycloalkenyl containing 1 heteroatom selected from O or N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond;
r is 0;
L₁ is a bond;
R¹ is C₆₋₂₀ aryl substituted with one or more R¹⁻¹, “5- to 12-membered heteroaryl containing 1-4 heteroatoms independently selected from O, S and N” or “5- to 12-membered heteroaryl containing 1-4 heteroatoms independently selected from O, S and N” substituted with one or more R¹⁻² provided that when multiple substituents are present, the substituents are the same or different;
R¹⁻¹ is independently halogen, —OR^{c}, C₂₋₆ alkynyl, C₁₋₆ alkyl-0 substituted with one or more R¹⁻¹⁻²;
R^{c} is hydrogen;
R¹⁻¹⁻² is independently C₁₋₆ alkyl-O—;
R¹⁻² is independently halogen or —NR¹²R¹³;
R¹² and R¹³ are independently hydrogen;
X¹ and X² are independently N;
L₂ is —O(R^{L-1})ₙ₁—; R^{L-1} is independently C₁₋₆ alkylene; n1 is 1;
R³ is “5- to 6-membered heterocycloalkyl containing 1-2 heteroatoms independently selected from O and N” substituted with one or more R³⁻² or —NR^{e1}R^{e2};
R³⁻², R^{e1} and R^{e2} are independently C₁₋₆ alkyl;
When 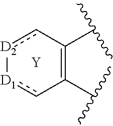
is “6-membered heterocycloalkenyl containing 1 heteroatom selected from O or N”, D¹ is CH, and in D₂, of Z₁ and Z₂, one is O, the other is a bond, 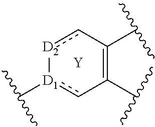
is 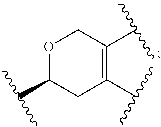 [SMILES: *C1=C(*)C[C@@H](*)OC1]
when n is 1, 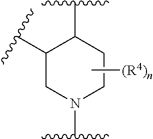
is 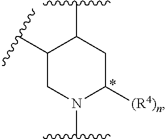
wherein, 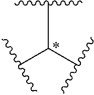 [SMILES: *C(*)*]
represents R configuration, S configuration or a mixture thereof;
solution 3:
“ [symbol] ” represents a single or double bond; 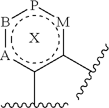
is nitrogen-containing 6-membered heteroaryl; P is N, A, B and M are independently CH, or, A is N, P, B and M are independently CH, or, B is N, A, P and M are independently CH;
m is 0 or 1;
R² is halogen or —OR^{2a}; R^{2a} is C₁₋₆ alkyl;
n is 0 or 1;
R⁴ is C₁₋₆ alkyl; 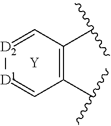
is phenyl, “6-membered heterocycloalkenyl containing 1 heteroatom selected from O or N”, wherein D¹ is C, and in D₂, of Z₁ and Z₂, one is CH, the other is a bond; or, D¹ is CH, and in D₂, of Z₁ and Z₂, one is O, the other is a bond; or, D¹ is N, and in D₂, of Z₁ and Z₂, one is CH₂, the other is a bond;
r is 0;
L₁ is a bond;
R¹ is C₆₋₂₀ aryl substituted with one or more R¹⁻¹; provided that when multiple substituents are present, the substituents are the same or different;
R¹⁻¹ is independently halogen, —OR^{c}, C₂₋₆ alkynyl, C₁₋₆ alkyl-O substituted with one or more R¹⁻¹⁻²;
R^{c} is hydrogen;
R¹⁻¹⁻² is independently C₁₋₆ alkyl-O—;
X¹ and X² are independently N;
L₂ is —O(R^{L-1})ₙ₁—; R^{L-1} is independently C₁₋₆ alkylene; n1 is 1;
R³ is “5- to 6-membered heterocycloalkyl containing 1 heteroatom being N” substituted with one or more R³⁻²;
R³⁻² is independently C₁₋₆ alkyl;
when 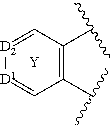
is “6-membered heterocycloalkenyl containing 1 heteroatom selected from O or N”, D¹ is CH, and in D₂, of Z₁ and Z₂, one is O, the other is a bond, 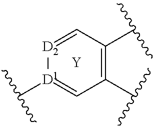
is 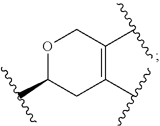 [SMILES: *C1=C(*)C[C@@H](*)OC1]
when n is 1, 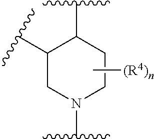
is 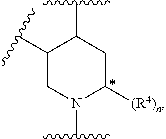
wherein, 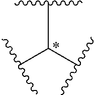 [SMILES: *C(*)*]
represents R configuration, S configuration or a mixture thereof;
solution 4:
“ [symbol] ” represents a single or double bond; 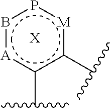
is nitrogen-containing 6-membered heteroaryl; P is N, A, B and M are independently CH, or, A is N, P, B and M are independently CH, or, B is N, A, P and M are independently CH;
m is 0 or 1;
R² is halogen or —OR^{2a}; R^{2a} is C₁₋₆ alkyl;
n is 0 or 1;
R⁴ is C₁₋₆ alkyl; 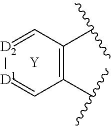
is “6-membered heterocycloalkenyl containing 1 heteroatom being N”, wherein D¹ is N, and in D₂, of Z₁ and Z₂, one is CH₂, the other is a bond;
r is 0;
L₁ is a bond;
R¹ is C₆₋₂₀ aryl substituted with one or more R¹⁻¹; provided that when multiple substituents are present, the substituents are the same or different;
R¹⁻¹ is independently halogen, —OR^{c} or C₂₋₆ alkynyl; R^{c} is hydrogen;
X¹ and X² are independently N;
L₂ is —O(R^{L-1})ₙ₁—; R^{L-1} is independently C₁₋₆ alkylene; n1 is 1;
R³ is “5- to 6-membered heterocycloalkyl containing 1 heteroatom being N” substituted with one or more R³⁻²;
R³⁻² is independently C₁₋₆ alkyl;
when n is 1, 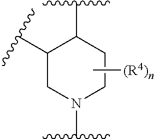
is 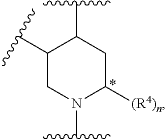
wherein, 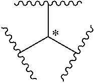 [SMILES: *C(*)*]
represents R configuration, S configuration or a mixture thereof;
solution 5:
“ [symbol] ” represents a single or double bond; 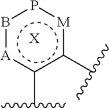
is nitrogen-containing 6-membered heteroaryl; P is N, A, B and M are independently CH;
m is 0 or 1;
R² is halogen or —OR^{2a}; R^{2a} is C₁₋₆ alkyl;
n is 0 or 1;
R⁴ is C₁₋₆ alkyl; 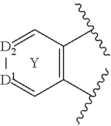
is “6-membered heterocycloalkenyl containing 1 heteroatom being N”, wherein D¹ is N, and in D₂, of Z₁ and Z₂, one is CH₂, the other is a bond;
r is 0;
L₁ is a bond;
R¹ is C₆₋₂₀ aryl substituted with one or more R¹⁻¹; provided that when multiple substituents are present, the substituents are the same or different;
R¹⁻¹ is independently halogen, —OR^{c} or C₂₋₆ alkynyl; R^{c} is hydrogen;
X¹ and X² are independently N;
L₂ is —O(R^{L-1})ₙ₁—; R^{L-1} is independently C₁₋₆ alkylene; n1 is 1;
R³ is “5-membered heterocycloalkyl containing 1 heteroatom being N” substituted with one or more R³⁻²;
R³⁻² is independently C₁₋₆ alkyl;
when n is 1, 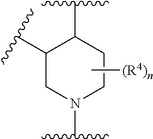
is 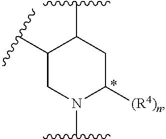
wherein, 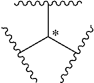 [SMILES: *C(*)*]
represents R configuration, S configuration or a mixture thereof.

5. The nitrogen-containing heterocyclic compound of formula I, the pharmaceutically acceptable salt thereof, the stereoisomer thereof, the tautomer thereof or the isotopically labeled compound thereof according to claim 1, wherein,
when R² is C₁₋₆ alkyl or C₁₋₆ alkyl substituted with one or more R²⁻¹, the C₁₋₆ alkyl and the C₁₋₆ alkyl in the C₁₋₆ alkyl substituted with one or more R²⁻¹ are methyl, ethyl, n-propyl, isopropyl, n-butyl, sec-butyl, isobutyl or tert-butyl;
and/or, when R² is C₂₋₆ alkenyl, the C₂₋₆ alkenyl is vinyl, propenyl or allyl;
and/or, when R² is C₂₋₆ alkynyl, the C₂₋₆ alkynyl is C₂₋₃ alkynyl;
and/or, when R² is halogen, the halogen is fluorine, chlorine, bromine or iodine;
and/or, when R² is C₃₋₁₀ cycloalkyl or C₃₋₁₀ cycloalkyl substituted with one or more R²⁻², the C₃₋₁₀ cycloalkyl and the C₃₋₁₀ cycloalkyl in the C₃₋₁₀ cycloalkyl substituted with one or more R²⁻² are cyclohexyl, cyclopentyl, cyclobutyl or cyclopropyl;
and/or, when R² is “4- to 10-membered heterocycloalkyl containing 1-3 heteroatoms independently selected from O and N” or “4- to 10-membered heterocycloalkyl containing 1-3 heteroatoms independently selected from O and N” substituted with one or more R²⁻³, the “4- to 10-membered heterocycloalkyl containing 1-3 heteroatoms independently selected from O and N” and the “4- to 10-membered heterocycloalkyl containing 1-3 heteroatoms independently selected from O and N” in the “4- to 10-membered heterocycloalkyl containing 1-3 heteroatoms independently selected from O and N” substituted with one or more R²⁻³ are 4- to 6-membered heterocycloalkyl containing 1 heteroatom of O or N;
and/or, when R² is C₆₋₂₀ aryl or C₆₋₂₀ aryl substituted with one or more R²⁻⁴, the C₆₋₂₀ aryl and the C₆₋₂₀ aryl in the C₆₋₂₀ aryl substituted with one or more R²⁻⁴ are C₆₋₁₀ aryl;
and/or, when R² is “5- to 12-membered heteroaryl containing 1-4 heteroatoms independently selected from O, S and N” or “5- to 12-membered heteroaryl containing 1-4 heteroatoms independently selected from O, S and N” substituted with one or more R²-5, the “5- to 12-membered heteroaryl containing 1-4 heteroatoms independently selected from O, S and N” and the “5- to 12-membered heteroaryl containing 1-4 heteroatoms independently selected from O, S and N” in the “5- to 12-membered heteroaryl containing 1-4 heteroatoms independently selected from O, S and N” substituted with one or more R²⁻⁵ are 5- to 6-membered heteroaryl containing 1 heteroatom of O, S or N;
and/or, when R²⁻¹, R²⁻², R²⁻³, R²⁻⁴ and R²⁻⁵ are independently halogen, the halogen is fluorine, chlorine, bromine or iodine;
and/or, when R²⁻¹, R²⁻², R²⁻³, R²⁻⁴ and R²⁻⁵ are independently C₁₋₆ alkyl, the C₁₋₆ alkyl is methyl, ethyl, n-propyl, isopropyl, n-butyl, sec-butyl, isobutyl or tert-butyl;
and/or, when R²⁻¹, R²⁻², R²⁻³, R²⁻⁴ and R²⁻⁵ are independently C₂₋₆ alkenyl, the C₂₋₆ alkenyl is vinyl, propenyl or allyl;
and/or, when R²⁻¹, R²⁻², R²⁻³, R²⁻⁴ and R²⁻⁵ are independently C₂₋₆ alkynyl, the C₂₋₆ alkynyl is ethynyl, propynyl or propargyl;
and/or, when R²⁻¹, R²⁻², R²⁻³, R²⁻⁴ and R²⁻⁵ are independently C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl;
and/or, when R^{2a}, R^{2b}, R^{2c1}, R^{2c2}, R^{2d}, R^{2e1} and R^{2e2} are independently C₁₋₆ alkyl, the C₁₋₆ alkyl is methyl, ethyl, n-propyl, isopropyl, n-butyl, sec-butyl, isobutyl or tert-butyl;
and/or, when R³¹, R³², R³³, R³⁴, R³⁵ and R³⁶ are independently C₁₋₆ alkyl, the C₁₋₆ alkyl is methyl, ethyl, n-propyl, isopropyl, n-butyl, sec-butyl, isobutyl, or tert-butyl;
and/or, when R⁴ is C₁₋₆ alkyl or C₁₋₆ alkyl substituted with one or more R⁴⁻¹, the C₁₋₆ alkyl and the C₁₋₆ alkyl in the C₁₋₆ alkyl substituted with one or more R⁴⁻¹ are methyl, ethyl, n-propyl, isopropyl, n-butyl, sec-butyl, isobutyl or tert-butyl;
and/or, when R⁴ is C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl;
and/or, when n is 2, 3, 4, 5 or 6, and two optional R⁴ are connected, together with the atoms on the ring to which they are attached, independently form 3- to 8-membered carbocyclic ring, the 3- to 8-membered carbocyclic ring is 3- to 6-membered carbocyclic ring, and the carbocyclic ring is a monocyclic or bridged cycloalkyl;
and/or, when n is 2, 3, 4, 5 or 6, and two optional R⁴ are connected, together with the atoms on the ring to which they are attached, independently form “3- to 8-membered heterocyclic ring containing 1-3 heteroatoms independently selected from O, S and N”, the “3- to 8-membered heterocyclic ring containing 1-3 heteroatoms independently selected from O, S and N” is 3- to 6-membered heterocyclic ring containing 1 heteroatom of O, S or N;
and/or, when R⁴⁻¹ is independently halogen, the halogen is fluorine, chlorine, bromine or iodine;
and/or, when R⁴⁻¹ is independently C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl;
and/or, when R^{4a}, R^{4b}, R^{4c}, R^{4d}, R^{4e}, R^{4f}, R⁴ⁱ and R^{4j} are independently C₁₋₆ alkyl, the C₁₋₆ alkyl is methyl, ethyl, n-propyl, isopropyl, n-butyl, sec-butyl, isobutyl or tert-butyl;
and/or, when 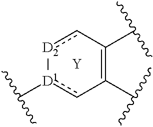
is “5- to 7-membered heterocycloalkenyl containing 1-3 heteroatoms independently selected from O, S and N”, the “5- to 7-membered heterocycloalkenyl containing 1-3 heteroatoms independently selected from O, S and N” is 6-membered heterocycloalkenyl containing 1-2 heteroatoms independently selected from O and N;
and/or, when 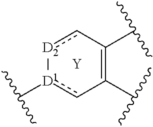
is “5- to 7-membered heteroaryl containing 1-3 heteroatoms independently selected from O, S and N”, the “5- to 7-membered heteroaryl containing 1-3 heteroatoms independently selected from O, S” and N is “6-membered heteroaryl containing 1-2 heteroatoms independently selected from O, S and N”;
and/or, when 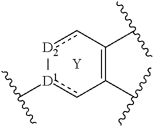
is 5- to 7-membered cycloalkenyl, the 5- to 7-membered cycloalkenyl is cyclopentenyl or cyclohexenyl;
and/or, when R⁵ is independently halogen, the halogen is fluorine, chlorine, bromine or iodine;
and/or, when R⁵ is independently C₁₋₆ alkyl, the C₁₋₆ alkyl is methyl, ethyl, n-propyl, isopropyl, n-butyl, sec-butyl, isobutyl or tert-butyl;
and/or, when L₁ is C₁₋₆ alkylene, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—;
and/or, when R¹ is C₆₋₂₀ aryl or C₆₋₂₀ aryl substituted with one or more R¹⁻¹, the C₆₋₂₀ aryl and the C₆₋₂₀ aryl in the C₆₋₂₀ aryl substituted with one or more R¹⁻¹ are C₆₋₁₀ aryl;
and/or, when R¹ is 5- to 12-membered heteroaryl containing “1-4 heteroatoms independently selected from O, S and N” or “5- to 12-membered heteroaryl containing 1-4 heteroatoms independently selected from O, S and N” substituted with one or more R¹⁻², the “5- to 12-membered heteroaryl containing 1-4 heteroatoms independently selected from O, S and N” and the “5- to 12-membered heteroaryl containing 1-4 heteroatoms independently selected from O, S and N” in the “5- to 12-membered heteroaryl containing 1-4 heteroatoms independently selected from O, S and N” substituted with one or more R¹⁻² are “5- to 10-membered heteroaryl containing 1-2 heteroatoms independently selected from O, S and N”;
and/or, when R¹ is C₈₋₁₁ benzocycloalkenyl, the C₈₋₁₁ benzocycloalkenyl is benzocyclobutenyl, benzocyclopentenyl or benzocyclohexenyl;
and/or, when R^{b}, R¹⁻¹ and R¹⁻² are independently halogen, the halogen is fluorine, chlorine, bromine or iodine;
and/or, when R^{b}, R¹⁻¹ and R¹⁻² are independently C₁₋₆ alkyl or C₁₋₆ alkyl substituted with one or more R¹⁻¹⁻¹, the C₁₋₆ alkyl and the C₁₋₆ alkyl in the C₁₋₆ alkyl substituted with one or more R¹⁻¹⁻¹ are methyl, ethyl, n-propyl, isopropyl, n-butyl, sec-butyl, isobutyl or tert-butyl;
and/or, when R^{b}, R¹⁻¹ and R¹⁻² are independently C₁₋₆ alkyl substituted with one or more R¹⁻¹, the more R¹⁻¹⁻¹ is two or three R¹⁻¹⁻¹;
and/or, when R^{b}, R¹⁻¹ and R¹⁻² are independently C₂₋₆ alkenyl, the C₂₋₆ alkenyl is vinyl, propenyl or allyl;
and/or, when R^{b}, R¹⁻¹ and R¹⁻² are independently C₂₋₆ alkynyl, the C₂₋₆ alkynyl is ethynyl, propynyl or propargyl;
and/or, when R^{b}, R¹⁻¹ and R¹⁻² are independently C₁₋₆ alkyl-O— substituted with one or more R¹⁻¹⁻² the C₁₋₆ alkyl in the C₁₋₆ alkyl-O— substituted with one or more R¹⁻¹⁻² is methyl, ethyl, n-propyl, isopropyl, n-butyl, sec-butyl, isobutyl or tert-butyl;
and/or, when R^{b}, R¹⁻¹ and R¹⁻² are independently C₃₋₁₀ cycloalkyl or C₃₋₁₀ cycloalkyl substituted with one or more R¹⁻¹⁻³, the C₃₋₁₀ cycloalkyl and the C₃₋₁₀ cycloalkyl in the C₃₋₁₀ cycloalkyl substituted with one or more R¹⁻¹-3 are cyclohexyl, cyclopentyl, cyclobutyl or cyclopropyl;
and/or, when R^{b}, R¹⁻¹ and R¹⁻² are independently “5- to 7-membered heterocycloalkyl containing 1 or 2 heteroatoms independently selected from O and N” or “5- to 7-membered heterocycloalkyl containing 1 or 2 heteroatoms independently selected from O and N” substituted with one or more R¹⁻¹⁻⁴, the “5- to 7-membered heterocycloalkyl containing 1 or 2 heteroatoms independently selected from O and N” and the “5- to 7-membered heterocycloalkyl containing 1 or 2 heteroatoms independently selected from O and N” in the “5- to 7-membered heterocycloalkyl containing 1 or 2 heteroatoms independently selected from O and N” substituted with one or more R¹⁻¹-4 are 5- to 6-membered heterocycloalkyl containing 1 heteroatom of O or N;
and/or, when R^{b}, R¹⁻¹ and R¹⁻² are independently C₆₋₂₀ aryl or C₆₋₂₀ aryl substituted with one or more R¹⁻¹⁻⁵, the C₆₋₂₀ aryl and the C₆₋₂₀ aryl in the C₆₋₂₀ aryl substituted with one or more R¹⁻¹⁻⁵ are C₆₋₁₀ aryl;
and/or, when R^{b}, R¹⁻¹ and R¹⁻² are independently “5- to 7-membered heteroaryl containing 1 or 2 heteroatoms independently selected from O and N” or “5- to 7-membered heteroaryl containing 1 or 2 heteroatoms independently selected from O and N” substituted with one or more R¹⁻¹⁻⁶, the “5 to 7-membered heteroaryl containing 1 or 2 heteroatoms independently selected from O and N” and “the 5- to 7-membered heteroaryl containing 1 or 2 heteroatoms independently selected from O and N” in the “5- to 7-membered heteroaryl containing 1 or 2 heteroatoms independently selected from O and N” substituted with one or more R¹⁻¹⁻⁶ are “5- to 6-membered heteroaryl containing 1 heteroatom of O or N”;
and/or, when two optional R¹⁻¹ or two optional R¹⁻² are connected, together with the atoms on the ring to which they are attached, independently form 3- to 8-membered cyclic olefin, the 3- to 8-membered cyclic olefin is cyclobutene, cyclopentene or cyclohexene;
and/or, when R^{c}, R¹² and R¹³ are independently C₁₋₆ alkyl, the C₁₋₆ alkyl is methyl, ethyl, n-propyl, isopropyl, n-butyl, sec-butyl, isobutyl or tert-butyl;
and/or, when R^{c}, R^{c2}, R^{c3} and R^{c4} are independently C₁₋₆ alkyl or C₁₋₆ alkyl substituted with one or more R⁴⁻¹, the C₁₋₆ alkyl and the C₁₋₆ alkyl in the C₁₋₆ alkyl substituted with one or more R¹⁻¹⁻¹ are methyl, ethyl, n-propyl, isopropyl, n-butyl, sec-butyl, isobutyl or tert-butyl;
and/or, when R^{c}, R^{c2}, R^{c3} and R^{c4} are independently C₃₋₁₀ cycloalkyl or C₃₋₁₀ cycloalkyl substituted with one or more R¹⁻¹⁻², the C₃₋₁₀ cycloalkyl and the C₃₋₁₀ cycloalkyl in the C₃₋₁₀ cycloalkyl substituted with one or more R¹⁻¹⁻² are cyclohexyl, cyclopentyl, cyclobutyl or cyclopropyl;
and/or, when R^{c}, R^{c2}, R^{c3} and R^{c4} are independently C₆₋₂₀ aryl or C₆₋₂₀ aryl substituted with one or more R¹⁻¹⁻⁴, the C₆₋₂₀ aryl and the C₆₋₂₀ aryl in the C₆₋₂₀ aryl substituted with one or more R¹⁻¹⁻⁴ are C₆₋₁₀ aryl;
and/or, when R¹⁻¹⁻¹, R¹⁻¹⁻², R¹⁻¹⁻³, R¹⁻¹⁻⁴, R¹⁻¹⁻⁵, R¹⁻¹⁻⁶, R¹⁻¹⁻¹, R¹⁻¹⁻², R¹⁻¹⁻³, R¹⁻¹⁻⁴ and R¹⁻¹⁻⁵ are independently halogen, the halogen is fluorine, chlorine, bromine or iodine;
and/or, when R¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R¹⁻¹⁻², R¹⁻¹⁻³, R¹⁻¹⁻⁴ and R¹⁻¹⁻⁵ are independently C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl;
and/or, when R¹⁻¹⁻¹, R¹⁻¹⁻², R¹⁻¹⁻³, R¹⁻¹⁻⁴, R¹⁻¹⁻⁵, R¹⁻¹⁻⁶, R¹⁻¹⁻¹, R¹⁻¹⁻², R¹⁻¹⁻³, R¹⁻¹⁻⁴ and R¹⁻¹⁻⁵ are independently C₁₋₆ alkyl, the C₁₋₆ alkyl is methyl, ethyl, n-propyl, isopropyl, n-butyl, sec-butyl, isobutyl or tert-butyl;
and/or, when R¹¹, R²¹, R²², R²³, R¹⁴, R²⁴, R¹⁵, R²⁵, R¹⁶ and R²⁶ are independently C₁₋₆ alkyl, the C₁₋₆ alkyl is methyl, ethyl, n-propyl, isopropyl, n-butyl, sec-butyl, isobutyl or tert-butyl;
and/or, when L₂ is C₁₋₆ alkylene, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—;
and/or, when R^{L-1}, R^{L-2} or R^{L-4} is independently C₁₋₆ alkylene, the C₁₋₆ alkylene is —CH₂—, —CH₂CH₂—, —CH₂CH₂CH₂—, —CH(CH₃)CH₂—, —CH₂CH₂CH₂CH₂—, —CH(CH₃)CH₂CH₂—, —CH₂CH(CH₃)CH₂— or —C(CH₃)₂CH₂—;
and/or, when R^{L-3} is C₁₋₆ alkyl, the C₁₋₆ alkyl is methyl, ethyl, n-propyl, isopropyl, n-butyl, sec-butyl, isobutyl or tert-butyl;
and/or, when R³ is C₃₋₁₂ cycloalkyl or C₃₋₁₂ cycloalkyl substituted with one or more R³⁻¹, the C₃₋₁₂ cycloalkyl and the C₃₋₁₂ cycloalkyl in the C₃₋₁₂ cycloalkyl substituted with one or more R³⁻¹ is C₃₋₁₀ cycloalkyl;
and/or, when R³ is C₃₋₁₂ cycloalkyl or C₃₋₁₂ cycloalkyl substituted with one or more R³⁻¹, the C₃₋₁₂ cycloalkyl and the C₃₋₁₂ cycloalkyl in the C₃₋₁₂ cycloalkyl substituted with one or more R³⁻¹ is a monocyclic alkyl, abridged cycloalkyl or a spiral cycloalkyl;
and/or, when R³ is “4- to 12-membered heterocycloalkyl containing 1-3 heteroatoms independently selected from O, S and N”, the “4- to 12-membered heterocycloalkyl containing 1-3 heteroatoms independently selected from O, S and N” is “5- to 8-membered heterocycloalkyl containing 1-2 heteroatoms independently selected from O and N”;
and/or, when R³ is “4- to 12-membered heterocycloalkyl containing 1-3 heteroatoms independently selected from O, S and N”, the “4- to 12-membered heterocycloalkyl containing 1-3 heteroatoms independently selected from O, S and N” is a monocyclic cycloalkyl, a spiral cycloalkyl or a fused cycloalkyl;
and/or, when R³ is “4- to 12-membered heterocycloalkyl containing 1-3 heteroatoms independently selected from O, S and N” substituted with one or more R³⁻², the “4- to 12-membered heterocycloalkyl containing 1-3 heteroatoms independently selected from O, S and N” in the “4- to 12-membered heterocycloalkyl containing 1-3 heteroatoms independently selected from O, S and N” substituted with one or more R³⁻² is “5- to 11-membered heterocycloalkyl containing 1-2 heteroatoms independently selected from O and N”;
and/or, when R³ is “4- to 12-membered heterocycloalkyl containing 1-3 heteroatoms independently selected from O, S and N” substituted with one or more R³⁻², the “4- to 12-membered heterocycloalkyl containing 1-3 heteroatoms independently selected from O, S and N” is a monocyclic alkyl, a spiral cycloalkyl or fused cycloalkyl;
and/or, when R³ is C₁₋₆ alkyl or C₁₋₆ alkyl substituted with one or more R³⁻³, the C₁₋₆ alkyl and the C₁₋₆ alkyl in the C₁₋₆ alkyl substituted with one or more R³⁻³ are methyl, ethyl, n-propyl, isopropyl, n-butyl, sec-butyl, isobutyl or tert-butyl;
and/or, when R³⁻¹, R³⁻² and R³⁻³ are independently C₁₋₆ alkyl or C₁₋₆ alkyl substituted with one or more R³⁻¹⁻¹, the C₁₋₆ alkyl and the C₁₋₆ alkyl in the C₁₋₆ alkyl substituted with one or more R³⁻¹⁻¹ are methyl, ethyl, n-propyl, isopropyl, n-butyl, sec-butyl, isobutyl or tert-butyl;
and/or, when R³⁻¹, R³⁻² and R³⁻³ are independently C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl;
and/or, when R³⁻¹, R³⁻² and R³⁻³ are independently halogen, the halogen is fluorine, chlorine, bromine or iodine;
and/or, when R^{d}, R^{d1}, R^{e1}, R^{e2}, R^{e3} and R^{e4} are independently C₁₋₆ alkyl or C₁₋₆ alkyl substituted with one or more R³⁻¹⁻², the C₁₋₆ alkyl and the C₁₋₆ alkyl in the C₁₋₆ alkyl substituted with one or more R³⁻¹⁻² are methyl, ethyl, n-propyl, isopropyl, n-butyl, sec-butyl, isobutyl or tert-butyl;
and/or, when R^{d}, R^{d1}, R^{e1}, R^{e2}, R^{e3} and R^{e4} are independently C₃₋₁₀ cycloalkyl, the C₃₋₁₀ cycloalkyl is cyclohexyl, cyclopentyl, cyclobutyl or cyclopropyl;
and/or, when R^{d}, R^{d1}, R^{e1}, R^{e2}, R^{e3} and R^{e4} are independently “4- to 10-membered heterocycloalkyl containing 1-3 heteroatoms independently selected from O and N”, the “4- to 10-membered heterocycloalkyl containing 1-3 heteroatoms independently selected from O and N” is “4- to 6-membered heterocycloalkyl containing 1-2 heteroatoms independently selected from O and N”;
and/or, when R³⁻¹⁻¹ and R³⁻¹⁻² are independently halogen, the halogen is fluorine, chlorine, bromine or iodine;
and/or, when R³⁻¹⁻¹ and R³⁻¹⁻² are independently C₁₋₆ alkyl-O—, the C₁₋₆ alkyl in the C₁₋₆ alkyl-O— is methyl, ethyl, n-propyl, isopropyl, n-butyl, sec-butyl, isobutyl or tert-butyl;
and/or, when R^{e5}, R^{e6}, R^{e7}, R^{e8}, R^{e9}, R^{e10}, R^{e11}, Re¹², R^{e13} and R^{e14} are independently C₁₋₆ alkyl, the C₁₋₆ alkyl is methyl, ethyl, n-propyl, isopropyl, n-butyl, sec-butyl, isobutyl or tert-butyl.

6. The nitrogen-containing heterocyclic compound of formula I, the pharmaceutically acceptable salt thereof, the stereoisomer thereof, the tautomer thereof or the isotopically labeled compound thereof according to claim 1, wherein, 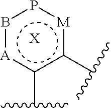
is 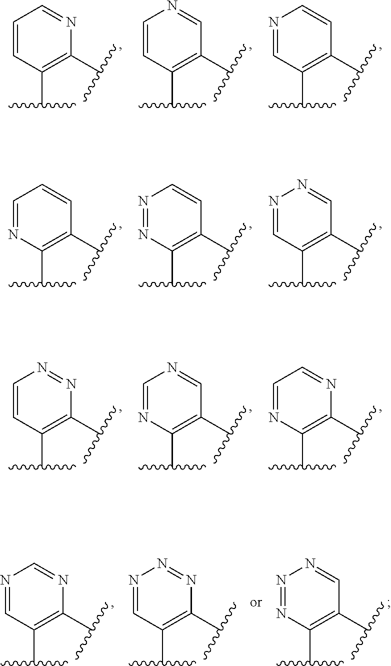
and/or, R² is —CH₃, 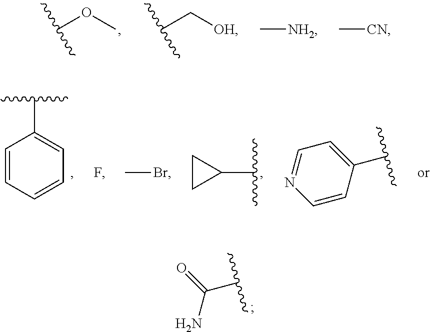
and/or, R⁴ is —CH₃, 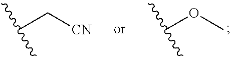
and/or, 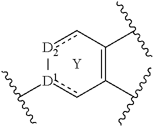
is 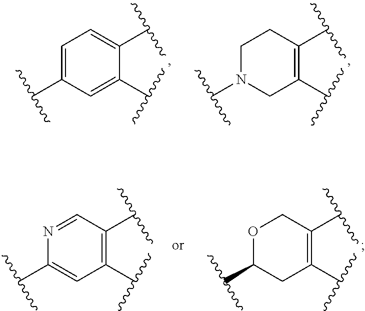 [SMILES: *c1ccc(*)c(*)c1 ; *C1=C(*)CN(*)CC1 ; *c1cc(*)c(*)cn1 ; *C1=C(*)C[C@@H](*)OC1]
and/or, 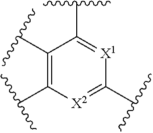
is 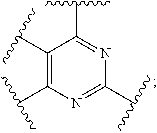 [SMILES: *c1nc(*)c(*)c(*)n1]
and/or, R¹ is 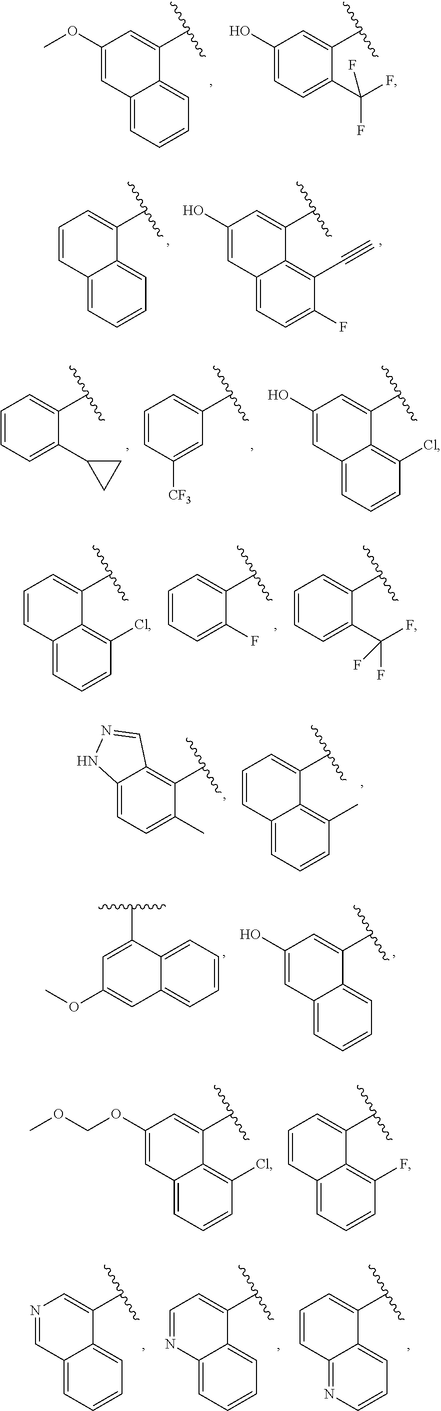 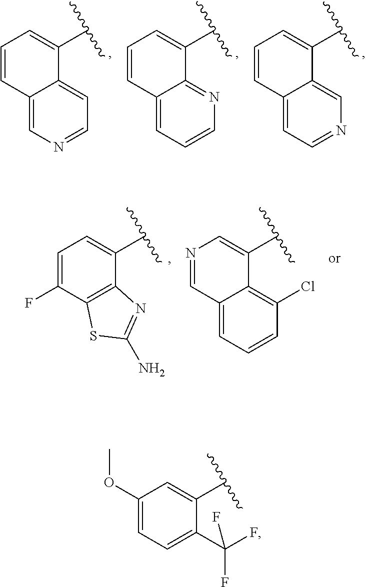
and/or, L² is 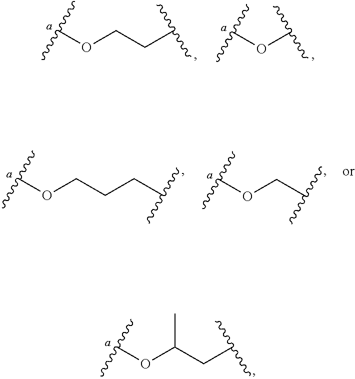
position a is connected with position b of ring 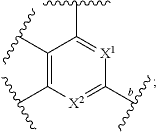
and/or, R³ is methyl,

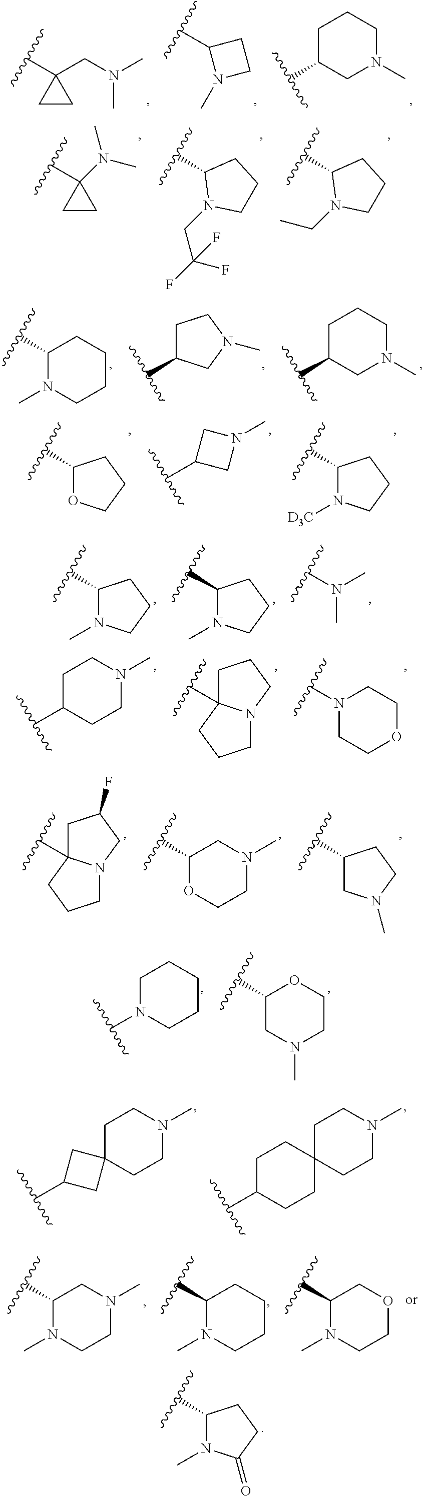

7. The nitrogen-containing heterocyclic compound of formula I, the pharmaceutically acceptable salt thereof, the stereoisomer thereof, the tautomer thereof or the isotopically labeled compound thereof according to claim 6, wherein,
When m is 1, 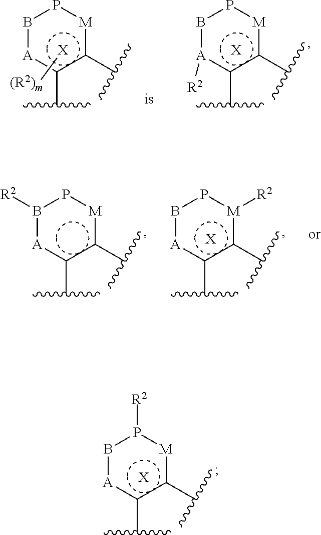
and/or, 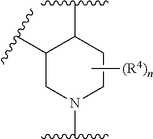
is 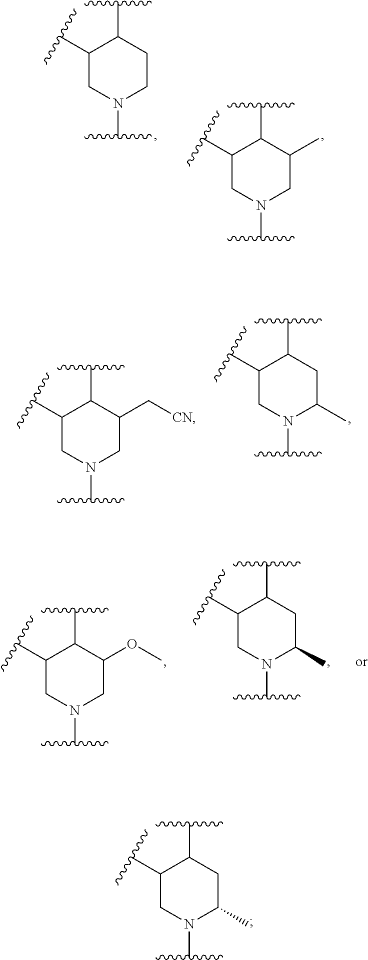
and/or, 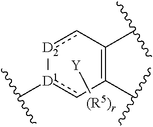
is 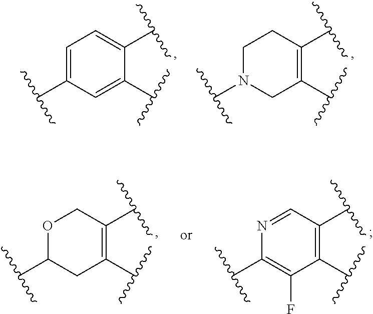 [SMILES: *c1ccc(*)c(*)c1 ; *C1=C(*)CN(*)CC1 ; *C1=C(*)CC(*)OC1 ; *c1cnc(*)c(F)c1*]
and/or, 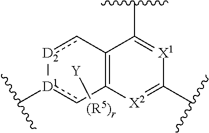
is 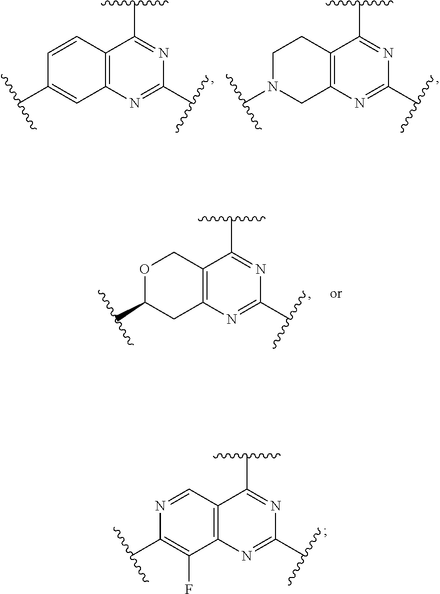 [SMILES: *c1ccc2c(*)nc(*)nc2c1 ; *c1nc(*)c2c(n1)CN(*)CC2 ; *c1nc(*)c2c(n1)C[C@@H](*)OC2 ; *c1nc(*)c2cnc(*)c(F)c2n1]
and/or, 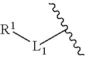
is 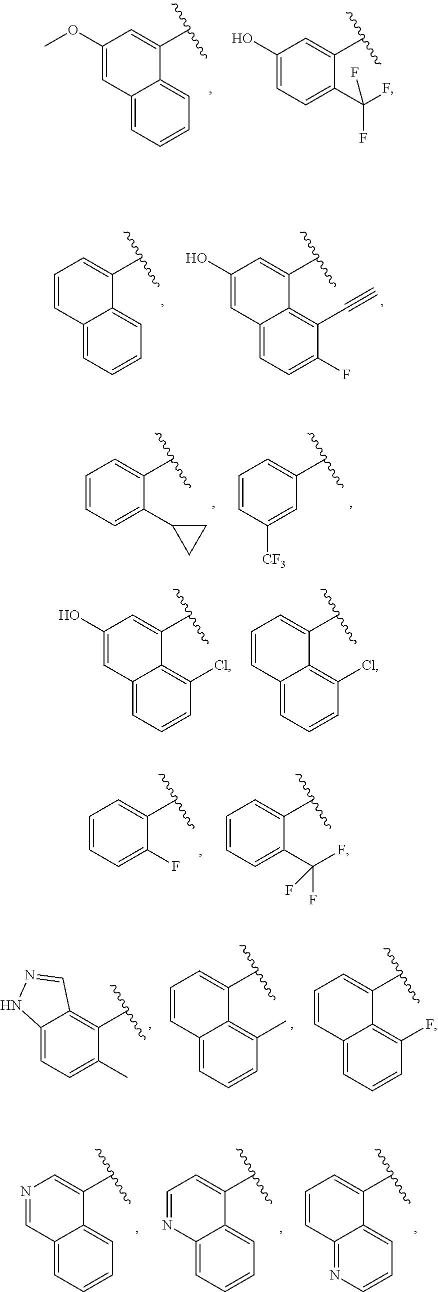 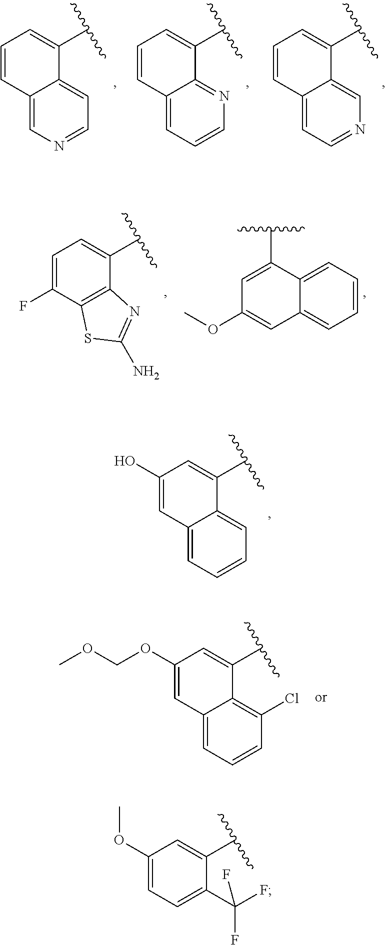
and/or, 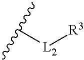
is

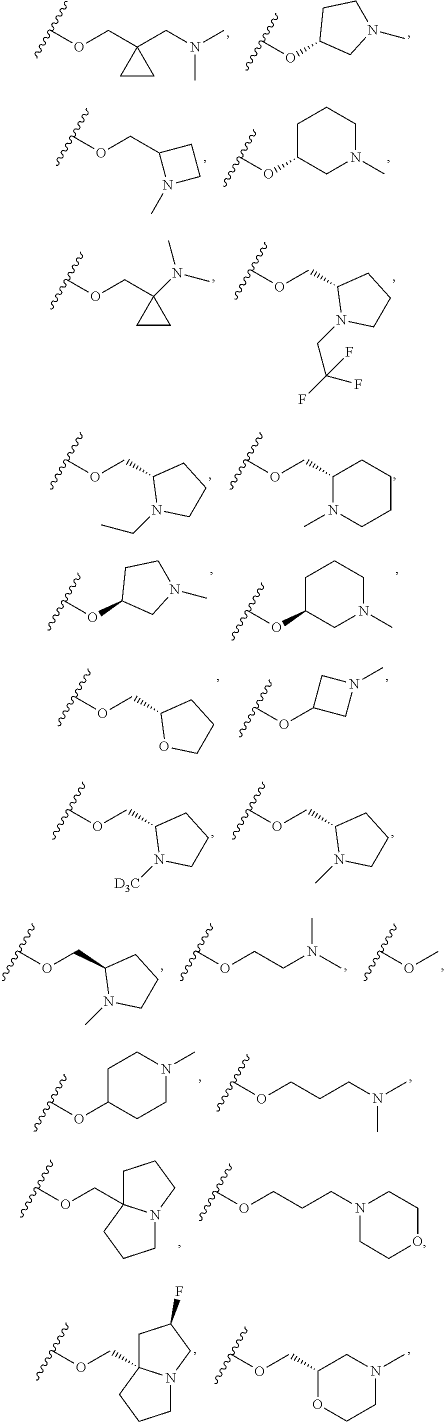

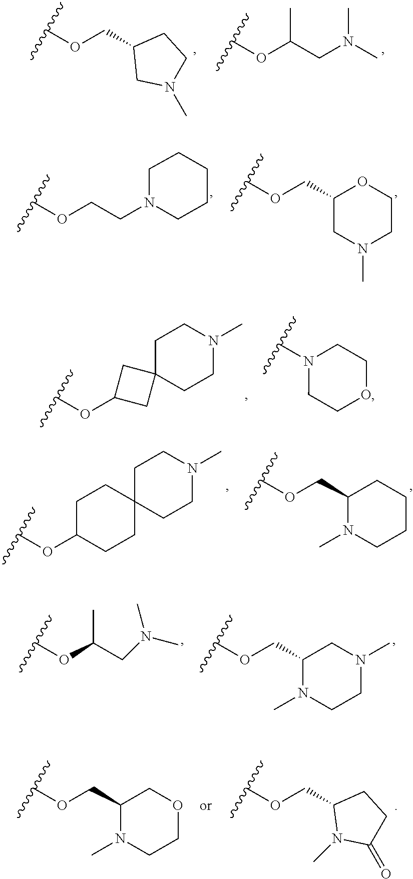

8. The nitrogen-containing heterocyclic compound of formula I, the pharmaceutically acceptable salt thereof, the stereoisomer thereof, the tautomer thereof or the isotopically labeled compound thereof according to claim 1, wherein the nitrogen-containing heterocyclic compound of formula I has any one of the following structures: 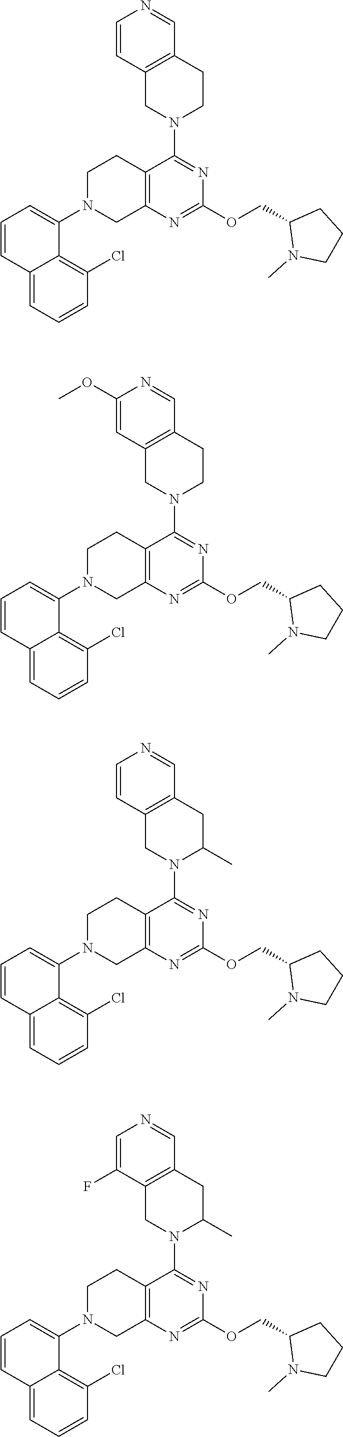 [SMILES: CN1CCC[C@H]1COc1nc2c(c(N3CCc4cnccc4C3)n1)CCN(c1cccc3cccc(Cl)c13)C2 ; COc1cc2c(cn1)CCN(c1nc(OC[C@@H]3CCCN3C)nc3c1CCN(c1cccc4cccc(Cl)c14)C3)C2 ; CC1Cc2cnccc2CN1c1nc(OC[C@@H]2CCCN2C)nc2c1CCN(c1cccc3cccc(Cl)c13)C2 ; CC1Cc2cncc(F)c2CN1c1nc(OC[C@@H]2CCCN2C)nc2c1CCN(c1cccc3cccc(Cl)c13)C2] 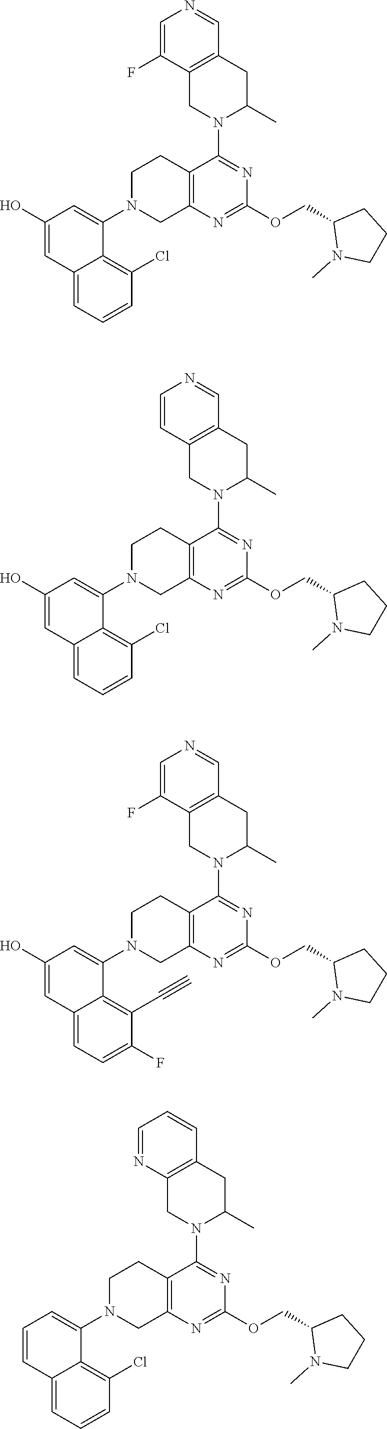 [SMILES: CC1Cc2cncc(F)c2CN1c1nc(OC[C@@H]2CCCN2C)nc2c1CCN(c1cc(O)cc3cccc(Cl)c13)C2 ; CC1Cc2cnccc2CN1c1nc(OC[C@@H]2CCCN2C)nc2c1CCN(c1cc(O)cc3cccc(Cl)c13)C2 ; C#Cc1c(F)ccc2cc(O)cc(N3CCc4c(nc(OC[C@@H]5CCCN5C)nc4N4Cc5c(F)cncc5CC4C)C3)c12 ; CC1Cc2cccnc2CN1c1nc(OC[C@@H]2CCCN2C)nc2c1CCN(c1cccc3cccc(Cl)c13)C2] 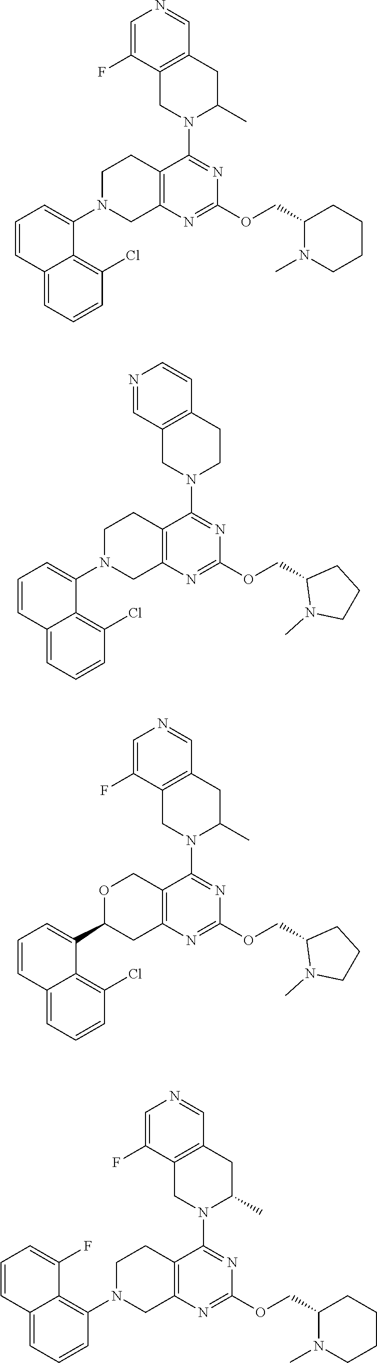 [SMILES: CC1Cc2cncc(F)c2CN1c1nc(OC[C@@H]2CCCCN2C)nc2c1CCN(c1cccc3cccc(Cl)c13)C2 ; CN1CCC[C@H]1COc1nc2c(c(N3CCc4ccncc4C3)n1)CCN(c1cccc3cccc(Cl)c13)C2 ; CC1Cc2cncc(F)c2CN1c1nc(OC[C@@H]2CCCN2C)nc2c1CO[C@H](c1cccc3cccc(Cl)c13)C2 ; C[C@H]1Cc2cncc(F)c2CN1c1nc(OC[C@@H]2CCCCN2C)nc2c1CCN(c1cccc3cccc(F)c13)C2] 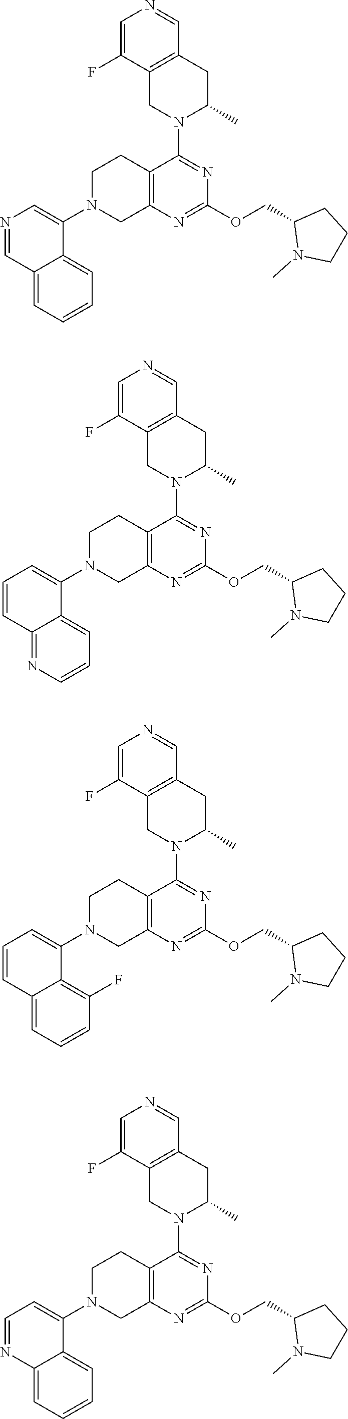 [SMILES: C[C@H]1Cc2cncc(F)c2CN1c1nc(OC[C@@H]2CCCN2C)nc2c1CCN(c1cncc3ccccc13)C2 ; C[C@H]1Cc2cncc(F)c2CN1c1nc(OC[C@@H]2CCCN2C)nc2c1CCN(c1cccc3ncccc13)C2 ; C[C@H]1Cc2cncc(F)c2CN1c1nc(OC[C@@H]2CCCN2C)nc2c1CCN(c1cccc3cccc(F)c13)C2 ; C[C@H]1Cc2cncc(F)c2CN1c1nc(OC[C@@H]2CCCN2C)nc2c1CCN(c1ccnc3ccccc13)C2] 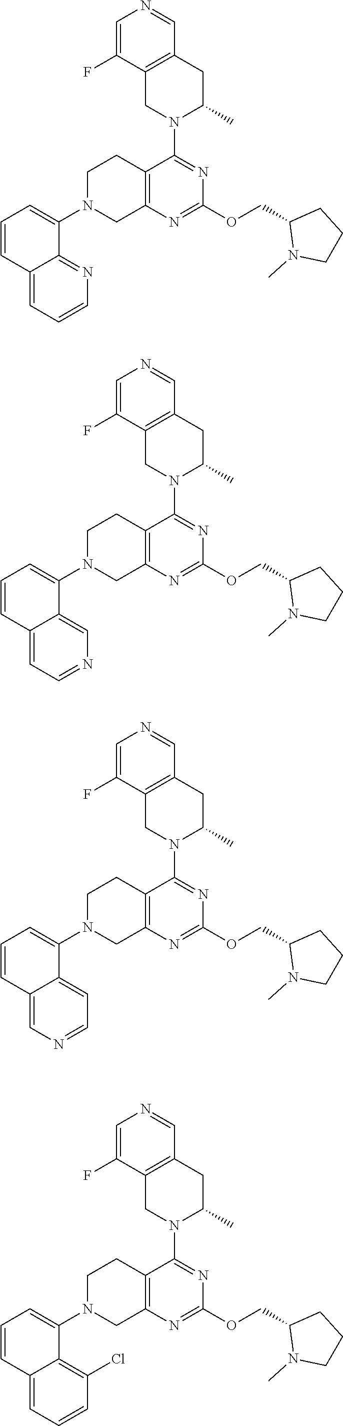 [SMILES: C[C@H]1Cc2cncc(F)c2CN1c1nc(OC[C@@H]2CCCN2C)nc2c1CCN(c1cccc3cccnc13)C2 ; C[C@H]1Cc2cncc(F)c2CN1c1nc(OC[C@@H]2CCCN2C)nc2c1CCN(c1cccc3ccncc13)C2 ; C[C@H]1Cc2cncc(F)c2CN1c1nc(OC[C@@H]2CCCN2C)nc2c1CCN(c1cccc3cnccc13)C2 ; C[C@H]1Cc2cncc(F)c2CN1c1nc(OC[C@@H]2CCCN2C)nc2c1CCN(c1cccc3cccc(Cl)c13)C2] 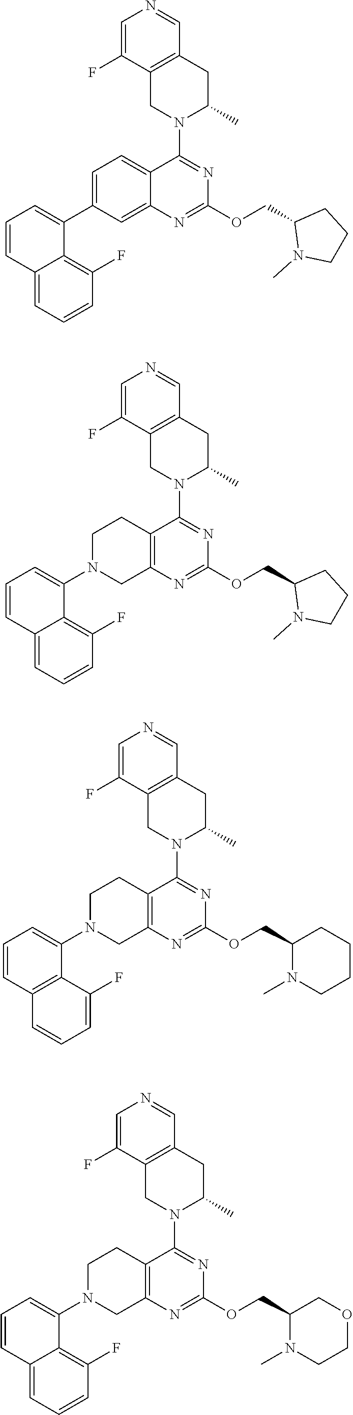 [SMILES: C[C@H]1Cc2cncc(F)c2CN1c1nc(OC[C@@H]2CCCN2C)nc2cc(-c3cccc4cccc(F)c34)ccc12 ; C[C@H]1Cc2cncc(F)c2CN1c1nc(OC[C@H]2CCCN2C)nc2c1CCN(c1cccc3cccc(F)c13)C2 ; C[C@H]1Cc2cncc(F)c2CN1c1nc(OC[C@H]2CCCCN2C)nc2c1CCN(c1cccc3cccc(F)c13)C2 ; C[C@H]1Cc2cncc(F)c2CN1c1nc(OC[C@H]2COCCN2C)nc2c1CCN(c1cccc3cccc(F)c13)C2] 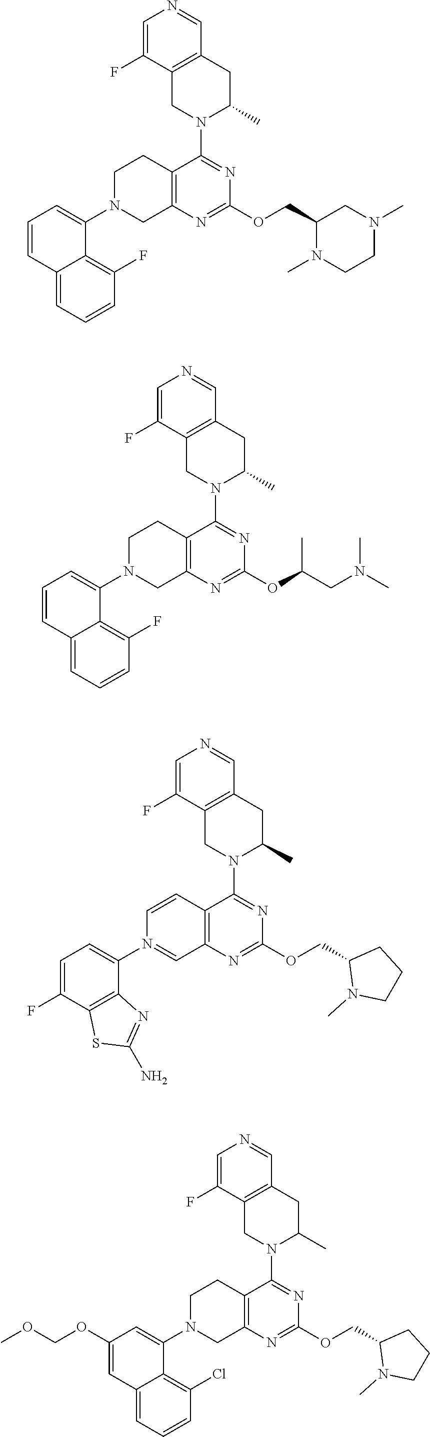 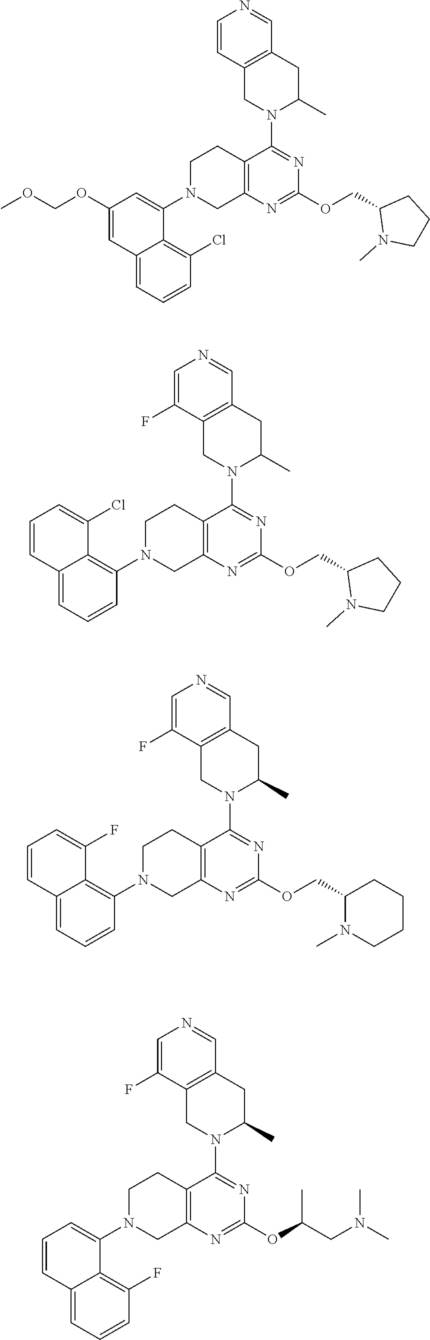 [SMILES: COCOc1cc(N2CCc3c(nc(OC[C@@H]4CCCN4C)nc3N3Cc4ccncc4CC3C)C2)c2c(Cl)cccc2c1 ; CC1Cc2cncc(F)c2CN1c1nc(OC[C@@H]2CCCN2C)nc2c1CCN(c1cccc3cccc(Cl)c13)C2 ; C[C@@H]1Cc2cncc(F)c2CN1c1nc(OC[C@@H]2CCCCN2C)nc2c1CCN(c1cccc3cccc(F)c13)C2 ; C[C@@H]1Cc2cncc(F)c2CN1c1nc(O[C@@H](C)CN(C)C)nc2c1CCN(c1cccc3cccc(F)c13)C2] 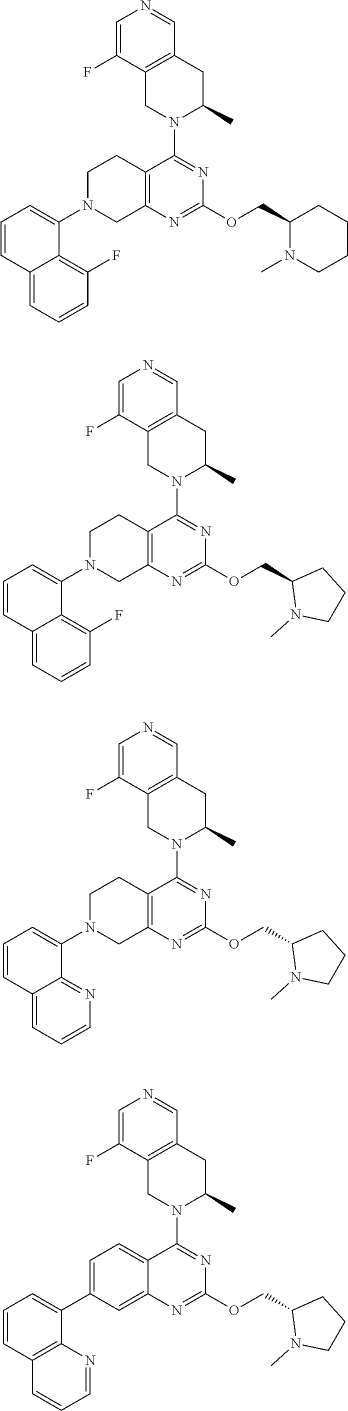 [SMILES: C[C@@H]1Cc2cncc(F)c2CN1c1nc(OC[C@H]2CCCCN2C)nc2c1CCN(c1cccc3cccc(F)c13)C2 ; C[C@@H]1Cc2cncc(F)c2CN1c1nc(OC[C@H]2CCCN2C)nc2c1CCN(c1cccc3cccc(F)c13)C2 ; C[C@@H]1Cc2cncc(F)c2CN1c1nc(OC[C@@H]2CCCN2C)nc2c1CCN(c1cccc3cccnc13)C2 ; C[C@@H]1Cc2cncc(F)c2CN1c1nc(OC[C@@H]2CCCN2C)nc2cc(-c3cccc4cccnc34)ccc12] 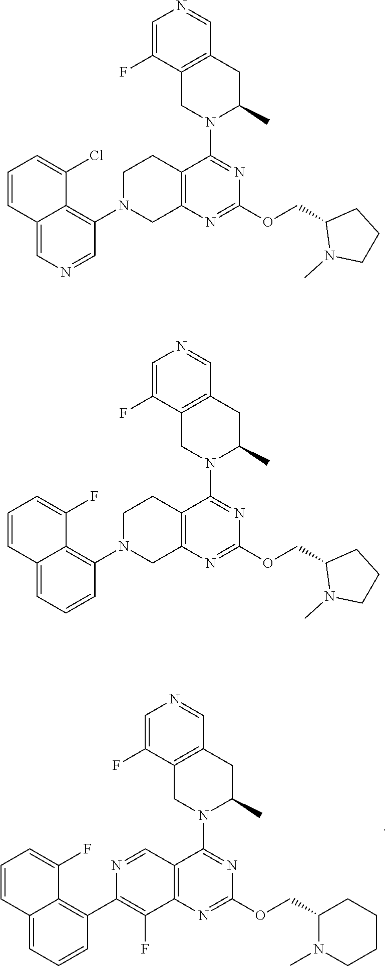 [SMILES: C[C@@H]1Cc2cncc(F)c2CN1c1nc(OC[C@@H]2CCCN2C)nc2c1CCN(c1cncc3cccc(Cl)c13)C2 ; C[C@@H]1Cc2cncc(F)c2CN1c1nc(OC[C@@H]2CCCN2C)nc2c1CCN(c1cccc3cccc(F)c13)C2 ; C[C@@H]1Cc2cncc(F)c2CN1c1nc(OC[C@@H]2CCCCN2C)nc2c(F)c(-c3cccc4cccc(F)c34)ncc12]
the pharmaceutically acceptable salt of the nitrogen-containing heterocyclic compound of formula I is trifluoroacetate having any one of the following structures:

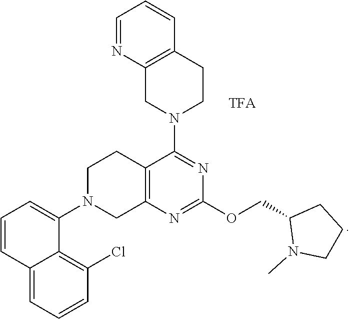 [SMILES: CN1CCC[C@H]1COc1nc2c(c(N3CCc4cccnc4C3)n1)CCN(c1cccc3cccc(Cl)c13)C2]

9. The nitrogen-containing heterocyclic compound of formula I, the pharmaceutically acceptable salt thereof, the stereoisomer thereof, the tautomer thereof or the isotopically labeled compound thereof according to claim 1, wherein the nitrogen-containing heterocyclic compound of formula I has any one of the following structures:
compound 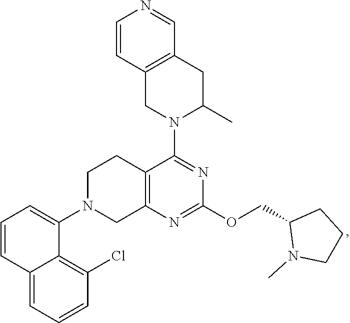 [SMILES: CC1Cc2cnccc2CN1c1nc(OC[C@@H]2CCCN2C)nc2c1CCN(c1cccc3cccc(Cl)c13)C2]
which has a retention time of 2.10 min under the following conditions: instrument: UPCC; chromatographic column: IH 4.6*100 mm, 5 um (Daicel); column temperature: 40° C.; mobile phase: CO₂/MeOH[0.2% NH₃(7M in MeOH)]=75/25; flow rate: 3.0 mL/min; wavelength: 214 nm; back pressure: 2000 psi;
compound 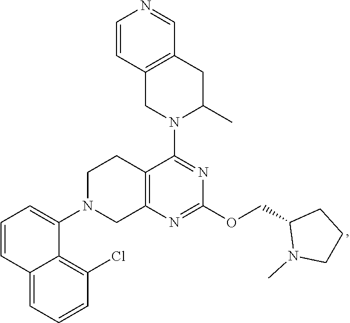 [SMILES: CC1Cc2cnccc2CN1c1nc(OC[C@@H]2CCCN2C)nc2c1CCN(c1cccc3cccc(Cl)c13)C2]
which has a retention time of 2.66 min under the following conditions: instrument: UPCC; chromatographic column: IH 4.6*100 mm, 5 um (Daicel); column temperature: 40° C.; mobile phase: CO₂/MeOH[0.2% NH₃(7M in MeOH)]=75/25; flow rate: 3.0 mL/min; wavelength: 214 nm; back pressure: 2000 psi;
compound 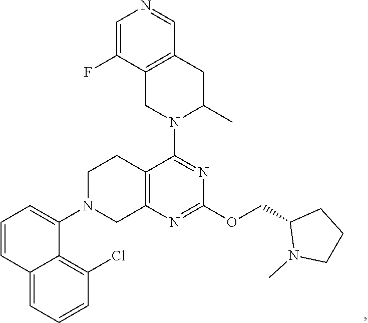 [SMILES: CC1Cc2cncc(F)c2CN1c1nc(OC[C@@H]2CCCN2C)nc2c1CCN(c1cccc3cccc(Cl)c13)C2]
which has a retention time of 15.7 min under the following conditions: instrument: Agilent1200; chromatographic column: IG 4.6*250 mm, 5 um (Daicel); column temperature: 40° C.; mobile phase: n-Hexane (0.1% DEA):EtOH (0.1% DEA)=70:30; flow rate: 1.0 m/min; wavelength: 254 nm&214 nm;
compound 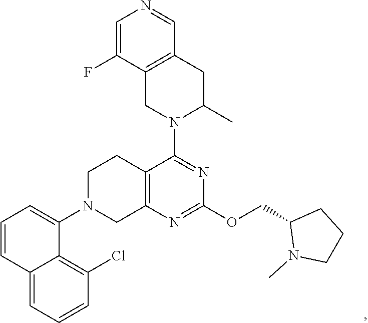 [SMILES: CC1Cc2cncc(F)c2CN1c1nc(OC[C@@H]2CCCN2C)nc2c1CCN(c1cccc3cccc(Cl)c13)C2]
which has a retention time of 20.39 min under the following conditions: instrument: Agilent1200; chromatographic column: IG 4.6*250 mm, 5 um (Daicel); column temperature: 40° C.; mobile phase: n-Hexane (0.1% DEA):EtOH (0.1% DEA)=70:30; flow rate: 1.0 m/min; wavelength: 254 nm&214 nm;
compound 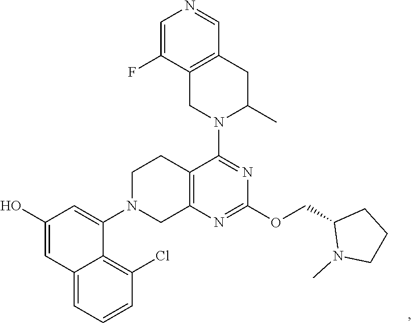 [SMILES: CC1Cc2cncc(F)c2CN1c1nc(OC[C@@H]2CCCN2C)nc2c1CCN(c1cc(O)cc3cccc(Cl)c13)C2]
which has a retention time of 1.25 min under the following conditions: instrument: UPCC; chromatographic column: AD-3 4.6*100 mm, 3 μm (Daicel); column temperature: 40° C.; mobile phase: CO₂/IPA[1% NH₃(7M in MeOH)]=55/45; flow rate: 3.0 mL/min; wavelength: 214 nm; back pressure: 2000 psi;
compound 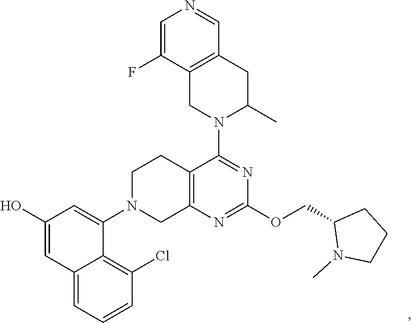 [SMILES: CC1Cc2cncc(F)c2CN1c1nc(OC[C@@H]2CCCN2C)nc2c1CCN(c1cc(O)cc3cccc(Cl)c13)C2]
which has a retention time of 1.99 min under the following conditions: instrument: UPCC; chromatographic column: AD-3 4.6*100 mm, 3 μm (Daicel); column temperature: 40° C.; mobile phase: CO₂/IPA[1% NH₃(7M in MeOH)]=55/45; flow rate: 3.0 mL/min; wavelength: 214 nm; back pressure: 2000 psi;
compound 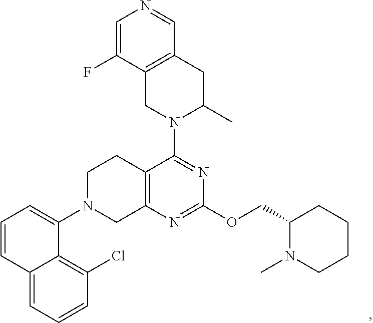 [SMILES: CC1Cc2cncc(F)c2CN1c1nc(OC[C@@H]2CCCCN2C)nc2c1CCN(c1cccc3cccc(Cl)c13)C2]
which has a retention time of 2.20 min under the following conditions: instrument: UPCC; chromatographic column: AS-H 4.6*100 mm, 5 μm (Daicel); column temperature: 40° C.; mobile phase: CO₂/EtOH [1% NH₃(7M in MeOH)]=80/20; flow rate: 3.0 mL/min; wavelength: 214 n; back pressure: 2000 psi;
compound 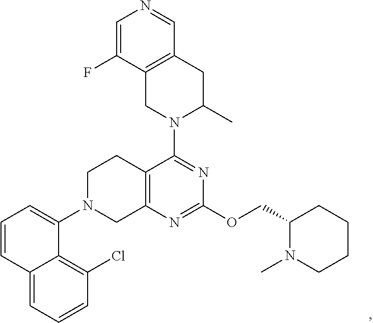 [SMILES: CC1Cc2cncc(F)c2CN1c1nc(OC[C@@H]2CCCCN2C)nc2c1CCN(c1cccc3cccc(Cl)c13)C2]
which has a retention time of 2.89 min under the following conditions: instrument: UPCC; chromatographic column: AS-H 4.6*100 mm, 5 μm (Daicel); column temperature: 40° C.; mobile phase: CO₂/EtOH [1% NH₃(7M in MeOH)]=80/20; flow rate: 3.0 m/min; wavelength: 214 nm; back pressure: 2000 psi;
compound 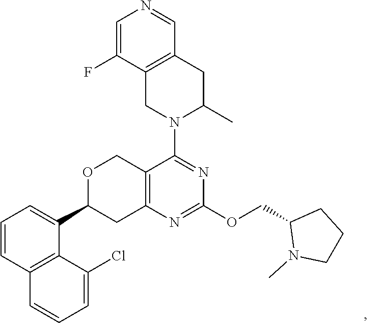 [SMILES: CC1Cc2cncc(F)c2CN1c1nc(OC[C@@H]2CCCN2C)nc2c1CO[C@H](c1cccc3cccc(Cl)c13)C2]
which has a retention time of 0.875 min under the following conditions: instrument: UPCC; chromatographic column: OJ-3 4.6*100 mm, 3 um (Daicel); column temperature: 40° C.; mobile phase: CO₂/Methanol[0.2% NH₃(7M in Methanol)]=55/45; flow rate: 3.0 mL/min; wavelength: 214 nm; back pressure: 2000 psi;
compound 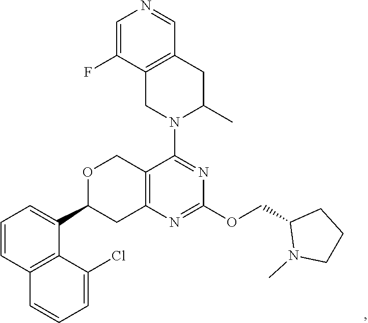 [SMILES: CC1Cc2cncc(F)c2CN1c1nc(OC[C@@H]2CCCN2C)nc2c1CO[C@H](c1cccc3cccc(Cl)c13)C2]
which has a retention time of 1.881 min under the following conditions: instrument: UPCC; chromatographic column: OJ-3 4.6*100 mm, 3 um (Daicel); column temperature: 40° C.; mobile phase: CO₂/Methanol[0.2% NH₃(7M in Methanol)]=55/45; flow rate: 3.0 mL/min; wavelength: 214 nm; back pressure: 2000 psi.

10. The nitrogen-containing heterocyclic compound of formula I, the pharmaceutically acceptable salt thereof, the stereoisomer thereof, the tautomer thereof or the isotopically labeled compound thereof according to claim 9, wherein the nitrogen-containing heterocyclic compound of formula I has any one of the following structures:

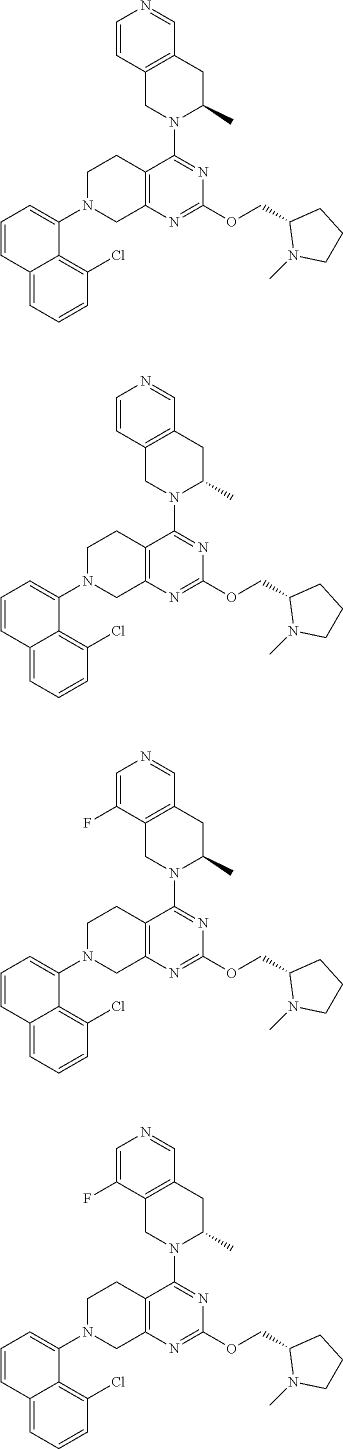 [SMILES: C[C@@H]1Cc2cnccc2CN1c1nc(OC[C@@H]2CCCN2C)nc2c1CCN(c1cccc3cccc(Cl)c13)C2 ; C[C@H]1Cc2cnccc2CN1c1nc(OC[C@@H]2CCCN2C)nc2c1CCN(c1cccc3cccc(Cl)c13)C2 ; C[C@@H]1Cc2cncc(F)c2CN1c1nc(OC[C@@H]2CCCN2C)nc2c1CCN(c1cccc3cccc(Cl)c13)C2 ; C[C@H]1Cc2cncc(F)c2CN1c1nc(OC[C@@H]2CCCN2C)nc2c1CCN(c1cccc3cccc(Cl)c13)C2]

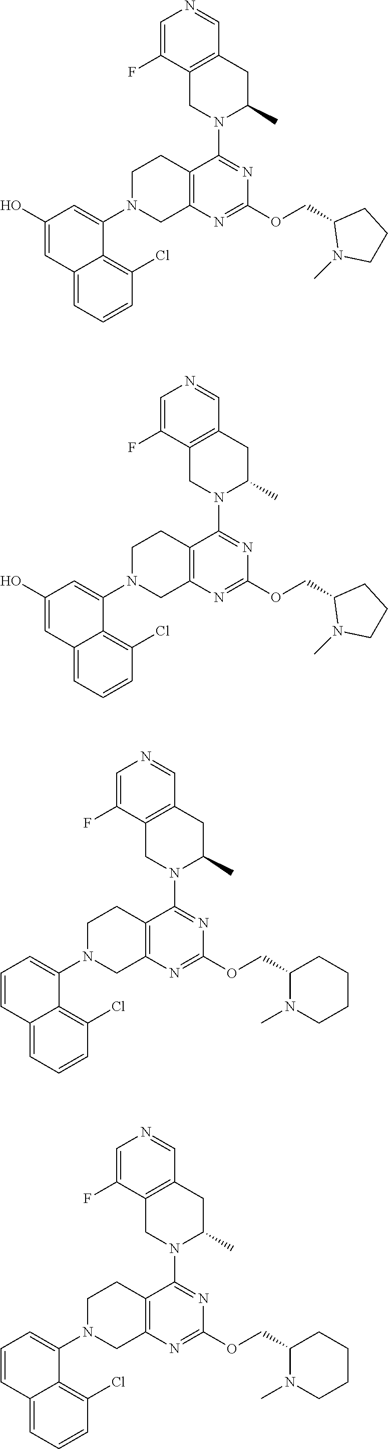 [SMILES: C[C@@H]1Cc2cncc(F)c2CN1c1nc(OC[C@@H]2CCCN2C)nc2c1CCN(c1cc(O)cc3cccc(Cl)c13)C2 ; C[C@H]1Cc2cncc(F)c2CN1c1nc(OC[C@@H]2CCCN2C)nc2c1CCN(c1cc(O)cc3cccc(Cl)c13)C2 ; C[C@@H]1Cc2cncc(F)c2CN1c1nc(OC[C@@H]2CCCCN2C)nc2c1CCN(c1cccc3cccc(Cl)c13)C2 ; C[C@H]1Cc2cncc(F)c2CN1c1nc(OC[C@@H]2CCCCN2C)nc2c1CCN(c1cccc3cccc(Cl)c13)C2]

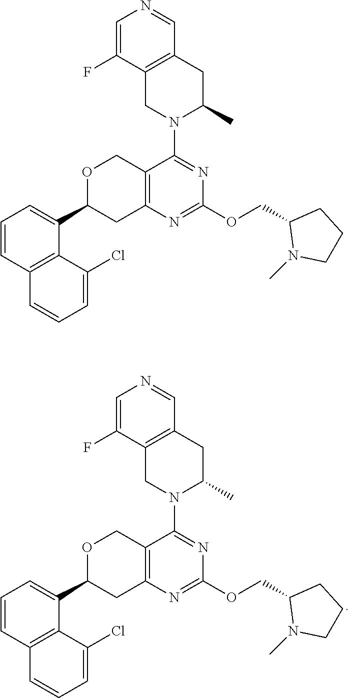 [SMILES: C[C@@H]1Cc2cncc(F)c2CN1c1nc(OC[C@@H]2CCCN2C)nc2c1CO[C@H](c1cccc3cccc(Cl)c13)C2 ; C[C@H]1Cc2cncc(F)c2CN1c1nc(OC[C@@H]2CCCN2C)nc2c1CO[C@H](c1cccc3cccc(Cl)c13)C2]

11. A compound,

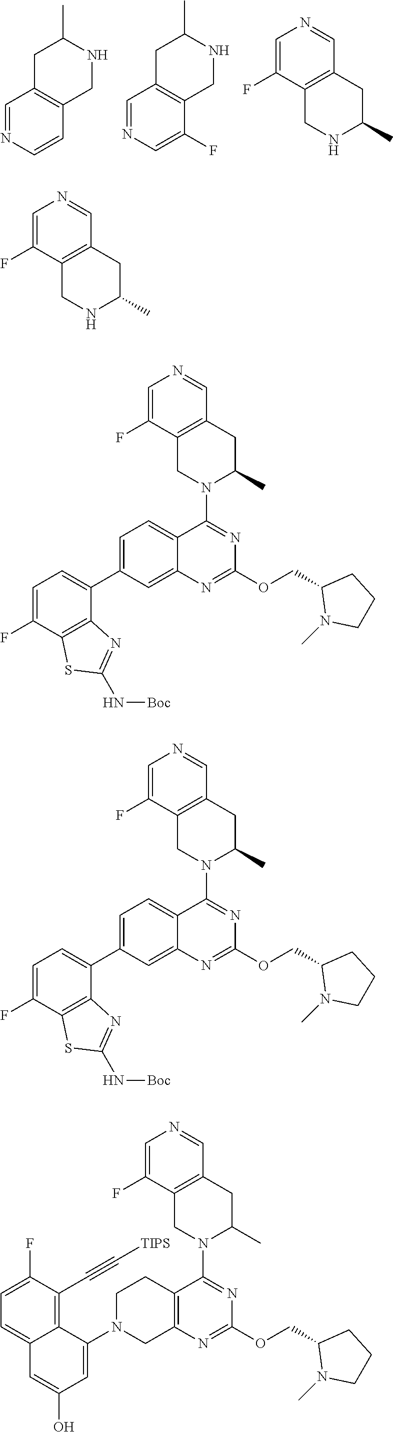

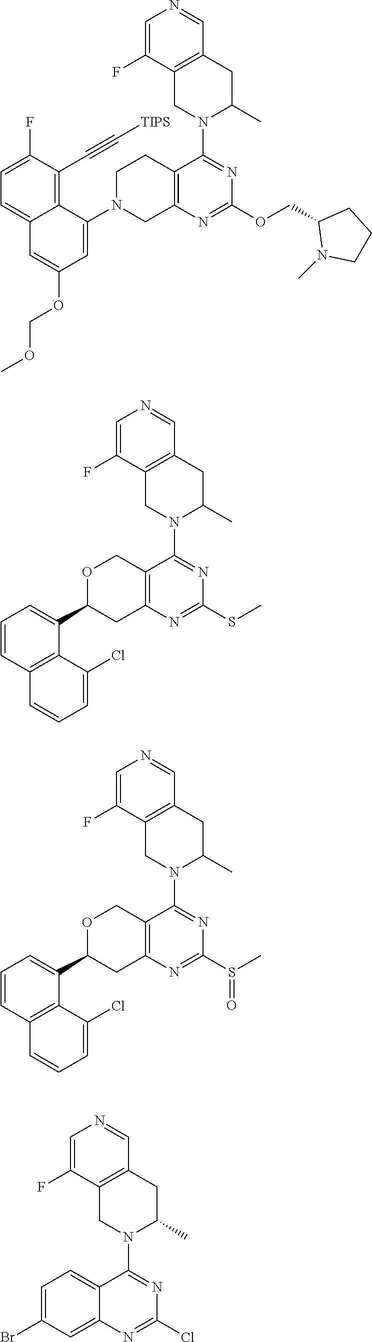

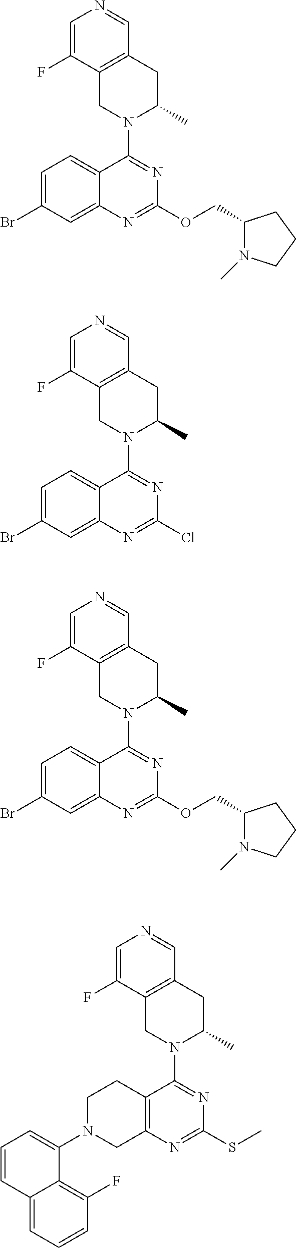 [SMILES: C[C@H]1Cc2cncc(F)c2CN1c1nc(OC[C@@H]2CCCN2C)nc2cc(Br)ccc12 ; C[C@@H]1Cc2cncc(F)c2CN1c1nc(Cl)nc2cc(Br)ccc12 ; C[C@@H]1Cc2cncc(F)c2CN1c1nc(OC[C@@H]2CCCN2C)nc2cc(Br)ccc12 ; CSc1nc2c(c(N3Cc4c(F)cncc4C[C@@H]3C)n1)CCN(c1cccc3cccc(F)c13)C2]

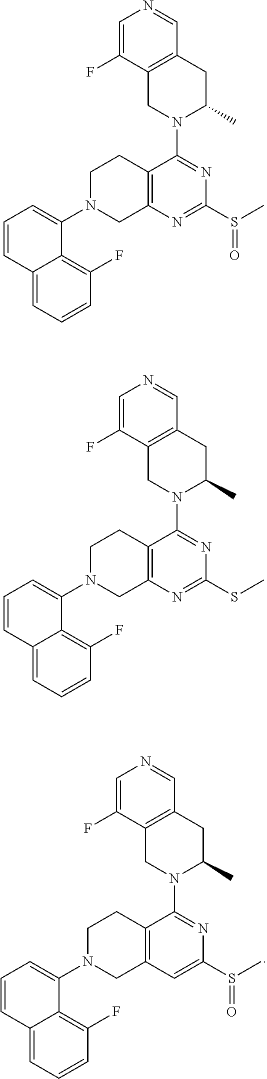 [SMILES: C[C@H]1Cc2cncc(F)c2CN1c1nc(S(C)=O)nc2c1CCN(c1cccc3cccc(F)c13)C2 ; CSc1nc2c(c(N3Cc4c(F)cncc4C[C@H]3C)n1)CCN(c1cccc3cccc(F)c13)C2 ; C[C@@H]1Cc2cncc(F)c2CN1c1nc(S(C)=O)cc2c1CCN(c1cccc3cccc(F)c13)C2]

12. A pharmaceutical composition, comprising substance A and a pharmaceutical adjuvant; the substance A is the nitrogen-containing heterocyclic compound represented by formula I as defined in any one of claim 1, a pharmaceutically acceptable salt thereof, a stereoisomer thereof, a tautomer thereof or an isotopic compound thereof.

13. A method for inhibiting RAS in a subject in need thereof, comprising: administering a therapeutically effective amount of a substance A to the subject, wherein the substance A is the nitrogen-containing heterocyclic compound of formula I, the pharmaceutically acceptable salt thereof, the stereoisomer thereof, the tautomer thereof or the isotopically labeled compound thereof according to claim 1; The RAS is a wild type RAS or a mutant RAS; the mutant RAS is a KRAS, HRAS or NRAS mutation.

14. The method according to claim 13, wherein the KRAS mutation is a G12, G13 or Q61 mutation; the HRAS mutation is a G12, G13 or Q61 mutation; the NRAS mutation is a G12, G13 or Q61 mutation.

15. A method for treating or preventing an RAS-related disease in a subject in need thereof, comprising: administering an effective amount of a substance A, wherein the substance A is the nitrogen-containing heterocyclic compound of formula I, the pharmaceutically acceptable salt thereof, the stereoisomer thereof, the tautomer thereof or the isotopically labeled compound thereof according to claim 1.

16. The method according to claim 15, wherein the mutant RAS is a KRAS, HRAS or NRAS mutation, wherein the KRAS mutation is KRAS G12C, KRAS G12D, KRAS G12S, KRAS G12A, KRAS G12V KRAS G13D, KRAS G12C, KRAS G12D or KRAS G12V; the HRAS mutation is a G12, G13 or Q61 mutation; the NRAS mutation is a G12, G13 or Q61 mutation;
and/or, RAS-related disease is cancer; the cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

17. A method for treating or preventing cancer in a subject in need thereof, comprising:
administering an effective amount of a substance A, wherein the substance A is the nitrogen-containing heterocyclic compound of formula I, the pharmaceutically acceptable salt thereof, the stereoisomer thereof, the tautomer thereof or the isotopically labeled compound thereof according to claim 1.

18. The method according to claim 17, wherein the cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.
